(19) United States
(12) Patent Application Publication
(10) Pub. No.: US 2024/0116850 A1
(43) Pub. Date: Apr. 11, 2024

(54) COMPOSITIONS AND METHODS RELATED TO BICYCLO[2.2.1] HEPTANAMINE-CONTAINING COMPOUNDS

(71) Applicants: The Broad Institute, Inc., Cambridge, MA (US); Instituto Carlos Slim de la Salud, A.C., Mexico, D.F. (MX); The Brigham and Women's Hospital, Inc., Boston, MA (US)

(72) Inventors: Brian T. Chamberlain, Cambridge, MA (US); Florence F. Wagner, Cambridge, MA (US); Maria Alimova, Cambridge, MA (US); Anna Greka, Boston, MA (US)

(21) Appl. No.: 18/269,168

(22) PCT Filed: Dec. 23, 2021
(86) PCT No.: PCT/US21/65049

§ 371 Date: Jun. 22, 2023

Related U.S. Application Data
(60) Provisional application No. 63/130,011, filed on Dec. 23, 2020.

(51) Int. Cl. C07C 211/38; C07C 205/05; C07C 209/34; C07C 209/40; C07C 233/06; C07C 271/24; C07C 311/20; C07D 209/48; C07D 333/20

(52) U.S. Cl. CPC C07C 211/38; C07C 205/05; C07C 209/34; C07C 209/40; C07C 233/06; C07C 271/24; C07C 311/20; C07D 209/48; C07D 333/20; C07B 2200/07; C07B 2200/13; C07C 2601/02; C07C 2601/04; C07C 2601/08; C07C 2601/14; C07C 2602/42

(57) ABSTRACT

The present disclosure relates to compositions and methods related to bicyclo[2.2.1]heptanamine-containing compounds and salts thereof.

### RELATED APPLICATION

This application claims the benefit of U.S. Provisional Application No. 63/130,011 filed on Dec. 23, 2020, the contents of which is fully incorporated by reference herein.

### BACKGROUND

BRD4780 also known as rac-3-exo-isopropylbicyclo[2.2.1]heptan-2-endo-amine hydrochloride (1) or AGN192403 has been reported as a selective imidazoline 1 receptor agent and an alpha-2-adrenergic blocking agent. BRD4780 was subsequently found to clear mutant frameshift Mucin 1 protein (MUC1) in vitro and in vivo and was demonstrated to be effective at removing aberrant protein accumulation in a variety of proteinopathies. The known synthetic route to BRD4780 (Scheme 1) proceeds through a Diels-Alder reaction of (E)-3-methyl-1-nitrobut-1-ene and cyclopentadiene followed by catalytic hydrogenation. Careful normal phase chromatography is required to isolate 5. Finally, the HCl salt of the target compound is prepared via precipitation of the free base from ethereal solution using HCl in diethyl ether.

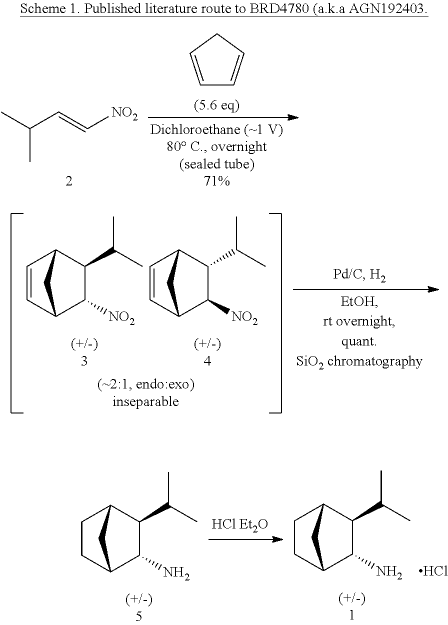

However, the method depicted in Scheme 1 has several drawbacks that complicate large scale preparation of BRD4780. For example, the Diels-Alder step is conducted at high reaction temperatures requiring that the reaction is performed in a sealed reaction vessel. The thermal risks of performing this reaction on a large scale may be significant. Next, the Diels-Alder reaction generates a 2:1 mixture of C2-endo-C3-exo (major) and C2-exo-C3-endo (minor) racemic products that must be separated by chromatography. Moreover, the literature route has only been demonstrated on a 2 g scale. Finally, the solid-state properties and the significance of the counterion have not been examined. Accordingly, new synthetic routes and determination of optimal counterion(s) of BRD4780 are required to overcome the aforementioned drawbacks.

### SUMMARY OF THE INVENTION

In one aspect, the present disclosure provides compounds having a structure represented by formula I:

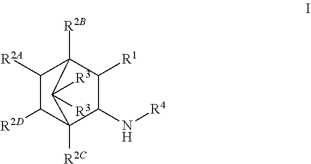

or a pharmaceutically acceptable salt thereof, wherein
R¹ is alkyl, cycloalkyl, aryl, or heteroaryl;
each of R^{2A}, R^{2B}, R^{2C}, and R^{2D} is independently selected from H or alkyl;
R³ are both H or both alkyl;
R⁴ is H or C(O)OR⁵ (i.e., to form a carbamate);
R⁵ is aralkyl or heteroaralkyl;
wherein
the compound is not selected from

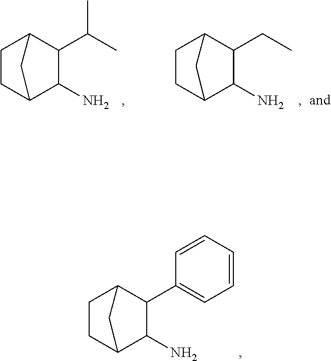

or a hydrochloride salt thereof.

In yet another aspect, the present disclosure provides crystalline forms of the structure represented by formula I.

In a further aspect, the present disclosure provides pharmaceutical compositions comprising a compound or a crystalline form of the disclosure and a pharmaceutically acceptable excipient.

In another aspect, the present disclosure provides methods of making a compound related to the compounds represented by formula I.

### BRIEF DESCRIPTION OF THE DRAWINGS

FIGS. 1A-C depict the properties of hydrochloride, maleate, fumarate, succinate, phosphate and malate salts of BRD4780.

FIGS. 2A-2R depict exemplary XRPD spectra of certain salts disclosed herein.

FIGS. 3A-3E depict the pharmacokinetic properties of certain salts disclosed herein following low dose i.v. and p.o. PK study in mice.

FIG. 4A depicts the TGA/DSC curves of HCl salt Type A. FIG. 4B depicts the TGA/DSC curves of maleate salt Type A. FIG. 4C depicts the TGA/DSC curves of re-prepared fumarate Type A. FIG. 4D depicts the TGA/DSC curves of re-prepared succinate salt Type A.

FIG. 4E depicts the TGA/DSC curves of phosphate salt Type B. FIG. 4F depicts the TGA/DSC curves of re-prepared L-malate Type C.

FIG. 5A depicts the calibration curve of standard samples to calculate solubility of HCl salt Type A (conc. range from 0.0059 mg/mL-0.044 mg/mL). FIG. 5B depicts the calibration curve of standard samples to calculate solubility of maleate Type A, fumarate Type A, succinate Type A, phosphate Type B and L-malate Type C (conc. range from 0.0044 mg/mL-0.035 mg/mL)

### DETAILED DESCRIPTION OF THE INVENTION

A solvent study to identify a solvent that would improve the reported ˜2:1 ratio of endo:exo isomers and could be conducted at near ambient condition was conducted. Through this process, hexafluoroisopropanol was identified as effective at inducing the Diels-Alder reaction at 40° C. in a high endo selectivity (ratio 3:4>21:1). Further, it was found that the Diels-Alder reaction of 2 and cyclopentadiene could be executed on the kilogram scale in 55% crude yield using this solvent. The resulting crude 3 could be advanced to the desired 1 which was obtained in 95% purity (Q-NMR) by precipitation as the HCl salt and trituration. The final compound did not possess any single impurity with >1% abundance by GC analysis. The sequence from 2 to 1 is executed without the use of chromatography and delivered 650 g desired 1 in this batch (Scheme 2).

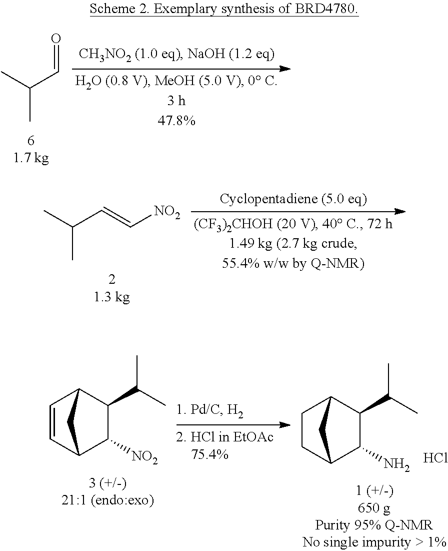

In one aspect, the present disclosure provides compounds having a structure represented by formula I:

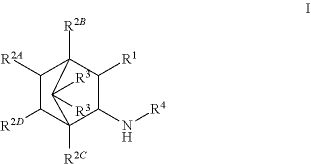

or a pharmaceutically acceptable salt thereof, wherein
R¹ is alkyl, cycloalkyl, aryl, or heteroaryl;
each of R^{2A}, R^{2B}, R^{2C}, and R^{2D} is independently selected from H or alkyl;
R³ are both H or both alkyl;
R⁴ is H or C(O)OR⁵ (i.e., to form a carbamate);
R⁵ is aralkyl or heteroaralkyl;
wherein
the compound is not selected from

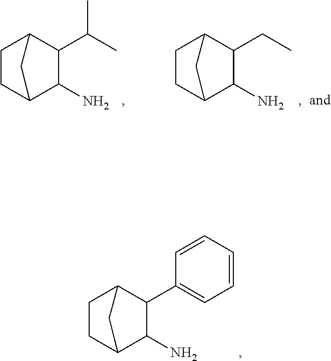

or a hydrochloride salt thereof.

In certain embodiments, the compound is not a hydrochloride salt (or in some embodiments not any salt) of

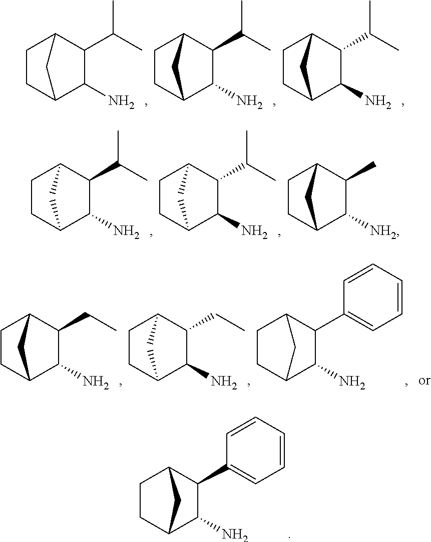

In certain embodiments, the compound has a structure represented by formula Ia, Ib, Ic, or Id:

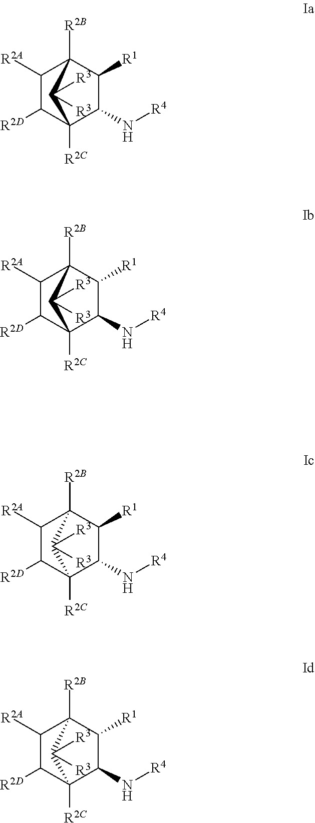

or a pharmaceutically acceptable salt thereof.

In certain embodiments, the compound has an enantiomeric excess (ee) or diastereomeric excess (de) greater than 95%, 96%, 97%, 98%, or 99%. In certain preferred embodiments, the compound is a single enantiomer or a single diastereomer. In certain embodiments, the compound is substantially free of one enantiomer or of one or more (preferably all) other diastereomers.

In certain embodiments, R³ are each H. In certain embodiments, R⁴ is H. In certain embodiments, R¹ is alkyl. In certain embodiments, R¹ is methyl, trifluoromethyl, ethyl, propyl, isopropyl, isobutyl, or tertiary-butyl. In other embodiments, R¹ is cycloalkyl. In certain embodiments, R¹ is cyclopropyl, cyclobutyl, cyclopentyl, or cyclohexyl. In yet other embodiments, R¹ is aryl. In certain embodiments, R¹ is phenyl. In yet other embodiments, R¹ is heteroaryl. In certain embodiments, R¹ is thiophenyl.

In certain embodiments, R¹ is substituted with alkyl, alkenyl, alkynyl, halo, hydroxyl, carboxyl, acyl, acetyl, ester, thioester, alkoxy, phosphoryl, amino, amide, cyano, nitro, azido, alkylthio, alkenyl, alkynyl, cycloalkyl, alkylsulfonyl, or sulfonamide.

In certain embodiments, R^{2A} is H. In certain embodiments, R^{2B} is H. In certain embodiments, R^{2C} is H. In certain embodiments, R^{2D} is H. In certain preferred embodiments, all of these selections occur simultaneously.

In certain embodiments, the compound is selected from

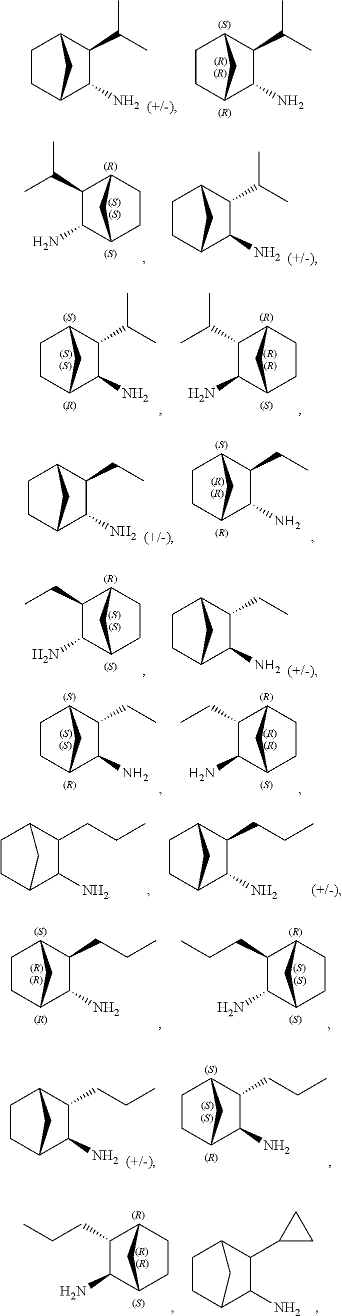

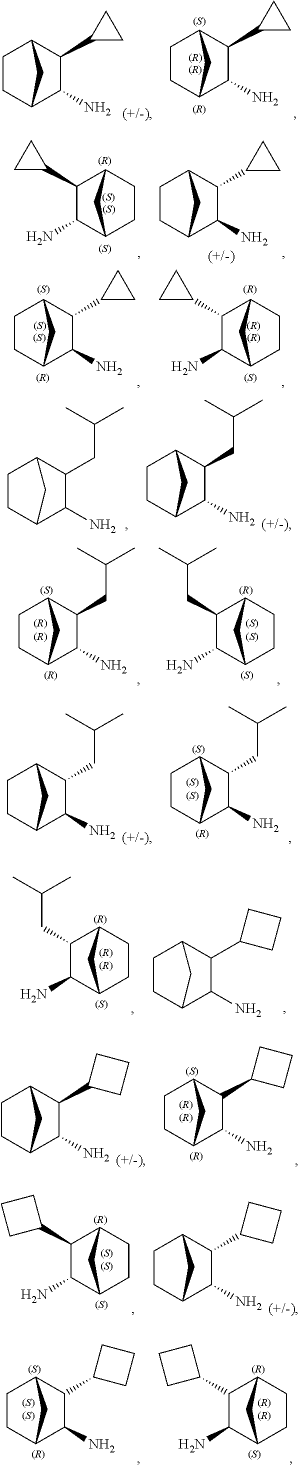

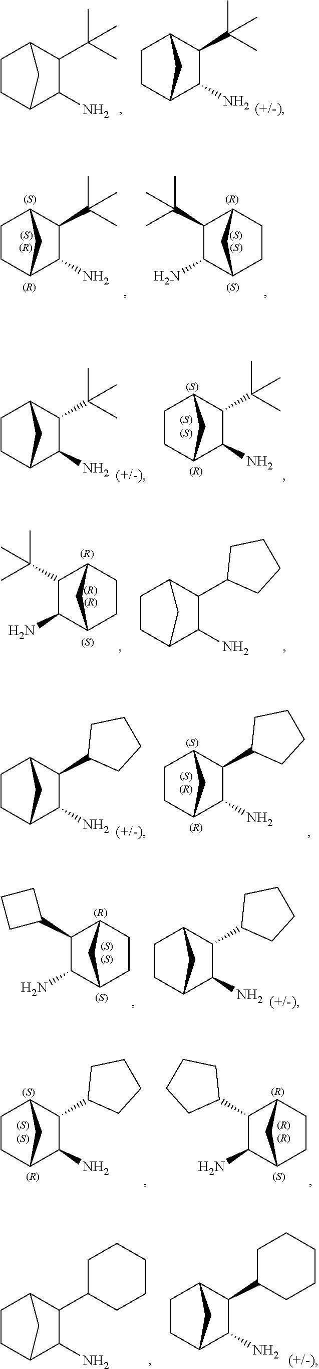

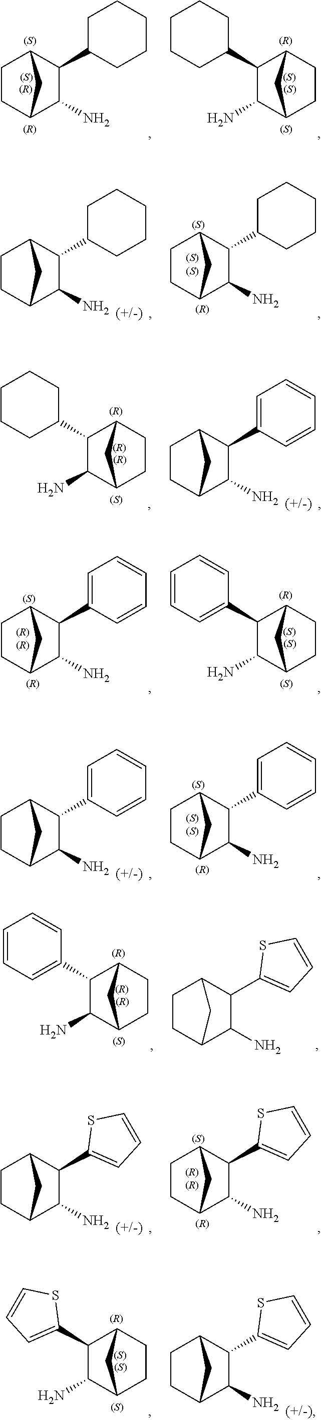

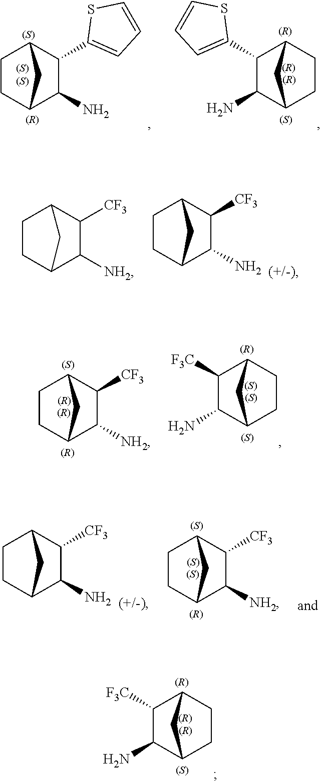

or a pharmaceutically acceptable salt thereof.

In certain embodiments, the salt is a hydrochloride, maleate, phosphate, fumarate, citrate, malate (e.g., L-malate), lactate, succinate, adipate, acetate, tosylate, mesylate, besylate, benzoate, hydrobromide, aspartate (e.g., L-aspartate), glutamate (e.g., L-glutamate), or tartrate (e.g., L-tartrate). In certain embodiments, the salt is a maleate, phosphate, fumarate, citrate, malate (e.g., L-malate), lactate, succinate, adipate, acetate, tosylate, mesylate, besylate, benzoate, hydrobromide, aspartate (e.g., L-aspartate), glutamate (e.g., L-glutamate), or tartrate (e.g., L-tartrate). In certain preferred embodiments, the compound is a maleate, fumarate, succinate, L-malate, or phosphate salt. In the most preferred embodiments, the compound is a maleate salt.

In yet another aspect, the present disclosure provides pharmaceutical compositions comprising a compound of formula I wherein R⁴ is H and a pharmaceutically acceptable excipient.

In yet another aspect, the present disclosure provides crystalline forms of

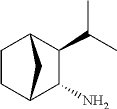

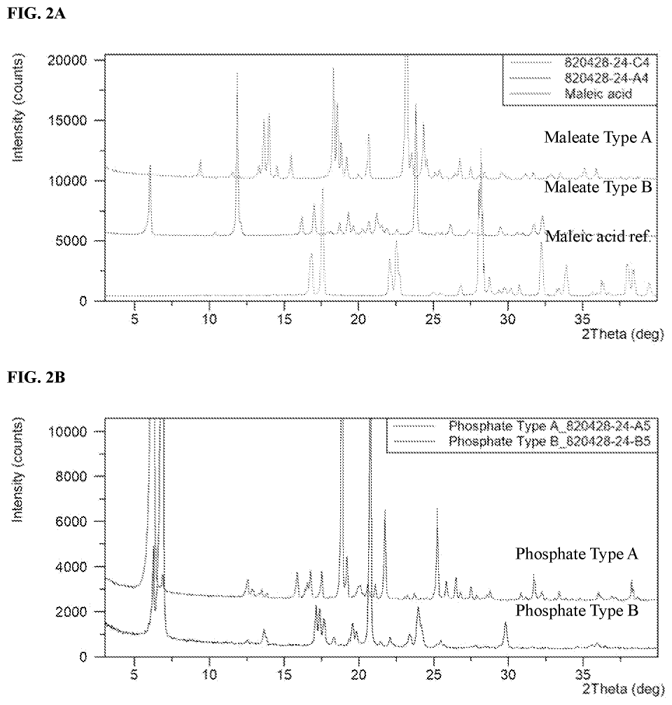

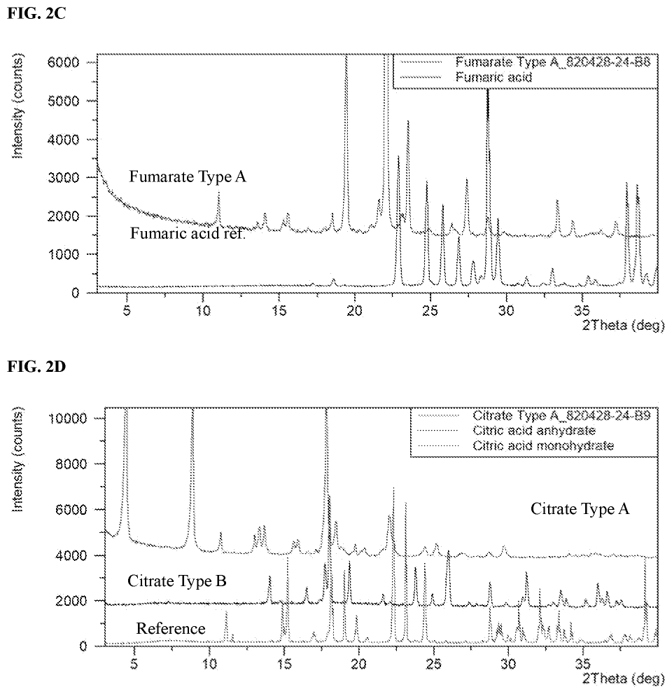

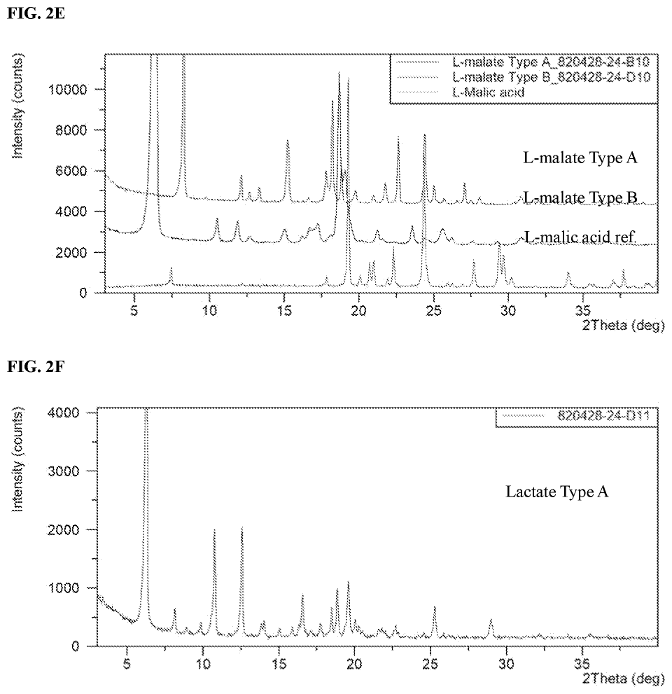

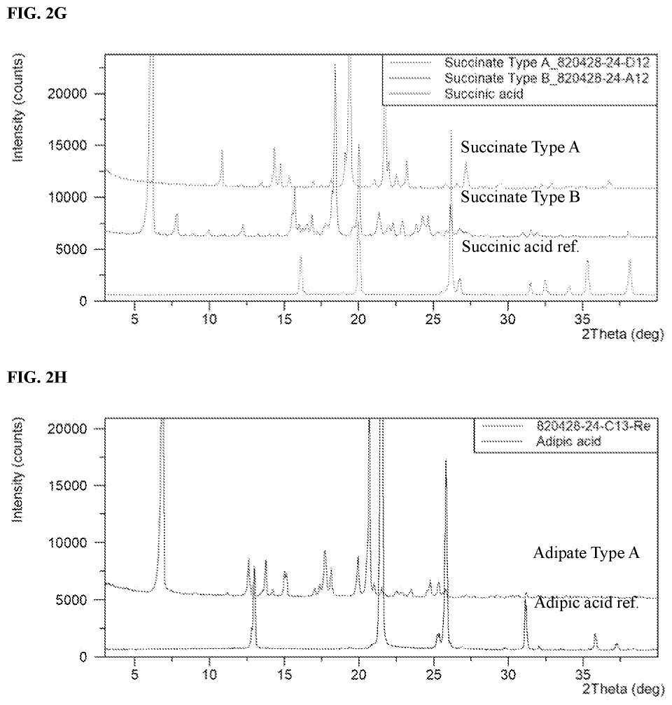

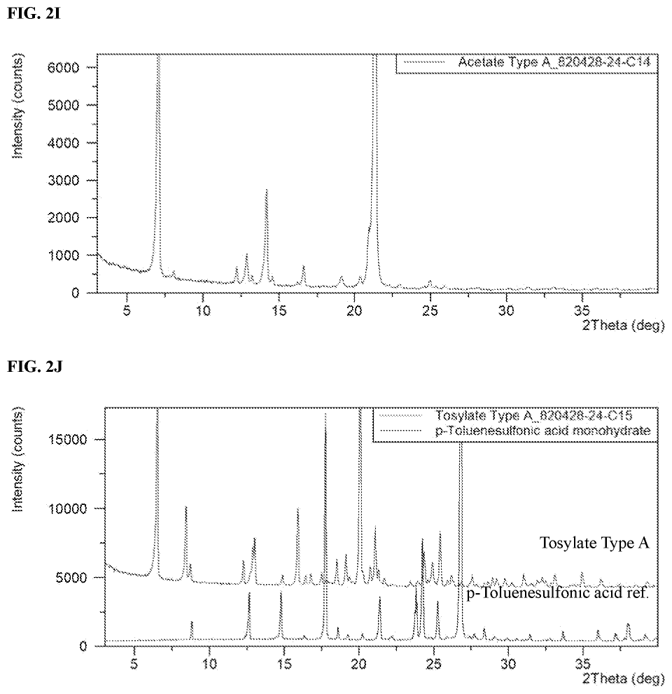

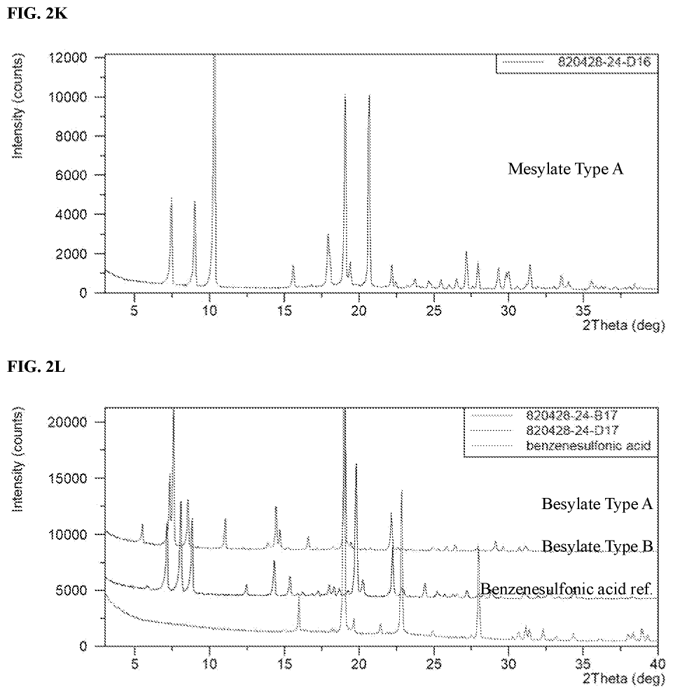

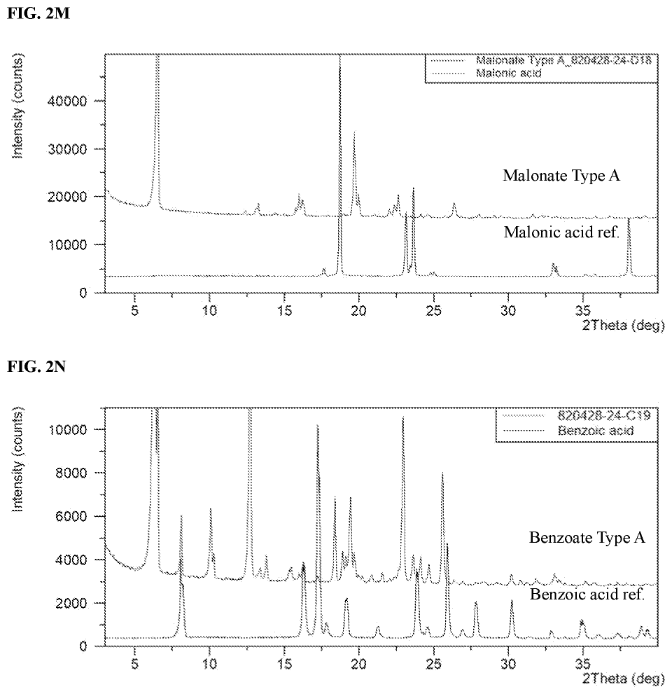

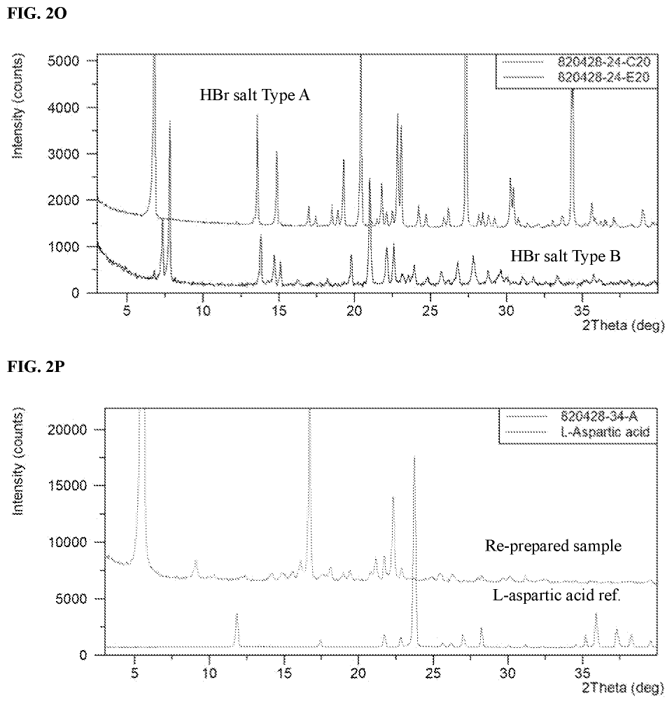

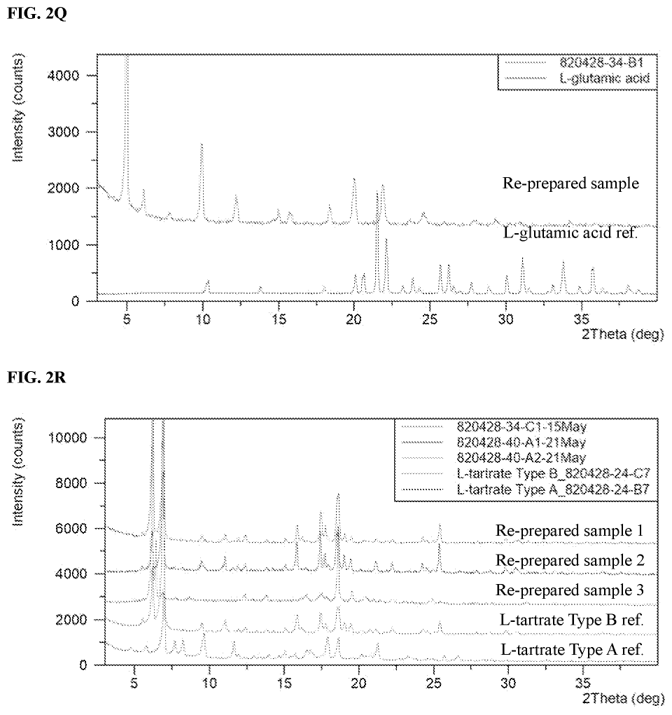

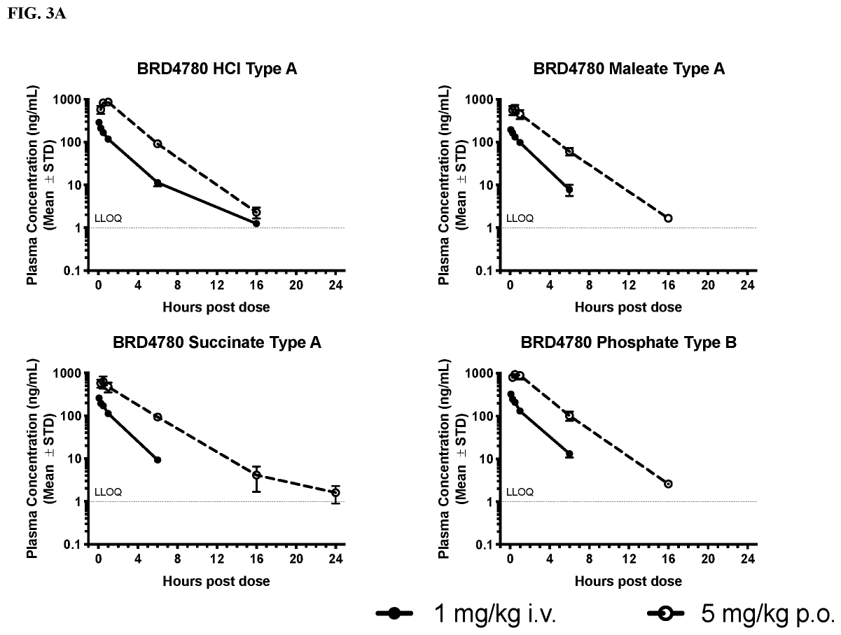

(e.g., anhydrous crystalline compounds or salts thereof) that possess superior properties (e.g., improved thermal properties, improved melting point, hygroscopic resistance, superior pharmacodynamics and/or pharmacokinetics) as compared to those known in the art. In certain embodiments, the melting point of the crystalline form is from about 135° C. to about 146° C. In certain embodiments, the melting point of the crystalline form is from about 136° C. to about 145° C. In certain embodiments, the melting point of the crystalline form is from about 138° C. to about 143° C. In certain preferred embodiments, the melting point of the crystalline form is from about 139 to about 141° C. In certain embodiments, the melting point of the crystalline form is about 139.0° C., 139.2° C., 139.4° C., about 139.6° C., about 139.8° C., about 140.0° C., about 140.2° C., about 140.4° C., about 140.6° C., about 140.8° C., or about 141.0° C. In certain aspects, the crystalline form has one or more 20 values selected from those recited in Table 7, 8, 9, 10, 11, 12, 13, 14, 15, 16, 17, 18, 19, 20, 21, 22, 23, 24, 25, 26, 27, 28, 29, 30, 31 and 32. In certain embodiments, the crystalline form has 3, 4, 5, 6, 7, 8, 9, or 10 2θ values selected from those recited in Table 7, 8, 9, 10, 11, 12, 13, 14, 15, 16, 17, 18, 19, 20, 21, 22, 23, 24, 25, 26, 27, 28, 29, 30, 31 and 32. In preferred embodiments, the crystalline form has 5, 6, 7, 8, 9, or 10 2θ values selected from those recited in Table 7, 8, 9, 10, 11, 12, 13, 14, 15, 16, 17, 18, 19, 20, 21, 22, 23, 24, 25, 26, 27, 28, 29, 30, 31 and 32. In further preferred embodiments, the crystalline form has 7, 8, 9, or 10 2θ values selected from those recited in Table 7, 8, 9, 10, 11, 12, 13, 14, 15, 16, 17, 18, 19, 20, 21, 22, 23, 24, 25, 26, 27, 28, 29, 30, 31 and 32. In certain embodiments, the crystalline form has 20 values substantially similar to those recited in Table 7, 8, 9, 10, 11, 12, 13, 14, 15, 16, 17, 18, 19, 20, 21, 22, 23, 24, 25, 26, 27, 28, 29, 30, 31 or 32. In certain embodiments, the crystalline form has an XRD pattern substantially similar to that depicted in FIG. 2A, 2B, 2C, 2D, 2E, 2F, 2G, 2H, 2I, 2J, 2K, 2L, 2M, 2N, 2O, 2P, 2Q, or 2R.

In one aspect, the present disclosure provides a crystalline form of a compound having a structure represented by formula IIa:

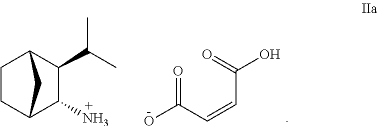

In certain embodiments, the crystalline form has 20 values of about 23.2, about 18.3, about 18.6, about 14.0, about 13.7, about 24.4, about 20.7, or about 18.9. In certain embodiments, the crystalline form has 20 values of about 23.2, about 18.3, about 18.6, about 14.0, and about 13.7. In certain embodiments, the crystalline form has 20 values of about 23.2, about 18.3, about 18.6, about 14.0, about 13.7, about 24.4, and about 20.7. In certain embodiments, the crystalline form has 20 values substantially similar to those recited in Table 22. In certain embodiments, the crystalline form has an XRD pattern substantially similar to that depicted in FIG. 2A. In certain embodiments, the melting point of the crystalline form is from about 136° C. to about 145° C. In certain embodiments, the melting point of the crystalline form is from about 138° C. to about 143° C. In certain preferred embodiments, the melting point of the crystalline form is from about 139 to about 141° C. In certain embodiments, the melting point of the crystalline form is about 139.0° C., 139.2° C., 139.4° C., about 139.6° C., about 139.8° C., about 140.0° C., about 140.2° C., about 140.4° C., about 140.6° C., about 140.8° C., or about 141.0° C.

In another aspect, the present disclosure provides a crystalline form of a compound having a structure represented by formula IIb:

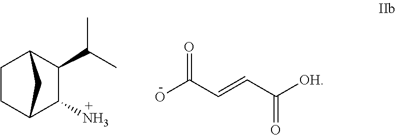

In certain embodiments, the crystalline form has 20 values of about 22.1, about 19.5, about 23.5, about 27.4, about 33.4, about 11.0, about 21.6, or about 18.5. In certain embodiments, the crystalline form has 20 values of about 22.1, about 19.5, about 23.5, about 27.4 and about 33.4. In certain embodiments, the crystalline form has 20 values of about 22.1, about 19.5, about 23.5, about 27.4, about 33.4, about 11.0, and about 21.6. In certain embodiments, the crystalline form has 20 values of about 22.1, about 19.5, about 23.5, about 27.4, about 33.4, about 11.0, about 21.6, and about 18.5. In certain embodiments, the crystalline form has 20 values substantially similar to those recited in Table 26. In certain embodiments, the crystalline form has an XRD pattern substantially similar to that depicted in FIG. 2C.

In yet another aspect, the present disclosure provides a crystalline form of a compound having a structure represented by formula IIc:

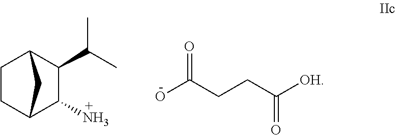

In certain embodiments, the crystalline form has 20 values of about 19.5, about 21.8, about 14.4, about 10.9, about 19.2, 14.8, 27.3, or about 22.1. In certain embodiments, the crystalline form has 20 values of about 19.5, about 21.8, about 14.4, about 10.9, and about 19.2. In certain embodiments, the crystalline form has 20 values of about 19.5, about 21.8, about 14.4, about 10.9, about 19.2, about 14.8, or about 27.3. In certain embodiments, the crystalline form has 20 values of about 19.5, about 21.8, about 14.4, about 10.9, about 19.2, about 14.8, about 27.3, and about 22.1. In certain embodiments, the crystalline form has 20 values substantially similar to those recited in Table 31. In certain embodiments, the crystalline form has an XRD pattern substantially similar to that depicted in FIG. 2G.

In yet another aspect, the present disclosure provides a crystalline form of a compound having a structure represented by formula IId:

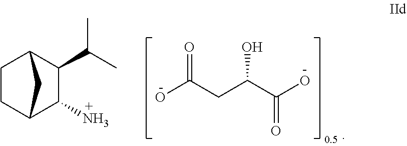

In certain embodiments, the crystalline form has 20 values of about 7.7, about 23.1, about 16.1, about 14.4, about 21.8, about 24.7, about 17.5, or about 12.4. In certain embodiments, the crystalline form has 20 values of about 7.7, about 23.1, about 16.1, about 14.4, and about 21.8. In certain embodiments, the crystalline form has 20 values of about 7.7, about 23.1, about 16.1, about 14.4, about 21.8, about 24.7, and about 17.5. In certain embodiments, has 20 values of about 7.7, about 23.1, about 16.1, about 14.4, about 21.8, about 24.7, about 17.5, and about 12.4. In certain embodiments, the crystalline form has 20 values substantially similar to those recited in Table 29. In certain embodiments, the crystalline form has an XRD pattern substantially similar to that depicted in FIG. 2E.

In yet another aspect, the present disclosure provides a crystalline form of a compound having a structure represented by formula IIe:

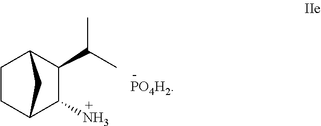

In certain embodiments, the crystalline form has 20 values of about 6.9, about 20.8, about 17.1, about 17.4, about 23.9, about 19.6, about 13.6, or about 19.9. In certain embodiments, the crystalline form has 20 values of about 6.9, about 20.8, about 17.1, about 17.4, and about 23.9. In certain embodiments, the crystalline form has 20 values of about 6.9, about 20.8, about 17.1, about 17.4, and about 23.9, about 19.6, and about 13.6. In certain embodiments, the crystalline form has 20 values of about 6.9, about 20.8, about 17.1, about 17.4, about 23.9, about 19.6, about 13.6, and about 19.9. In certain embodiments, the crystalline form has 20 values substantially similar to those recited in Table 25. In certain embodiments, the crystalline form has an XRD pattern substantially similar to that depicted in FIG. 2B.

Methods of Synthesis

In another one aspect, the present disclosure provides methods of making a compound represented by formula V according to Scheme I:

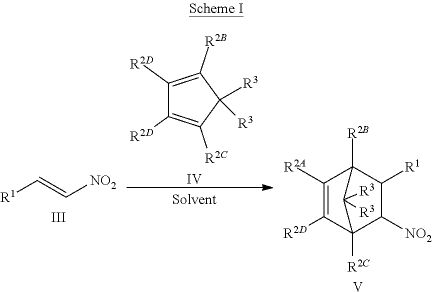

wherein
R¹ is alkyl, cycloalkyl, aryl, or heteroaryl;
each of R^{2A}, R^{2B}, R^{2C}, and R^{2D} is independently selected from H or alkyl; and
the solvent is an aromatic solvent, a heteroaromatic solvent, an organic acid, an alcohol, halogenated alcohol, an ether, a nitrile, a formamide, an alkylamine, or a protic polar solvent.

In certain embodiments, the method is represented by Scheme Ia:

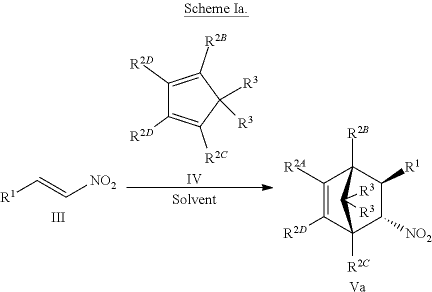

In other embodiments, the method is represented by Scheme Ib:

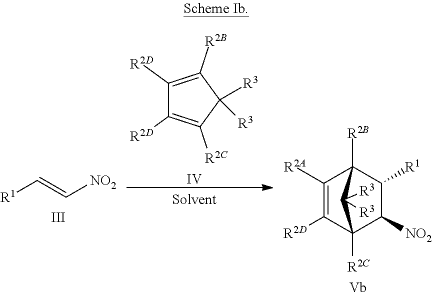

In yet other embodiments, the method is represented by Scheme Ic:

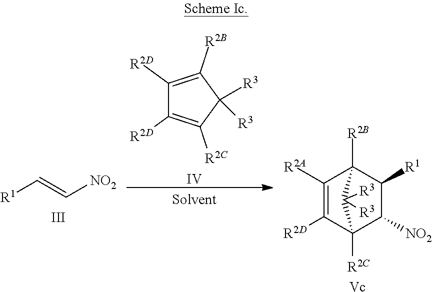

In yet other embodiments, the method is represented by Scheme Id:

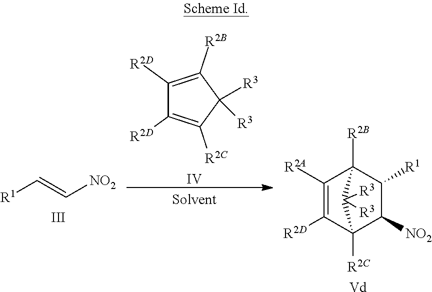

In certain embodiments, the solvent is an aromatic solvent. In certain embodiments, the aromatic solvent is toluene. In other embodiments, the solvent is a heteroaromatic solvent. In certain embodiments, the heteroaromatic solvent is pyridine. In yet other embodiments, the solvent is an organic acid. In certain embodiments, the organic acid is acetic acid, trifluoroacetic acid butyric acid, tertiary butyric acid, toluenesulfonic acid, or benzoic acid. In yet other embodiments, the solvent is an alcohol. In certain embodiments, the alcohol is methanol, ethanol, or isopropanol. In yet other embodiments, the solvent is a halogenated alcohol. In certain embodiments, the halogenated alcohol is a fluorinated alcohol. In certain embodiments, the halogenated alcohol is difluoroethanol or hexafluoroisopropanol. In certain preferred embodiments, the halogenated alcohol is hexafluoroisopropanol. In yet other embodiments, the solvent is an ether. In certain embodiments, the ether is tetrahydrofuran. In certain embodiments, the solvent is a nitrile. In certain embodiments, the nitrile is acetonitrile.

In yet other embodiments, the solvent is a formamide. In certain embodiments, the formamide is dimethyl formamide. In yet other embodiments, the solvent is an alkylamine. In certain embodiments, the alkylamine is diethylamine, trimethylamine, or 1,8-diazabicyclo(5.4.0)undec-7-ene (DBU). In yet other embodiments, the solvent is a protic polar solvent. In certain embodiments, the protic polar solvent is water.

In certain embodiments, the solvent further comprises an additive. In certain embodiments, the additive is an organic acid. In certain embodiments, the organic acid is acetic acid.

In certain embodiments, the method is performed in a temperature range from 20° C. to 60° C. In certain embodiments, the method is performed at about 30° C., about 35° C., about 40° C., about 45° C., or about 50° C. In certain embodiments, the method is performed at about 40° C.

In certain embodiments, the method is performed for between 24 to 144 hours. In certain embodiments, the method is performed for between 48 to 96 hours, e.g., about 72 hours.

In certain embodiments, the method produces the compound of V (e.g., formula Va) at a ratio of about 15:1, about 17.5:1, about 20:1, about 22.5:1, or about 25:1 as compared to the exo isomer. In certain embodiments, the method produces the compound of V (e.g., formula Va) at a ratio of about 21:1 as compared to the exo isomer.

In certain embodiments, the method further comprises a step represented by Scheme II:

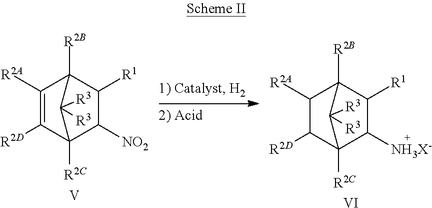

wherein
X⁻ is an organic or inorganic anion;
the catalyst is a noble metal oxide (e.g., platinum oxide), a nickel-aluminum alloy (e.g., Raney nickel), or a noble metal on carbon (e.g., palladium on carbon); and
the acid is a Brønsted acid.

In certain embodiments, the method further comprises a step represented by Scheme IIa:

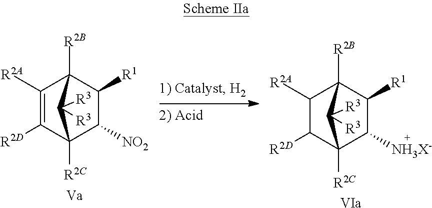

wherein
X⁻ is an organic or inorganic anion;
the catalyst is a noble metal oxide (e.g., platinum oxide), a nickel-aluminum alloy (e.g., Raney nickel), or a noble metal on carbon (e.g., palladium on carbon); and
the acid is a Brønsted acid.

In certain embodiments, the method further comprises a step represented by Scheme IIb:

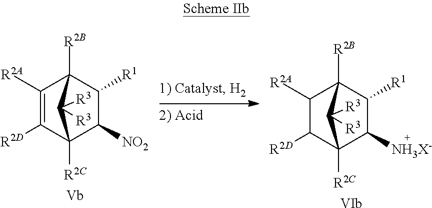

wherein
X⁻ is an organic or inorganic anion;
the catalyst is a noble metal oxide (e.g., platinum oxide), a nickel-aluminum alloy (e.g., Raney nickel), or a noble metal on carbon (e.g., palladium on carbon); and
the acid is a Brønsted acid.

In certain embodiments, the method further comprises a step represented by Scheme IIc:

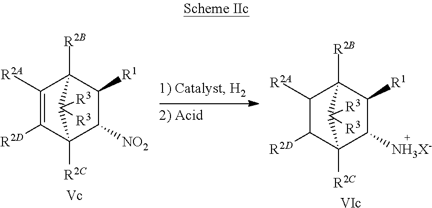

wherein
X⁻ is an organic or inorganic anion;
the catalyst is a noble metal oxide (e.g., platinum oxide), a nickel-aluminum alloy (e.g., Raney nickel), or a noble metal on carbon (e.g., palladium on carbon); and the acid is a Brønsted acid.

In certain embodiments, the method further comprises a step represented by Scheme IId:

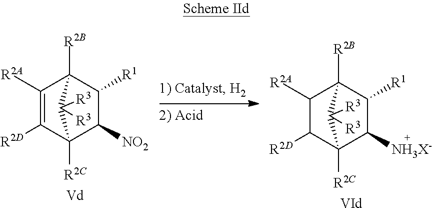

wherein
X⁻ is an organic or inorganic anion;
the catalyst is a noble metal oxide (e.g., platinum oxide), a nickel-aluminum alloy (e.g., Raney nickel), or a noble metal on carbon (e.g., palladium on carbon); and
the acid is a Brønsted acid.

In certain embodiments, the catalyst is palladium on carbon.

In certain embodiments, the acid is a hydrogen halide. In certain preferred embodiments, the acid is HCl (e.g., HCl in ethyl acetate).

In another aspect, the present disclosure provides methods of making a compound represented by formula VV according to Scheme III:

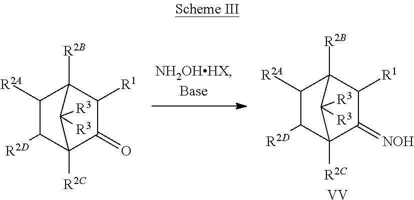

wherein
R¹ is alkyl, cycloalkyl, aryl, or heteroaryl;
each of R^{2A}, R^{2B}, R^{2C}, and R^{2D} is independently selected from H or alkyl;
R³ are both H or both alkyl; and
X is an inorganic anion (e.g., chloride); and
the base is an inorganic base (e.g., sodium acetate).

In another aspect, the present disclosure provides methods of making a compound represented by formula VVa according to Scheme IIIa:

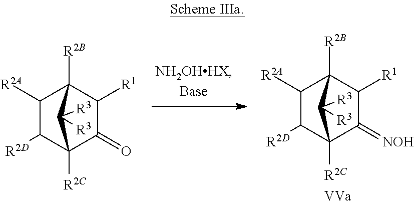

In another aspect, the present disclosure provides methods of making a compound represented by formula VVb according to Scheme IIIb:

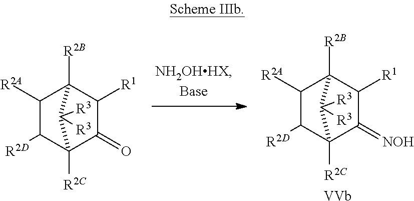

In another aspect, the present disclosure provides methods of making a compound represented by formula VI according to Scheme IV:

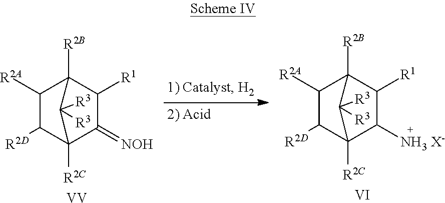

wherein
R¹ is alkyl, cycloalkyl, aryl, or heteroaryl;
each of R^{2A}, R^{2B}, R^{2C}, and R^{2D} is independently selected from H or alkyl;
R³ are both H or both alkyl; and
the catalyst is a noble metal oxide (e.g., platinum oxide); and
the acid is a Brønsted acid.

In certain embodiments, the method is represented by Scheme IVa:

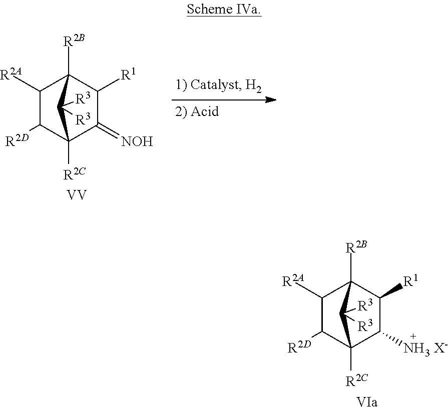

In other embodiments, the method is represented by Scheme IVb:

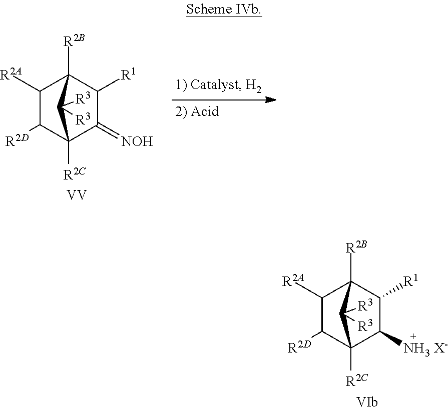

In yet other embodiments, the method is represented by Scheme IVc:

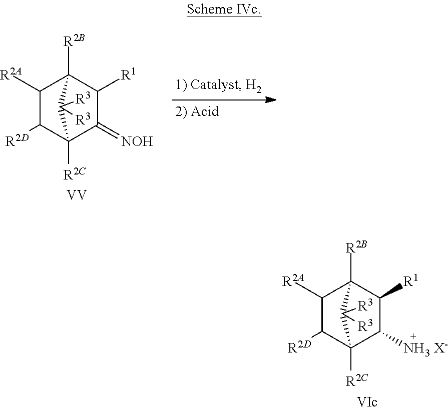

In yet other embodiments, the method is represented by Scheme IVd:

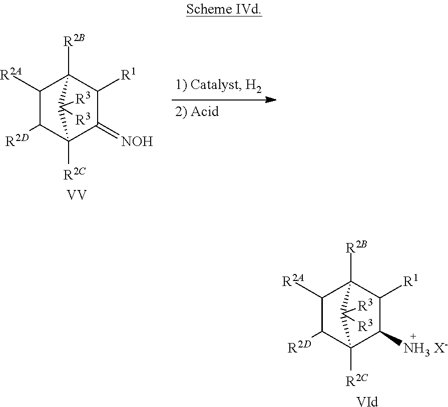

In certain embodiments, the catalyst is platinum oxide.

In certain embodiments, the acid is an organic acid (e.g., acetic acid).

In certain embodiments, the compound has an enantiomeric excess (ee) or diastereomeric excess (de) greater than 95%, 96%, 97%, 98%, or 99%. In certain preferred embodiments, the compound is a single enantiomer or a single diastereomer. In certain embodiments, the compound is substantially free of one enantiomer or of one or more (preferably all) other diastereomers.

In certain embodiments, R¹ is alkyl. In certain embodiments, R¹ is methyl, trifluoromethyl, ethyl, propyl, isopropyl, isobutyl, or tertiary-butyl. In other embodiments, R¹ is cycloalkyl. In certain embodiments, R¹ is cyclopropyl, cyclobutyl, cyclopentyl, or cyclohexyl. In yet other embodiments, R¹ is aryl. In certain embodiments, R¹ is phenyl. In yet other embodiments, R¹ is heteroaryl. In certain embodiments, R¹ is thiophenyl.

In certain embodiments, R¹ is substituted with alkyl, alkenyl, alkynyl, halo, hydroxyl, carboxyl, acyl, acetyl, ester, thioester, alkoxy, phosphoryl, amino, amide, cyano, nitro, azido, alkylthio, alkenyl, alkynyl, cycloalkyl, alkylsulfonyl, or sulfonamide.

In certain embodiments, R^{2A} is H. In certain embodiments, R^{2B} is H. In certain embodiments, R^{2C} is H. In certain embodiments, R^{2D} is H. In certain embodiments, R³ are both H. In certain preferred embodiments, all of these selections occur simultaneously.

In certain embodiments, X⁻ is X⁻ is chloride, maleate, phosphate, fumarate, citrate, malate (e.g., L-malate), lactate, succinate, adipate, acetate, tosylate, mesylate, besylate, benzoate, hydrobromide, aspartate (e.g., L-aspartate), glutamate (e.g., L-glutamate), or tartrate (e.g., L-tartrate). In certain embodiments, X⁻ is Cl⁻, maleate, fumarate, succinate, phosphate, or L-malate. In certain preferred embodiments, X⁻ is Cl⁻. In other preferred embodiments, X⁻ is maleate.

In certain embodiments, the compound represented by formula VI is a salt of a compound selected from

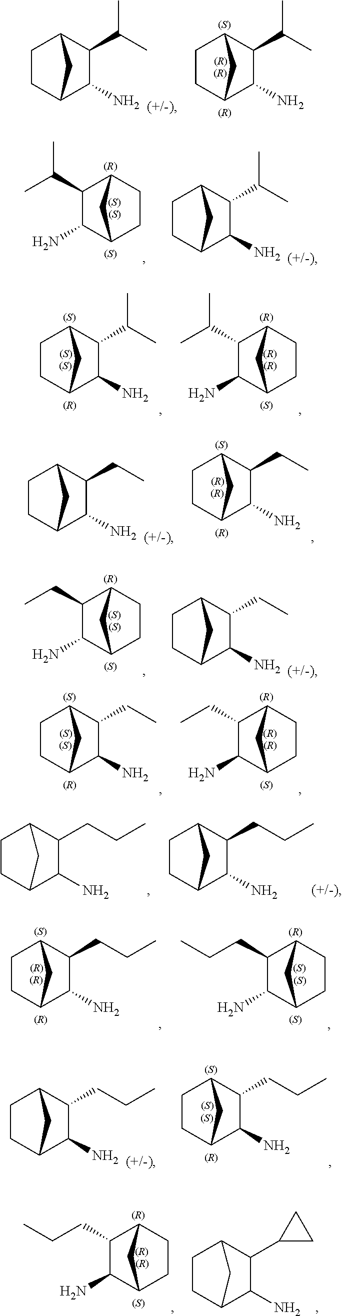

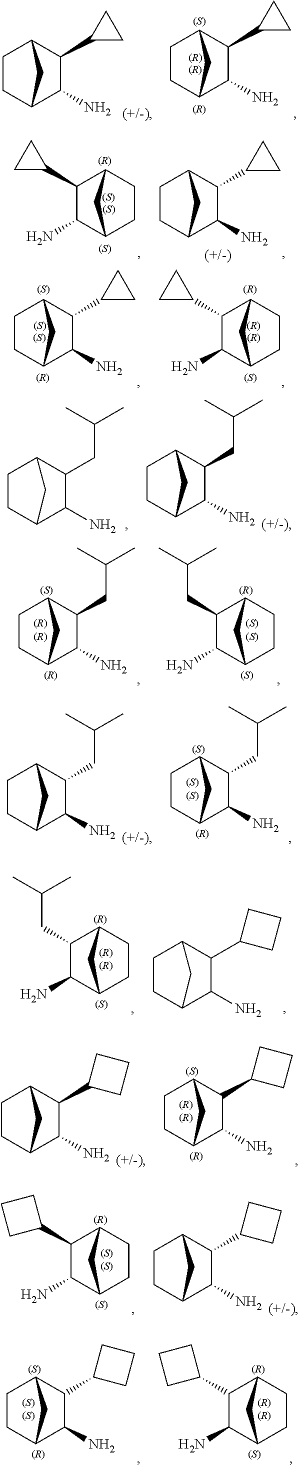

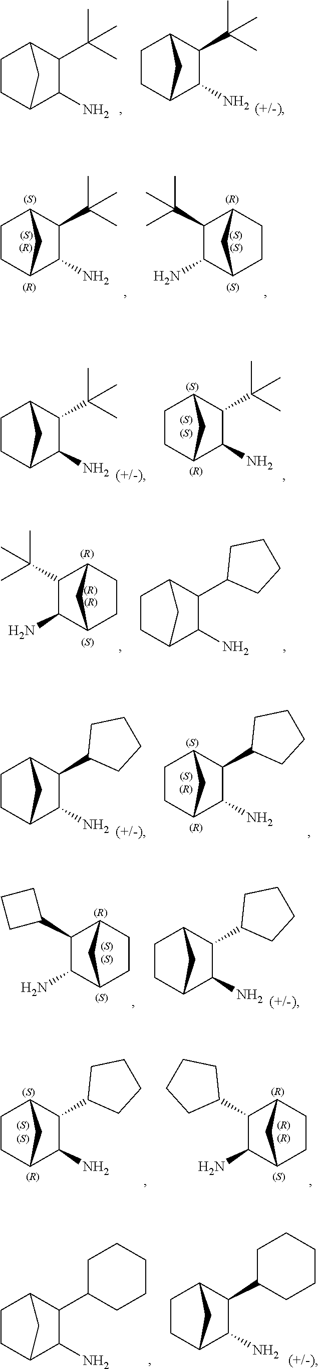

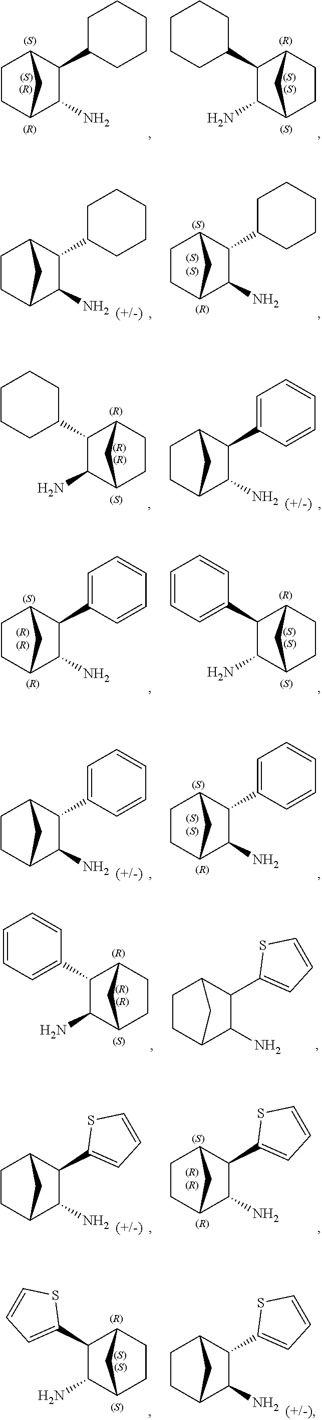

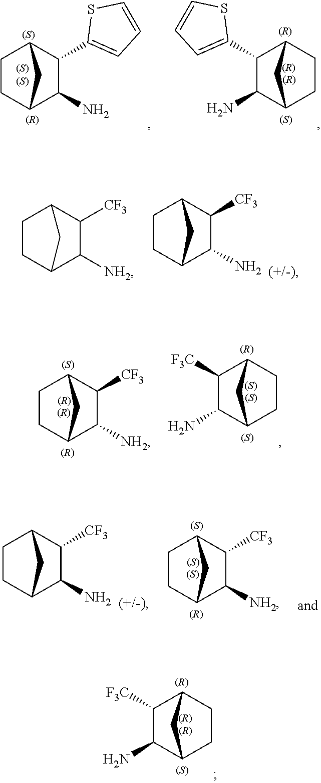

or a pharmaceutically acceptable salt thereof. In certain preferred embodiments, the salt is a maleate salt.

In certain embodiments, the method further comprises a step represented by Scheme V:

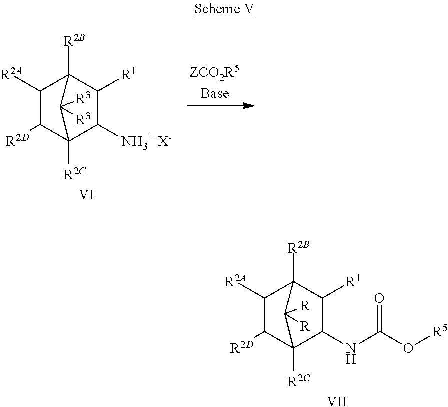

wherein
Z is halo;
R⁵ is aralkyl or heteroaralkyl;

In certain embodiments, the method further comprises a step represented by Scheme Va:

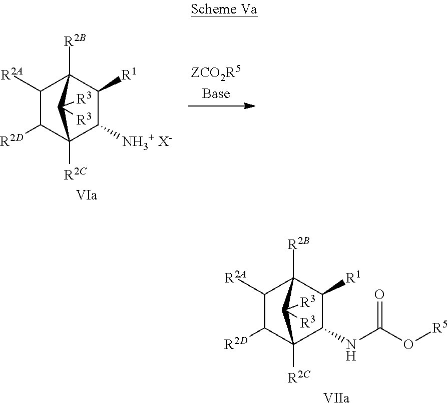

wherein
Z is halo;
R⁵ is aralkyl or heteroaralkyl;
Base is carbonate base or a nitrogenous base.

In certain embodiments, the method further comprises a step represented by Scheme Vb:

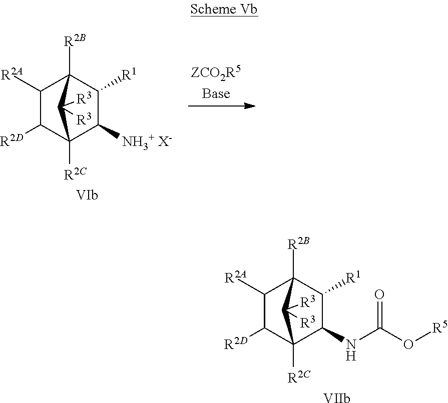

wherein
Z is halo;
R⁵ is aralkyl or heteroaralkyl;
Base is a carbonate base or a nitrogenous base.

In certain embodiments, the method further comprises a step represented by Scheme Vc:

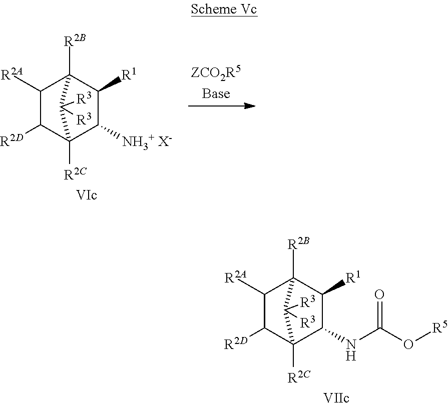

wherein
Z is halo;
R⁵ is aralkyl or heteroaralkyl;
Base is a carbonate base or a nitrogenous base.

In certain embodiments, the method further comprises a step represented by Scheme Vd:

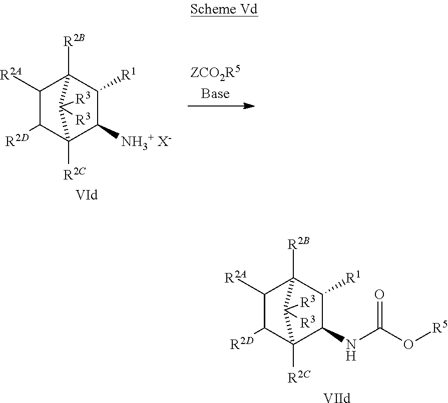

wherein
Z is halo;
R⁵ is aralkyl or heteroaralkyl;
Base is a carbonate base or a nitrogenous base.

In certain preferred embodiments, Z is chloro.

In certain embodiments, R⁵ is aralkyl. In certain embodiments, R⁵ is benzyl. In certain embodiments, R⁵ is substituted with alkyl, alkenyl, alkynyl, halo, hydroxyl, carboxyl, acyl, acetyl, ester, thioester, alkoxy, phosphoryl, amino, amide, cyano, nitro, azido, alkylthio, alkenyl, alkynyl, cycloalkyl, alkylsulfonyl, or sulfonamide. In certain preferred embodiments, R⁵ is substituted with nitro. In certain embodiments, R⁵ is substituted with nitro at the para-position.

In certain embodiments, the nitrogenous base is a secondary or tertiary alkylamine (e.g., trimethylamine or diisopropylamine) In certain embodiments, the carbonate base is sodium carbonate, potassium carbonate, calcium carbonate, or cesium carbonate. In certain preferred embodiments, the carbonate base is sodium carbonate.

In certain embodiments, the method further comprises a solvent. In certain preferred embodiments, the solvent is a mixture of water and a halogenated solvent. In certain embodiments, the halogenated solvent is dichloromethane.

Treatment Selection

The compositions and methods described herein can be used for selecting, and then optionally administering, an optimal treatment (e.g., a compound as disclosed herein, alone (as a mixture of enantiomers (racemic or non-racemic) or diastereomers, or as one enantiomer or diastereomer) or in combination with other agents). Generally, the methods include administering a therapeutically effective amount of a treatment as described herein to a subject who is in need of, or who has been determined to be in need of, such treatment. Therapeutic applications and other uses for the compounds disclosed herein are expressly contemplated to include, without limitation, the full range of applications described in PCT/US2020/038847.

Methods of Treatment

As used in this context, to “treat” means to ameliorate at least one symptom of a proteinopathy. For example, a treatment can result in improved kidney function and/or amelioration in the rate of decline of kidney function that would occur in the absence of treatment, improved neurodegenerative disease and/or eye functions and/or amelioration in the rate of neurodegeneration and/or the rate of declining eye function in a subject having or at risk of a toxic proteinopathy resulting from mutant protein accumulation in the early secretory pathway, or in other organelles of the secretory pathway.

Exemplary neurodegenerative diseases of the instant disclosure include, without limitation, Alzheimer's disease (AD) and other dementias; Parkinson's disease (PD) and PD-related disorders; prion disease (including, e.g., Creutzfeldt-Jakob Disease, variant Creutzfeldt-Jakob Disease, Bovine Spongiform Encephalopathy, Kuru, Gerstmann-Straussler-Scheinker disease, fatal familial insomnia (FFI), scrapie, and other animal TSEs); motor neuron diseases (MND; including, e.g., Amyotrophic Lateral Sclerosis (ALS), Primary Lateral Sclerosis (PLS), Progressive Bulbar Palsy (PBP), Pseudobulbar Palsy, Progressive Muscular Atrophy, Spinal Muscular Atrophy (Type 1, Type 2, Type 3, Type 4), and Kennedy's Disease); and spinocerebellar ataxia (SCA).

In certain embodiments, the methods of the instant disclosure can include selecting and/or administering a treatment that includes a therapeutically effective amount of a therapeutic compound disclosed herein. A therapeutic compound of the instant disclosure may be administered alone to a subject, or, optionally, the compound may be administered in combination with an additional therapeutic agent. Without limitation, specifically contemplated combination therapies for MUC1-associated kidney disease (MKD) include administration of a compound disclosed herein and any of the following: vitamin D in any or all of its forms (e.g., ergocalciferol, cholecalciferol, others), phosphate binders, blood pressure medications and diuretics. Specific examples of phosphate binders, blood pressure medications and diuretics include the following, with exemplary dosages also indicated:

Phosphate Binders:
Calcium Acetate (PhosLo, Calphron, Eliphos, PhosLo Gelcap, and Phoslyra)—667 mg or 667 mg in 5 ml (oral solution)
Sevelamer (Renagel and Renvela)—400 mg (Renagel tablet), 800 mg (Renagel tablet, Renvela tablet, and Renvela powder packet), and 2400 mg (Renvela powder packet)
Ferric Citrate (Auryxia)—210 mg (tablet)
Lanthanum Carbonate (Fosrenol)—Tablet (500 mg, 750 mg, and 1000 mg) and Oral powder (750 mg and 1000 mg).
Sucroferric Oxyhydroxide (Velphoro)—500 mg
Aluminum Hydroxide (Amphojel)—320 mg/5 ml oral suspension

Diuretics:
Bumetanide (Bumex)—0.5 mg (light green), 1 mg (yellow) and 2 mg (peach) tablets for oral administration.
Chlorthalidone (Thalitone)—Oral Tablet: 15 mg, 25 mg, 50 mg
Chlorothiazide (Diuril)—Adults (500 or 1000 mg IV/Tablet)
Ethacrynate (Edecrin)—25 mg tablets for oral use
Furosemide (Lasix)—Tablets 20, 40, and 80 mg
Hydrochlorothiazide HCTZ (Esidrix, Hydrodiuril, Microzide)—25 mg; 50 mg; 100 mg; 50 mg/5 mL; 12.5 mg
Indapamide (Lozol)—2.5 mg orally once a day.
Methyclothiazide (Enduron)—2.5 to 5 mg orally once a day
Metolazone (Mykroz, Zaroxolyn)—2.5 mg orally once a day (Zaroxolyn) or
0.5 mg orally once a day (Mykrox).

Torsemide (Demadex)—5 mg orally once a day; if diuresis remains inadequate after 4 to 6 weeks, titrate up to 10 mg orally once a day; if diuresis remains inadequate with 10 mg, an additional antihypertensive is added.

Blood Pressure Medications:

Beta Blockers:
acebutolol (Sectral)—200 mg or 400 mg tablet
atenolol (Tenormin)—25 mg, 50 mg, and 100 mg tablet.
betaxolol (Kerlone)—10 mg or 20 mg.
bisoprolol (Zebeta)—5 mg or 10 mg tablet.
bisoprolol/hydrochlorothiazide (Ziac)—Bisprolol (2.5 mg)/hydrochloride (6.25 mg) tablet.
metoprolol tartrate (Lopressor)—100 mg tablet daily
metoprolol succinate (Toprol-XL)—25 mg to 100 mg daily.
nadolol (Corgard)—40 mg tablet daily.
pindolol (Visken)—5 mg tablet initial dose.
propranolol (Inderal)—40 mg twice daily.
solotol (Betapace)—80 mg-240 mg tablets in 80 mg increments.
timolol (Blocadren)—5 mg, 10 mg, and 20 mg tablet.

ACE Inhibitors:
benazepril (Lotensin)—10 mg, 20 mg, and 40 mg tablets.
captopril (Capoten)—12.5 mg, 25 mg, 50 mg, and 100 mg tablet.
enalapril (Vasotec)—5 mg initial daily dose.
fosinopril (Monopril)—10 mg once a day
lisinopril (Prinivil, Zestril)—2.5 mg, 5 mg, 10 mg, 20 mg, 30 mg, 40 mg tablets lmg/ml oral solution.
moexipril (Univasc)—7.5 mg and 15 mg tablets.
perindopril (Aceon)—2 mg, 4 mg, and 8 mg tablets.
quinapril (Accupril)—5 mg, 10 mg, 20 mg, and 40 mg tablets.
ramipril (Altace)—1.25 mg tablet, 2.5 mg, and 5 mg tablet.
trandolapril (Mavik)—1 mg, 2 mg, and 4 mg tablet.

Calcium Channel Blockers
amlodipine (Norvasc, Lotrel)—10 mg orally
diltiazem (Cardizem CD, Cardizem SR, Dilacor XR, Tiazac)—20 mg average adult dose
felodipine (Plendil)—2.5 mg, 5 mg, and 10 mg oral tablet.
isradipine (DynaCirc, DynaCirc CR)—7.5 mg daily
nicardipine (Cardene SR)—20 mg and 30 mg capsule.
nifedipine (Adalat CC, Procardia XL)—10 mg, 20 mg, 30 mg, 60 mg, and 90 mg tablet.
nisoldipine (Sular)—17 mg orally daily.
verapamil (Calan SR, Covera HS, Isoptin SR, Verelan)—100 mg/200 mg daily.

Alpha Blockers
doxazosin (Cardura)—2 mg, 4 mg, and 8 mg daily.
prazosin (Minipress)—20 mg total daily dose.
terazosin (Hytrin)—1 mg at bedtime

Alpha-Beta-Blockers
carvedilol (Coreg)—3.125 mg, 6.25 mg, 12.5 mg, and 25 mg tablet.
labetalol (Normodyne, Trandate)—100 mg, 200 mg, and 300 mg taken orally. Central Agonists
methyldopa (Aldomet)—125 mg, 250 mg, and 500 mg tablet.
clonidine (Catapres)—0.1 mg, 0.2 mg, and 0.3 mg.
guanfacine (Tenex)—1 mg, 2 mg, 3 mg, and 4 mg.

Vasodilators
hydralazine (Apresoline)—25 mg, 50 mg, 10 mg, 100 mg, and 20 mg/ml.
minoxidil (Loniten)—2.5 mg and 10 mg tablet.

Without wishing to be bound by theory, though compounds of the instant disclosure have been primarily identified for effect upon the early secretory pathway, it is contemplated that actions of the compounds disclosed herein upon the late secretory pathway could also exert a beneficial effect. Thus, it is contemplated that the compositions and methods of the instant disclosure could also address proteinopathy and related effects in organelles of the late secretory pathway including, without limitation, post-Golgi trafficking vesicles (whether directed to the endosome, including, e.g., ESCRT-II complex vesicles, and/or endosome-bypassing lysosomal transport vesicles and/or cell surface-directed vesicles), the endosome, and/or post-endosomal transport vesicles, including, without limitation, endosome-to-lysosome vesicles, endosome-to-cell surface transport vesicles (including, e.g., synaptic vesicles) and cell surface-to-endosome vesicles, and the lysosome.

Combination Treatments

The compositions and methods of the present disclosure may be used in the context of a number of therapeutic or prophylactic applications. In order to increase the effectiveness of a treatment with the compositions of the present disclosure, e.g., a compound of the instant disclosure selected and/or administered as a single agent, can be selected and/or administered with another agent or therapy, optionally to augment the efficacy of another therapy (second therapy). Thus, it may be desirable to combine these compositions and methods with one another, or with other agents and methods effective in the treatment, amelioration, or prevention of diseases and pathologic conditions, for example, toxic proteinopathies resulting from mutant protein accumulation in the early secretory pathway, such as a neurodegenerative disease, MKD, an autosomal dominant kidney disease caused by uromodulin mutation, a form of retinitis pigmentosa caused by rhodopsin mutation, etc.

Administration of a composition of the present disclosure to a subject will follow general protocols for the administration described herein, and the general protocols for the administration of a particular secondary therapy will also be followed, taking into account the toxicity, if any, of the treatment. It is expected that the treatment cycles would be repeated as necessary. It also is contemplated that various standard therapies may be applied in combination with the described therapies.

### Definitions

Unless otherwise defined herein, scientific and technical terms used in this application shall have the meanings that are commonly understood by those of ordinary skill in the art. Generally, nomenclature used in connection with, and techniques of, chemistry, cell and tissue culture, molecular biology, cell and cancer biology, neurobiology, neurochemistry, virology, immunology, microbiology, pharmacology, genetics and protein and nucleic acid chemistry, described herein, are those well-known and commonly used in the art.

The methods and techniques of the present disclosure are generally performed, unless otherwise indicated, according to conventional methods well known in the art and as described in various general and more specific references that are cited and discussed throughout this specification. See, e.g. “Principles of Neural Science”, McGraw-Hill Medical, New York, N.Y. (2000); Motulsky, “Intuitive Biostatistics”, Oxford University Press, Inc. (1995); Lodish et al., “Molecular Cell Biology, 4th ed.”, W. H. Freeman & Co., New York (2000); Griffiths et al., “Introduction to Genetic Analysis, 7th ed.”, W. H. Freeman & Co., N.Y. (1999); and Gilbert et al., “Developmental Biology, 6th ed.”, Sinauer Associates, Inc., Sunderland, MA (2000).

Chemistry terms used herein, unless otherwise defined herein, are used according to conventional usage in the art, as exemplified by “The McGraw-Hill Dictionary of Chemical Terms”, Parker S., Ed., McGraw-Hill, San Francisco, C.A. (1985).

All of the above, and any other publications, patents and published patent applications referred to in this application are specifically incorporated by reference herein. In case of conflict, the present specification, including its specific definitions, will control.

The term “agent” is used herein to denote a chemical compound (such as an organic or inorganic compound, a mixture of chemical compounds), a biological macromolecule (such as a nucleic acid, an antibody, including parts thereof as well as humanized, chimeric and human antibodies and monoclonal antibodies, a protein or portion thereof, e.g., a peptide, a lipid, a carbohydrate), or an extract made from biological materials such as bacteria, plants, fungi, or animal (particularly mammalian) cells or tissues. Agents include, for example, agents whose structure is known, and those whose structure is not known. The ability of such agents to inhibit AR or promote AR degradation may render them suitable as “therapeutic agents” in the methods and compositions of this disclosure.

A “patient,” “subject,” or “individual” are used interchangeably and refer to either a human or a non-human animal. These terms include mammals, such as humans, primates, livestock animals (including bovines, porcines, etc.), companion animals (e.g., canines, felines, etc.) and rodents (e.g., mice and rats).

“Treating” a condition or patient refers to taking steps to obtain beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment.

The term “preventing” is art-recognized, and when used in relation to a condition, such as a local recurrence (e.g., pain), a disease such as cancer, a syndrome complex such as heart failure or any other medical condition, is well understood in the art, and includes administration of a composition which reduces the frequency of, or delays the onset of, symptoms of a medical condition in a subject relative to a subject which does not receive the composition. Thus, prevention of cancer includes, for example, reducing the number of detectable cancerous growths in a population of patients receiving a prophylactic treatment relative to an untreated control population, and/or delaying the appearance of detectable cancerous growths in a treated population versus an untreated control population, e.g., by a statistically and/or clinically significant amount.

“Administering” or “administration of” a substance, a compound or an agent to a subject can be carried out using one of a variety of methods known to those skilled in the art.

For example, a compound or an agent can be administered, intravenously, arterially, intradermally, intramuscularly, intraperitoneally, subcutaneously, ocularly, sublingually, orally (by ingestion), intranasally (by inhalation), intraspinally, intracerebrally, and transdermally (by absorption, e.g., through a skin duct). A compound or agent can also appropriately be introduced by rechargeable or biodegradable polymeric devices or other devices, e.g., patches and pumps, or formulations, which provide for the extended, slow or controlled release of the compound or agent. Administering can also be performed, for example, once, a plurality of times, and/or over one or more extended periods.

Appropriate methods of administering a substance, a compound or an agent to a subject will also depend, for example, on the age and/or the physical condition of the subject and the chemical and biological properties of the compound or agent (e.g., solubility, digestibility, bioavailability, stability and toxicity). In some embodiments, a compound or an agent is administered orally, e.g., to a subject by ingestion. In some embodiments, the orally administered compound or agent is in an extended release or slow release formulation, or administered using a device for such slow or extended release.

As used herein, the phrase “conjoint administration” refers to any form of administration of two or more different therapeutic agents such that the second agent is administered while the previously administered therapeutic agent is still effective in the body (e.g., the two agents are simultaneously effective in the patient, which may include synergistic effects of the two agents). For example, the different therapeutic compounds can be administered either in the same formulation or in separate formulations, either concomitantly or sequentially. Thus, an individual who receives such treatment can benefit from a combined effect of different therapeutic agents.

A “therapeutically effective amount” or a “therapeutically effective dose” of a drug or agent is an amount of a drug or an agent that, when administered to a subject will have the intended therapeutic effect. The full therapeutic effect does not necessarily occur by administration of one dose, and may occur only after administration of a series of doses. Thus, a therapeutically effective amount may be administered in one or more administrations. The precise effective amount needed for a subject will depend upon, for example, the subject's size, health and age, and the nature and extent of the condition being treated, such as cancer or MDS. The skilled worker can readily determine the effective amount for a given situation by routine experimentation.

As used herein, the terms “optional” or “optionally” mean that the subsequently described event or circumstance may occur or may not occur, and that the description includes instances where the event or circumstance occurs as well as instances in which it does not. For example, “optionally substituted alkyl” refers to the alkyl may be substituted as well as where the alkyl is not substituted.

It is understood that substituents and substitution patterns on the compounds of the present invention can be selected by one of ordinary skilled person in the art to result chemically stable compounds which can be readily synthesized by techniques known in the art, as well as those methods set forth below, from readily available starting materials. If a substituent is itself substituted with more than one group, it is understood that these multiple groups may be on the same carbon or on different carbons, so long as a stable structure results.

As used herein, the term “optionally substituted” refers to the replacement of one to six hydrogen radicals in a given structure with the radical of a specified substituent including, but not limited to: hydroxyl, hydroxyalkyl, alkoxy, halogen, alkyl, nitro, silyl, acyl, acyloxy, aryl, cycloalkyl, heterocyclyl, amino, aminoalkyl, cyano, haloalkyl, haloalkoxy, —OCO—CH₂—O-alkyl, —OP(O)(O-alkyl)₂ or —CH₂—OP(O)(O-alkyl)₂. Preferably, “optionally substituted” refers to the replacement of one to four hydrogen radicals in a given structure with the substituents mentioned above. More preferably, one to three hydrogen radicals are replaced by the substituents as mentioned above. It is understood that the substituent can be further substituted.

As used herein, the term “alkyl” refers to saturated aliphatic groups, including but not limited to C₁-C₁₀ straight-chain alkyl groups or C₁-C₁₀ branched-chain alkyl groups. Preferably, the “alkyl” group refers to C₁-C₆ straight-chain alkyl groups or C₁-C₆ branched-chain alkyl groups. Most preferably, the “alkyl” group refers to C₁-C₄ straight-chain alkyl groups or C₁-C₄ branched-chain alkyl groups. Examples of “alkyl” include, but are not limited to, methyl, ethyl, 1-propyl, 2-propyl, n-butyl, sec-butyl, tert-butyl, 1-pentyl, 2-pentyl, 3-pentyl, neo-pentyl, 1-hexyl, 2-hexyl, 3-hexyl, 1-heptyl, 2-heptyl, 3-heptyl, 4-heptyl, 1-octyl, 2-octyl, 3-octyl or 4-octyl and the like. The “alkyl” group may be optionally substituted.

The term “acyl” is art-recognized and refers to a group represented by the general formula hydrocarbylC(O)—, preferably alkylC(O)—.

The term “acylamino” is art-recognized and refers to an amino group substituted with an acyl group and may be represented, for example, by the formula hydrocarbylC(O)NH—.

The term “acyloxy” is art-recognized and refers to a group represented by the general formula hydrocarbylC(O)O—, preferably alkylC(O)O—.

The term “alkoxy” refers to an alkyl group having an oxygen attached thereto.

Representative alkoxy groups include methoxy, ethoxy, propoxy, tert-butoxy and the like.

The term “alkoxyalkyl” refers to an alkyl group substituted with an alkoxy group and may be represented by the general formula alkyl-O-alkyl.

The term “alkyl” refers to saturated aliphatic groups, including straight-chain alkyl groups, branched-chain alkyl groups, cycloalkyl (alicyclic) groups, alkyl-substituted cycloalkyl groups, and cycloalkyl-substituted alkyl groups. In preferred embodiments, a straight chain or branched chain alkyl has 30 or fewer carbon atoms in its backbone (e.g., C₁₋₃₀ for straight chains, C₃₋₃₀ for branched chains), and more preferably 20 or fewer.

Moreover, the term “alkyl” as used throughout the specification, examples, and claims is intended to include both unsubstituted and substituted alkyl groups, the latter of which refers to alkyl moieties having substituents replacing a hydrogen on one or more carbons of the hydrocarbon backbone, including haloalkyl groups such as trifluoromethyl and 2,2,2-trifluoroethyl, etc.

The term “C_{x-y}” or “Cₓ-C_{y}”, when used in conjunction with a chemical moiety, such as, acyl, acyloxy, alkyl, alkenyl, alkynyl, or alkoxy is meant to include groups that contain from x to y carbons in the chain. C₀alkyl indicates a hydrogen where the group is in a terminal position, a bond if internal. A C₁₋₆alkyl group, for example, contains from one to six carbon atoms in the chain.

The term “alkylamino”, as used herein, refers to an amino group substituted with at least one alkyl group.

The term “alkylthio”, as used herein, refers to a thiol group substituted with an alkyl group and may be represented by the general formula alkylS-.

The term “amide”, as used herein, refers to a group

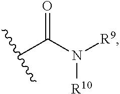

wherein R⁹ and R¹⁰ each independently represent a hydrogen or hydrocarbyl group, or R⁹ and R¹⁰ taken together with the N atom to which they are attached complete a heterocycle having from 4 to 8 atoms in the ring structure.

The terms “amine” and “amino” are art-recognized and refer to both unsubstituted and substituted amines and salts thereof, e.g., a moiety that can be represented by

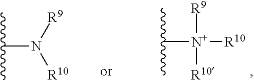

wherein R⁹, R¹⁰, and R^{10′} each independently represent a hydrogen or a hydrocarbyl group, or R⁹ and R¹⁰ taken together with the N atom to which they are attached complete a heterocycle having from 4 to 8 atoms in the ring structure.

The term “aminoalkyl”, as used herein, refers to an alkyl group substituted with an amino group.

The term “aralkyl”, as used herein, refers to an alkyl group substituted with an aryl group.

The term “aryl” as used herein include substituted or unsubstituted single-ring aromatic groups in which each atom of the ring is carbon. Preferably the ring is a 5- to 7-membered ring, more preferably a 6-membered ring. The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Aryl groups include benzene, naphthalene, phenanthrene, phenol, aniline, and the like.

The term “carbamate” is art-recognized and refers to a group

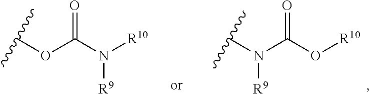

wherein R⁹ and R¹⁰ independently represent hydrogen or a hydrocarbyl group.

The term “carbocyclylalkyl”, as used herein, refers to an alkyl group substituted with a carbocycle group.

The term “carbocycle” includes 5-7 membered monocyclic and 8-12 membered bicyclic rings. Each ring of a bicyclic carbocycle may be selected from saturated, unsaturated and aromatic rings. Carbocycle includes bicyclic molecules in which one, two or three or more atoms are shared between the two rings. The term “fused carbocycle” refers to a bicyclic carbocycle in which each of the rings shares two adjacent atoms with the other ring. Each ring of a fused carbocycle may be selected from saturated, unsaturated and aromatic rings. In an exemplary embodiment, an aromatic ring, e.g., phenyl, may be fused to a saturated or unsaturated ring, e.g., cyclohexane, cyclopentane, or cyclohexene. Any combination of saturated, unsaturated and aromatic bicyclic rings, as valence permits, is included in the definition of carbocyclic. Exemplary “carbocycles” include cyclopentane, cyclohexane, bicyclo[2.2.1]heptane, 1,5-cyclooctadiene, 1,2,3,4-tetrahydronaphthalene, bicyclo[4.2.0]oct-3-ene, naphthalene and adamantane. Exemplary fused carbocycles include decalin, naphthalene, 1,2,3,4-tetrahydronaphthalene, bicyclo[4.2.0]octane, 4,5,6,7-tetrahydro-1H-indene and bicyclo[4.1.0]hept-3-ene. “Carbocycles” may be substituted at any one or more positions capable of bearing a hydrogen atom.

The term “carbocyclylalkyl”, as used herein, refers to an alkyl group substituted with a carbocycle group.

The term “carbonate” is art-recognized and refers to a group —OCO₂—.

The term “carboxy”, as used herein, refers to a group represented by the formula —CO₂H.

The term “cycloalkyl” includes substituted or unsubstituted non-aromatic single ring structures, preferably 4- to 8-membered rings, more preferably 4- to 6-membered rings. The term “cycloalkyl” also includes polycyclic ring systems having two or more cyclic rings in which two or more carbons are common to two adjoining rings wherein at least one of the rings is cycloalkyl and the substituent (e.g., R¹⁰°) is attached to the cycloalkyl ring, e.g., the other cyclic rings can be cycloalkyls, cycloalkenyls, cycloalkynyls, aryls, heteroaryls, and/or heterocyclyls. Heteroaryl groups include, for example, pyrrole, furan, thiophene, imidazole, oxazole, thiazole, pyrazole, pyridine, pyrazine, pyridazine, pyrimidine, denzodioxane, tetrahydroquinoline, and the like.

The term “ester”, as used herein, refers to a group —C(O)OR⁹ wherein R⁹ represents a hydrocarbyl group.

The term “ether”, as used herein, refers to a hydrocarbyl group linked through an oxygen to another hydrocarbyl group. Accordingly, an ether substituent of a hydrocarbyl group may be hydrocarbyl-O—. Ethers may be either symmetrical or unsymmetrical. Examples of ethers include, but are not limited to, heterocycle-O-heterocycle and aryl-O-heterocycle. Ethers include “alkoxyalkyl” groups, which may be represented by the general formula alkyl-O-alkyl.

The terms “halo” and “halogen” as used herein means halogen and includes chloro, fluoro, bromo, and iodo.

The terms “hetaralkyl” and “heteroaralkyl”, as used herein, refers to an alkyl group substituted with a hetaryl group.

The terms “heteroaryl” and “hetaryl” include substituted or unsubstituted aromatic single ring structures, preferably 5- to 7-membered rings, more preferably 5- to 6-membered rings, whose ring structures include at least one heteroatom, preferably one to four heteroatoms, more preferably one or two heteroatoms. The terms “heteroaryl” and “hetaryl” also include polycyclic ring systems having two or more cyclic rings in which two or more carbons are common to two adjoining rings wherein at least one of the rings is heteroaromatic, e.g., the other cyclic rings can be cycloalkyls, cycloalkenyls, cycloalkynyls, aryls, heteroaryls, and/or heterocyclyls. Heteroaryl groups include, for example, pyrrole, furan, thiophene, imidazole, oxazole, thiazole, pyrazole, pyridine, pyrazine, pyridazine, and pyrimidine, and the like.

The term “heteroatom” as used herein means an atom of any element other than carbon or hydrogen. Preferred heteroatoms are nitrogen, oxygen, and sulfur.

The term “heterocyclylalkyl”, as used herein, refers to an alkyl group substituted with a heterocycle group.

The terms “heterocyclyl”, “heterocycle”, and “heterocyclic” refer to substituted or unsubstituted non-aromatic ring structures, preferably 3- to 10-membered rings, more preferably 3- to 7-membered rings, whose ring structures include at least one heteroatom, preferably one to four heteroatoms, more preferably one or two heteroatoms. The terms “heterocyclyl” and “heterocyclic” also include polycyclic ring systems having two or more cyclic rings in which two or more carbons are common to two adjoining rings wherein at least one of the rings is heterocyclic, e.g., the other cyclic rings can be cycloalkyls, cycloalkenyls, cycloalkynyls, aryls, heteroaryls, and/or heterocyclyls. Heterocyclyl groups include, for example, piperidine, piperazine, pyrrolidine, morpholine, lactones, lactams, and the like.

The term “hydrocarbyl”, as used herein, refers to a group that is bonded through a carbon atom that does not have a ═O or ═S substituent, and typically has at least one carbon-hydrogen bond and a primarily carbon backbone, but may optionally include heteroatoms. Thus, groups like methyl, ethoxyethyl, 2-pyridyl, and even trifluoromethyl are considered to be hydrocarbyl for the purposes of this application, but substituents such as acetyl (which has a ═O substituent on the linking carbon) and ethoxy (which is linked through oxygen, not carbon) are not. Hydrocarbyl groups include, but are not limited to aryl, heteroaryl, carbocycle, heterocycle, alkyl, alkenyl, alkynyl, and combinations thereof.

The term “hydroxyalkyl”, as used herein, refers to an alkyl group substituted with a hydroxy group.

The term “lower” when used in conjunction with a chemical moiety, such as, acyl, acyloxy, alkyl, alkenyl, alkynyl, or alkoxy is meant to include groups where there are ten or fewer atoms in the substituent, preferably six or fewer. A “lower alkyl”, for example, refers to an alkyl group that contains ten or fewer carbon atoms, preferably six or fewer. In certain embodiments, acyl, acyloxy, alkyl, alkenyl, alkynyl, or alkoxy substituents defined herein are respectively lower acyl, lower acyloxy, lower alkyl, lower alkenyl, lower alkynyl, or lower alkoxy, whether they appear alone or in combination with other substituents, such as in the recitations hydroxyalkyl and aralkyl (in which case, for example, the atoms within the aryl group are not counted when counting the carbon atoms in the alkyl substituent).

The terms “polycyclyl”, “polycycle”, and “polycyclic” refer to two or more rings (e.g., cycloalkyls, cycloalkenyls, cycloalkynyls, aryls, heteroaryls, and/or heterocyclyls) in which two or more atoms are common to two adjoining rings, e.g., the rings are “fused rings”. Each of the rings of the polycycle can be substituted or unsubstituted. In certain embodiments, each ring of the polycycle contains from 3 to 10 atoms in the ring, preferably from 5 to 7.

The term “sulfate” is art-recognized and refers to the group —OSO₃H, or a pharmaceutically acceptable salt thereof.

The term “sulfonamide” is art-recognized and refers to the group represented by the general formulae

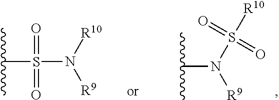

wherein R⁹ and R¹⁰ independently represents hydrogen or hydrocarbyl.

The term “sulfoxide” is art-recognized and refers to the group-S(O)—.

The term “sulfonate” is art-recognized and refers to the group SO₃H, or a pharmaceutically acceptable salt thereof.

The term “sulfone” is art-recognized and refers to the group —S(O)₂—.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate.

The term “thioalkyl”, as used herein, refers to an alkyl group substituted with a thiol group.

The term “thioester”, as used herein, refers to a group —C(O)SR⁹ or —SC(O)R⁹ wherein R⁹ represents a hydrocarbyl.

The term “thioether”, as used herein, is equivalent to an ether, wherein the oxygen is replaced with a sulfur.

The term “urea” is art-recognized and may be represented by the general formula

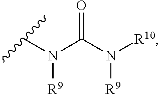

wherein R⁹ and R¹⁰ independently represent hydrogen or a hydrocarbyl.

The term “modulate” as used herein includes the inhibition or suppression of a function or activity (such as cell proliferation) as well as the enhancement of a function or activity.

The phrase “pharmaceutically acceptable” is art-recognized. In certain embodiments, the term includes compositions, excipients, adjuvants, polymers and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable salt” or “salt” is used herein to refer to an acid addition salt or a basic addition salt which is suitable for or compatible with the treatment of patients.

The term “pharmaceutically acceptable acid addition salt” as used herein means any non-toxic organic or inorganic salt of any base compounds represented by Formula I. Illustrative inorganic acids which form suitable salts include hydrochloric, hydrobromic, sulfuric and phosphoric acids, as well as metal salts such as sodium monohydrogen orthophosphate and potassium hydrogen sulfate. Illustrative organic acids that form suitable salts include mono-, di-, and tricarboxylic acids such as glycolic, lactic, pyruvic, malonic, succinic, glutaric, fumaric, malic, tartaric, citric, ascorbic, maleic, benzoic, phenylacetic, cinnamic and salicylic acids, as well as sulfonic acids such as p-toluene sulfonic and methanesulfonic acids. Either the mono or di-acid salts can be formed, and such salts may exist in either a hydrated, solvated or substantially anhydrous form. In general, the acid addition salts of compounds of Formula I are more soluble in water and various hydrophilic organic solvents, and generally demonstrate higher melting points in comparison to their free base forms. The selection of the appropriate salt will be known to one skilled in the art. Other non-pharmaceutically acceptable salts, e.g., oxalates, may be used, for example, in the isolation of compounds of Formula I for laboratory use, or for subsequent conversion to a pharmaceutically acceptable acid addition salt.

The term “pharmaceutically acceptable basic addition salt” as used herein means any non-toxic organic or inorganic base addition salt of any acid compounds represented by Formula I or any of their intermediates. Illustrative inorganic bases which form suitable salts include lithium, sodium, potassium, calcium, magnesium, or barium hydroxide. Illustrative organic bases which form suitable salts include aliphatic, alicyclic, or aromatic organic amines such as methylamine, trimethylamine and picoline or ammonia. The selection of the appropriate salt will be known to a person skilled in the art.

Many of the compounds useful in the methods and compositions of this disclosure have at least one stereogenic center in their structure. This stereogenic center may be present in a R or a S configuration, said R and S notation is used in correspondence with the rules described in Pure Appl. Chem. (1976), 45, 11-30. The disclosure contemplates all stereoisomeric forms such as enantiomeric and diastereoisomeric forms of the compounds, salts, prodrugs or mixtures thereof (including all possible mixtures of stereoisomers). See, e.g., WO 01/062726.

In certain embodiments, compounds of the disclosure may be racemic. In certain embodiments, compounds of the disclosure may be enriched in one enantiomer. For example, a compound of the disclosure may have greater than about 30% ee, 40% ee, 50% ee, 60% ee, 70% ee, 80% ee, 90% ee, 95%, 96% ee, 97% ee, 98% ee, 99% ee, or greater ee. In certain embodiments, compounds of the invention may have more than one stereocenter. In certain such embodiments, compounds of the invention may be enriched in one or more diastereomers. For example, a compound of the invention may have greater than about 30% de, about 40% de, about 50% de, about 60% de, about 70% de, about 80% de, about 90% de, or even about 95% or greater de.

In certain embodiments, a composition may be enriched to provide predominantly one enantiomer of a compound. An enantiomerically enriched composition may comprise, for example, at least about 60 mol percent of one enantiomer, or more preferably at least about 75, about 90, about 95, or even about 99 mol percent. In certain embodiments, the composition enriched in one enantiomer is substantially free of the other enantiomer, wherein substantially free means that the substance in question makes up less than about 10%, or less than about 5%, or less than about 4%, or less than about 3%, or less than about 2%, or less than about 1% as compared to the amount of the other enantiomer, e.g., in the composition or compound mixture. For example, if a composition or compound mixture contains about 98 grams of a first enantiomer and about 2 grams of a second enantiomer, it would be said to contain about 98 mol percent of the first enantiomer and only about 2% of the second enantiomer.

In certain embodiments, the composition may be enriched to provide predominantly one diastereomer of a compound, e.g., relative to other diastereomers. A diastereomerically enriched composition may comprise, for example, at least about 60 mol percent of one diastereomer, or more preferably at least about 75, about 90, about 95, or even about 99 mol percent.

Furthermore, certain compounds which contain alkenyl groups may exist as Z (zusammen) or E (entgegen) isomers. In each instance, the disclosure includes both mixture and separate individual isomers.

Some of the compounds may also exist in tautomeric forms. Such forms, although not explicitly indicated in the formulae described herein, are intended to be included within the scope of the present disclosure.

“Prodrug” or “pharmaceutically acceptable prodrug” refers to a compound that is metabolized, for example hydrolyzed or oxidized, in the host after administration to form the compound of the present disclosure (e.g., compounds of formula I). Typical examples of prodrugs include compounds that have biologically labile or cleavable (protecting) groups on a functional moiety of the active compound. Prodrugs include compounds that can be oxidized, reduced, aminated, deaminated, hydroxylated, dehydroxylated, hydrolyzed, dehydrolyzed, alkylated, dealkylated, acylated, deacylated, phosphorylated, or dephosphorylated to produce the active compound. Examples of prodrugs using ester or phosphoramidate as biologically labile or cleavable (protecting) groups are disclosed in U.S. Pat. Nos. 6,875,751, 7,585,851, and 7,964,580, the disclosures of which are incorporated herein by reference. The prodrugs of this disclosure are metabolized to produce a compound of Formula I. The present disclosure includes within its scope, prodrugs of the compounds described herein. Conventional procedures for the selection and preparation of suitable prodrugs are described, for example, in “Design of Prodrugs” Ed. H. Bundgaard, Elsevier, 1985.

The phrase “pharmaceutically acceptable carrier” as used herein means a pharmaceutically acceptable material, composition or vehicle, such as a liquid or solid filter, diluent, excipient, solvent or encapsulating material useful for formulating a drug for medicinal or therapeutic use.

The term “Log of solubility”, “Log S” or “log S” as used herein is used in the art to quantify the aqueous solubility of a compound. The aqueous solubility of a compound significantly affects its absorption and distribution characteristics. A low solubility often goes along with a poor absorption. Log S value is a unit stripped logarithm (base 10) of the solubility measured in mol/liter.

Pharmaceutical Compositions

The compositions and methods of the present invention may be utilized to treat an individual in need thereof. In certain embodiments, the individual is a mammal such as a human, or a non-human mammal. When administered to an animal (e.g., a mammal), such as a human, the composition or the compound is preferably administered as a pharmaceutical composition comprising, for example, a compound of the invention and a pharmaceutically acceptable carrier. Pharmaceutically acceptable carriers are well known in the art and include, for example, aqueous solutions such as water or physiologically buffered saline or other solvents or vehicles such as glycols, glycerol, oils such as olive oil, or injectable organic esters. In preferred embodiments, when such pharmaceutical compositions are for human administration, particularly for invasive routes of administration (i.e., routes, such as injection or implantation, that circumvent transport or diffusion through an epithelial barrier), the aqueous solution is pyrogen-free, or substantially pyrogen-free. The excipients can be chosen, for example, to effect delayed release of an agent or to selectively target one or more cells, tissues or organs. The pharmaceutical composition can be in dosage unit form such as tablet, capsule (including sprinkle capsule and gelatin capsule), granule, lyophile for reconstitution, powder, solution, syrup, suppository, injection or the like. The composition can also be present in a transdermal delivery system, e.g., a skin patch. The composition can also be present in a solution suitable for topical administration, such as a lotion, cream, or ointment.

A pharmaceutically acceptable carrier can contain physiologically acceptable agents that act, for example, to stabilize, increase solubility or to increase the absorption of a compound such as a compound of the invention. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The preparation or pharmaceutical composition can be a selfemulsifying drug delivery system or a selfmicroemulsifying drug delivery system. The pharmaceutical composition (preparation) also can be a liposome or other polymer matrix, which can have incorporated therein, for example, a compound of the invention. Liposomes, for example, which comprise phospholipids or other lipids, are nontoxic, physiologically acceptable and metabolizable carriers that are relatively simple to make and administer.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

A pharmaceutical composition (preparation) can be administered to a subject by any of a number of routes of administration including, for example, orally (for example, drenches as in aqueous or non-aqueous solutions or suspensions, tablets, capsules (including sprinkle capsules and gelatin capsules), boluses, powders, granules, pastes for application to the tongue); absorption through the oral mucosa (e.g., sublingually); subcutaneously; transdermally (for example as a patch applied to the skin); and topically (for example, as a cream, ointment or spray applied to the skin). The compound may also be formulated for inhalation. In certain embodiments, a compound may be simply dissolved or suspended in sterile water. Details of appropriate routes of administration and compositions suitable for same can be found in, for example, U.S. Pat. Nos. 6,110,973, 5,763,493, 5,731,000, 5,541,231, 5,427,798, 5,358,970 and 4,172,896, as well as in patents cited therein.

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an active compound, such as a compound of the invention, with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules (including sprinkle capsules and gelatin capsules), cachets, pills, tablets, lozenges (using a flavored basis, usually sucrose and acacia or tragacanth), lyophile, powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. Compositions or compounds may also be administered as a bolus, electuary or paste.

To prepare solid dosage forms for oral administration (capsules (including sprinkle capsules and gelatin capsules), tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof, (10) complexing agents, such as, modified and unmodified cyclodextrins; and (11) coloring agents. In the case of capsules (including sprinkle capsules and gelatin capsules), tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions, such as dragees, capsules (including sprinkle capsules and gelatin capsules), pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms useful for oral administration include pharmaceutically acceptable emulsions, lyophiles for reconstitution, microemulsions, solutions, suspensions, syrups and elixirs. In addition to the active ingredient, the liquid dosage forms may contain inert diluents commonly used in the art, such as, for example, water or other solvents, cyclodextrins and derivatives thereof, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Dosage forms for the topical or transdermal administration include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be required.

The ointments, pastes, creams and gels may contain, in addition to an active compound, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an active compound, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the active compound in the proper medium. Absorption enhancers can also be used to increase the flux of the compound across the skin. The rate of such flux can be controlled by either providing a rate controlling membrane or dispersing the compound in a polymer matrix or gel.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion. Pharmaceutical compositions suitable for parenteral administration comprise one or more active compounds in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsulated matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

For use in the methods of this invention, active compounds can be given per se or as a pharmaceutical composition containing, for example, 0.1 to 99.5% (more preferably, 0.5 to 90%) of active ingredient in combination with a pharmaceutically acceptable carrier.

Methods of introduction may also be provided by rechargeable or biodegradable devices. Various slow release polymeric devices have been developed and tested in vivo in recent years for the controlled delivery of drugs, including proteinaceous biopharmaceuticals. A variety of biocompatible polymers (including hydrogels), including both biodegradable and non-degradable polymers, can be used to form an implant for the sustained release of a compound at a particular target site.

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound or combination of compounds employed, or the ester, salt or amide thereof, the route of administration, the time of administration, the rate of excretion of the particular compound(s) being employed, the duration of the treatment, other drugs, compounds and/or materials used in combination with the particular compound(s) employed, the age, sex, weight, condition, general health and prior medical history of the patient being treated, and like factors well known in the medical arts.

A physician or veterinarian having ordinary skill in the art can readily determine and prescribe the therapeutically effective amount of the pharmaceutical composition required. For example, the physician or veterinarian could start doses of the pharmaceutical composition or compound at levels lower than that required in order to achieve the desired therapeutic effect and gradually increase the dosage until the desired effect is achieved. By “therapeutically effective amount” is meant the concentration of a compound that is sufficient to elicit the desired therapeutic effect. It is generally understood that the effective amount of the compound will vary according to the weight, sex, age, and medical history of the subject. Other factors which influence the effective amount may include, but are not limited to, the severity of the patient's condition, the disorder being treated, the stability of the compound, and, if desired, another type of therapeutic agent being administered with the compound of the invention. A larger total dose can be delivered by multiple administrations of the agent. Methods to determine efficacy and dosage are known to those skilled in the art (Isselbacher et al. (1996) Harrison's Principles of Internal Medicine 13 ed., 1814-1882, herein incorporated by reference).

In general, a suitable daily dose of an active compound used in the compositions and methods of the invention will be that amount of the compound that is the lowest dose effective to produce a therapeutic effect. Such an effective dose will generally depend upon the factors described above.

If desired, the effective daily dose of the active compound may be administered as one, two, three, four, five, six or more sub-doses administered separately at appropriate intervals throughout the day, optionally, in unit dosage forms. In certain embodiments of the present invention, the active compound may be administered two or three times daily. In preferred embodiments, the active compound will be administered once daily.

The patient receiving this treatment is any animal in need, including primates, in particular humans; and other mammals such as equines, cattle, swine, sheep, cats, and dogs; poultry; and pets in general.

In certain embodiments, compounds of the invention may be used alone or conjointly administered with another type of therapeutic agent.

The present disclosure includes the use of pharmaceutically acceptable salts of compounds of the invention in the compositions and methods of the present invention. In certain embodiments, contemplated salts of the invention include, but are not limited to, alkyl, dialkyl, trialkyl or tetra-alkyl ammonium salts. In certain embodiments, contemplated salts of the invention include, but are not limited to, L-arginine, benenthamine, benzathine, betaine, calcium hydroxide, choline, deanol, diethanolamine, diethylamine, 2-(diethylamino)ethanol, ethanolamine, ethylenediamine, N-methylglucamine, hydrabamine, 1H-imidazole, lithium, L-lysine, magnesium, 4-(2-hydroxyethyl)morpholine, piperazine, potassium, 1-(2-hydroxyethyl)pyrrolidine, sodium, triethanolamine, tromethamine, and zinc salts. In certain embodiments, contemplated salts of the invention include, but are not limited to, Na, Ca, K, Mg, Zn or other metal salts. In certain embodiments, contemplated salts of the invention include, but are not limited to, 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acid, 1-ascorbic acid, 1-aspartic acid, benzenesulfonic acid, benzoic acid, (+)-camphoric acid, (+)-camphor-10-sulfonic acid, capric acid (decanoic acid), caproic acid (hexanoic acid), caprylic acid (octanoic acid), carbonic acid, cinnamic acid, citric acid, cyclamic acid, dodecylsulfuric acid, ethane-1,2-disulfonic acid, ethanesulfonic acid, formic acid, fumaric acid, galactaric acid, gentisic acid, d-glucoheptonic acid, d-gluconic acid, d-glucuronic acid, glutamic acid, glutaric acid, glycerophosphoric acid, glycolic acid, hippuric acid, hydrobromic acid, hydrochloric acid, isobutyric acid, lactic acid, lactobionic acid, lauric acid, maleic acid, 1-malic acid, malonic acid, mandelic acid, methanesulfonic acid, naphthalene-1,5-disulfonic acid, naphthalene-2-sulfonic acid, nicotinic acid, nitric acid, oleic acid, oxalic acid, palmitic acid, pamoic acid, phosphoric acid, proprionic acid, 1-pyroglutamic acid, salicylic acid, sebacic acid, stearic acid, succinic acid, sulfuric acid, 1-tartaric acid, thiocyanic acid, p-toluenesulfonic acid, trifluoroacetic acid, and undecylenic acid acid salts.

The pharmaceutically acceptable acid addition salts can also exist as various solvates, such as with water, methanol, ethanol, dimethylformamide, and the like. Mixtures of such solvates can also be prepared. The source of such solvate can be from the solvent of crystallization, inherent in the solvent of preparation or crystallization, or adventitious to such solvent.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like.

### EXAMPLES

The invention now being generally described, it will be more readily understood by reference to the following examples which are included merely for purposes of illustration of certain aspects and embodiments of the present invention, and are not intended to limit the invention.

### Example 1: Exemplary Solvent Study

A GC method (details are listed in Table 1) was developed to determine the ratio of endo, 3, to exo, 3-1, products resulting from the Diels-Alder reaction between (E)-3-methyl-1-nitrobut-1-ene, 1, and cyclopentadiene, 2 (Scheme 1) and identify a condition with improved selectivity for the desired 3. Retention times of the products of interest were verified by acquiring the gas chromatogram of authentic standards. The retention time, as determined using the conditions reported in Table 1, of the endo product was 13.0 min. The retention time, as determined using the conditions reported in Table 1, of the exo_product was 12.7 min. This resolution was sufficient to determine the ratio of endo to exo products. The conversion of starting material to products was not quantified at this stage of the study. A series of solvent conditions were profiled with 50 mg of 1 and 5 equivalents of freshly cracked cyclopentadiene, 2, at 40° C. with the volume of solvent and time indicated in the table 1. For select conditions, increased temperature was also profiled, as indicated. The results of this study demonstrate that fluorinated alcohols have the unique ability to favor endo product formation with the ratio of 3:3-1 increasing with increasing degrees of fluorination. For some examples, the effect of raising the temperature was probed. In every case, increased temperature increased the ratio of exo product relative to endo product. This effect was pronounced for TFA and DMF and significant enrichment in the exo product 3-1 was observed in these cases. Scheme 1. Diels-Alder reaction between 1 and 2.

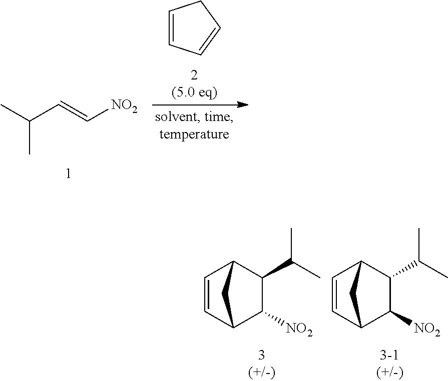

TABLE 1
GC method for monitoring ratio of endo to exo products

| System: | GC2010Plus (CA-022) | | |
|---|---|---|---|
| Column name: | DB-624 | | |
| Column size: | 30 m*0.320 mm*1.80 um | | |
| Injection mode: | Split | | |
| SPL Temperature: | 260° C. | | |
| Flow control mode | Linear velocity | | |
| Pressure: | 48.0 kPa | | |
| Column flow: | 1.02 mL/min | | |
| Split ratio: | 15 | | |
| DET#4 Temperature: | 300° C. | | |
| Makeup gas: | N₂ | | |
| Makeup flow: | 30.0 mL/min | | |
| Flow(H2): | 40.0 mL/min | | |
| Flow(N2): | 400.0 mL/min | | |
| Oven Temperature: | 120° C. | | |
| Equilibration time: | 3.0/min | | |
| Injection: | 0.5 mL | | |
| Run time: | 17 min | | |
| | Rate | Temperature | Hold time |
| Temperature | N/A | 120 | 2 |
| program | 12 | 240 | 5 |

TABLE 2
Solvents and conditions examined by GC to
determine endo and exo ratio of products

| | Solvent | | | | |
|---|---|---|---|---|---|
| | excess | Additive | Time | Temp. | Endo:exo |
| Solvent | (volumes) | volumes | (h) | (° C.) | (GC) |
| AcOH | 1 | | 18 | 40 | 2:1 |
| None | NA | | 18 | 40 | 1.4:1 |
| Toluene | 10 | AcOH | 18 | 40 | 1.54:1 |
| | | (1 V) | | | |
| Toluene | 10 | AcOH | 18 | 100 | 1.37:1 |
| | | (1 V) | | | |
| THF | 10 | AcOH | 18 | 40 | 1.67:1 |
| | | (1 V) | | | |
| MeCN | 10 | | 18 | 40 | 2.21:1 |
| MeOH | 10 | | 18 | 40 | 2.45:1 |
| EtOH | 10 | | 18 | 40 | 2.02:1 |
| iPrOH | 10 | | 18 | 40 | 1.96:1 |
| DMF | 10 | AcOH | 18 | 40 | 2.5:1 |
| | | (1 V) | | | |
| DMF | 10 | AcOH | 18 | 80 | 0.86:1 |
| | | (1 V) | | | |
| DMF | 10 | AcOH | 18 | 100 | 0.27:1 |
| | | (1 V) | | | |
| H₂O | 10 | AcOH | 18 | 40 | 1.53:1 |
| | | (1 V) | | | |
| TFA | 1 | | 72 | 40 | 1.77:1 |
| TFA | 1 | | 18 | 100 | 0.17:1 |
| EtCOOH | 1 | | 72 | 40 | 1.85:1 |
| tBuCOOH | 1 | | 72 | 40 | 1.49:1 |
| TsOH | 1 | | 72 | 40 | No product |
| PhCOOH | 1 | | 72 | 40 | 1.27:1 |
| NEt₃ | 20 | | 18 | 40 | No product |
| DEA | 20 | | 18 | 40 | No product |
| Pyr | 20 | | 18 | 40 | 1.7:1 |
| DBU | 20 | | 18 | 40 | No product |
| CF₂HCH₂OH | 20 | | 18 | 40 | 6.36:1 |
| CF₂HCH₂OH | 20 | | 72 | 40 | 7.45:1 |
| (CF₃)₂CHOH | 20 | | 18 | 40 | 21:1 |
| (CF₃)₂CHOH | 20 | | 18 | 80 | 11.6:1 |

### Example 2: Exemplary Improved Scalable Synthesis of BRD4780

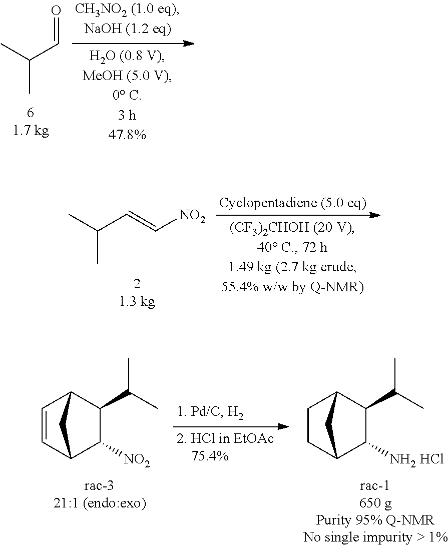

(E)-3-methyl-1-nitrobut-1-ene, 2. To a stirred solution of 6 (1.70 kg, 23.4 mol, 1.00 eq) and CH₃NO₂ (1.40 kg, 23.4 mol, 1.00 eq) in MeOH (8.50 L) was added a solution of NaOH (1.13 kg, 28.2 mol, 1.20 eq) in H₂O (1.70 L) dropwise by dropping funnel at 0° C. (temperature range is −1 to 1° C.) for 5 hrs. After addition, the reaction slowly turned to room temperature (20° C.) in 3 hrs. The reaction was stirred at 20° C. for 12 hrs. TLC (Petroleum ether/EtOAc=3/1, R_{f}=0.60, KMnO₄) indicated Compound 6 was consumed completely. Water (10.0 L) was added in one portion and the temperature turned to 23° C. from 20° C., the reaction turned into a clear solution. The clear solution was then slowly poured into a solution of HCl (conc. HCl (13.6 L) in ice-water (20.4 L)) at room temperature (20° C.) and stirred for 15 min. The aqueous layer was extracted with DCM (10.0 L×3). The combined organic was dried over anhydrous Na₂SO₄ (˜2.00 kg), filtered and concentrated. The residue was purified by column chromatography (SiO₂, 3.50 kg, 200-300 mesh), Petroleum ether/EtOAc=1000/1 to 1/1, total −45.0 L of fraction), the fraction of column was concentrated at 30° C. under vacuum, and the evaporated solvent was re-concentration. Combined the products to give Compound 2 (1.30 kg, 47.8% yield) as a yellow oil. GC: 86.1% purity. (Synthesis, 1995, 12, 1545-1548). ¹H NMR (400 MHz, CDCl₃) δ: 7.25 (dd, J=13.5, 7.0 Hz, 1H), 6.93 (dd, J=13.4, 1.5 Hz, 1H), 2.58 (ddhept., J=13.6, 6.8, 1.5 Hz, 1H), 1.14 (d, J=6.80 Hz, 6H). There is the opportunity to optimize this step and perform a purification by distillation or other means. DSC analysis of 2: onset: 202.0° C., peak: 236.0° C., end: 270.0° C., area: −2098 J/g.

rac-5-isopropyl-6-nitrobicyclo[2.2.1]hept-2-ene, 3. To a solution of 2 (1.30 kg, 11.2 mol, 1.00 eq) in HFIP (26.0 L) was added in one portion freshly cracked cyclopenta-1,3-diene (3.72 kg, 56.2 mol, 5.00 eq, fresh cyclopenta-1,3-diene was cracked at 220° C., and collected the fraction when the temperature is 145-148° C. by distillation). The reaction mass was stirred at 40° C. for 12 hrs. TLC (Petroleum ether/EtOAc=10/1, R_{f}=0.65, PMA) showed that the reaction was completed, GC showed that the ratio of the endo and exo was ˜21:1. The reaction mixture was cooled to 25° C., and then poured into ice-water (w/w=1/1) (10.0 L), the temperature of mixture was up to 27° C. The aqueous phase was extracted with EtOAc for two rounds (10.0 L for the first round, 3.00 L for the second round). The combined organic phase was washed with brine (5.00 L), dried with anhydrous Na₂SO₄ (4.20 kg), filtered and concentrated in vacuum at 40° C. Considering the low boiling point of Compound 3 (76-80° C. @ 3 mbar, Broad Institute data on pure 3), the evaporated solvent was analyzed by TLC and almost no Compound 3 was detected. Compound 3 (2.70 kg, crude) was obtained as a yellow oil. Q-NMR analysis using benzyl benzoate as a standard indicated that the crude material contained 55.4 w/w % of Compound 3. A crude yield of 72.8% is determined assuming pure 2. 1H-NMR obtained on a sample purified by HPLC. ¹H NMR (400 MHz, Chloroform-d) δ: 6.47 (dd, J=5.7, 3.3 Hz, 1H), 5.96 (dd, 5.7, 2.7 Hz, 1H), 4.65 (dd, 3.9, 3.9 Hz, 1H), 3.47 (m, 1H), 2.88 (m, 1H), 1.81 (ddd, 10.5, 3.9, 2.0 Hz, 1H), 1.60-1.48 (3H), 1.03 (d, J=6.4 Hz, 3H), 1.02 (d, J=6.4 Hz, 3H).

GC data: RTs (determined with authentic standard): tran-iPr-NO₂ alkene 4.2 min; CPD, 5.0 min; 3 exo, 8.5 min; 3 endo 8.8 min. DSC of 3: Two exotherms observed (exotherm 1: onset: 108.1° C., peak: 143.3° C., end: 176.9° C., area: −47.7 J/g; exotherm 2: onset: 249.5° C., peak 282.6° C., end: 345.3° C., area: −362.4 J/g)

GC Protocol

| Method name: | 250_1.5_100_50-250_280 |
|---|---|
| Instrument: | Shimadzu GC2030 |
| Column: | Agilent J&W HP-5MS |
| | 30 M*0.25 mmID*0.25 um film |
| | thickness |
| Injector Temp.: | 250° C. |
| Split Ratio: | 100:1 |
| Control Mode: | Linear Velocity |
| Flow rate: | 1.5 mL/min |
| Carrier Gas: | N₂ |
| Detector Temp.: | 280° C. |
| | Rate(° C./min) |
| Column Temp. | 30 |
| Program | 10 |
| GC Run Time: | 19.67 min |

Rac-BRD4780 HCL. To a solution of Compound 3 (800 g, 2.43 mol, 55% QNMR purity, 1.00 eq) in MeOH (8 L) in 10 L autoclave was added Pd/C (240 g, 10% purity) under Ar. The suspension was degassed under vacuum and purged with H₂ for three times. The mixture was stirred under H₂ (50 psi) at 25° C. for 47 hrs. (Note: the H₂ is replenished every 0.5 h during daytime based on the safety guideline). Taking a small amount for analytical characterization, the sample was filtered directly for GC detection, the sample was concentrated in vacuum for HNMR detection. Both GC and HNMR indicated the reaction was complete. Care must be taken to complete the hydrogenation as the hydroxylamine intermediate can accumulate during the course of the reaction. Another batch with 700 g scale (2.12 mol, 55% QNMR purity, 1.00 eq) in 10 L autoclave was carried out following the procedure of 800 g batch.

Two batches of 700 g and 800 g (total 1.50 kg, 4.55 mol, 55% QNMR purity, 1.00 eq) were combined for work up. The reaction mixture was filtered through celite (700 g) and the filter cake was washed with MeOH (12.0 L), and then HCl/EtOAc (4 M, 2.00 L) was added directly to the filtrate, the filtrate was concentrate in vacuum at 40° C. to give the crude product (1.60 kg) as a slurry. The residual was suspended with EtOAc (2.00 L), and stirred for 1 h at room temperature (20° C.), then filtered and washed the filter cake with EtOAc (500 mL), the filter cake was collected and dried under vacuum at 40° C. to give BRD4780 (650 g, 3.43 mol, 75.4% yield) as a white solid. Q-NMR analysis indicated that BRD4780 HCl was present in 94.9% w/w. Chiral GC was required to separate the endo from the exo products. The GC chromatogram demonstrated >99% purity by peak area. BRD4780-HCl: ¹H NMR (400 MHz, Chloroform-d) δ: 8.51 (s, 3H), 3.20 (ddq(broad), J=4.8, −3, −5 Hz, 1H, C₂-exo-H), 2.63 (dd, J=4.8, −4.5 Hz, 1H, C₁-bridgehead), 2.20 (d, J=3.6 Hz, 1H, C₄-bridgehead), 1.94-1.88 (m, 1H), 1.45-1.62 (m, 5H), 1.27 (d, J=10.0 Hz, 1H), 1.17 (m, 1H), 1.03 (d, J=6.8 Hz, 3H), 0.95 (d, J=6.8 Hz, 3H). 13C NMR (101 MHz, Chloroform-d) δ 57.5, 55.4, 40.4, 39.5, 36.0, 31.4, 29.7, 21.6, 21.2, 20.7. HRMS (ESI/Q-TOF) m/z: [M+H]⁺ Calcd for C₁₀H₁₉N₂+H: 154.1595; Found: 154.1587. GC for monitoring hydrogenation; RT: 6.61 min=hydroxyl amine; RT: 8.60=desired. Note: a chiral GC method was used to separate the endo (major; rt: 14.3 and 14.7 min) from the exo impurities (minor; rt: 14.1 and 15.4 min).

GC Protocol

| Column: | Agilent J&W CYCLOSIL-B 30 m × 0.25 mm | | |
|---|---|---|---|
| | ID × 0.25 μm film thickness | | |
| Injector Temp.: | 200° C. | | |
| Split Ratio: | 30:1 | | |
| Control Mode: | Pressure | | |
| Flow: | 0.8 mL/min | | |
| Carrier Gas: | He | | |
| Detector: | Flame Ionization Detector (FID) | | |
| Detector temp.: | 240° C. | | |
| Makeup (He) Flow: | 30 mL/min | | |
| H₂ Flow: | 40 mL/min | | |
| Air Flow: | 400 mL/min | | |
| | Rate | Temp. | Hold Time |
| Column Temp. Program: | | 120° C. | 30 min |

| | |
|---|---|
| GC Run Time: | 30 min |

### Example 3: Preparation of Exemplary Compounds of the Disclosure

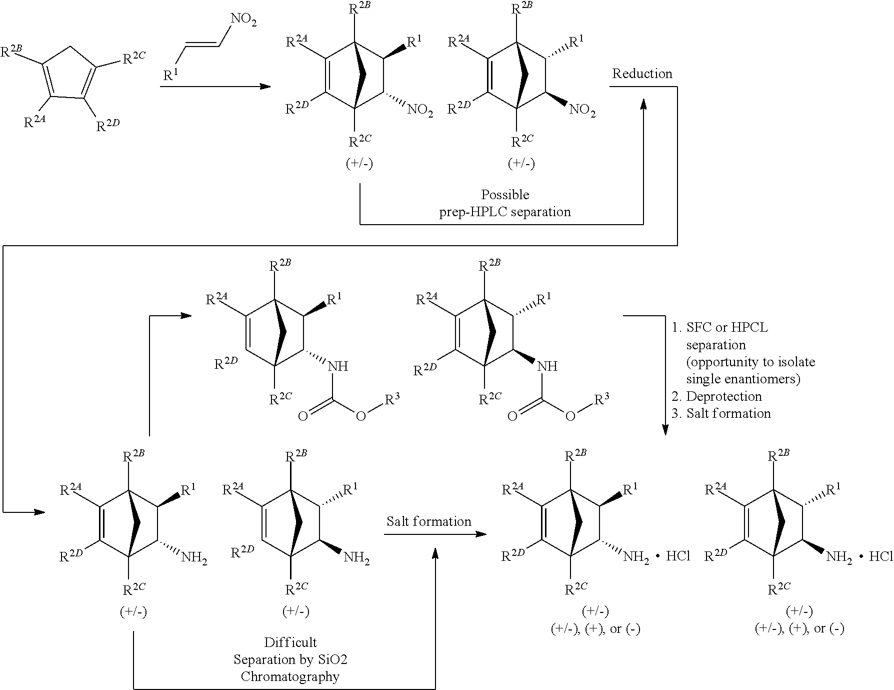

BRD4780 analogs with C₂ and C₃ substituents arranged trans are prepared following the route described as route A. Diels-alder reaction with cracked cyclopentadiene and a trans alkene delivered the corresponding C₅-C₆ trans substituted norborene as a racemic mixture of the C₆-endo and C₆-exo products. The ratio of C₆-endo and C₆-exo products was dependent on the identity of the R group and the solvent and temperature employed. Separation of the endo and exo products was often times challenging but could be affected by preparative reverse-phase chromatography at the norborene stage (PDA detector), careful normal phase silica gel chromatography following reduction to the norborane, or by preparative reverse phase HPLC or SFC with detection and separation enabled by incorporation of an amino protecting group (typically Cbz) followed by deprotection. Assignments of endo and exo products are supported by chemical shift and proton-proton coupling constants of the bridgehead protons as well as COSY NMR that displays a cross-peak of the exo-proton with the neighboring bridgehead proton (similar COSY crosspeak is not observed between the bridgehead proton and the neighboring endo proton). All examples that were tested as a single isomer were prepared by protection of the amino group by protecting group with UV absorbance and separated using chiral SFC chromatography.

### Representative Procedure for Endo-Amino-Exo-Alkyl/Aryl Using Diels-Alder Reaction (Representative Procedure A)

### Synthesis of (1R,2R,3R,4S)-3-ethylbicyclo[2.2.1]heptan-2-amine hydrochloride (105-P1), (1S,2S,3R,4R)-3-ethylbicyclo[2.2.1]heptan-2-amine hydrochloride (105-P2)

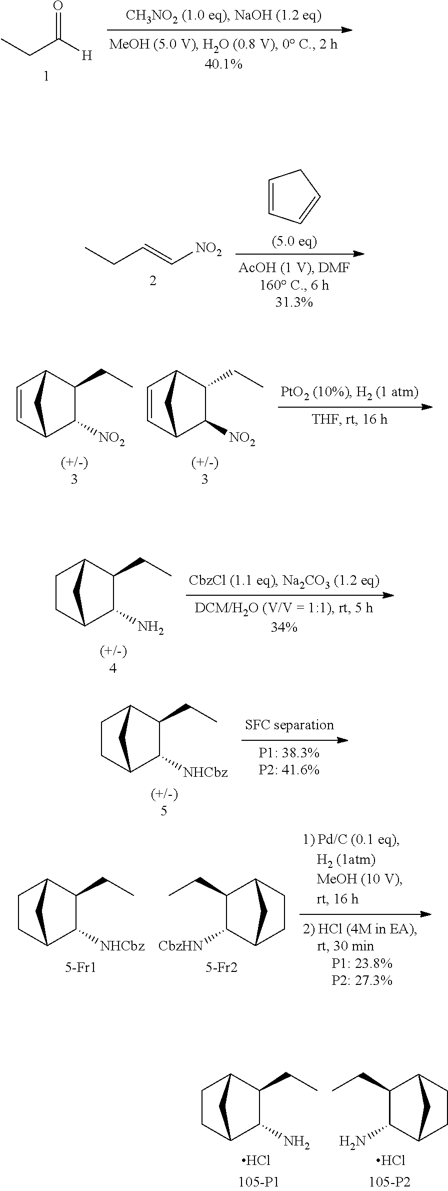

Step 1. To a stirred solution of cyclopropanecarbaldehyde (10 g, 172.3 mmol, 1 eq) and nitromethane (10.5 g, 172.3 mmol, 1 eq) in methanol (100 mL) at 0° C. was added dropwise a solution of sodium hydroxide (8.3 g, 206.8 mmol, 1.2 eq) in water (14 mL). The reaction mixture was then stirred at 0° C. for 1 h and a white suspension was obtained during the process. TLC was done to detect the process of the reaction. Once the reaction finished, water (50 mL) was added to the suspension and a clear solution was obtained, which was then poured into a solution of HCl (conc. HCl 40 mL and water 70 mL) and stirred for 15 min. The aqueous layer was extracted with DCM (5×50 mL). All the organic phases were combined, dried over Na₂SO₄, and filtered. The filtration was concentrated under vacuum to provide the crude, which was purified by silica column chromatography eluting with 2% EtOAc in PE to give (E)-1-nitrobut-1-ene (7.0 g, 69.1 mmol, 40.1%) as a yellow oil. 1H NMR (400 MHz, CDCl₃) δ 7.32 (dt, J=24.2, 8.7 Hz, 1H), 6.98 (dt, J=13.4, 1.7 Hz, 1H), 2.41-2.26 (m, 2H), 1.15 (t, J=7.4 Hz, 3H).

Step 2. To a solution of (E)-1-nitrobut-1-ene (4.2 g, 42 mmol, 1 eq) in AcOH (5 mL) and DMF (5 mL) was added cyclopenta-1,3-diene (14 g, 210 mmol, 5 eq) and the reaction mixture was stirred at 140° C. for 8 h in a sealed tube. Once TLC showed the reaction finished, the mixture was cooled to room temperature and water (50 mL) was added. The reaction mixture was then extracted with EA (3×50 mL). All the organic phases were collected, washed with brine 10 mL, dried over Na₂SO₄, and filtered. The filtration was concentrated under vacuum to give the crude, which was then purified by prep-HPLC eluting with 0-90% ACN in water (0.1% TFA) to give (1S,4R,5R,6R)-5-ethyl-6-nitrobicyclo[2.2.1]hept-2-ene (2.2 g, 13.17 mmol, 31.3%) as a yellow oil. 1H NMR (400 MHz, CDCl₃) δ 6.45 (dd, J=5.6, 3.2 Hz, 1H), 6.00 (dd, J=5.7, 2.7 Hz, 1H), 4.52 (t, J=3.7 Hz, 1H), 3.45 (s, 1H), 2.71 (d, J=1.4 Hz, 1H), 2.01 (tdd, J=7.7, 3.5, 1.7 Hz, 1H), 1.75-1.42 (m, 4H), 1.04 (t, J=7.4 Hz, 3H).

Step 3. To a stirred solution of rac-5-exo-ethyl-6-endo-nitrobicyclo[2.2.1]hept-2-ene (1.3 g, 7.8 mmol, 1 eq) in THE (10 mL) was added PtO₂/C (130 mg, 0.1 wt), and the reaction mixture was stirred at room temperature under H₂ atmosphere (1 atm) overnight. Once LCMS showed the reaction finished, reaction mixture was filtered and the filtration was concentrated to give rac-3-exo-ethylbicyclo[2.2.1]heptan-2-endo-amine (700 mg crude) as a yellow oil. LCMS [M+H]: 140.2.

Step 4. To a solution of (1R,2R,3R,4S)-3-ethylbicyclo[2.2.1]heptan-2-amine (700 mg, 6.5 mmol, 1.0 eq) in solvent (THF/H₂O=2:1; 10 mL in total) was added sodium bicarbonate (1.1 g, 13.0 mmol, 2.0 eq) and benzyl chloroformate (1.7 g, 9.75 mmol, 1.5 eq). The whole reaction mixture was then stirred at room temperature overnight. Once LCMS showed the reaction finished, solvent was removed under vacuum to get a residue, which was diluted and extracted with EtOAc (5×50 mL). The organic phases were collected, washed with brine (2×20 mL), dried over Na₂SO₄, and filtered. The filtration was then concentrated under vacuum to get the crude, which was purified by prep-HPLC eluting with 0-90% ACN in water (0.1% TFA) to give rac-benzyl (3-exo-ethylbicyclo[2.2.1]heptan-2-endo-yl)carbamate (600 mg, 34%) as a colorless semi-solid. 1H NMR (400 MHz, CDCl₃) δ 7.39-7.31 (m, 5H), 5.15-5.04 (m, 2H), 3.48 (d, J=5.5 Hz, 1H), 2.41 (s, 1H), 1.95 (s, 1H), 1.61-1.30 (m, 7H), 1.23-1.13 (m, 2H), 0.88 (dd, J=9.3, 5.3 Hz, 3H), 0.75 (dd, J=12.2, 5.4 Hz, 1H).

Step 5. SFC separation was carried out for compound 6 (600 mg). The SFC separation information are shown as following:

Analytical separation method: Instrument: Waters UPCC, Column: ChiralPak AY, 250×4.6 mm, 5 μm, Mobile phase: A for C₀₂ and B for EtOH (0.04% DEA), Gradient: B 0-40%, Flow rate: 2.8 mL/min, Back pressure: 100 bar, Column temperature: 35° C., Wavelength: 214 nm

Preparative separation method: Instrument: Waters SFC80, Column: ChiralPak AY, 250×25 mm, 10 μm, Mobile phase: A for C₀₂ and B for EtOH (0.04% DEA), Gradient: B 40%, Flow rate: 2.8 ml/min, Back pressure: 100 bar, Column temperature: 35° C., Wavelength: 214 nm, Cycle time: 7 min, Sample preparation: Compound was dissolved in 15 mL methanol, Injection: 3 ml per injection.

After separation, benzyl ((1R,2R,3R,4S)-3-ethylbicyclo[2.2.1]heptan-2-yl)carbamate (compound 5-Fr1; 230 mg, 38.3%, 100% ee) was obtained as a colorless solid, and benzyl ((1S,2S,3S,4R)-3-ethylbicyclo[2.2.1]heptan-2-yl)carbamate (compound 5-Fr2; 250 mg, 41.6%, 98.64% ee) was obtained as a colorless solid. The absolute configuration of the compounds is unknown and peak 1 is arbitrarily assigned as C1-R.

Step 6. To a solution of benzyl ((1R,2R,3R,4S)-3-ethylbicyclo[2.2.1]heptan-2-yl)carbamate (230 mg, 0.84 mmol, 1.0 eq) in EtOAc (5.0 mL) was added Pd/C (23 mg, 10% wt), and the reaction mixture was stirred at room temperature under H₂ atmosphere (1 atm) overnight. Once LCMS showed the reaction finished, solvent was removed to get the crude, which was then purified by prep-HPLC eluting with 0-90% ACN in water (0.1% TFA), and substituted by HCl to give 105-P1 (35.08 mg, 23.8%) as a white solid. LCMS [M+H]: 140.3. 1H NMR (400 MHz, CD3OD) δ 3.00 (t, J=4.0 Hz, 1H), 2.45 (s, 1H), 2.13 (d, J=3.7 Hz, 1H), 1.76-1.65 (m, 1H), 1.57 (dt, J=11.0, 8.6 Hz, 3H), 1.38 (m, 4H), 1.18-1.10 (m, 1H), 0.97 (t, J=7.4 Hz, 3H).

To a solution of benzyl ((1S,2S,3S,4R)-3-ethylbicyclo[2.2.1]heptan-2-yl)carbamate (250 mg, 0.91 mmol, 1.0 eq) in EtOAc (5.0 mL) was added Pd/C (25 mg, 10% wt), and the reaction mixture was stirred at room temperature under H₂ atmosphere (1 atm) overnight. Once LCMS showed the reaction finished, solvent was removed to get the crude, which was then purified by prep-HPLC eluting with 0-90% ACN in water (0.1% TFA), and substituted by HCl to give 105-P2 (43.47 mg, 27.3%) as a white solid. LCMS [M+H]: 140.3 1H NMR (400 MHz, CD3OD) δ 3.00 (t, J=3.7 Hz, 1H), 2.45 (s, 1H), 2.13 (d, J=3.8 Hz, 1H), 1.76-1.65 (m, 1H), 1.63-1.50 (m, 3H), 1.48-1.29 (m, 4H), 1.14 (ddd, J=10.8, 7.5, 3.0 Hz, 1H), 0.97 (t, J=7.4 Hz, 3H).

### Representative Procedure for Exo-Amino-Endo-Alkyl/Aryl Using Diels-Alder Reaction (Representative Procedure B)

### Synthesis of (1R,2S,3S,4S)-3-cyclopropylbicyclo[2.2.1]heptan-2-amine hydrochloride (110-P1), (1S,2R,3R,4R)-3-cyclopropylbicyclo[2.2.1]heptan-2-amine hydrochloride (110-P2)

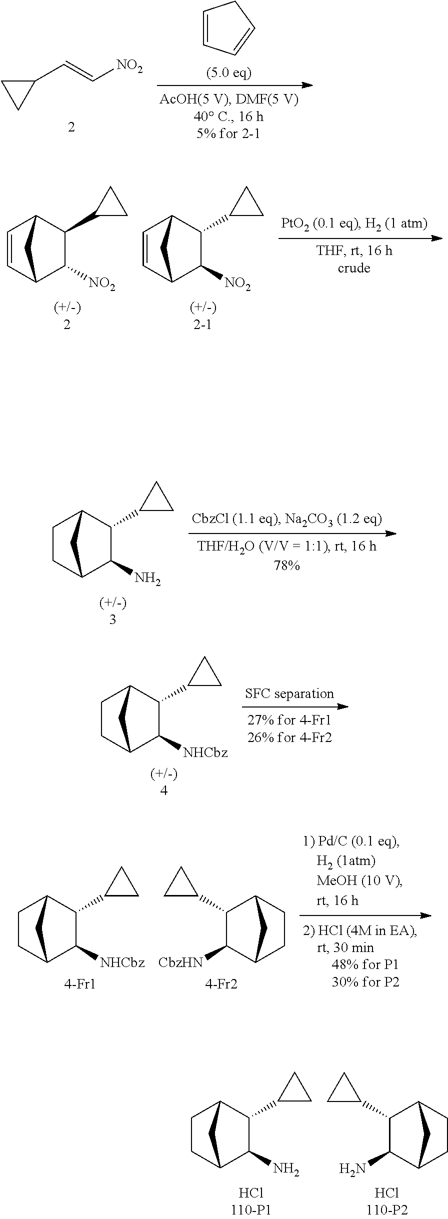

Step 1. To a solution of (E)-(2-nitrovinyl)cyclopropane (2.0 g, 17.7 mmol, 1 eq) in AcOH (5 mL) and DMF (5 mL) was added cyclopenta-1,3-diene (6.0 g, 88.5 mmol, 5 eq) and the reaction mixture was stirred at 140° C. for 8 h in a sealed tube. Once TLC showed the reaction finished, the mixture was cooled to room temperature and water (50 mL) was added. The reaction mixture was then extracted with EA (3×50 mL). All the organic phases were collected, washed with brine 10 mL, dried over Na₂SO₄, and filtered. The filtration was concentrated under vacuum to give the crude, which was then purified by prep-HPLC eluting with 0-90% ACN in water (0.1% TFA) to give rac-5-endo-cyclopropyl-6-exo-nitrobicyclo[2.2.1]hept-2-ene (150 mg, 7.26 mmol, 5%) as a yellow oil. 1H NMR (400 MHz, CDCl₃) δ 6.42 (dd, J=5.6, 2.9 Hz, 1H), 6.19 (dd, J=5.6, 3.3 Hz, 1H), 4.13 (dd, J=3.9, 1.5 Hz, 1H), 3.33 (d, J=1.4 Hz, 1H), 2.92 (s, 1H), 1.93 (d, J=9.1 Hz, 1H), 1.84 (dt, J=9.2, 3.6 Hz, 1H), 1.68 (dd, J=9.1, 1.5 Hz, 1H), 0.51-0.38 (m, 3H), 0.26 (dddd, J=10.7, 9.1, 7.3, 3.4 Hz, 2H).

Step 2. To a stirred solution of rac-5-endo-cyclopropyl-6-exo-nitrobicyclo[2.2.1]hept-2-ene (150 mg, 0.84 mmol, 1 eq) in THE (10 mL) was added PtO₂/C (50 mg, 0.1 wt), and the reaction mixture was stirred at room temperature under H₂ atmosphere (1 atm) overnight. Once LCMS showed the reaction finished, reaction mixture was filtered and the filtration was concentrated to get rac-3-endo-cyclopropylbicyclo[2.2.1]heptan-2-endo-amine (130 mg crude) as a yellow oil. LCMS [M+H]: 152.2.

Step 3. To a solution of rac-3-endo-cyclopropylbicyclo[2.2.1]heptan-2-endo-amine (130 mg, 0.85 mmol, 1.0 eq) in THF:H₂O=2:1 (10 mL) was added sodium bicarbonate (193 mg, 2.6 mmol, 2.0 eq) and benzyl chloroformate (332 mg, 1.95 mmol, 1.5 eq). The whole reaction mixture was then stirred at room temperature overnight. Once LCMS showed the reaction finished, solvent was removed under vacuum to get a residue, which was diluted and extracted with EtOAc (3×50 mL). The organic phases were collected, washed with brine (2×20 mL), dried over Na₂SO₄, and filtered. The filtration was then concentrated under vacuum to get the crude, which was purified by prep-HPLC eluting with 0-90% ACN in water (0.1% TFA) to give rac-benzyl (3-endo-cyclopropylbicyclo[2.2.1]heptan-2-exo-yl)carbamate (190 mg, 78%) as a colorless semi-solid. 1H NMR (400 MHz, CDCl₃) δ 7.39-7.33 (m, 5H), 5.08 (s, 2H), 3.21 (s, 1H), 2.18 (s, 2H), 1.70-1.49 (m, 3H), 1.37-1.20 (m, 5H), 0.67 (s, 2H), 0.55-0.40 (m, 2H), 0.18 (dd, J=9.0, 4.5 Hz, 1H).

Step 4. SFC separation was carried out for compound 4 (800 mg). The SFC separation information are shown as following:

Analytical separation method: Instrument: Waters UPCC, Column: ChiralPak AY, 250×4.6 mm, 5 μm, Mobile phase: A for CO₂ and B for EtOH (0.04% DEA), Gradient: B 0-40%, Flow rate: 2.8 mL/min, Back pressure: 100 bar, Column temperature: 35° C., Wavelength: 214 nm

Preparative separation method: Instrument: Waters SFC80, Column: ChiralPak AY, 250×25 mm, 10 μm, Mobile phase: A for CO₂ and B for EtOH (0.04% DEA), Gradient: B 40%, Flow rate: 70 g/min, Back pressure: 100 bar, Column temperature: 35° C., Wavelength: 214 nm, Cycle time: 10 min, Sample preparation: Compound was dissolved in 15 mL methanol, Injection: 3 ml per injection.

After separation, benzyl ((1R,2S,3S,4S)-3-cyclopropylbicyclo[2.2.1]heptan-2-yl)carbamate (compound 4-Fr1; 52 mg, 27%, 100% ee) was obtained as a colorless solid, and benzyl ((1S,2R,3R,4R)-3-cyclopropylbicyclo[2.2.1]heptan-2-yl)carbamate (compound 4-Fr2; 50 mg, 26%, 100% ee) was obtained as a colorless solid. The absolute configuration of the compounds is unknown and peak 1 is arbitrarily assigned as C1-R.

Step 5. To a solution of benzyl ((1R,2S,3S,4S)-3-cyclopropylbicyclo[2.2.1]heptan-2-yl)carbamate (52 mg, 0.18 mmol, 1.0 eq) in EtOAc (5.0 mL) was added Pd/C (5 mg, 10% wt), and the reaction mixture was stirred at room temperature under H₂ atmosphere (1 atm) overnight. Once LCMS showed the reaction finished, catalyst was filtered through celite and the filtration was concentrated to get the crude, which was then purified by prep-HPLC eluting with 0-90% ACN in water (0.1% TFA), and substituted by HCl to give 110-P1 (16.23 mg, 48%) as a white solid. LCMS [M+H]: 152.2. 1H NMR (400 MHz, CD3OD) δ 2.81 (dd, J=4.0, 1.3 Hz, 1H), 2.35-2.27 (m, 2H), 1.78-1.68 (m, 2H), 1.63 (d, J=10.6 Hz, 1H), 1.49-1.37 (m, 2H), 1.31-1.26 (m, 1H), 0.97 (dd, J=8.3, 4.2 Hz, 1H), 0.76-0.67 (m, 1H), 0.60-0.50 (m, 2H), 0.33 (td, J=9.2, 4.8 Hz, 1H), 0.10 (dt, J=9.6, 4.6 Hz, 1H).

To a solution of benzyl ((1S,2R,3R,4R)-3-cyclopropylbicyclo[2.2.1]heptan-2-yl)carbamate (50 mg, 0.18 mmol, 1.0 eq) in EtOAc (5.0 mL) was added Pd/C (5 mg, 10% wt), and the reaction mixture was stirred at room temperature under H₂ atmosphere (1 atm) overnight. Once LCMS showed the reaction finished, catalyst was filtered through celite and the filtration was concentrated to get the crude, which was then purified by prep-HPLC eluting with 0-90% ACN in water (0.1% TFA), and substituted by HCl to give 110-P2 (10.14 mg, 30%) as a white solid. LCMS [M+H]: 152.2. 1H NMR (400 MHz, CD3OD) δ 2.81 (dd, J=4.0, 1.3 Hz, 1H), 2.31 (dd, J=8.1, 4.4 Hz, 2H), 1.80-1.61 (m, 3H), 1.50-1.35 (m, 2H), 1.28 (ddd, J=8.9, 6.8, 3.6 Hz, 1H), 0.98 (dd, J=9.1, 5.1 Hz, 1H), 0.78-0.66 (m, 1H), 0.62-0.46 (m, 2H), 0.33 (td, J=9.2, 4.8 Hz, 1H), 0.10 (td, J=9.3, 5.1 Hz, 1H).

### Representative Procedure C for Synthesis and Separation of Functionalized Amines Formed by Reductive Amination

### Synthesis of (1R,2R,3R,4S)-3-methylbicyclo[2.2.1]heptan-2-amine hydrochloride (104-P1), (1S,2S,3S,4R)-3-methylbicyclo[2.2.1]heptan-2-amine hydrochloride (104-P2)

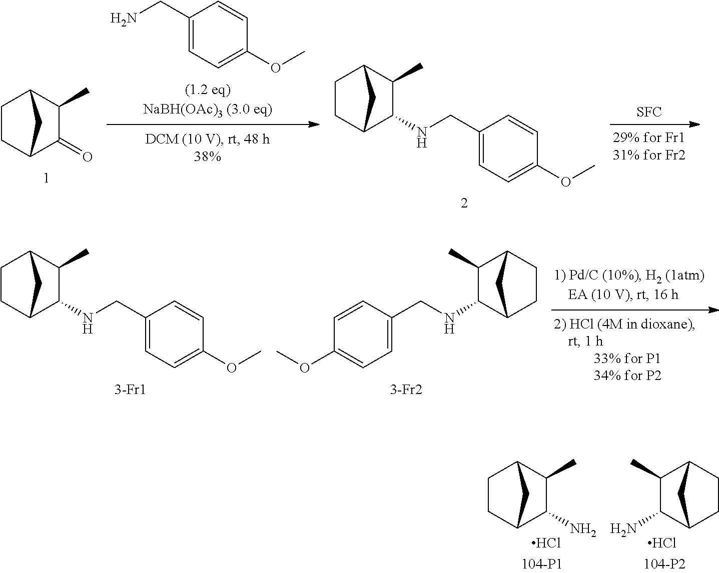

Step 1. To a solution of rac-3-exo-methylbicyclo[2.2.1]heptan-2-one (1.0 g, 8.06 mmol, 1.0 eq) in DCM (10 mL) was added (4-methoxyphenyl)methanamine (1.32 g, 9.68 mmol, 1.0 eq). After stirring at room temperature overnight, sodium triacetoxyborohydride (2.3 g, 16.12 mmol, 3.0 eq) was added at 0° C. and the mixture was then stirred at room temperature overnight. TLC and LCMS were done to detect the reaction process. Once no starting material was detected, solid was filtered and the filtration was concentrated under vacuum to provide the crude, which was purified by prep-HPLC to get the product rac-N-(4-methoxybenzyl)-3-exo-methylbicyclo[2.2.1]heptan-2-endo-amine as a yellow oil (700 mg, 38%). LCMS [M+H]: 246.2.

Step 2. SFC separation was carried out for compound 2 (700 mg). The SFC separation information are shown as following:

Analytical separation method: Instrument: Waters UPCC, Column: ChiralPak AY, 250×4.6 mm, 5 μm, Mobile phase: A for CO₂ and B for EtOH (0.03% DEA), Gradient: B 0˜30%, Flow rate: 2.8 mL/min, Back pressure: 100 bar, Column temperature: 35° C., Wavelength: 214 nm.

Preparative separation method: Instrument: Waters SFC80, Column: ChiralPak AY, 250×25 mm, 10 μm, Mobile phase: A for CO₂ and B for EtOH (0.03% DEA), Gradient: B 30%, Flow rate: 70 g/min, Back pressure: 100 bar, Column temperature: 35° C., Wavelength: 214 nm, Cycle time: 10 min, Sample preparation: Compound was dissolved in 15 mL methanol, Injection: 3 ml per injection.

After separation, (1R,2R,3R,4S)—N-(4-methoxybenzyl)-3-methylbicyclo[2.2.1]heptan-2-amine (compound 3-Fr1; 200 mg, 29%, 100% ee) was obtained as a colorless solid, and (1S,2S,3S,4R)—N-(4-methoxybenzyl)-3-methylbicyclo[2.2.1]heptan-2-amine (compound 3-Fr2; 220 mg, 31%, 100% ee) was obtained as a colorless solid. Compound 3-Fr1: 1H NMR (400 MHz, CDCl₃) δ 7.37 (d, J=8.5 Hz, 2H), 6.80 (d, J=8.6 Hz, 2H), 3.79-3.64 (m, 5H), 2.54 (t, J=3.4 Hz, 1H), 2.31 (s, 1H), 1.77 (t, J=5.9 Hz, 2H), 1.54-1.31 (m, 5H), 1.19-1.14 (m, 1H), 0.92 (d, J=7.0 Hz, 3H). Compound 3-Fr2: 1H NMR (400 MHz, CDCl₃) δ 7.24 (d, J=8.5 Hz, 2H), 6.84-6.72 (m, 2H), 3.72-3.54 (m, 6H), 2.48 (t, J=3.4 Hz, 1H), 2.25 (s, 1H), 1.74 (d, J=3.7 Hz, 1H), 1.63 (dd, J=17.2, 7.7 Hz, 1H), 1.50-1.38 (m, 2H), 1.30-1.04 (m, 5H), 0.91 (t, J=6.3 Hz, 3H). The absolute configuration of the compounds is unknown and peak 1 is arbitrarily assigned as C1-R.

Step 3. To a solution of (1R,2R,3R,4S)—N-(4-methoxybenzyl)-3-methylbicyclo[2.2.1]heptan-2-amine (200 mg, 1.0 eq) in EtOAc (5.0 mL) was added Pd/C (23 mg, 10% wt), and the reaction mixture was stirred at room temperature under H₂ atmosphere (1 atm) overnight. Once LCMS showed the reaction finished, solvent was removed to get the crude, which was then purified by prep-HPLC eluting with 0-90% ACN in water (0.1% TFA), and substituted by HCl to give 104-P1 (51 mg, 33%) as a white solid. LCMS [M+H]: 126.3. 1H NMR (400 MHz, CD3OD) δ 2.94 (s, 1H), 2.45 (s, 1H), 1.97 (d, J=3.5 Hz, 1H), 1.76-1.61 (m, 2H), 1.60-1.44 (m, 2H), 1.35 (t, J=18.3 Hz, 3H), 1.07 (d, J=7.0 Hz, 3H).

To a solution of (1S,2S,3S,4R)—N-(4-methoxybenzyl)-3-methylbicyclo[2.2.1]heptan-2-amine (230 mg, 1.0 eq) in EA (5.0 mL) was added Pd/C (23 mg, 10% wt), and the reaction mixture was stirred at room temperature under H₂ atmosphere (1 atm) overnight. Once LCMS showed the reaction finished, solvent was removed to get the crude, which was then purified by prep-HPLC eluting with 0-90% ACN in water (0.1% TFA), and substituted by HCl to give 104-P2 (58 mg, 34%) as a white solid. LCMS [M+H]: 126.3. 1H NMR (400 MHz, CD3OD) δ 2.94 (s, 1H), 2.45 (s, 1H), 1.96 (s, 1H), 1.67 (dd, J=10.8, 4.3 Hz, 2H), 1.54 (dd, J=13.9, 8.8 Hz, 2H), 1.37 (d, J=10.7 Hz, 3H), 1.07 (d, J=7.0 Hz, 3H).

### Representative Procedure D for Synthesis and Separation of Functionalized Amines Formed Via Oximes

### Synthesis of (1R,2R,3R,4S)-3-isopropylbicyclo[2.2.1]heptan-2-amine hydrochloride & (1R,2R,3R,4S)-3-isopropylbicyclo[2.2.1]heptan-2-amine hydrochloride & (1S,2S,3S,4R)-3-isopropylbicyclo[2.2.1]heptan-2-amine hydrochloride (103 & 103-P1 & 103-P2)

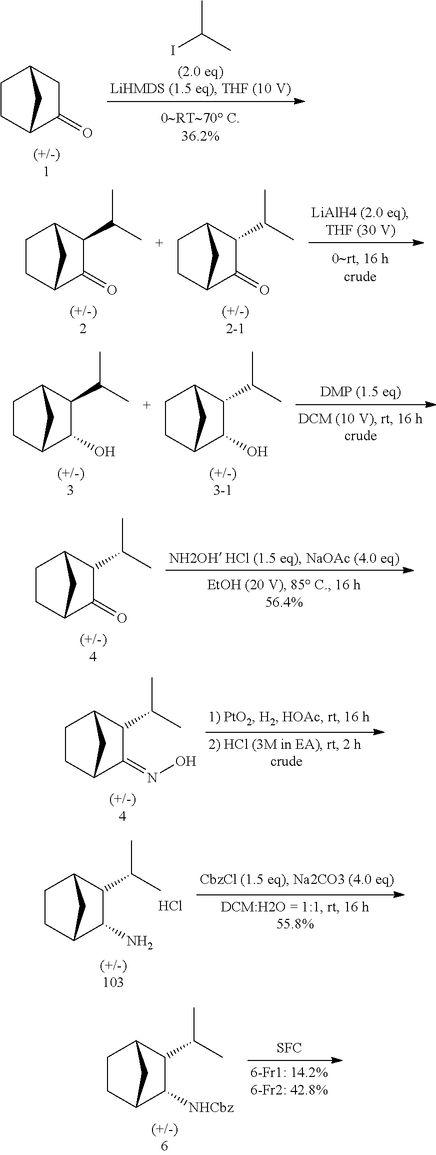

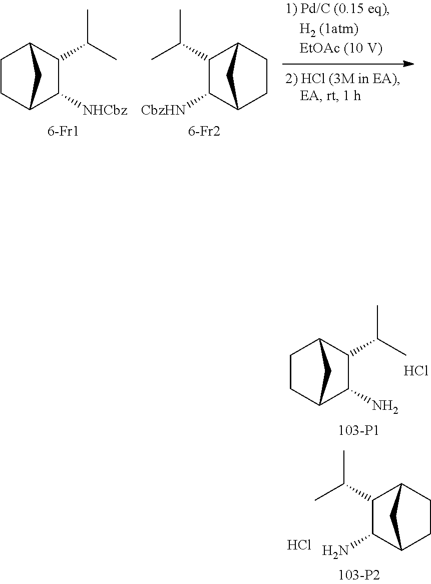

Step 1. To a stirred solution of bicyclo[2.2.1]heptan-2-one (25.0 g, 223 mmol, 1.0 eq) in anhydrous THE (50 mL) at 30° C. was added LiHMDS (1.0 M in THF; 204 mL, 204 mmol, 1.5 eq) under N₂ protection. The corresponding reaction mixture was then stirred at room temperature for 2 hours under N₂ followed by the addition of 2-iodopropane (75.9 g, 446 mmol, 2.0 eq). The mixture was gradually warmed to 70° C. and stirred overnight under N2 protection. Once TLC showed the reaction finished, the reaction mixture was quenched with sat. aq. NH₄Cl (250 mL) at 0° C. and stirred for 10 minutes followed by the extraction with EtOAc (3×100 mL). The organic phase was then washed with brine (100 mL), dried over Na₂SO₄, and filtered and concentrated under vacuum to give the crude, which was then purified by silica column chromatography eluting with 2% EA in PE to give a crude mixture of 3-(but-3-en-1-yl)bicyclo[2.2.1]heptan-2-one (22 g, 144 mmol, 36.2%) as a dark red oil.

Step 2. To a solution of LiAlH₄ (5.0 g, 131.38 mmol, 2.0 eq) in THE (100 mL) was added dropwise a mixture of crude rac-3-isopropylbicyclo[2.2.1]heptan-2-one (22 g, 144.7 mmol, 1.0 eq) in THE (50 mL) at 0° C. The mixture was stirred at room temperature for 16 hours. TLC was done to detect the process of the reaction. Once the reaction finished, 11 mL of H₂O, 11 mL of NaOH (15%) and 33 mL of H₂O was added in sequence at 0° C. to quench the reaction and the corresponding mixture was then stirred at room temperature for another 30 mins. Solid was filtered, and the filtration was dried over Na₂SO₄ to get rid of residual water and filtered. The filtration was then concentrated under vacuum to provide the crude, which was purified by silica column chromatography eluting with 2% EtOAc in PE to give rac-3-exo-isopropylbicyclo[2.2.1]heptan-2-endo-ol (compound 3) (4 g, 25.9 mmol, 11.6% from rac-bicyclo[2.2.1]heptan-2-one) as a colorless oil and rac-3-endo-isopropylbicyclo[2.2.1]heptan-2-endo-ol (compound 3-1) (8.5 g, 55.1 mmol, 24.7% from rac-bicyclo[2.2.1]heptan-2-one) as a colorless oil. 1H NMR (400 MHz, CDCl₃) δ 5.94-5.93 (m, 1H), 2.64-2.60 (m, 3H), 2.42-2.40 (m, 2H), 1.18 (d, J=6.8 Hz, 6H).

Step 3. To a solution of rac-3-endo-isopropylbicyclo[2.2.1]heptan-2-endo-ol (8.5 g, 58.02 mmol, 1.0 eq) in DCM (400 mL) was added Dess-Martin periodinane, DMP, (37 g, 87.03 mmol, 1.5 eq) portionwise at 0° C. The mixture was stirred at room temperature for 16 hours. TLC was done to detect the process of the reaction. Once the reaction finished, the mixture was filtered through celite pad, and the filtration was then concentrated under vacuum. The residue was purified by silica column chromatography eluting with 3% EtOAc in PE to give rac-3-endo-isopropylbicyclo[2.2.1]heptan-2-one (4.5 g, 29.6 mmol) as a colorless oil. 1H NMR (400 MHz, CD3OD) δ 2.70-2.69 (m, 1H), 2.52 (d, J=5.2 Hz, 1H), 1.91-1.83 (m, 2H), 1.71-1.57 (m, 7H), 1.46-1.36 (m, 3H), 1.21-1.16 (m, 4H), 0.98-0.92 (m, 4H).

Step 4. To a solution of rac-3-endo-isopropylbicyclo[2.2.1]heptan-2-one (3.6 g, 23.7 mmol, 1.0 eq) in EtOH (30 mL) was added hydroxylamine hydrochloride (2.5 g, 35.5 mmol, 1.5 eq) and NaOAc (7.7 g, 94.8 mmol, 4.0 eq), and the reaction mixture was stirred at 85° C. overnight. Once LCMS showed the reaction finished, solvent was removed under vacuum to get a residue, which was diluted and extracted with EtOAc (3×25 mL). The organic phases were collected, washed with brine (3×35 mL), dried over Na₂SO₄, and filtered. The filtration was then purified by column chromatography eluting with 0-4% EA in PE to give (1R,3R,4S,Z)-3-isopropylbicyclo[2.2.1]heptan-2-one oxime (2.2 g, 13.1 mmol, 56.4%) as a white solid. 1H NMR (400 MHz, CD3OD) δ 3.37 (d, J=4.0 Hz, 1H), 2.37 (d, J=1.6 Hz, 1H), 1.85-1.75 (m, 1H), 1.60-1.36 (m, 4H), 1.30-1.19 (m, 4H), 1.06 (d, J=6.4 Hz, 3H), 2.37 (d, J=6.4 Hz, 1H).

Step 5. To a solution of rac-3-endo-isopropylbicyclo[2.2.1]heptan-2-one oxime (2.2 g, 13.17 mmol, 1.0 eq) in HOAc (10.0 mL) was added PtO₂ (200 mg, 0.1 eq). The reaction mixture was stirred at room temperature under H₂ atmosphere (1 atm) overnight. Once LCMS showed the reaction finished, reaction mixture was filtered and the filtration was concentrated to give rac-3-endo-isopropylbicyclo[2.2.1]heptan-2-endo-amine hydrochloride (2.5 g, crude) as a white solid. 1H NMR (400 MHz, CD3OD) δ 3.54-3.50 (m, 1H), 2.45 (s, 1H), 2.21 (s, 1H), 1.71-1.63 (m, 1H), 1.58-1.52 (m, 1H), 1.46-1.28 (m, 7H), 0.89 (d, J=6.4 Hz, 3H), 0.84 (d, J=6.4 Hz, 3H).

Step 6. To a solution of (1R,2R,3R,4S)-3-isopropylbicyclo[2.2.1]heptan-2-amine hydrochloride (2.5 g, 13.18 mmol, 1.0 eq) in DCM:H2O=1:1 (40 mL) was added Na₂CO₃ (5.59 g, 52.72 mmol, 4.0 eq) and benzyl chloroformate (3.37 g, 19.77 mmol, 1.5 eq). The whole reaction mixture was then stirred at room temperature overnight. Once LCMS showed the reaction finished, solvent was removed under vacuum to get a residue, which was diluted and extracted with EA (3×20 mL). The organic phases were collected, washed with brine (20 mL), dried over Na2SO4, and filtered. The filtration was then purified by column chromatography eluting with 0-5% EA in PE to benzyl ((1R,2R,3R,4S)-3-isopropylbicyclo[2.2.1]heptan-2-yl)carbamate (2.11 g, 7.35 mmol, 55.8% yield) an a white solid. 1H NMR (400 MHz, CD₃OD) δ 7.36-7.27 (m, 5H), 5.12-5.02 (m, 2H), 4.10-4.07 (m, 1H), 2.23 (s, 1H), 2.18 (s, 1H), 1.64-1.59 (m, 2H), 1.50-1.30 (m, 7H), 0.92-0.79 (m, 6H).

Step 7. To a solution of rac-benzyl (3-endo-isopropylbicyclo[2.2.1]heptan-2-endo-yl)carbamate (33 mg, 0.11 mmol, 1.0 eq) in EA (5.0 mL) was added Pd/C (10 mg, 10% wt), and the reaction mixture was stirred at room temperature under H2 atmosphere (1 atm) overnight. Once LCMS showed the reaction finished, solvent was removed to get the crude, which was then purified by prep-HPLC eluting with 0-90% ACN in water (0.1% TFA), and substituted by HCl to give 103 (4.55 mg, 21.6%) as a white solid. LCMS [M+H]: 154.1. 1H NMR (400 MHz, CD3OD) δ 3.52-3.48 (m, 1H), 2.42 (s, 1H), 2.22 (s, 1H), 1.62-1.54 (m, 2H), 1.42-1.35 (m, 6H), 0.89-0.84 (m, 6H). The individual enantiomers could be obtained by SFC chromatoprahy followed by hydrogenation as described above.

TABLE 3
C2-amino-C3-monosubstituted BRD4780 analogs. (Note, P1/P2 indicates a single
enantiomer compound of undetermined absolute stereochemistry)

| Compound | | 1H-NMR, 400 | LCMS | ee | |
|---|---|---|---|---|---|
| No. # | Structure | MHz | [M + H]⁺ | (%) | Procedure |
| 101 | 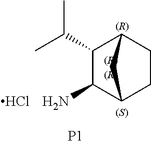 | (CDCl₃) δ 8.38 (bs, 3H), 2.75 (m, 1H), 2.56 (d, J = 5.0 Hz, 1H), 2.39 (dd, J = ~4.5, ~4.5, 1H), 2.02 (dt, J = 10.7, 1.9 Hz, 1H), 1.72- 1.58 (m, 1H), 1.54 (dt, J = 10.3, 3.3 Hz, | 154.2 | >99 | B |
| | | 1H), 1.48- | | | |
| | | 1.25 (m, 4H), 1.18- | | | |
| | | 1.11 (m, 1H), 1.14 | | | |
| | | (d, J = 6.4 Hz, 3H), | | | |
| | | 0.90 (d, J = 6.4 Hz, | | | |
| | | 3H). | | | |
| 102 | 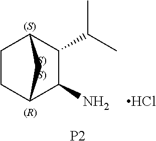 | (CDCl₃) δ 8.38 (bs, 3H), 2.75 (m, 1H), 2.56 (d, J = 5.0 Hz, 1H), 2.39 (dd, J = ~4.5, ~4.5, 1H), 2.02 (dt, J = 10.7, 1.9 Hz, 1H), 1.72- 1.58 (m, 1H), 1.54 (dt, J = 10.3, 3.3 Hz, | 154.2 | 97.2 | B |
| | | 1H), 1.48- | | | |
| | | 1.25 (m, 4H), 1.18- | | | |
| | | 1.11 (m, 1H), 1.14 | | | |
| | | (d, J = 6.4 Hz, 3H), | | | |
| | | 0.90 (d, J = 6.4 Hz, | | | |
| | | 3H). | | | |
| 103 | 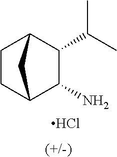 | (CD₃OD) δ 3.52- 3.48 (m, 1H), 2.42 (s, 1H), 2.22 (s, 1H), 1.62-1.54 (m, 2H), 1.42-1.35 (m, 6H), 0.89-0.84 (m, 6H). | 154.1 | NA | D |
| 103-P1 | 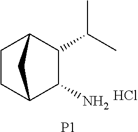 | (CD₃OD) δ 3.60 (d, J = 6.4 Hz, 1H), 2.53 (s, 1H), 2.31 (s, 1H), 1.74-1.65 (m, 2H), 1.51-1.45 (m, 6H), 0.99-0.93 (m, 6H). | 154.1 | 100 | D |
| 103-P2 | 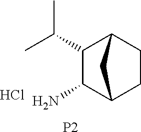 | (CD₃OD) δ 3.62- 3.58 (m, 1H), 2.52 (s, 1H), 2.31 (s, 1H), 1.76-1.62 (m, 2H), 1.51-1.44 (m, 6H), 0.98-0.93 (m, 6H). | 154.1 | 100 | D |
| 104-P1 | 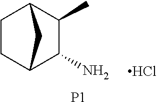 | (CD₃OD) δ 2.94 (s, 1H), 2.45 (s, 1H), 1.97 (d, J = 3.5 Hz, 1H), 1.76-1.61 (m, 2H), 1.60- 1.44 (m, 2H), 1.35 (t, J = 18.3 Hz, | 126.3 | 100 | C |
| | | 3H), 1.07 (d, J = | | | |
| | | 7.0 Hz, 3H). | | | |
| 104-P2 | 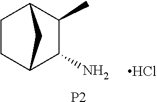 | (CD₃OD) δ 2.94 (s, 1H), 2.45 (s, 1H), 1.96 (s, 1H), 1.67 (dd, J = 10.8, 4.3 Hz, 2H), 1.54 (dd, J = 13.9, 8.8 Hz, 2H), 1.37 (d, J = | 126.3 | 100 | C |
| | | 10.7 Hz, 3H), 1.07 | | | |
| | | (d, J = 7.0 Hz, 3H). | | | |
| 105-P1 | 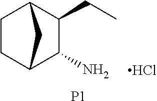 | (CD₃OD) δ 3.00 (t, J = 4.0 Hz, 1H), 2.45 (s, 1H), 2.13 (d, J = 3.7 Hz, 1H), 1.76-1.65 (m, 1H), 1.57 (dt, J = 11.0, 8.6 Hz, 3H), 1.38 | 140.3 | 100 | A |
| | | (m, 4H), 1.18-1.10 | | | |
| | | (m, 1H), 0.97 (t, J = | | | |
| | | 7.4 Hz, 3H). | | | |
| 105-P2 | 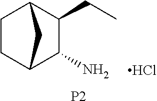 | (CD₃OD) δ 3.00 (t, J = 3.7 Hz, 1H), 2.45 (s, 1H), 2.13 (d, J = 3.8 Hz, 1H), 1.76-1.65 (m, 1H), 1.63-1.50 (m, 3H), 1.48-1.29 (m, 4H), | 140.3 | 98.6 | A |
| | | 1.14 (ddd, J = 10.8, | | | |
| | | 7.5, 3.0 Hz, 1H), | | | |
| | | 0.97 (t, J = 7.4 Hz, | | | |
| | | 3H). | | | |
| 106-P1 | 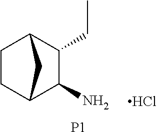 | (CDCl₃) δ 2.56 (s, 1H), 2.37 (s, 1H), 2.30 (d, J = 4.0 Hz, 1H), 1.67 (m, 4H), 1.55-1.48 (m, 1H), 1.45-1.32 (m, 3H), 1.26-1.17 (m, 1H), 1.02-0.92 (m, 3H). | 140.3 | 100 | B |
| 106-P2 | 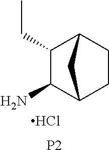 | (CDCl₃) δ 2.56 (s, 1H), 2.37 (s, 1H), 2.30 (s, 1H), 1.80- 1.58 (m, 4H), 1.50 (d, J = 8.9 Hz, 1H), 1.47-1.32 (m, 3H), 1.26-1.16 (m, 1H), 0.96 (t, J = 7.2 Hz, 3H). | 140.3 | 100 | B |
| 107-rac | 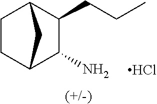 | (CDCl₃) δ 8.48 (brs, 3H), 3.03 (s, 1H), 2.57 (s, 1H), 2.05 (s, 1H), 1.83 (s, 1H), 1.62-1.26 (m, 10H), 0.93- 0.90 (m, 3H). | 154.3 | N | A |
| 107-P1 | 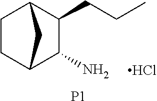 | (CDCl₃) δ 8.49 (brs, 3H), 3.03 (s, 1H), 2.56 (s, 1H), 2.05 (d, J = 3.2 Hz, 1H), 1.83-1.80 (m, 3H), 1.52-1.38 (m, 4H), 1.31-1.25 (m, 3H), 0.93-0.89 (m, 3H). | 154.3 | 100 | A |
| 107-P2 | 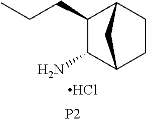 | (CDCl₃) δ 8.43 (brs, 3H), 3.02 (d, J = 2.8 Hz, 1H), 2.53 (s, 1H), 2.05 (d, J = 3.2 Hz, 1H), 1.83- 1.24 (m, 11H), 0.93-0.89 (m, 3H). | 154.3 | 100 | A |
| 108-rac | 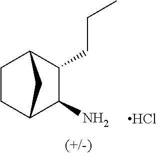 | (CD₃OD) δ 2.54 (d, J = 4.0 Hz, 1H), 2.32 (s, 1H), 2.27 (d, J = 4.0 Hz, 1H), 1.55-1.53 (m, 3H), 1.52-1.48 (m, 2H), 1.43-1.29 (m, 5H), 1.22-1.19 (m, 1H), 1.11-1.01 (m, 1H), 0.97-0.93 (m, 3H) | 154.1 | NA | B |
| 108-P1 | 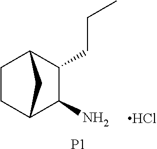 | (CD₃OD) δ 2.54 (d, J = 4.0 Hz, 1H), 2.32 (s, 1H), 2.27 (d, J = 4.0 Hz, 1H), 1.55-1.53 (m, 3H), 1.52-1.48 (m, 2H), 1.43-1.29 (m, 5H), 1.22-1.19 (m, 1H), 1.11-1.01 (m, 1H), 0.97-0.93 (m, 3H) | 154.1 | 100 | B |
| 108-P2 | 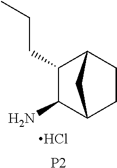 | (CD₃OD) δ 2.54 (d, J = 4.0 Hz, 1H), 2.32 (s, 1H), 2.27 (d, J = 4.0 Hz, 1H), 1.71-1.63 (m, 3H), 1.60-1.46 (m, 2H), 1.45-1.26 (m, 5H), 1.25-1.16 (m, 1H), 0.97-0.93 (m, 3H). | 154.1 | 99 | B |
| 109 | 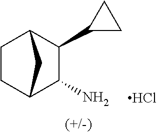 | (CDCl₃) δ 8.49 (brs, 3H), 3.31 (brs, 1H), 2.60 (s, 1H), 2.17 (d, J = 3.2 Hz, 1H), 1.82-1.77 (m, 1H), 1.63-1.61 (m, 1H), 1.56-1.44 (m, 2H) 1.37 (d, J = 10.0 Hz, 1H), 1.29- | 152.2 | NA | A |
| | | 1.26 (m, 1H), 0.82- | | | |
| | | 0.81 (m, 1H), 0.66- | | | |
| | | 0.61 (m, 1H), 0.55- | | | |
| | | 0.44 (m, 3H), 0.20- | | | |
| | | 0.14 (m, 1H). | | | |
| 109-P1 | 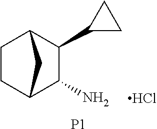 | (CDCl₃) δ 3.30-3.26 (m, 1H), 2.50 (s, 1H), 2.20 (s, 1H), 1.69 (ddd, J = 13.9, 11.5, 7.8 Hz, 2H), 1.61-1.49 (m, 2H), 1.42 (dd, J = 10.5, 1.7 Hz, 1H), 1.34- | 152.3 | 100 | A |
| | | 1.27 (m, 1H), 0.73 | | | |
| | | (ddd, J = 9.7, 7.8, | | | |
| | | 4.8 Hz, 1H), 0.66- | | | |
| | | 0.61 (m, 1H), 0.57- | | | |
| | | 0.49 (m, 2H), 0.35- | | | |
| | | 0.26 (m, 1H), 0.20- | | | |
| | | 0.13 (m, 1H). | | | |
| 109-P2 | 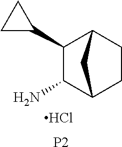 | (CDCl₃) δ 3.28 (s, 1H), 2.49 (s, 1H), 2.20 (s, 1H), 1.69 (ddd, J = 12.8, 11.4, 6.8 Hz, 2H), 1.62-1.48 (m, 2H), 1.42 (d, J = 10.5 Hz, 1H), 1.30 (s, 1H), 0.74 (qd, J = | 152.3 | 100 | A |
| | | 9.2, 4.6 Hz, 1H), | | | |
| | | 0.63 (dd, J = 6.0, | | | |
| | | 3.1 Hz, 1H), 0.59- | | | |
| | | 0.47 (m, 2H), 0.33- | | | |
| | | 0.24 (m, 1H), 0.16 | | | |
| | | (dd, J = 8.4, 4.6 Hz, | | | |
| | | 1H). | | | |
| 110-P1 | 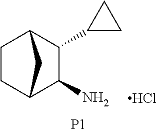 | (CD₃OD) δ 2.81 (dd, J = 4.0, 1.3 Hz, 1H), 2.35-2.27 (m, 2H), 1.78-1.68 (m, 2H), 1.63 (d, J = 10.6 Hz, 1H), 1.49-1.37 (m, 2H), 1.31-1.26 (m, 1H), | 152.2 | 100 | B |
| | | 0.97 (dd, J = 8.3, | | | |
| | | 4.2 Hz, 1H), 0.76- | | | |
| | | 0.67 (m, 1H), 0.60- | | | |
| | | 0.50 (m, 2H), 0.33 | | | |
| | | (td, J = 9.2, 4.8 Hz, | | | |
| | | 1H), 0.10 (dt, J = | | | |
| | | 9.6, 4.6 Hz, 1H). | | | |
| 110-P2 | 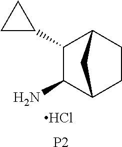 | (CD₃OD) δ 2.81 (dd, J = 4.0, 1.3 Hz, 1H), 2.31 (dd, J = 8.1, 4.4 Hz, 2H), 1.80-1.61 (m, 3H), 1.50- 1.35 (m, 2H), 1.28 (ddd, J = 8.9, 6.8, 3.6 Hz, 1H), 0.98 (dd, J = 9.1, 5.1 Hz, | 152.3 | 100 | B |
| | | 1H), 0.78-0.66 | | | |
| | | (m, 1H), 0.62-0.46 | | | |
| | | (m, 2H), 0.33 (td, | | | |
| | | J = 9.2, 4.8 Hz, 1H), | | | |
| | | 0.10 (td, J = 9.3, | | | |
| | | 5.1 Hz, 1H). | | | |
| 111 | 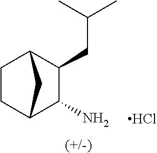 | (CDCl₃) δ 8.53 (brs, 3H), 2.98 (brs, 1H), 2.55 (s, 1H), 2.03 (d, J = 2.8 Hz, 1H), 1.87-1.82 (m, 1H), 1.71-1.50 (m, 6H), 1.35-1.28 (m, 2H), 1.21-1.13 (m, 1H), 0.91 (t, J = 6.0 Hz, 6H). | 168.3 | NA | A |
| 111-P1 | 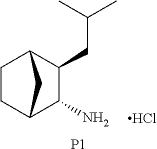 | (CDCl₃) δ 8.53 (brs, 3H), 2.98 (brs, 1H), 2.55 (s, 1H), 2.03 (d, J = 2.8 Hz, 1H), 1.87-1.82 (m, 1H), 1.71-1.50 (m, 6H), 1.35-1.28 (m, 2H), 1.21-1.13 (m, 1H), 0.91 (t, J = 6.0 Hz, 6H). | 168.3 | 100 | A |
| 111-P2 | 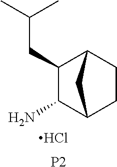 | (CDCl₃) δ 8.53 (brs, 3H), 2.98 (brs, 1H), 2.55 (s, 1H), 2.03 (d, J = 2.8 Hz, 1H), 1.87-1.82 (m, 1H), 1.71-1.50 (m, 6H), 1.35-1.28 (m, 2H), 1.21-1.13 (m, 1H), 0.91 (t, J = 6.0 Hz, 6H). | 168.3 | 100 | A |
| 112 | 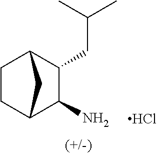 | (CDCl₃) δ 8.53 (brs, 3H), 2.50 (brs, 1H), 2.38 (s, 1H), 2.20 (s, 1H), 1.95- 1.93 (m, 2H), 1.54- 1.46 (m, 4H), 1.35- 1.27 (m, 3H), 1.12- 1.02 (m, 2H), 0.86- 0.83 (m, 6H). | 168.3 | NA | B |
| 112-P1 | 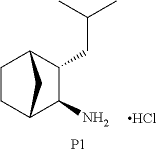 | (CDCl₃) δ 8.35 (brs, 3H), 2.50 (brs, 1H), 2.45 (s, 1H), 2.27 (s, 1H), 1.95- 1.93 (m, 2H), 1.54- 1.46 (m, 4H), 1.35- 1.27 (m, 3H), 1.12- 1.02 (m, 2H), 0.86- 0.83 (m, 6H). | 168.3 | 100 | B |
| 112-P2 | 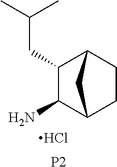 | (CDCl₃) δ 8.37 (brs, 3H), 2.56 (brs, 1H), 2.44 (s, 1H), 2.20 (s, 1H), 1.95- 1.93 (m, 2H), 1.54- 1.46 (m, 4H), 1.35- 1.27 (m, 3H), 1.12- 1.02 (m, 2H), 0.86- 0.83 (m, 6H). | 168.3 | 99.4 | B |
| 113 | 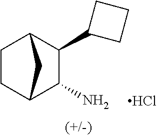 | (CD₃OD) δ 2.99 (d, J = 3.6 Hz, 1H), 2.44 (s, 1H), 2.27- 2.01 (m, 4H), 1.96- 1.63 (m, 5H), 1.62- 1.47 (m, 3H), 1.38- 1.24 (m, 3H). | 166.3 | NA | A |
| 113-P1 | 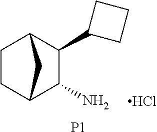 | (CD₃OD) δ 2.99 (s, 1H), 2.44 (s, 1H), 2.24-2.03 (m, 4H), 1.94-1.76 (m, 2H), 1.75-1.63 (m, 3H), 1.61-1.47 (m, 3H), 1.36-1.25 (m, 3H). | 166.3 | 100 | A |
| 113-P2 | 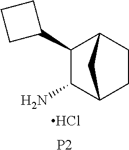 | (CD₃OD) δ 2.99 (s, 1H), 2.44 (s, 1H), 2.24-2.03 (m, 4H), 1.84 (ddt, J = 10.9, 8.0, 7.3 Hz, 2H), 1.76-1.64 (m, 3H), 1.61-1.47 (m, 3H), 1.37-1.25 (m, 3H). | 166.3 | 98.7 | A |
| 114-rac | 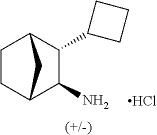 | (CD₃OD) δ 2.59 (s, 1H), 2.27 (dd, J = 16.5, 11.3 Hz, 3H), 2.19-2.11 (m, 1H), 2.05-1.90 (m, 2H), 1.82 (dt, J = 14.0, 8.3 Hz, 2H), 1.73- 1.63 (m, 4H), 1.36 (tdd, J = 16.6, 12.8, | 166.1 | NA | B |
| | | 6.6 Hz, 3H), 1.22- | | | |
| | | 1.12 (m, 1H). | | | |
| 114-P1 | 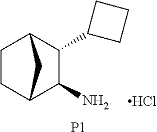 | (CD₃OD) δ 2.58 (d, J = 2.5 Hz, 1H), 2.33-2.11 (m, 4H), 2.03-1.78 (m, 4H), 1.75-1.61 (m, 4H), 1.37 (ddd, J = 16.6, 12.9, 6.8 Hz, 3H), 1.22-1.13 (m, 1H). | 166.3 | 100 | B |
| 114-P2 | 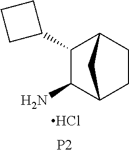 | (CD₃OD) δ 2.58 (d, J = 2.5 Hz, 1H), 2.32-2.12 (m, 4H), 2.05-1.75 (m, 4H), 1.75-1.58 (m, 4H), 1.47-1.28 (m, 3H), 1.22-1.12 (m, 1H). | 166.3 | 100 | B |
| 115 | 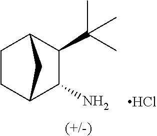 | (CDCl₃) δ 7.81- 7.80 (m, 1H), 3.23 (s, 1H), 2.47 (s, 1H), 2.21 (s, 1H), 1.96-1.69 (m, 4H), 1.62-1.57 (m, 1H), 1.53-1.46 (m, 1H), 1.37-1.30 (m, 1H), 1.24-1.17 (m, 1H), | 168.2 | NA | A |
| | | 1.12-1.11 (m, 1H), | | | |
| | | 0.88 (s, 9H). | | | |
| 116-P1 | 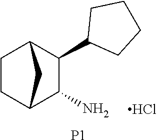 | (CDCl₃) δ 8.47 (brs, 3H), 3.19 (s, 1H), 2.62 (s, 1H), 2.13 (s, 1H), 1.85- 1.95 (m, 2H), 1.75- 1.83 (m, 1H), 1.73- 1.45 (m, 10H), 1.37-1.25 (m, 3H), 1.20-1.10 (m, 1H). | 180.2 | 100 | A |
| 116-P2 | 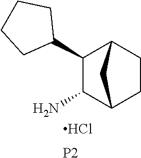 | (CD₃OD) δ 3.16 (t, J = 4.0 Hz, 1H), 2.44 (s, 1H), 2.17 (d, J = 4.0 Hz, 1H), 1.88-1.83 (m, 2H), 1.71-1.61 (m, 4H), 1.62-1.52 (m, 5H), 1.37-1.28 (m, 2H), 1.17-1.10 (m, 3H). | 180.2 | 99.5 | A |
| 117-P1 | 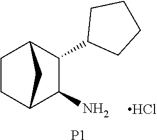 | (CDCl₃) δ 2.65 (s, 1H), 2.43-2.44 (d, 1H), 2.22 (s, 1H), 1.87-2.02 (m, 3H), 1.42-1.64 (m, 20H), 1.08-1.36 (m, 8H), 1.02-1.06 (m, 3H), 0.79-0.81 (m, 2H). | 180.2 | 100 | B |
| 117-P2 | 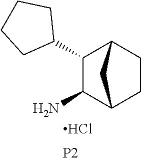 | (CDCl₃) δ 2.91 (s, 1H), 2.83 (s, 1H), 2.68 (s,1H), 2.59 (s, 5H), 2.48 (s, 1H), 2.23 (s, 1H), 1.87-1.93 (m, 2H), 1.49-1.63 (m, 10H), 2.20 (s, 1H), 1.19-1.39 (m, 2H), 1.04-1.08 (t, 2H). | 180.2 | 97.8 | B |
| 118 | 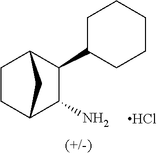 | (DMSO-d₆) δ 8.10 (s, 3H), 3.09 (s, 1H), 2.37 (s, 1H), 2.16 (s, 1H), 1.79- 1.68 (m, 4H), 1.62- 1.53 (m, 3H), 1.41- 1.39 (m, 2H), 1.25- 1.24 (m, 1H), 1.18- 1.08 (m, 5H), 1.02- 1.01 (m, 1H), 0.87- 0.80 (m, 2H). | 194.2 | NA | A |
| 118-P1 | 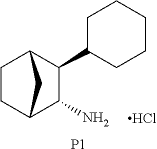 | (CD₃OD) δ 3.22 (t, J = 4.0 Hz, 1H), 2.45 (s, 1H), 2.30 (d, J = 4.0 Hz, 1H), 1.90-1.87 (m, 1H), 1.81-1.78 (m, 3H), 1.75-1.65 (m, 2H), 1.58-1.52 (m, 3H), 1.34-1.26 (m, 3H), 1.23-1.05 (m, 3 H), | 194.2 | 100 | A |
| | | 0.96-0.89 (m, 3H). | | | |
| 118-P2 | 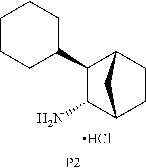 | (CD₃OD) δ 3.22 (t, J = 4.0 Hz, 1H), 2.45 (s, 1H), 2.30 (d, J = 3.6 Hz, 1H), 1.90-1.87 (m, 1H), 1.78-1.75 (m, 3H), 1.72-1.65 (m, 2H), 1.57-1.50 (m, 3H), 1.34-1.26 (m, 3H), 1.23-1.10 (m, 3, | 194.2 | 99.5 | A |
| | | H), 0.96-0.87 (m, | | | |
| | | 3H). | | | |
| 119 | 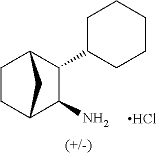 | (CDCl₃) δ 8.29 (s, 3H), 2.72 (s, 1H), 2.54 (d, J = 4.4 Hz, 1H), 2.37 (s, 1H), 1.98-1.96 (m, 1H), 1.91-1.88 (m, 1H), 1.81-1.78 (m, 1H), 1.75-1.7 (m, 1H), 1.61-1.57 (m, 4H), 1.37-1.24 (m, 3H), | 194.2 | NA | B |
| | | 1.18-1.04 (m, 6H), | | | |
| | | 0.91-0.88 (m, 1H). | | | |
| 119-P1 | 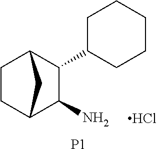 | (CD₃OD) δ 2.71 (d, J = 2.8 Hz, 1H), 2.39 (s, 1H), 2.26 (d, J = 4.8 Hz, 1H), 1.84-1.62 (m, 7H), 1.48-1.37 (m, 3H), 1.34-1.28 (m, 3H), 1.26-1.14 (m, 4H), 1.11-1.01 (m, 1H), 0.94-0.85 (m, 1H). | 194.2 | 100 | B |
| 119-P2 | 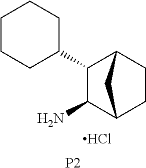 | (CD₃OD) δ 2.70 (d, J = 2.4 Hz, 1H), 2.38 (s, 1H), 2.26 (d, J = 5.2 Hz, 1H), 1.84-1.63 (m, 7H), 1.48-1.37 (m, 3H), 1.34-1.28 (m, 3H), 1.26-1.12 (m, 4H), 1.07-1.97 (m, 1H), 0.94-0.84 (m, 1H). | 194.2 | 100 | B |
| 120 | 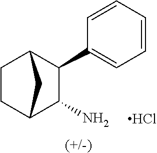 | (CDCl₃) δ 8.80 (s, 3H), 7.35-7.26 (m, 4H), 7.19 (t, J = 7.0 Hz, 1H), 3.57 (s, 1H), 2.86 (s, 1H), 2.73 (s, 1H), 2.56 (s, 1H), 2.10-1.95 (m, 1H), 1.76 (dd, J = 25.4, 7.4 Hz, 5H), 1.64 (d, J = | 188.2 | NA | A |
| | | 9.4 Hz, 2H), 1.47 | | | |
| | | (d, J = 10.3 Hz, | | | |
| | | 1H). | | | |
| 120-P1 | 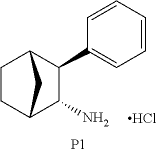 | (CD₃OD) δ 7.40- 7.21 (m, 5H), 3.69 (t, J = 4.4 Hz, 1H), 2.64-2.59 (m, 2H), 2.34 (d, J = 2.8 Hz, 1H), 1.93-1.90 (m, 1H), 1.79-1.74 (m, 1H), 1.70-1.67 (m, 2H), 1.63-1.57 (m, 1H), 1.50-1.47 (m, | 188.2 | 100 | A |
| | | 1H). | | | |
| 120-P2 | 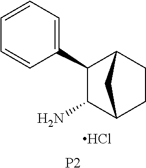 | (CD₃OD) δ 7.36- 7.23 (m, 5H), 3.69 (t, J = 4.4 Hz, 1H), 2.63 (s, 1H), 2.60- 2.58 (m, 1H), 2.34 (d, J = 2.8 Hz, 1H), 1.92 (d, J = 10.8 Hz, 1H), 1.80-1.77 (m, 1H), 1.75- 1.66 (m, 2H), 1.63- 1.57 (m, 1H), 1.50- | 188.2 | 92.1 | A |
| | | 1.47 (m, 1H). | | | |
| 121-P1 | 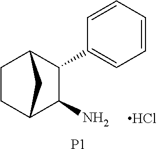 | (CDCl₃) δ 8.53 (s, 3H), 7.32-7.16 (m, 5H), 3.40-3.29 (d, 2H), 2.61-2.58 (m, 2H), 2.23-2.20 (d, 1H), 1.47-1.45 (d, 1H), 1.29-1.21 (m, 3H). | 188.2 | 95.9 | B |
| 121-P2 | 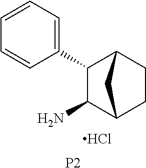 | (CDCl₃) δ 8.48 (s, 3H), 7.31-7.16 (m, 5H), 3.40-3.29 (m, 2H), 2.61-2.57 (m, 2H), 2.21-2.19 (m, 1H), 1.61 (s, 1H), 1.45-1.43 (m, 2H), 1.29-1.19 (m, 3H). | 188.2 | 93.4 | B |
| 122 | 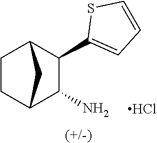 | (CDCl₃) δ 8.79 (s, 3H), 7.12 (d, J = 4.2 Hz, 1H), 7.02 (s, 1H), 6.92 (s, 1H), 3.64 (s, 1H), 3.10 (s, 1H), 2.74 (s, 1H), 2.52 (s, 1H), 1.96 (s, 1H), 1.83 (d, J = 10.1 Hz, | 194.2 | | A |
| | | 1H), 1.62 (s, | | | |
| | | 2H), 1.48 (d, J = | | | |
| | | 9.9 Hz, 1H). | | | |
| 123 | | (CD₃OD) δ 3.63 | 180.1 | NA | A |
| | | (brs, 1H), 2.73- | | | |
| | | 2.69 (m, 2H), 2.61 | | | |
| | | (brs, 1H), 2.55 (d, | | | |
| | | J = 3.2 Hz, 1H), 2.49 | | | |
| | | (d, J = 4.4 Hz, 1H), | | | |
| | | 2.33-2.22 (m, 1H), | | | |
| | | 1.85-1.73 (m, 4H), | | | |
| | | 1.69-1.60 (m, 3H), | | | |
| | | 1.54-1.46 (m, 4H), | | | |
| | | 1.36-1.29 (m, 2H). | | | |
| | | (ratio of endo:exo ~ | | | |
| | | 1:1). | | | |

### Example 4: Exemplary Biological Properties of Compounds of the Disclosure

P cells were seeded 24 hr prior to compound treatment at a density of 12,000 cells/well in 384 well Cell Carrier Ultra plates (6057308, Perkin Elmer), pre-coated with 0.25 mg/mL Synthemax 11 SC Substrate (3535, Corning). Compounds were used at 5 doses (35, 3.5, 0.35, 0.035 and 0.0035 μM) for the primary screen and 10 doses (16, 5.6, 1.8, 0.6, 0.21, 0.07, 0.02, 0.008, 0.002 and 0.0008 μM) for the following screens. The compounds, in two replicates, were transferred from compound source plates to the cell plates using the HighRes Pin Tool. DMSO was used as a negative control and JQ1 (250 nM) (a bromodomain inhibitor) was chosen as a positive control, based on earlier studies showing its potent effect on reducing total MUC1 mRNA levels (data not shown). After 48 hr incubation, cells were fixed for 20 min in 4% PFA (Electron Microscopy Sciences) in PBS, washed twice, then permeabilized (10 min) with 0.5% Triton-X100 (X100-100MVL, Sigma-Aldrich) in PBS and washed once more. Cells were blocked for 10 min at RT with Blocking solution (100 mM Tris HCL pH8; l150 mM NaCL; 5 g/L Blocking Reagent [11096176001, Roche]), then incubated 90 min at RT with one of the following primary antibodies in Roche Blocking solution: 1:500, monoclonal Fab-A-V5H anti-MUC1-fs, AbD22655.2, Bio-Rad; 1:2000, monoclonal mouse anti-MUC1 (214D4), 05-652-KC, Millipore; 1:1000, monoclonal, Rabbit anti-GM130 (D6B1) XP, 12480, Cell signaling technology. The primary antibody cocktail was incubated at RT for 1.5 hr, followed by four PBS wash cycles. The secondary antibody cocktail contained four components that were all prepared at a 1:1000 dilution in the Roche blocking solution and consisted of Alexa Fluor® 488-conjugated AffiniPure F(ab′)₂ Fragment Goat anti-Human IgG, 109-546-097, Jackson Immunoresearch; Alexa Fluor® 647-conjugated Goat anti-Rabbit IgG, A-21246, Thermo Fisher Scientific®; Alexa Fluor® 546 Goat anti-mouse IgG, A-21123, Thermo Fisher Scientific® and Hoechst 33342 stain (62249, Thermo Fisher Scientific®). The secondary antibody cocktail was incubated at RT for 45 min, followed by four PBS wash cycles. Finally, plates were sealed with a Plate Loc plate and stored in Liconic incubator at 10° C. until imaging. Following image analysis, three parameters were selected, i) MUC1-fs and ii) MUC1-wt total cytoplasm intensity (averaged per cell) and iii) cell number as was detected by Hoechst 33342 stained nuclei. The levels of MUC1-fs and MUC1-wt found following DMSO and JQ1 were defined as 0 and −100% activity, respectively. The values obtained for all compounds were normalized accordingly. Cell number was normalized to DMSO control.

TABLE 4

| Compound | EC₅₀ | EC_{Max} | WT EC₅₀ | WT EC_{Max} |
|---|---|---|---|---|
| No. | (μM) | (%) | (Cytoplasm μM) | (%) |
| 104-P1 | 19.6 | −47.64 | >32 | −13.75 |
| 104-P2 | 4.5 | −78.16 | >32 | −14.86 |
| 105-P1 | NA | NA | NA | NA |
| 105-P2 | NA | NA | NA | NA |
| 106-P1 | NA | NA | NA | NA |
| 106-P2 | NA | NA | NA | NA |
| 107-rac | >32 | 10.08 | >32 | 8.64 |
| 107-P1 | 25.8101 | −15.72 | >32 | 14.07 |
| 107-P2 | >32 | −9.6 | >32 | 8.96 |
| 108-rac | >32 | −8.01 | >32 | −4.32 |
| 108-P1 | 29.9 | −24.9 | >32 | −12.05 |
| 108-P2 | 5.6 | 22.43 | >32 | 16.1 |
| 101 | 0.524 | −68.74 | 12.3 | 18.4 |
| 102 | 8.63 | −60.66 | >32 | −16.83 |
| 103 | 2.14 | −83.25 | >32 | −13.31 |
| 109 | 0.45 | −73.56 | >32 | −12.5 |
| 109-P1 | >32 | −32.29 | >32 | 9.06 |
| 109-P2 | 0.52 | −74.31 | >32 | −14.32 |
| 110-P1 | 8.49 | −50.96 | >32 | −8.95 |
| 110-P2 | 3.38 | −47.09 | >32 | −12.31 |
| 111 | >32 | −9.02 | >32 | 10.58 |
| 111-P1 | >32 | −12.73 | >32 | −10.33 |
| 111-P2 | >32 | −11.51 | >32 | −9.06 |
| 112 | >32 | 18.48 | >32 | −8.84 |
| 112-P1 | >32 | 26.46 | <32 | 9.15 |
| 112-P2 | >32 | 13.14 | >32 | 7.52 |
| 113 | 0.478 | −46.53 | >32 | 13.12 |
| 113-P1 | >32 | −13.24 | >32 | −9.71 |
| 114-rac | 12.9 | −44.74 | >32 | 18.3 |
| 114-P1 | 29.9 | −28.1 | >32 | 5.99 |
| 114-P2 | >32 | −15.43 | >32 | 9.94 |
| 115 | 0.295 | −79.02 | 12.26 | 71.79 |
| 116-P1 | >32 | −10.6 | >32 | 18.63 |
| 116-P2 | >32 | −16.96 | >32 | −19.89 |
| 117-P1 | 25.9 | −16.91 | >32 | 7.72 |
| 117-P2 | >32 | −15.88 | >32 | −15.43 |
| 118 | >32 | −9.08 | >32 | −8.52 |
| 118-P1 | >32 | −7.97 | >32 | 9.54 |
| 118-P2 | >32 | 0 | >32 | 0 |
| 119 | >32 | 20.39 | >32 | 14.48 |
| 119-P1 | >32 | −14.14 | >32 | −14.31 |
| 119-P2 | >32 | 0 | >32 | 0 |
| 120 | >32 | −27.71 | >32 | 15.88 |
| 121-P1 | NA | NA | NA | NA |
| 120-P2 | >32 | −24.61 | >32 | −11.21 |
| 121-P1 | NA | NA | NA | NA |
| 121-P2 | >32 | −9.03 | >32 | 7.35 |
| 122 | >32 | −12.17 | >32 | −10.53 |
| 123 | >32 | 0.49 | >32 | 1.83 |
| 124 | 3.9 | −76.8 | >32 | 7.5 |
| 125 | 0.2 | −88.7 | 14.9 | 58.0 |

### Example 5: Preparation and Characterization of Exemplary Salts

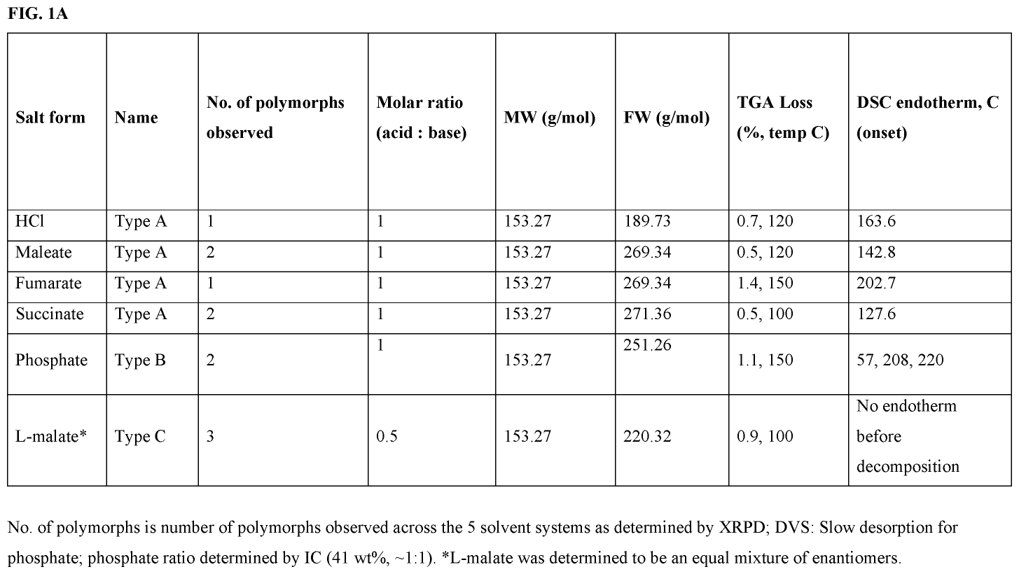

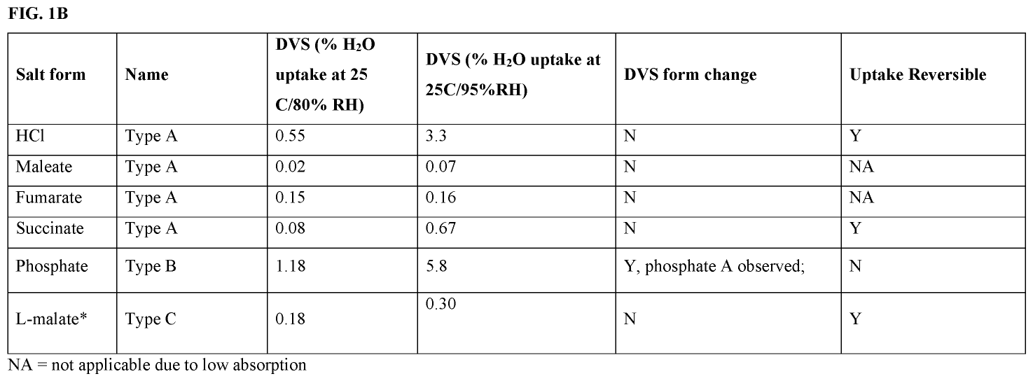

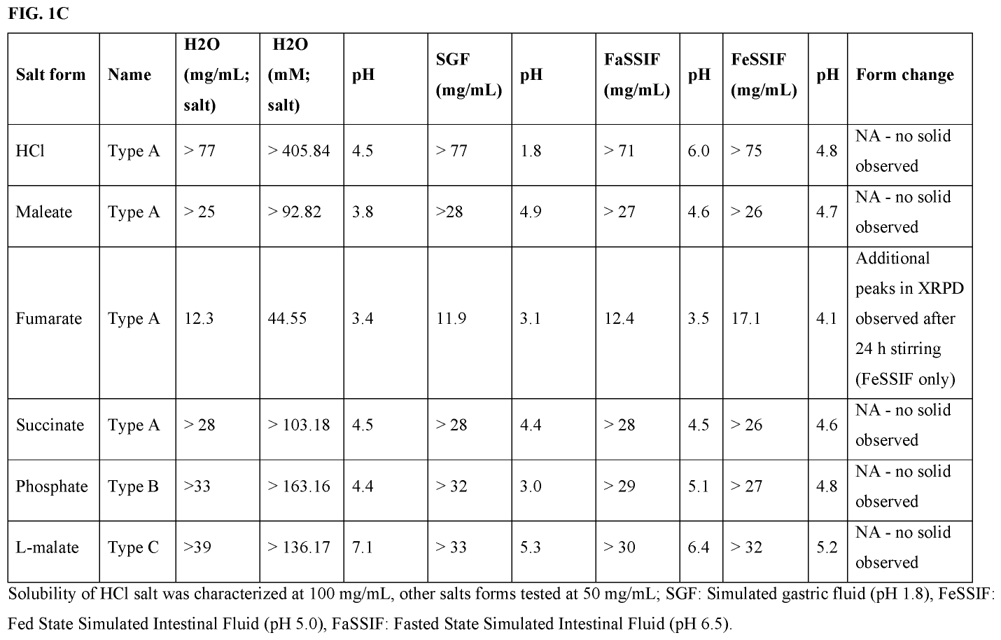

A salt screen was conducted in order to identify other counter ions that may possess advantageous properties relative to the HCl salt. A panel of 20 acids was added to the BRD4780 freebase dissolved in 5 different solvents (100 conditions in total, see table 5 for conditions). 80 of these conditions generated solids that were analyzed by XRPD, TGA, DSC, NMR and/or IC (to confirm molar ratio and presence of BRD4780). From this analysis, 5 salts were chosen for reparation based on the TGA and DSC data for further profiling. Procedures for re-preparation of the salts are described provided. The melting points of the solids were determined. These were then characterized for solubility in water, SGF, FASSIF, FESSIF; for hygroscopicity using DVS; for stability at 25° C./60% RH and 40° C./75% RH as analyzed by LCMS and XRPD. Finally, the new salt forms were characterized in a low dose mouse PK study to determine oral bioavailability. Although the HCl salt displays desirable properties, several other salts including the maleate, fumarate and succinate salts show improvements while providing good overall properties. See FIG. 1.

Exemplary Procedure for the Preparation Of BRD4780 Freebase

To a vial charged with BRD4780 HCl (5.0 g, 0.26 mmol) was added water (80 mL) to give a clear solution. A 1.0 M aq. NaOH solution (27 mL) was added dropwise under stirring, upon which a white precipitate appeared. EtOAc was added (80 mL) under vigorous stirring, yielding two clear layers which were stirred for 5 min and then separated. The aqueous phase was extracted with EtOAc (3×50 mL) and the combined organic extracts were dried over Na₂SO₄, filtered and concentrated carefully (the free amine proved to be volatile (64-68° C. @6 mbar), so concentration on the rotavap was done with care, with the water bath set to 28° C. and the vacuum going down to 90 mbar, then at 40 mbar for 5 min and on the Schlenk line for 5 min) to give the desired free amine (3.90 g, 25.5 mmol, 97%) as a light yellow oil.

Exemplary Salt Screening Procedure

According to the approximate solubility of BRD4780 freebase in 12 solvents and pKa of BRD4780 freebase (10.52, predicted by Marvin software), salt screening was performed under 100 conditions using 20 acids in 5 solvent systems. About 460 mg of BRD4780 freebase was dissolved in each solvent to prepare clear stock solution, and the solution was then distributed to 20 vials. The acids were mixed with freebase in a molar ratio of 1:1 in five solvents, and then stirred at RT for about 2 days. After centrifugation, resulting solids were dried under vacuum at RT, and then analyzed by XRPD. Clear/gel/oil samples were stirred at 5° C., and transferred to evaporation at RT if still no solids.

TABLE 5
Approximate solubility of isolated BRD4780 freebase at RT

| Solvent | Solubility (mg/mL) | Solvent | Solubility (mg/mL) |
|---|---|---|---|
| EtOH | S > 40.0 | IPAC | S > 82.0 |
| IPA | S > 48.0 | MTBE | S > 68.0 |
| Acetone | S > 134.0 | 2-MeTHF | S > 34.0 |
| MEK | S > 36.0 | Toluene | S > 122.0 |
| ACN | S > 72.0 | n-Heptane | S > 36.0 |
| EtOAc | S > 36.0 | H₂O | S < 2.6 |

TABLE 6
Summary of salt screening for BRD4780 freebase

| | | Solvents | | | | |
|---|---|---|---|---|---|---|
| | | A | | | | |
| | | IPA/H₂O | B | C | D | E |
| Acids | | (19:1, v/v) | Acetone | EtOAc | MTBE | Toluene |
| 0 | Blank | Gel | Gel | Gel | Gel | Gel |
| 1 | Hydrochloric | Gel | HCl salt Type | HCl salt Type | HCl salt Type | HCl salt Type |
| | acid | | A* | A | A* | A |
| 2 | Sulfuric acid | Gel | Gel | Gel | Gel | Gel |
| 3 | L-aspartic | L-aspartate | L-aspartate | L-aspartate | L-aspartate | L-aspartate |
| | acid | Type A + acid | Type A + acid | Type A + acid** | Type A + acid | Type A + acid |
| 4 | Maleic acid | Maleate Type | Gel | Maleate Type | Maleate Type | Maleate Type |
| | | B^{#} | | A | A | A + acid |
| 5 | Phosphoric | Phosphate | Phosphate | Phosphate | Phosphate | Phosphate |
| | acid | Type A | Type B | Type B + peaks | Type B | Type A + peaks |
| 6 | L-glutamic | Glutamate | Glutamate | Glutamate | Glutamate | Glutamate |
| | acid | Type | Type | Type | Type | Type |
| | | A + acid** | A + B + acid | B + acid* | B + acid | B + acid |
| 7 | L-tartaric acid | L-tartrate | L-tartrate | L-tartrate | L-tartrate | L-tartrate |
| | | Type | Type A | Type B | Type | Type |
| | | B + acid* | | | B + acid | A + B + peaks^{#} |
| 8 | Fumaric acid | Fumarate | Fumarate | Fumarate | Fumarate | Fumarate |
| | | Type A | Type A | Type A + acid | Type A + acid | Type A* |
| 9 | Citric acid | Citrate Type | Citrate Type | Citrate Type | Citrate Type | Citrate Type |
| | | A + acid | A | A + acid + peaks | A + peaks* | A + peaks^{#} |
| 10 | L-malic acid | L-malate Type | L-malate Type | L-malate Type | L-malate Type | L-malate Type |
| | | A + B + C* | A | A + B | B | A + B^{#} |
| 11 | Lactic acid | Gel | Gel | Lactate Type | Lactate Type | Gel |
| | | | | A* | A* | |
| 12 | Succinic acid | Succinate | Succinate | Succinate | Succinate | Succinate |
| | | Type B^{#} | Type A + acid | Type A + acid | Type A | Type A + acid |
| 13 | Adipic acid | Gel | Adipate Type | Adipate Type | Adipate Type | Adipate Type |
| | | | A + acid | A** | A + acid | A + acid |
| 14 | Acetic acid | Acetate Type | Acetate Type | Acetate Type | Acetate Type | Acetate Type |
| | | A^{#} | A | A | A | A |
| 15 | p- | Tosylate Type | Tosylate Type | Tosylate Type | Tosylate Type | Tosylate Type |
| | toluenesulfonic | A^{#} | A* | A | A | A |
| | acid | | | | | |
| 16 | Methanesulfonic | Gel | Gel | Mesylate | Mesylate | Gel |
| | acid | | | Type A | Type A | |
| 17 | Benzenesulfonic | Besylate Type | Besylate Type | Besylate Type | Besylate Type | Besylate Type |
| | acid | B^{#} | A | B | B | B* |
| 18 | Malonic acid | Gel | Gel | Gel | Malonate | Gel |
| | | | | | Type A | |
| 19 | Benzoic acid | Benzoate | Benzoate | Benzoate | Benzoate | Benzoate |
| | | Type A + acid | Type A + acid* | Type A* | Type A + acid* | Type A* |
| 20 | Hydrobromic | Gel | Gel | HBr salt Type | HBr salt Type | HBr salt Type |
| | acid | | | A | A | B* |

*Solids were obtained from slurry at 5° C.;
^{#}Solids were obtained from evaporation at RT;
**A mixture of salt and acid was obtained, and additional freeform was added.

Results from Salt Screen

TGA and DSC

TGA data were collected using a TA Discovery5500/Q5000 TGA from TA Instruments. DSC was performed using a TA Discovery2500/Q2000 DSC from TA Instruments. Detailed parameters used are listed in Table 7.

TABLE 7
Parameters for TGA and DSC test

| Parameters | TGA | DSC |
|---|---|---|
| Method | Ramp | Ramp |
| Sample pan | Aluminum, open | Aluminum, crimped/open |
| Temperature | RT - desired temperature | 25° C. - desired temperature |
| Heating rate | 10° C./min | 10° C./min |
| Purge gas | N₂ | N₂ |

NMR
¹H Solution NMR was collected on a Bruker 400 MHz NMR spectrometer using MeOD as solvent. For malonate Type A, D₂O was used as solvent to avoid signal overlap.

Detailed chromatographic conditions for counter-ion stoichiometric ratio measurement are listed in Table 8.

TABLE 8
Chromatographic conditions and parameters for IC test

| Parameters | Ion Chromatography (Thermo ICS1100) |
|---|---|
| Mode | Anion |
| Column | IonPac AS18 Analytical Column (4 × 250 mm) |
| Mobile phase | 25 mM NaOH |
| Injection volume | 25 μL |
| Flow rate | 1.0 mL/min |
| Cell Temp. | 35° C. |
| Column Temp. | 35° C. |
| Current | 80 Ma |
| Run Time | 7 min (Cl⁻), 15 min (Br⁻) and 40 min (PO₄³⁻) |

TABLE 9
Summary of characterization results for salt hits

| | | TGA loss | DSC Endotherm | Molar ratio | Solvent |
|---|---|---|---|---|---|
| Solid Form | | (%, temperature) | (° C., peak) | (acid:base) | (wt %) |
| HCl Salt | Type A | 0.7 (120° C.) | 159.8* | 1.0^{#} | No |
| L-aspartate | Type A | 3.2 (100° C.) | No | 1.0 | No |
| Maleate | Type A | 1.4 (120° C.) | 68.3*, 133.6* | 1.0 | No |
| | Type B | 1.0 (100° C.) | 84.2, 125.0, | 1.2 | No |
| | | | 136.8, 160.7 | | |
| Phosphate | Type A | 5.8 (120° C.) | 57.3, 81.6 | 1.1^{#} | No |
| | Type B | 2.2 (120° C.) | 108.3*, 216.9 | 1.1^{#} | No |
| L-glutamate | Type A | 15.7 (90° C.) | 83.2* | 1.0 | 7.4 |
| | | | | | (IPA) |
| | Type B | 2.2 (100° C.) | No | 1.0 | No |
| L-tartrate | Type A | 3.4 (130° C.) | 94.4, 111.3, | 1.1 | 0.7 |
| | | | 158.3 | | (acetone) |
| | Type B | 5.3 (120° C.) | 98.5, 157.6 | 1.1 | No |
| Fumarate | Type A | 0.8 (120° C.) | 184.9, 200.2 | 1.0 | 0.2 |
| | | | | | (acetone) |
| Citrate | Type A | 5.9 (120° C.) | 91.7* | 1.0 | 0.2 |
| | | | | | (acetone) |
| L-malate | Type A | 1.4 (100° C.) | 123.0* | 1.0 | No |
| | Type B | 1.8 (100° C.) | 105.4* | 1.0 | No |
| | Type C | 0.7 (100° C.) | No | 0.5 | No |
| Lactate | Type A | 0.8 (70° C.) | 87.8* | 0.9 | No |
| Succinate | Type A | 0.8 (100° C.) | 129.7* | 1.0 | No |
| | Type B | 0.3 (100° C.) | 111.4, 126.4 | 1.3 | No |
| Adipate | Type A | 0.7 (100° C.) | 76.2* | 0.5 | No |
| Acetate | Type A | 1.7 (70° C.) | No | 1.0 | No |
| Tosylate | Type A | 2.2 (200° C.) | 75.5, 139.3, | 1.0 | 0.3 |
| | | | 192.4 | | (EtOAc) |
| Mesylate | Type A | 4.4 (150° C.) | 92.8 | 1.2 | No |
| Besylate | Type A | 3.3 (200° C.) | 85.2, 167.9 | 1.3 | No |
| | Type B | 3.3 (150° C.) | 75.5, 149.5, | 1.0 | No |
| | | | 187.2 | | |
| Malonate | Type A | 1.1 (100° C.) | 83.5* | 0.2 | No |
| Benzoate | Type A | 3.6 (80° C.) | 107.3 | 1.0 | No |
| HBr salt | Type A | 3.3 (150° C.) | 90.3, 167.4 | 0.8^{#} | No |
| | Type B | 11.7 (150° C.) | 55.4, 121.7, | 1.0^{#} | No |
| | | | 165.7 | | |

*onset temperature;
^{#}Ion content was determined by IC, and the molar ratio was calculated.

Exemplary Salts

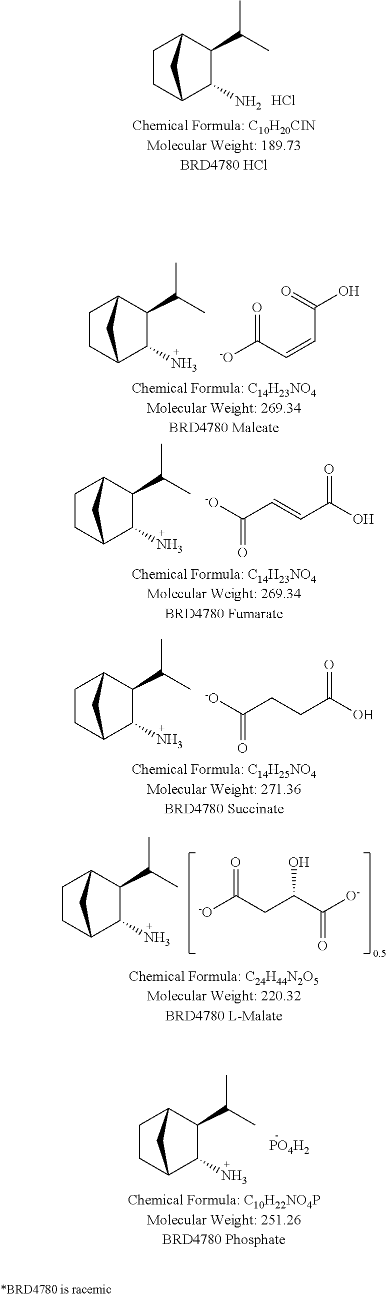

Physical and Chemical Stability

To compare the physical/chemical stability of the salt candidates, approximate 10 mg of each solid sample was added to a 5-mL glass vial covered with Parafilm® with several pin-holes on it, and then stored under 25° C./60% RH and 40° C./75% RH. After one week storage, solids were taken out for LC-MS and XRPD test to evaluate physical and chemical stability. HCl salt Type A was additionally stored at 60° C. for 24 h and tested by LC-MS and XRPD. Stability results are summarized in Table 106. No significantly new peak was observed for all salt candidates on LC-MS chromatogram after stored under the conditions of 25° C./60% RH and 40° C./75% RH for one week. For XRPD results, new peaks were observed for succinate Type A at 25° C./60% RH for 1 week. Phosphate Type A partly converted to phosphate Type B at 25° C./60% RH and 40° C./75% RH for 1 week, and L-malate Type C partly converted to L-malate Type B at 40° C./75% RH for 1 week. For HCl salt Type A, a new peak was observed on the LC-MS chromatogram after stored at 60° C. for 24 h. XRPD results are displayed in FIGS. 2A-R.

TABLE 10
Summary of stability results for salt candidates

| Form | Condition | Time Point | | Solid Form |
|---|---|---|---|---|
| HCl salt | 25° C./60% RH | 1 | week | HCl salt Type A |
| Type A | 40° C./75% RH | | | HCl salt Type A |
| | 60° C. | 24 | h | HCl salt Type A |
| Maleate | 25° C./60% RH | 1 | week | Maleate Type A |
| Type A | 40° C./75% RH | | | Maleate Type A |
| Fumarate | 25° C./60% RH | 1 | week | Fumarate Type A |
| Type A | 40° C./75% RH | | | Fumarate Type A |
| Succinate | 25° C./60% RH | 1 | week | Succinate Type A + peaks |
| Type A | 40° C./75% RH | | | Succinate Type A |
| Phosphate | 25° C./60% RH | 1 | week | Phosphate Type A + B |
| Type B | 40° C./75% RH | | | Phosphate Type A + B |
| L-malate | 25° C./60% RH | 1 | week | L-malate Type C |
| Type C | 40° C./75% RH | | | L-malate Type B + C |

* The peak area of API was 8150730 and a new peak at RT-2.891 min was observed, with area of 39030.9 (0.48 area %).

TABLE 11
Summary of stability results for salt candidates

| Form | Condition | Time Point | | Solid Form |
|---|---|---|---|---|
| HCl salt | 25° C./60% RH | 1 | week | HCl salt Type A |
| Type A | 40° C./75% RH | | | HCl salt Type A |
| | 60° C. | 24 | h | HCl salt Type A |
| Maleate | 25° C./60% RH | 1 | week | Maleate Type A |
| Type A | 40° C./75% RH | | | Maleate Type A |
| Fumarate | 25° C./60% RH | 1 | week | Fumarate Type A |
| Type A | 40° C./75% RH | | | Fumarate Type A |
| Succinate | 25° C./60% RH | 1 | week | Succinate Type A + peaks |
| Type A | 40° C./75% RH | | | Succinate Type A |
| Phosphate | 25° C./60% RH | 1 | week | Phosphate Type A + B |
| Type B | 40° C./75% RH | | | Phosphate Type A + B |
| L-malate | 25° C./60% RH | 1 | week | L-malate Type C |
| Type C | 40° C./75% RH | | | L-malate Type B + C |

* The peak area of API was 8150730 and a new peak at RT-2.891 min was observed, with area of 39030.9 (0.48 area %).

Melting Point Characterization

Instrument

SRS Melting Point Apparatus MPA100.

Capillaries

Kimble Brosilicate Glass, cat. 34505-99, 1.5-1.8×90 mm. Accuracy of the instrument was performed by measuring the melting point of DMAP (Sigma-Aldrich, cat. 39405, purum ≥98%, vendor reported m.p.=111-114° C., measured m.p.=111-114° C.).

Sample Preparation

The sample was prepared by pressing the capillary gently into the fine white powder and pushed to the bottom of the tube by repeatedly tapping the bottom of the capillary against the bench hard surface, providing good packing of the sample. The sample height was between 2 and 3 mm. The outside surface of the tube was wiped with a clean cloth before being inserted into the heating stand.

Procedure

Initial scouting run: 110° C./stop temp.: 300° C./ramp: 10° C./min. Precise determination: ramp 1° C./min.

TABLE 12
Thermal Properties of Exemplary Salts

| Salt | MP (° C.) | MP Observations |
|---|---|---|
| HCl | 215-270, | Slow onset of decomposition (slow darkening of the |
| Type A | decomp. | white powder) starting around 215° C. (small black |
| | | spots observed within the white powder at first, then |
| | | the color change generalized to become darker and |
| | | finally become charcoal black). By the time the |
| | | temperature reaches 270° C., the sample is fully |
| | | decomposed into a black substance. |
| Fumarate | 200, decomp. | At 200° C., the solid quickly melted while |
| Type A | | simultaneously turning yellow and bubbling strongly |
| | | (signs of decomposition), the liquid raising in the |
| | | capillary. |
| Succinate | 140-159 | Onset temperature observed at 140° C., meniscus |
| Type A | | temperature observed at 145° C., clear point reached at |
| | | 159° C. with bubbling observed in the capillary (no |
| | | change of color observed). |
| Maleate | 139.7-141.0 | Sharp well defined melting point: 139.7-141.0° C. |
| Type A | | |
| L-Malate | 194, decomp | At 194° C., the solid quickly melted while |
| Type C | | simultaneously turning yellow and bubbling strongly |
| | | (signs of decomposition), the liquid raising in the |
| | | capillary. |
| Phosphate | NA | The solid started to turn yellow, then brown starting at |
| Type B | | 220° C. The experiment was discontinued at 250° C. |
| | | Material observed as a viscous paste. |

Solubility

During the evaluation of equilibrium solubility in bio-relevant media for salt candidates, freebase concentration was calculated with a calibration curve using a series of diluted standard samples. 2.162 mg HCl salt Type A was added into 10-mL volumetric flask and add ACN/H₂O (1:1, v:v) to dissolve the solid and to the target volume, which was labeled as std. A series of standard solutions were diluted (std-2˜8), and the LC-MS of standard samples were displayed in Tables 9 and 10. The two calibration curves were used for solubility calculation of HCl salt Type A and other forms, respectively.

TABLE 13
LC-MS results of standard samples for HCl salt Type A

| Sample | Dilution factor | Conc.(mg/mL)* | Area (mAU*S) |
|---|---|---|---|
| Std | 1 | 0.1758 | 20360133 |
| Std-2 | 4 | 0.0439 | 8204110 |
| Std-3 | 5 | 0.0352 | 7044260 |
| Std-4 | 10 | 0.0176 | 4381010 |
| Std-5 | 20 | 0.0088 | 2498580 |
| Std-6 | 30 | 0.0059 | 1876200 |
| Std-7 | 40 | 0.0044 | 1450070 |
| Std-8 | 50 | 0.0035 | 1259190 |

*The conc. was calculated by weight of HCl salt/volume × (1-TGA weight loss-Cl⁻ content).

TABLE 14
LC-MS results of standard samples for solubility
of maleate Type A, fumarate Type A, succinate
Type A, phosphate Type B and L-malate Type C

| Sample | Dilution factor | Conc.(mg/mL)* | Area (mAU*S) |
|---|---|---|---|
| Std | 1 | 0.1758 | 23684100 |
| Std-2 | 4 | 0.0439 | 10080000 |
| Std-3 | 5 | 0.0352 | 8812230 |
| Std-4 | 10 | 0.0176 | 5408610 |
| Std-5 | 20 | 0.0088 | 3266750 |
| Std-6 | 30 | 0.0059 | 2411340 |
| Std-7 | 40 | 0.0044 | 1917700 |
| Std-8 | 50 | 0.0035 | 1619240 |

*The conc. was calculated by weight of HCl salt/volume × (1-TGA weight loss-Cl⁻ content).

SGF

200.8 mg of sodium chloride and 100.9 mg of Triton X-100 were weighted into a 100 mL volumetric flask. Purified water was added and the mixture was sonicated until all solids were completely dissolved. The Approximately 1.632 mL of 1 M HCl and sufficient purified water were added to pH 1.8. Purified water was added and the mixture was mixed and the pH was determined to be 1.78 with a pH meter.

FaSSIF

Preparation of FaSSIF dissolving buffer: 340.7 mg of sodium phosphate monobasic, 42.6 mg of sodium hydroxide and 620.0 mg of sodium chloride were weighed into a 100-mL volumetric flask. Purified water as added until the solids were completely dissolved. Purified water was added and the pH was adjusted to 6.5. The Add sufficient purified water closely to the target volume and adjust to pH 6.5. Purified water was added and the mixture was mixed and the pH was determined to be 6.47 with a pH meter.

Preparation of FaSSIF: 111.1 mg of SIF powder was weighed into a 50-mL volumetric flask. FaSSIF dissolving buffer was added and the mixture was sonicated until the SIF powder was complete dissolved. The FaSSIF solution could be stored at 4° C. for 7 days and was equilibrated for 2 hours to RT before use.

FeSSIF

Preparation of FeSSIF dissolving buffer: 0.82 mL of glacial acetic acid, 405.4 mg of sodium hydroxide and 1187.9 mg of sodium chloride were transferred into a 100-mL volumetric flask. The solids were then dissolved with purified water. Purifeid water was added and the pH was adjusted to 5.0. The mixture was mixed and the pH was determined to be 4.47 with a pH meter.

Preparation of FeSSIF: 561.1 mg of SIF powder was transferred into a 50-mL volumetric flask. FeSSIF dissolving buffer was added and the mixture was sonicated until SIF powder was completely dissolved. FeSSIF dissolving buffer was added and the mixture was mixed. The FaSSIF solution could be stored at 4° C. for 7 days and was equilibrated for 2 hours to RT before use.

Exemplary Solubility Results

TABLE 15
LC-MS results of standard samples for solubility
of maleate Type A, fumarate Type A, succinate
Type A, phosphate Type B and L-malate Type C

| | | H₂O | H₂O | | SGF |
|---|---|---|---|---|---|
| Salt form | Name | (mg/mL; salt) | (mM; salt) | pH | (mg/mL) |
| HCl | Type A | >77 | >405.84 | 4.5 | >77 |
| Maleate | Type A | >25 | >92.82 | 3.8 | >28 |
| Fumarate | Type A | 12.3 | 44.55 | 3.4 | 11.9 |
| Succinate | Type A | >28 | >103.18 | 4.5 | >28 |
| Phosphate | Type B | >33 | >163.16 | 4.4 | >32 |
| L-malate | Type C | >39 | >136.17 | 7.1 | >33 |

| | | FaSSIF | | FeSSIF | |
|---|---|---|---|---|---|
| Salt form | pH | (mg/mL) | pH | (mg/mL) | pH |
| HCl | 1.8 | >71 | 6.0 | >75 | 4.8 |
| Maleate | 4.9 | >27 | 4.6 | >26 | 4.7 |
| Fumarate | 3.1 | 12.4 | 3.5 | 17.1 | 4.1 |
| Succinate | 4.4 | >28 | 4.5 | >26 | 4.6 |
| Phosphate | 3.0 | >29 | 5.1 | >27 | 4.8 |
| L-malate | 5.3 | >30 | 6.4 | >32 | 5.2 |

Solubility of HCl salt was characterized at 100 mg/mL, other salts forms tested at 50 mg/mL; SGF: Simulated gastric fluid (pH 1.8), FeSSIF: Fed State Simulated Intestinal Fluid (pH 5.0), FaSSIF: Fasted State Simulated Intestinal Fluid (pH 6.5).

Exemplary DVS Testing

Dynamic Vapor Sorption (DVS) was measured via a SMS (Surface Measurement Systems) DVS Intrinsic. The relative humidity at 25° C. were calibrated against the deliquescence point of LiCl, Mg(NO₃)₂ and KCl. Parameters for DVS test are listed in Table 16.

TABLE 16
Parameters for DVS testing

| Parameters | DVS |
|---|---|
| Temperature | 25° C. |
| Sample size | 10~20 mg |
| Gas and flow rate | N₂, 200 mL/min |
| dm/dt | 0.002%/min |
| Min. | 10 min |
| dm/dtstabilityduration | |
| Max. equilibrium time | 180 min |
| RH range | 0% RH-95% RH-0% RH |
| RH step size | 10% (90% RH-0% RH-90% RH) |
| | 5% (95% RH-90% RH and 90% RH-95% RH) |

TABLE 17
Parameters for DVS test

| | | DVS (% | DVS (% | | |
|---|---|---|---|---|---|
| | | H₂O | H₂O | | |
| | | uptake at | uptake at | | |
| | | 25 C./ | 25 C./ | DVS form | Uptake |
| Salt form | Name | 80% RH) | 95% RH) | change | Reversible |
| HCl | Type A | 0.55 | 3.3 | N | Y |
| Maleate | Type A | 0.02 | 0.07 | N | NA |
| Fumarate | Type A | 0.15 | 0.16 | N | NA |
| Succinate | Type A | 0.08 | 0.67 | N | Y |
| Phosphate | Type B | 1.18 | 5.8 | Y, phosphate | N |
| | | | | A observed; | |
| L-malate * | Type C | 0.18 | 0.30 | N | Y |

NA = not applicable due to low absorption

### Summary

Solubility

The solubility of all materials is high with all test articles other than the fumarate salt being more soluble than 50 mg/mL. Additional peaks in the XRPD spectra were observed for the Fumarate after stirring for 24 hours in FeSSIF. The salts exhibited different effects on the pH of the solutions tested. The L-malate salt gave higher pH of the final solutions across the four test systems while the fumarate displayed the lowest across the four test systems.

Melting Point

The maleate salt was the only test article examined that has a sharp, well-defined melting point (139.7-141.0° C.).

Characterizations

TGA/DSC. Good thermal characteristics (low loss of mass by TGA during well-defined endotherm observed in DSC) were observed for the HCl and the maleate forms. Fumarate and succinate salts displayed endotherms coinciding with TGA mass loss with the fumarate displaying the highest temperature endotherm. The phosphate displayed 3 endotherms the last of which occurred with significant mass loss by TGA. The L-malate did not display an endotherm prior to decomposition.

Hygroscopicity

The BRD4780 HCl salt is classified as slightly hygroscopic due to its reversible absorption of 0.55 wt % H₂O at 80% RH. This material absorbs 3.3 wt % H₂O at 90% RH. The BRD4780 maleate, fumarate, succinate and L-malate salts are all classified as not hygroscopic absorbing <0.2 wt % H₂O at 80% RH. The phosphate salt hygroscopic and did not fully desorb the absorbed H₂O.

Stability

The HCl, maleate, and fumarate salts of BRD4780 did not display decomposition or solid form change during the 1 week stability assay. There were new peaks observed in the succinate and form change observed in the phosphate and L-malate salts.

TABLE 18
Stability information

| | | Stability: 25/60% RH; | |
|---|---|---|---|
| Salt form | Name | 40 C./75% RH; 1 week | Form change? |
| HCl | Type A | No significant impurities | No change |
| Maleate | Type A | No significant impurities | No change |
| Fumarate | Type A | No significant impurities | No change |
| Succinate | Type A | No significant impurities | New Peaks |
| Phosphate | Type B | No significant impurities | Phosphate A |
| | | | observed |
| L-malate | Type C | No significant impurities | Type B observed |

Impurities were estimated by LCMS by observation of the TIC.

TABLE 19
Matrix of results from solid form characterization

| | | | | | Melting |
|---|---|---|---|---|---|
| Salt form | Solubility | TGA/DSC | DVS | Stability | Point |
| HCl | Good | Good | Moderate | Good | Poor |
| Maleate | Good | Good | Good | Good | Good |
| Fumarate | Moderate | Good | Good | Good | Moderate |
| Succinate | Good | Good | Good | Moderate | Moderate |
| Phosphate | Good | Poor | Poor | Poor | Moderate |
| L-malate | Good | Poor | Good | Poor | Poor |

### Example 6: XRPD Characterization of Exemplary Salts

PANalytical Empyrean and X' Pert3 X-ray powder diffractometers were used. The XRPD parameters used are listed in Table 20. Stoichiometry of counter-ions was confirmed by NMR and/or IC.

TABLE 20
XRPD parameters

| Parameters | Empyrean | X′ Pert3 |
|---|---|---|
| X-Ray wavelength | Cu, Kα; | Cu, Kα; |
| | Kα1 (Å): 1.540598 | Kα1 (Å): 1.540598 |
| | Kα2 (Å): 1.544426 | Kα2 (Å): 1.544426 |
| | intensity ratio | intensity ratio |
| | Kα2/Kα1: 0.50 | Kα2/Kα1: 0.50 |
| X-Ray tube setting | 45 kV, 40 mA | 45 kV, 40 mA |
| Divergence slit | Automatic | ⅛° |
| Scan mode | Continuous | Continuous |
| Scan range (2θ/°) | 3°~40° | 3°~40° |
| Step size (2θ/°) | 0.0167° | 0.0263° |
| Scan step time (s) | 17.780 | 46.665 |
| Test time (s) | About 5 min 30 s | About 5 min |

TABLE 21
HCl Salt Type A

| | | FWHM | | |
|---|---|---|---|---|
| Pos. [°2θ] | Height [cts] | Left [°2θ] | d-spacing [Å] | Rel. Int. [%] |
| 6.87 | 10051.56 | 0.1023 | 12.87 | 100.00 |
| 12.56 | 95.70 | 0.1023 | 7.05 | 0.95 |
| 13.69 | 4538.20 | 0.1023 | 6.47 | 45.15 |
| 15.16 | 4322.00 | 0.1023 | 5.84 | 43.00 |
| 15.53 | 2643.60 | 0.1023 | 5.71 | 26.30 |
| 17.60 | 709.14 | 0.1023 | 5.04 | 7.05 |
| 18.60 | 351.29 | 0.1023 | 4.77 | 3.49 |
| 19.18 | 514.64 | 0.1279 | 4.63 | 5.12 |
| 19.69 | 1312.62 | 0.1279 | 4.51 | 13.06 |
| 20.56 | 1813.98 | 0.1279 | 4.32 | 18.05 |
| 21.14 | 491.69 | 0.1023 | 4.20 | 4.89 |
| 21.40 | 163.40 | 0.1023 | 4.15 | 1.63 |
| 21.82 | 704.53 | 0.1279 | 4.07 | 7.01 |
| 22.84 | 370.09 | 0.1023 | 3.89 | 3.68 |
| 23.14 | 2046.21 | 0.1023 | 3.84 | 20.36 |
| 23.82 | 2399.31 | 0.1535 | 3.74 | 23.87 |
| 24.30 | 145.32 | 0.0768 | 3.66 | 1.45 |
| 25.18 | 281.24 | 0.1279 | 3.54 | 2.80 |
| 26.53 | 147.95 | 0.1023 | 3.36 | 1.47 |
| 26.98 | 450.98 | 0.1023 | 3.31 | 4.49 |
| 27.50 | 1331.12 | 0.1023 | 3.24 | 13.24 |
| 28.27 | 130.28 | 0.1279 | 3.16 | 1.30 |
| 28.58 | 537.51 | 0.1279 | 3.12 | 5.35 |
| 29.09 | 327.90 | 0.1279 | 3.07 | 3.26 |
| 29.49 | 152.28 | 0.1023 | 3.03 | 1.51 |
| 30.46 | 563.01 | 0.1023 | 2.93 | 5.60 |
| 31.21 | 836.66 | 0.1535 | 2.87 | 8.32 |
| 32.11 | 116.21 | 0.0768 | 2.79 | 1.16 |
| 33.53 | 125.44 | 0.1279 | 2.67 | 1.25 |
| 34.06 | 115.22 | 0.1535 | 2.63 | 1.15 |
| 34.54 | 885.28 | 0.1535 | 2.60 | 8.81 |
| 35.08 | 79.92 | 0.1535 | 2.56 | 0.80 |
| 36.38 | 375.98 | 0.1535 | 2.47 | 3.74 |
| 37.34 | 231.20 | 0.1023 | 2.41 | 2.30 |
| 38.21 | 70.54 | 0.2047 | 2.36 | 0.70 |
| 39.15 | 189.40 | 0.1279 | 2.30 | 1.88 |
| 39.73 | 154.99 | 0.0768 | 2.27 | 1.54 |

Maleate

In salt screening experiments, two forms of the BRD4780 maleate salt were observed in different solvents, which were named as maleate Type A and maleate Type B. Maleate Type A (was obtained by stirring BRD4780 freebase (and equimolar maleic acid in EtOAc at RT for 2 days. Maleate Type B was obtained by stirring BRD4780 freebase and equimolar maleic acid in IPA/H₂O (19:1, v:v) at RT for 2 days, followed by stirring at 5° C. for 10 days and then evaporation at RT. Competitive slurry experiments indicated that Type A was the thermodynamically stable form.

TABLE 22
Maleate Type A

| | | FWHM | | |
|---|---|---|---|---|
| Pos. [°2θ] | Height [cts] | Left [°2θ] | d-spacing [Å] | Rel. Int. [%] |
| 9.42 | 1067.95 | 0.1023 | 9.39 | 18.93 |
| 11.59 | 126.18 | 0.1023 | 7.63 | 2.24 |
| 11.99 | 63.14 | 0.1535 | 7.38 | 1.12 |
| 13.32 | 522.93 | 0.0768 | 6.65 | 9.27 |
| 13.68 | 2557.08 | 0.1023 | 6.47 | 45.32 |
| 14.01 | 4110.17 | 0.1023 | 6.32 | 72.84 |
| 14.54 | 706.02 | 0.1023 | 6.09 | 12.51 |
| 15.50 | 1412.05 | 0.1023 | 5.72 | 25.02 |
| 16.40 | 24.18 | 0.1791 | 5.41 | 0.43 |
| 18.36 | 5642.84 | 0.1023 | 4.83 | 100.00 |
| 18.61 | 3490.29 | 0.1023 | 4.77 | 61.85 |
| 18.87 | 2051.91 | 0.1023 | 4.70 | 36.36 |
| 19.23 | 934.36 | 0.1279 | 4.61 | 16.56 |
| 20.00 | 241.57 | 0.1023 | 4.44 | 4.28 |
| 20.71 | 1760.46 | 0.1279 | 4.29 | 31.20 |
| 23.02 | 747.42 | 0.0768 | 3.86 | 13.25 |
| 23.26 | 5210.02 | 0.1023 | 3.82 | 92.33 |
| 23.59 | 1057.60 | 0.1279 | 3.77 | 18.74 |
| 24.39 | 1452.79 | 0.1023 | 3.65 | 25.75 |
| 24.61 | 972.08 | 0.1023 | 3.62 | 17.23 |
| 25.15 | 387.94 | 0.1023 | 3.54 | 6.87 |
| 25.43 | 480.80 | 0.1023 | 3.50 | 8.52 |
| 25.85 | 109.27 | 0.0768 | 3.45 | 1.94 |
| 26.48 | 227.82 | 0.0768 | 3.37 | 4.04 |
| 26.80 | 928.55 | 0.1279 | 3.33 | 16.46 |
| 27.52 | 352.90 | 0.1023 | 3.24 | 6.25 |
| 28.45 | 316.56 | 0.1279 | 3.14 | 5.61 |
| 29.58 | 229.62 | 0.1023 | 3.02 | 4.07 |
| 29.80 | 150.40 | 0.1023 | 3.00 | 2.67 |
| 30.06 | 91.24 | 0.0768 | 2.97 | 1.62 |
| 31.20 | 267.13 | 0.1279 | 2.87 | 4.73 |
| 31.72 | 323.65 | 0.1023 | 2.82 | 5.74 |
| 32.39 | 61.17 | 0.1023 | 2.76 | 1.08 |
| 32.70 | 118.83 | 0.1279 | 2.74 | 2.11 |
| 32.93 | 238.22 | 0.1279 | 2.72 | 4.22 |
| 33.51 | 301.69 | 0.1535 | 2.67 | 5.35 |
| 34.31 | 79.25 | 0.1023 | 2.61 | 1.40 |
| 34.90 | 126.01 | 0.0768 | 2.57 | 2.23 |
| 35.16 | 193.84 | 0.1279 | 2.55 | 3.44 |
| 35.95 | 223.78 | 0.1535 | 2.50 | 3.97 |
| 36.32 | 83.87 | 0.2047 | 2.47 | 1.49 |
| 37.10 | 57.85 | 0.1279 | 2.42 | 1.03 |
| 37.54 | 134.45 | 0.1023 | 2.40 | 2.38 |
| 38.18 | 74.98 | 0.2558 | 2.36 | 1.33 |
| 38.69 | 117.01 | 0.0768 | 2.33 | 2.07 |

TABLE 23
Maleate Type B

| | | FWHM | | |
|---|---|---|---|---|
| Pos. [°2θ] | Height [cts] | Left [°2θ] | d-spacing [Å] | Rel. Int. [%] |
| 6.05 | 4897.15 | 0.1023 | 14.61 | 45.47 |
| 10.40 | 250.32 | 0.1023 | 8.50 | 2.32 |
| 11.87 | 10769.38 | 0.1023 | 7.46 | 100.00 |
| 12.11 | 896.04 | 0.0512 | 7.31 | 8.32 |
| 16.20 | 1324.96 | 0.1279 | 5.47 | 12.30 |
| 17.01 | 2211.64 | 0.1279 | 5.21 | 20.54 |
| 17.88 | 231.26 | 0.1023 | 4.96 | 2.15 |
| 18.14 | 218.96 | 0.0768 | 4.89 | 2.03 |
| 18.75 | 935.08 | 0.1279 | 4.73 | 8.68 |
| 19.33 | 1670.84 | 0.1279 | 4.59 | 15.51 |
| 19.66 | 644.26 | 0.1279 | 4.51 | 5.98 |
| 20.27 | 477.47 | 0.1279 | 4.38 | 4.43 |
| 20.68 | 997.12 | 0.1279 | 4.30 | 9.26 |
| 21.22 | 1537.84 | 0.1023 | 4.19 | 14.28 |
| 21.58 | 738.66 | 0.0768 | 4.12 | 6.86 |
| 21.89 | 523.69 | 0.1791 | 4.06 | 4.86 |
| 22.58 | 439.04 | 0.1279 | 3.94 | 4.08 |
| 23.84 | 9382.62 | 0.1279 | 3.73 | 87.12 |
| 24.85 | 131.75 | 0.1279 | 3.58 | 1.22 |
| 25.27 | 195.64 | 0.1023 | 3.52 | 1.82 |
| 25.59 | 190.11 | 0.1023 | 3.48 | 1.77 |
| 26.17 | 784.89 | 0.1535 | 3.41 | 7.29 |
| 27.34 | 395.94 | 0.1535 | 3.26 | 3.68 |
| 27.72 | 185.44 | 0.1535 | 3.22 | 1.72 |
| 28.30 | 82.75 | 0.3070 | 3.15 | 0.77 |
| 29.49 | 679.48 | 0.1535 | 3.03 | 6.31 |
| 29.97 | 122.16 | 0.1535 | 2.98 | 1.13 |
| 30.62 | 251.13 | 0.1279 | 2.92 | 2.33 |
| 30.97 | 182.84 | 0.1279 | 2.89 | 1.70 |
| 31.72 | 786.06 | 0.1535 | 2.82 | 7.30 |
| 32.30 | 1415.62 | 0.1791 | 2.77 | 13.14 |
| 33.81 | 245.95 | 0.1279 | 2.65 | 2.28 |
| 34.35 | 397.45 | 0.1279 | 2.61 | 3.69 |
| 35.03 | 279.37 | 0.1791 | 2.56 | 2.59 |
| 36.08 | 210.42 | 0.1023 | 2.49 | 1.95 |
| 37.81 | 69.75 | 0.2558 | 2.38 | 0.65 |

Phosphate

In salt screening experiments, two forms of BRD4780 phosphate salt were observed in different solvents, which were named as phosphate Type A and phosphate Type B. Phosphate Type A was obtained by stirring and equimolar phosphoric acid in IPA/1H₂O (19:1, v:v) at RT for 2 days. Phosphate Type B was obtained by stirring BRD4780 freebase and equimolar maleic acid in acetone at RT for 2 days.

TABLE 24
Phosphate Type A

| | | FWHM | | |
|---|---|---|---|---|
| Pos. [°2θ] | Height [cts] | Left [°2θ] | d-spacing [Å] | Rel. Int. [%] |
| 6.28 | 94894.93 | 0.0768 | 14.07 | 100.00 |
| 6.89 | 741.08 | 0.1023 | 12.83 | 0.78 |
| 12.56 | 767.05 | 0.1023 | 7.05 | 0.81 |
| 12.86 | 369.28 | 0.1279 | 6.89 | 0.39 |
| 13.51 | 379.94 | 0.0768 | 6.56 | 0.40 |
| 13.85 | 206.18 | 0.0768 | 6.40 | 0.22 |
| 15.87 | 1125.61 | 0.1279 | 5.58 | 1.19 |
| 16.38 | 387.53 | 0.0768 | 5.41 | 0.41 |
| 16.54 | 663.30 | 0.0512 | 5.36 | 0.70 |
| 16.75 | 1234.97 | 0.0768 | 5.29 | 1.30 |
| 17.51 | 1154.76 | 0.0768 | 5.06 | 1.22 |
| 18.87 | 21652.63 | 0.1023 | 4.70 | 22.82 |
| 19.22 | 1807.55 | 0.1023 | 4.62 | 1.90 |
| 19.94 | 516.16 | 0.1535 | 4.45 | 0.54 |
| 20.12 | 560.20 | 0.1023 | 4.41 | 0.59 |
| 20.58 | 600.80 | 0.1023 | 4.32 | 0.63 |
| 21.11 | 662.50 | 0.1023 | 4.21 | 0.70 |
| 21.75 | 3835.21 | 0.1023 | 4.09 | 4.04 |
| 22.44 | 57.02 | 0.1535 | 3.96 | 0.06 |
| 23.08 | 94.82 | 0.0768 | 3.85 | 0.10 |
| 23.28 | 176.86 | 0.1023 | 3.82 | 0.19 |
| 23.73 | 268.25 | 0.0768 | 3.75 | 0.28 |
| 24.55 | 81.05 | 0.1023 | 3.63 | 0.09 |
| 25.25 | 3578.43 | 0.1279 | 3.53 | 3.77 |
| 25.86 | 808.83 | 0.1279 | 3.45 | 0.85 |
| 26.49 | 970.29 | 0.1023 | 3.37 | 1.02 |
| 26.79 | 293.59 | 0.1023 | 3.33 | 0.31 |
| 27.49 | 543.52 | 0.1023 | 3.24 | 0.57 |
| 27.87 | 208.00 | 0.1023 | 3.20 | 0.22 |
| 28.55 | 223.58 | 0.0768 | 3.13 | 0.24 |
| 28.78 | 396.79 | 0.1023 | 3.10 | 0.42 |
| 29.02 | 101.57 | 0.1023 | 3.08 | 0.11 |
| 30.81 | 305.72 | 0.1279 | 2.90 | 0.32 |
| 31.15 | 140.89 | 0.0768 | 2.87 | 0.15 |
| 31.69 | 1141.33 | 0.1279 | 2.82 | 1.20 |
| 32.21 | 399.66 | 0.1279 | 2.78 | 0.42 |
| 32.55 | 107.22 | 0.0768 | 2.75 | 0.11 |
| 33.40 | 357.28 | 0.1023 | 2.68 | 0.38 |
| 33.83 | 92.62 | 0.0768 | 2.65 | 0.10 |
| 34.27 | 43.05 | 0.1535 | 2.62 | 0.05 |
| 34.80 | 105.04 | 0.1023 | 2.58 | 0.11 |
| 36.02 | 348.70 | 0.1279 | 2.49 | 0.37 |
| 36.93 | 160.98 | 0.0768 | 2.43 | 0.17 |
| 37.19 | 116.45 | 0.1023 | 2.42 | 0.12 |
| 38.23 | 852.88 | 0.1279 | 2.35 | 0.90 |
| 38.60 | 133.09 | 0.1535 | 2.33 | 0.14 |

TABLE 25
Phosphate Type B

| | | FWHM | | |
|---|---|---|---|---|
| Pos. [°2θ] | Height [cts] | Left [°2θ] | d-spacing [Å] | Rel. Int. [%] |
| 6.25 | 648.36 | 0.0768 | 14.14 | 3.75 |
| 6.90 | 17274.73 | 0.0768 | 12.80 | 100.00 |
| 11.52 | 134.29 | 0.1023 | 7.68 | 0.78 |
| 13.65 | 1195.32 | 0.1023 | 6.49 | 6.92 |
| 14.22 | 93.44 | 0.1535 | 6.23 | 0.54 |
| 17.13 | 2874.41 | 0.1023 | 5.18 | 16.64 |
| 17.36 | 1903.70 | 0.1023 | 5.11 | 11.02 |
| 17.66 | 1000.27 | 0.1279 | 5.02 | 5.79 |
| 18.33 | 384.64 | 0.1279 | 4.84 | 2.23 |
| 18.80 | 133.39 | 0.0768 | 4.72 | 0.77 |
| 19.32 | 659.87 | 0.0768 | 4.59 | 3.82 |
| 19.57 | 1503.52 | 0.1023 | 4.54 | 8.70 |
| 19.85 | 1039.86 | 0.1279 | 4.47 | 6.02 |
| 20.77 | 3659.33 | 0.1279 | 4.28 | 21.18 |
| 21.49 | 432.14 | 0.1023 | 4.14 | 2.50 |
| 22.10 | 694.75 | 0.1023 | 4.02 | 4.02 |
| 23.13 | 204.85 | 0.1023 | 3.85 | 1.19 |
| 23.39 | 343.94 | 0.1279 | 3.80 | 1.99 |
| 23.94 | 1587.16 | 0.1535 | 3.72 | 9.19 |
| 24.23 | 523.69 | 0.1535 | 3.67 | 3.03 |
| 25.48 | 545.62 | 0.1023 | 3.50 | 3.16 |
| 25.67 | 245.31 | 0.1023 | 3.47 | 1.42 |
| 27.46 | 100.77 | 0.1535 | 3.25 | 0.58 |
| 28.67 | 149.37 | 0.1535 | 3.11 | 0.86 |
| 29.43 | 128.19 | 0.1535 | 3.03 | 0.74 |
| 29.85 | 592.16 | 0.2047 | 2.99 | 3.43 |
| 31.51 | 139.08 | 0.1279 | 2.84 | 0.81 |
| 32.11 | 66.85 | 0.1279 | 2.79 | 0.39 |
| 32.52 | 101.37 | 0.2047 | 2.75 | 0.59 |
| 34.63 | 86.12 | 0.1535 | 2.59 | 0.50 |
| 34.96 | 99.51 | 0.1791 | 2.57 | 0.58 |
| 35.48 | 98.86 | 0.1535 | 2.53 | 0.57 |
| 35.96 | 148.75 | 0.1535 | 2.50 | 0.86 |
| 37.25 | 70.18 | 0.1535 | 2.41 | 0.41 |
| 38.56 | 24.71 | 0.3070 | 2.33 | 0.14 |

Fumarate

In salt screening experiments, BRD4780 fumarate salt Type A was obtained by stirring BRD4780 freebase and equimolar fumuric acid in acetone at RT for 2 days.

TABLE 26
Fumarate Type A

| | | FWHM | | |
|---|---|---|---|---|
| Pos. [°2θ] | Height [cts] | Left [°2θ] | d-spacing [Å] | Rel. Int. [%] |
| 35.56 | 53.32 | 0.3070 | 2.52 | 0.37 |
| 29.80 | 63.97 | 0.2047 | 3.00 | 0.44 |
| 16.93 | 71.49 | 0.1279 | 5.24 | 0.49 |
| 24.92 | 102.48 | 0.2558 | 3.57 | 0.70 |
| 36.23 | 107.39 | 0.1023 | 2.48 | 0.74 |
| 13.61 | 116.75 | 0.1023 | 6.50 | 0.80 |
| 26.41 | 220.16 | 0.1279 | 3.37 | 1.51 |
| 34.38 | 257.94 | 0.1791 | 2.61 | 1.77 |
| 37.21 | 276.05 | 0.2047 | 2.42 | 1.90 |
| 15.60 | 312.21 | 0.1023 | 5.68 | 2.15 |
| 28.80 | 322.92 | 0.1791 | 3.10 | 2.22 |
| 21.61 | 561.97 | 0.1279 | 4.11 | 3.86 |
| 33.39 | 646.60 | 0.1535 | 2.68 | 4.44 |
| 27.38 | 1005.53 | 0.1535 | 3.26 | 6.91 |
| 11.03 | 628.30 | 0.1023 | 8.02 | 4.32 |
| 14.08 | 285.68 | 0.1023 | 6.29 | 1.96 |
| 15.30 | 192.32 | 0.1535 | 5.79 | 1.32 |
| 18.00 | 64.87 | 0.1535 | 4.93 | 0.45 |
| 18.54 | 356.05 | 0.0768 | 4.79 | 2.45 |
| 19.46 | 3868.35 | 0.1279 | 4.56 | 26.58 |
| 20.34 | 49.11 | 0.2047 | 4.37 | 0.34 |
| 21.08 | 143.18 | 0.1791 | 4.22 | 0.98 |
| 22.09 | 14553.92 | 0.1023 | 4.02 | 100.00 |
| 23.09 | 339.39 | 0.1023 | 3.85 | 2.33 |
| 23.53 | 2005.89 | 0.1535 | 3.78 | 13.78 |

Citrate

In salt screening experiments, BRD4780 citrate salt Type A was obtained by stirring BRD4780 freebase and equimolar citric acid in acetone at RT for 2 days.

TABLE 27
Citrate Type A

| | | FWHM | | |
|---|---|---|---|---|
| Pos. [°2θ] | Height [cts] | Left [°2θ] | d-spacing [Å] | Rel. Int. [%] |
| 4.44 | 11354.27 | 0.1023 | 19.89 | 100.00 |
| 8.88 | 5845.20 | 0.1279 | 9.96 | 51.48 |
| 10.77 | 715.59 | 0.0768 | 8.21 | 6.30 |
| 13.02 | 664.86 | 0.1023 | 6.80 | 5.86 |
| 13.34 | 939.79 | 0.1279 | 6.64 | 8.28 |
| 13.68 | 985.43 | 0.1023 | 6.47 | 8.68 |
| 15.63 | 445.80 | 0.1023 | 5.67 | 3.93 |
| 15.91 | 511.20 | 0.1279 | 5.57 | 4.50 |
| 16.56 | 116.77 | 0.1279 | 5.35 | 1.03 |
| 17.12 | 164.36 | 0.1023 | 5.18 | 1.45 |
| 17.82 | 5295.71 | 0.1535 | 4.98 | 46.64 |
| 18.48 | 1184.33 | 0.1279 | 4.80 | 10.43 |
| 18.90 | 281.64 | 0.1279 | 4.70 | 2.48 |
| 19.77 | 376.48 | 0.1023 | 4.49 | 3.32 |
| 20.14 | 168.48 | 0.0768 | 4.41 | 1.48 |
| 20.39 | 260.76 | 0.1279 | 4.36 | 2.30 |
| 21.48 | 180.22 | 0.1023 | 4.14 | 1.59 |
| 22.03 | 1419.10 | 0.2047 | 4.03 | 12.50 |
| 22.35 | 487.16 | 0.0768 | 3.98 | 4.29 |
| 23.17 | 34.61 | 0.1535 | 3.84 | 0.30 |
| 24.42 | 328.28 | 0.1791 | 3.65 | 2.89 |
| 25.23 | 460.30 | 0.1535 | 3.53 | 4.05 |
| 26.72 | 85.10 | 0.5117 | 3.34 | 0.75 |
| 27.90 | 44.66 | 0.1535 | 3.20 | 0.39 |
| 28.70 | 185.88 | 0.1279 | 3.11 | 1.64 |
| 29.67 | 407.98 | 0.1791 | 3.01 | 3.59 |
| 31.41 | 20.68 | 0.3070 | 2.85 | 0.18 |
| 34.08 | 119.22 | 0.1279 | 2.63 | 1.05 |
| 34.47 | 91.51 | 0.1279 | 2.60 | 0.81 |
| 35.54 | 156.70 | 0.1023 | 2.53 | 1.38 |
| 36.04 | 111.54 | 0.3070 | 2.49 | 0.98 |
| 36.98 | 112.00 | 0.2047 | 2.43 | 0.99 |
| 37.46 | 75.92 | 0.2047 | 2.40 | 0.67 |
| 38.70 | 72.38 | 0.5117 | 2.33 | 0.64 |

L-Malate

During the salt screen, two solid forms of the BRD4780 L-malate salt were observed (Type A and Type B). During reparation of L-malate Type A, a mixture of Type A and Type C was observed, which was fully converted to Type C by addition of excess BRD4780 freebase. BRD4780 L-malate Type C was determined to have a favorable DSC/TGA profile and was advanced to further characterization. L-malate Type C was determined to be an equal mixture of enantiomers by derivatization with Cbz-C₁ and analysis by chiral SFC chromatography.

501.4 mg of BRD4780 freebase and 438.5 mg L-malic acid (molar ratio of 1:1, acid/freebase) was stirred in 10 mL acetone at RT for 1 day. The solids were filtered and dried at RT under vacuum for 8 h, and XRPD result showed L-malate Type A+C was obtained. Then 329.6 mg of L-malate Type A+C was mixed with 245.1 mg freeform and stirred in 5 mL acetone at RT for 1 day. The solids were filtered and dried at RT under vacuum for 4 h. 428.0 mg solids were obtained (yield ˜71%). The XRPD diffraction peaks were listed in Table 24. The ¹H NMR analysis showed the molar ratio of acid/base was 0.5, and no residual acetone was observed.

TABLE 28
L-malate Type A

| | | FWHM | | |
|---|---|---|---|---|
| Pos. [°2θ] | Height [cts] | Left [°2θ] | d-spacing [Å] | Rel. Int. [%] |
| 8.30 | 9816.91 | 0.1023 | 10.66 | 100.00 |
| 9.74 | 46.34 | 0.3070 | 9.08 | 0.47 |
| 12.14 | 1055.69 | 0.1023 | 7.29 | 10.75 |
| 12.68 | 380.42 | 0.1023 | 6.98 | 3.88 |
| 13.34 | 594.97 | 0.1279 | 6.64 | 6.06 |
| 15.26 | 2525.67 | 0.1791 | 5.81 | 25.73 |
| 16.61 | 170.47 | 0.1023 | 5.34 | 1.74 |
| 17.79 | 1296.77 | 0.1279 | 4.99 | 13.21 |
| 18.24 | 4401.96 | 0.1279 | 4.86 | 44.84 |
| 18.85 | 1456.33 | 0.1279 | 4.71 | 14.83 |
| 19.81 | 480.04 | 0.1279 | 4.48 | 4.89 |
| 21.00 | 330.10 | 0.1535 | 4.23 | 3.36 |
| 21.78 | 833.10 | 0.1279 | 4.08 | 8.49 |
| 22.65 | 2838.37 | 0.1279 | 3.93 | 28.91 |
| 24.40 | 3009.67 | 0.1535 | 3.65 | 30.66 |
| 25.03 | 748.01 | 0.1023 | 3.56 | 7.62 |
| 25.71 | 232.24 | 0.1279 | 3.46 | 2.37 |
| 26.59 | 126.53 | 0.0768 | 3.35 | 1.29 |
| 27.08 | 789.53 | 0.1279 | 3.29 | 8.04 |
| 27.48 | 144.85 | 0.1279 | 3.25 | 1.48 |
| 28.04 | 312.18 | 0.1791 | 3.18 | 3.18 |
| 30.31 | 92.30 | 0.1279 | 2.95 | 0.94 |
| 30.83 | 341.34 | 0.1791 | 2.90 | 3.48 |
| 31.63 | 200.48 | 0.1279 | 2.83 | 2.04 |
| 32.01 | 166.46 | 0.2303 | 2.80 | 1.70 |
| 32.97 | 141.77 | 0.2558 | 2.72 | 1.44 |
| 34.03 | 100.98 | 0.2047 | 2.63 | 1.03 |
| 34.56 | 167.07 | 0.1535 | 2.60 | 1.70 |
| 36.13 | 144.38 | 0.1535 | 2.49 | 1.47 |
| 36.82 | 147.17 | 0.1279 | 2.44 | 1.50 |
| 37.59 | 78.21 | 0.2047 | 2.39 | 0.80 |
| 38.19 | 191.06 | 0.1535 | 2.36 | 1.95 |
| 39.04 | 83.54 | 0.1279 | 2.31 | 0.85 |

TABLE 29
L-Malate Type C

| | | FWHM | | |
|---|---|---|---|---|
| Pos. [°2θ] | Height [cts] | Left [°2θ] | d-spacing [Å] | Rel. Int. [%] |
| 7.67 | 29334.90 | 0.1023 | 11.53 | 100.00 |
| 11.03 | 657.66 | 0.1023 | 8.02 | 2.24 |
| 12.42 | 1161.75 | 0.1279 | 7.13 | 3.96 |
| 14.38 | 3918.06 | 0.1023 | 6.16 | 13.36 |
| 14.59 | 469.27 | 0.0768 | 6.07 | 1.60 |
| 15.32 | 564.83 | 0.1023 | 5.78 | 1.93 |
| 16.08 | 4871.90 | 0.1023 | 5.51 | 16.61 |
| 16.87 | 426.73 | 0.1023 | 5.25 | 1.45 |
| 17.46 | 1312.33 | 0.1279 | 5.08 | 4.47 |
| 19.95 | 970.22 | 0.1023 | 4.45 | 3.31 |
| 20.26 | 644.24 | 0.1279 | 4.38 | 2.20 |
| 21.83 | 2088.56 | 0.1279 | 4.07 | 7.12 |
| 22.15 | 298.81 | 0.1023 | 4.01 | 1.02 |
| 23.05 | 7582.92 | 0.1279 | 3.86 | 25.85 |
| 23.53 | 118.82 | 0.1023 | 3.78 | 0.41 |
| 24.70 | 1526.43 | 0.1279 | 3.60 | 5.20 |
| 25.78 | 354.75 | 0.1279 | 3.46 | 1.21 |
| 26.71 | 57.36 | 0.2558 | 3.34 | 0.20 |
| 27.13 | 334.80 | 0.1279 | 3.29 | 1.14 |
| 27.93 | 210.73 | 0.1279 | 3.19 | 0.72 |
| 28.97 | 630.57 | 0.1535 | 3.08 | 2.15 |
| 29.67 | 113.91 | 0.1791 | 3.01 | 0.39 |
| 30.88 | 721.21 | 0.2303 | 2.90 | 2.46 |
| 32.68 | 170.70 | 0.1023 | 2.74 | 0.58 |
| 33.47 | 135.40 | 0.2558 | 2.68 | 0.46 |
| 34.63 | 402.63 | 0.1535 | 2.59 | 1.37 |
| 35.97 | 69.79 | 0.3070 | 2.50 | 0.24 |
| 36.97 | 46.36 | 0.2047 | 2.43 | 0.16 |
| 38.79 | 60.71 | 0.2558 | 2.32 | 0.21 |

Lactate

In salt screening experiments, BRD4780 lactate salt Type A was obtained by stirring BRD4780 freebase and equimolar DL-lactic acid in MTBE at RT for 2 days.

TABLE 30
Lactate Type A

| | | FWHM | | |
|---|---|---|---|---|
| Pos. [°2θ] | Height [cts] | Left [°2θ] | d-spacing [Å] | Rel. Int. [%] |
| 6.30 | 8321.99 | 0.1023 | 14.04 | 100.00 |
| 8.15 | 395.85 | 0.0768 | 10.85 | 4.76 |
| 8.90 | 81.64 | 0.0768 | 9.94 | 0.98 |
| 9.87 | 199.56 | 0.0768 | 8.96 | 2.40 |
| 10.77 | 1786.10 | 0.1279 | 8.21 | 21.46 |
| 12.58 | 1785.54 | 0.1279 | 7.04 | 21.46 |
| 13.84 | 194.72 | 0.0768 | 6.40 | 2.34 |
| 14.02 | 285.65 | 0.0768 | 6.32 | 3.43 |
| 15.06 | 128.51 | 0.1279 | 5.88 | 1.54 |
| 15.89 | 161.28 | 0.1023 | 5.58 | 1.94 |
| 16.30 | 200.44 | 0.1023 | 5.44 | 2.41 |
| 16.57 | 710.11 | 0.1279 | 5.35 | 8.53 |
| 17.08 | 101.51 | 0.1023 | 5.19 | 1.22 |
| 17.74 | 229.67 | 0.1023 | 5.00 | 2.76 |
| 18.49 | 514.34 | 0.1023 | 4.80 | 6.18 |
| 18.87 | 826.48 | 0.1279 | 4.70 | 9.93 |
| 19.61 | 949.99 | 0.1279 | 4.53 | 11.42 |
| 20.05 | 290.54 | 0.1023 | 4.43 | 3.49 |
| 20.30 | 192.03 | 0.1023 | 4.37 | 2.31 |
| 20.56 | 98.95 | 0.1023 | 4.32 | 1.19 |
| 21.58 | 141.15 | 0.0768 | 4.12 | 1.70 |
| 21.80 | 159.19 | 0.1023 | 4.08 | 1.91 |
| 22.70 | 189.38 | 0.0768 | 3.92 | 2.28 |
| 24.19 | 29.77 | 0.1535 | 3.68 | 0.36 |
| 24.55 | 61.33 | 0.1535 | 3.63 | 0.74 |
| 25.28 | 511.06 | 0.1279 | 3.52 | 6.14 |
| 26.85 | 34.32 | 0.1279 | 3.32 | 0.41 |
| 28.98 | 311.97 | 0.2047 | 3.08 | 3.75 |
| 32.10 | 43.71 | 0.3070 | 2.79 | 0.53 |
| 34.06 | 54.18 | 0.1535 | 2.63 | 0.65 |
| 35.50 | 60.89 | 0.2047 | 2.53 | 0.73 |

Succinate

In salt screening experiments, two forms of BRD4780 succinate salt were observed in different solvents, which were named as succinate Type A and succinate Type B. BRD4780 Succinate salt Type A was obtained by stirring BRD4780 freebase and equimolar succinic acid in MTBE at RT for 2 days. BRD4780 Succinate salt Type B was obtained by stirring BRD4780 freebase and equimolar succinic acid in IPA/H₂O (19:1, v:v) at RT for 2 days, followed by stirring at 5° C. for 10 days and then evaporation at RT.

TABLE 31
Succinate Type A

| | | FWHM | | |
|---|---|---|---|---|
| Pos. [°2θ] | Height [cts] | Left [°2θ] | d-spacing [Å] | Rel. Int. [%] |
| 7.50 | 43.68 | 0.3070 | 11.79 | 0.35 |
| 10.89 | 1912.10 | 0.1023 | 8.12 | 15.18 |
| 12.19 | 113.44 | 0.0768 | 7.26 | 0.90 |
| 13.55 | 193.23 | 0.1023 | 6.54 | 1.53 |
| 14.39 | 2343.54 | 0.1279 | 6.16 | 18.61 |
| 14.82 | 1379.21 | 0.1023 | 5.98 | 10.95 |
| 15.41 | 471.39 | 0.1279 | 5.75 | 3.74 |
| 17.03 | 242.43 | 0.1023 | 5.21 | 1.92 |
| 17.58 | 81.29 | 0.1535 | 5.05 | 0.65 |
| 18.26 | 461.41 | 0.1023 | 4.86 | 3.66 |
| 19.15 | 1583.28 | 0.1023 | 4.63 | 12.57 |
| 19.50 | 12595.95 | 0.1535 | 4.55 | 100.00 |
| 20.55 | 109.96 | 0.1535 | 4.32 | 0.87 |
| 21.13 | 461.65 | 0.1279 | 4.20 | 3.67 |
| 21.84 | 4704.16 | 0.1535 | 4.07 | 37.35 |
| 22.08 | 1197.42 | 0.0768 | 4.03 | 9.51 |
| 22.65 | 694.30 | 0.1535 | 3.93 | 5.51 |
| 23.32 | 1054.23 | 0.1535 | 3.81 | 8.37 |
| 24.18 | 166.70 | 0.0768 | 3.68 | 1.32 |
| 25.89 | 198.96 | 0.1023 | 3.44 | 1.58 |
| 26.70 | 191.14 | 0.2047 | 3.34 | 1.52 |
| 27.29 | 1347.86 | 0.2303 | 3.27 | 10.70 |
| 28.29 | 142.45 | 0.1279 | 3.15 | 1.13 |
| 28.55 | 123.22 | 0.1279 | 3.13 | 0.98 |
| 29.61 | 222.18 | 0.2303 | 3.02 | 1.76 |
| 31.05 | 42.76 | 0.1535 | 2.88 | 0.34 |
| 31.95 | 142.65 | 0.1279 | 2.80 | 1.13 |
| 32.31 | 223.82 | 0.1279 | 2.77 | 1.78 |
| 32.99 | 232.72 | 0.1279 | 2.71 | 1.85 |
| 34.10 | 124.08 | 0.1535 | 2.63 | 0.99 |
| 35.24 | 104.91 | 0.2558 | 2.55 | 0.83 |
| 36.34 | 102.01 | 0.1535 | 2.47 | 0.81 |
| 36.76 | 472.73 | 0.1023 | 2.45 | 3.75 |
| 38.17 | 88.51 | 0.2047 | 2.36 | 0.70 |

TABLE 32
Succinate Type B

| | | FWHM | | |
|---|---|---|---|---|
| Pos. [°2θ] | Height [cts] | Left [°2θ] | d-spacing [Å] | Rel. Int. [%] |
| 6.15 | 45063.71 | 0.1023 | 14.37 | 100.00 |
| 6.61 | 120.91 | 0.0768 | 13.37 | 0.27 |
| 7.85 | 1253.19 | 0.1791 | 11.27 | 2.78 |
| 8.40 | 50.93 | 0.1535 | 10.53 | 0.11 |
| 8.96 | 126.59 | 0.0768 | 9.87 | 0.28 |
| 9.96 | 329.12 | 0.1023 | 8.88 | 0.73 |
| 10.77 | 83.46 | 0.0768 | 8.22 | 0.19 |
| 11.02 | 50.28 | 0.0768 | 8.03 | 0.11 |
| 11.54 | 65.11 | 0.1279 | 7.67 | 0.14 |
| 12.25 | 648.69 | 0.1023 | 7.22 | 1.44 |
| 13.24 | 128.04 | 0.1279 | 6.69 | 0.28 |
| 14.03 | 152.47 | 0.1023 | 6.31 | 0.34 |
| 14.58 | 136.89 | 0.1279 | 6.08 | 0.30 |
| 15.52 | 1319.00 | 0.0768 | 5.71 | 2.93 |
| 15.72 | 2796.28 | 0.0768 | 5.64 | 6.21 |
| 16.03 | 714.75 | 0.1023 | 5.53 | 1.59 |
| 16.24 | 461.76 | 0.0768 | 5.46 | 1.02 |
| 16.56 | 756.95 | 0.1023 | 5.35 | 1.68 |
| 16.89 | 1212.02 | 0.1023 | 5.25 | 2.69 |
| 17.23 | 257.61 | 0.1023 | 5.15 | 0.57 |
| 17.76 | 705.53 | 0.0768 | 5.00 | 1.57 |
| 18.22 | 2409.18 | 0.0768 | 4.87 | 5.35 |
| 18.44 | 9907.51 | 0.1279 | 4.81 | 21.99 |
| 19.57 | 450.64 | 0.0768 | 4.54 | 1.00 |
| 20.04 | 5046.32 | 0.1279 | 4.43 | 11.20 |
| 21.39 | 1284.73 | 0.2047 | 4.15 | 2.85 |
| 21.97 | 637.53 | 0.1535 | 4.04 | 1.41 |
| 22.32 | 771.12 | 0.1279 | 3.98 | 1.71 |
| 22.95 | 968.48 | 0.1535 | 3.88 | 2.15 |
| 23.87 | 732.12 | 0.1279 | 3.73 | 1.62 |
| 24.29 | 1171.71 | 0.1535 | 3.66 | 2.60 |
| 24.66 | 1185.91 | 0.1023 | 3.61 | 2.63 |
| 25.32 | 266.07 | 0.2047 | 3.52 | 0.59 |
| 25.88 | 324.50 | 0.1023 | 3.44 | 0.72 |
| 26.15 | 1745.68 | 0.1023 | 3.41 | 3.87 |
| 26.73 | 463.71 | 0.0768 | 3.34 | 1.03 |
| 27.25 | 230.38 | 0.2047 | 3.27 | 0.51 |
| 27.81 | 75.48 | 0.1279 | 3.21 | 0.17 |
| 28.16 | 89.42 | 0.1023 | 3.17 | 0.20 |
| 28.59 | 129.43 | 0.0768 | 3.12 | 0.29 |
| 29.55 | 68.34 | 0.0768 | 3.02 | 0.15 |
| 30.93 | 231.00 | 0.1791 | 2.89 | 0.51 |
| 31.52 | 358.84 | 0.1279 | 2.84 | 0.80 |
| 31.94 | 240.75 | 0.0768 | 2.80 | 0.53 |
| 32.76 | 26.92 | 0.1791 | 2.73 | 0.06 |
| 33.52 | 119.48 | 0.1023 | 2.67 | 0.27 |
| 34.36 | 110.76 | 0.0768 | 2.61 | 0.25 |
| 34.54 | 54.76 | 0.4093 | 2.60 | 0.12 |
| 36.91 | 68.49 | 0.1535 | 2.44 | 0.15 |
| 38.04 | 262.17 | 0.0768 | 2.37 | 0.58 |

Adipate

In salt screening experiments, BRD4780 adipate salt Type A was obtained by stirring BRD4780 freebase and equimolar adipic acid in EtOAc at RT for 2 days, followed by adding additional BRD4780 freebase and continue stirring for 6 days at RT.

TABLE 33
Adipate Type A

| | | FWHM | | |
|---|---|---|---|---|
| Pos. [°2θ] | Height [cts] | Left [°2θ] | d-spacing [Å] | Rel. Int. [%] |
| 6.89 | 27471.78 | 0.1023 | 12.83 | 100.00 |
| 11.18 | 152.61 | 0.1023 | 7.91 | 0.56 |
| 12.62 | 1685.37 | 0.1023 | 7.02 | 6.13 |
| 13.01 | 134.35 | 0.1023 | 6.81 | 0.49 |
| 13.77 | 1718.62 | 0.1023 | 6.43 | 6.26 |
| 14.23 | 309.07 | 0.1023 | 6.23 | 1.13 |
| 15.01 | 1090.62 | 0.0768 | 5.90 | 3.97 |
| 15.16 | 976.96 | 0.0512 | 5.85 | 3.56 |
| 17.02 | 396.52 | 0.1023 | 5.21 | 1.44 |
| 17.37 | 564.81 | 0.1023 | 5.11 | 2.06 |
| 17.70 | 2066.69 | 0.1791 | 5.01 | 7.52 |
| 18.14 | 1284.28 | 0.1023 | 4.89 | 4.67 |
| 19.95 | 1787.39 | 0.1279 | 4.45 | 6.51 |
| 20.70 | 9239.09 | 0.1023 | 4.29 | 33.63 |
| 21.01 | 612.44 | 0.1023 | 4.23 | 2.23 |
| 21.53 | 447.57 | 0.1023 | 4.13 | 1.63 |
| 22.48 | 260.25 | 0.1023 | 3.96 | 0.95 |
| 23.48 | 367.32 | 0.1535 | 3.79 | 1.34 |
| 24.55 | 312.51 | 0.1023 | 3.63 | 1.14 |
| 24.76 | 763.66 | 0.1023 | 3.60 | 2.78 |
| 25.33 | 737.81 | 0.1279 | 3.52 | 2.69 |
| 25.80 | 392.54 | 0.0768 | 3.45 | 1.43 |
| 27.17 | 123.23 | 0.1279 | 3.28 | 0.45 |
| 27.70 | 70.76 | 0.1535 | 3.22 | 0.26 |
| 28.91 | 69.29 | 0.1535 | 3.09 | 0.25 |
| 29.22 | 142.07 | 0.1279 | 3.06 | 0.52 |
| 30.12 | 75.38 | 0.3070 | 2.97 | 0.27 |
| 30.62 | 96.99 | 0.2047 | 2.92 | 0.35 |
| 31.18 | 235.40 | 0.1023 | 2.87 | 0.86 |
| 32.11 | 91.47 | 0.3070 | 2.79 | 0.33 |
| 33.92 | 113.61 | 0.1023 | 2.64 | 0.41 |
| 35.73 | 60.68 | 0.1535 | 2.51 | 0.22 |
| 38.53 | 58.49 | 0.3070 | 2.34 | 0.21 |

Acetate

In salt screening experiments, BRD4780 acetate salt Type A was obtained by stirring BRD4780 freebase and equimolar acetic acid in EtOAc at RT for 2 days.

TABLE 34
Acetate Type A

| | | FWHM | | |
|---|---|---|---|---|
| Pos. [°2θ] | Height [cts] | Left [°2θ] | d-spacing [Å] | Rel. Int. [%] |
| 7.08 | 10512.05 | 0.1023 | 12.48 | 76.94 |
| 8.07 | 166.45 | 0.0768 | 10.96 | 1.22 |
| 12.23 | 425.38 | 0.0768 | 7.24 | 3.11 |
| 12.89 | 791.19 | 0.1279 | 6.87 | 5.79 |
| 13.25 | 214.68 | 0.1023 | 6.68 | 1.57 |
| 14.19 | 2515.70 | 0.1279 | 6.24 | 18.41 |
| 14.58 | 208.72 | 0.1023 | 6.08 | 1.53 |
| 16.21 | 94.66 | 0.0768 | 5.47 | 0.69 |
| 16.63 | 540.81 | 0.1279 | 5.33 | 3.96 |
| 19.16 | 280.23 | 0.1279 | 4.63 | 2.05 |
| 20.40 | 297.78 | 0.1279 | 4.35 | 2.18 |
| 20.91 | 1444.16 | 0.1023 | 4.25 | 10.57 |
| 21.36 | 13662.64 | 0.1535 | 4.16 | 100.00 |
| 23.00 | 103.41 | 0.1023 | 3.87 | 0.76 |
| 24.98 | 219.31 | 0.1535 | 3.56 | 1.61 |
| 25.37 | 82.35 | 0.1535 | 3.51 | 0.60 |
| 25.97 | 74.36 | 0.2047 | 3.43 | 0.54 |
| 28.10 | 45.99 | 0.3070 | 3.18 | 0.34 |
| 30.17 | 31.30 | 0.2558 | 2.96 | 0.23 |
| 30.89 | 37.63 | 0.2047 | 2.90 | 0.28 |
| 31.40 | 65.89 | 0.2047 | 2.85 | 0.48 |
| 33.14 | 75.94 | 0.2047 | 2.70 | 0.56 |
| 35.94 | 45.38 | 0.2558 | 2.50 | 0.33 |
| 37.23 | 32.61 | 0.2047 | 2.42 | 0.24 |

Tosylate

In salt screening experiments, BRD4780 tosylate salt Type A was obtained by stirring BRD4780 freebase and equimolar p-toluenesulfonic acid in EtOAc at RT for 2 days.

TABLE 35
Tosylate Type A

| | | FWHM | | |
|---|---|---|---|---|
| Pos. [°2θ] | Height [cts] | Left [°2θ] | d-spacing [Å] | Rel. Int. [%] |
| 6.52 | 8431.77 | 0.1023 | 13.55 | 72.40 |
| 8.44 | 3206.42 | 0.0768 | 10.47 | 27.53 |
| 8.74 | 746.92 | 0.0768 | 10.12 | 6.41 |
| 12.29 | 924.84 | 0.0768 | 7.20 | 7.94 |
| 12.89 | 1512.63 | 0.0512 | 6.87 | 12.99 |
| 13.04 | 1912.90 | 0.0768 | 6.79 | 16.43 |
| 14.89 | 400.33 | 0.1023 | 5.95 | 3.44 |
| 15.91 | 3216.01 | 0.1023 | 5.57 | 27.62 |
| 16.42 | 399.07 | 0.0768 | 5.40 | 3.43 |
| 16.78 | 460.00 | 0.0768 | 5.28 | 3.95 |
| 17.50 | 537.58 | 0.1023 | 5.07 | 4.62 |
| 17.79 | 203.39 | 0.1279 | 4.99 | 1.75 |
| 18.54 | 1094.85 | 0.1023 | 4.79 | 9.40 |
| 19.15 | 1314.57 | 0.1023 | 4.63 | 11.29 |
| 19.37 | 386.19 | 0.0768 | 4.58 | 3.32 |
| 20.10 | 11645.83 | 0.1023 | 4.42 | 100.00 |
| 20.77 | 832.55 | 0.1023 | 4.28 | 7.15 |
| 21.11 | 2477.40 | 0.1023 | 4.21 | 21.27 |
| 21.32 | 710.59 | 0.0768 | 4.17 | 6.10 |
| 21.70 | 329.45 | 0.0768 | 4.10 | 2.83 |
| 22.21 | 81.81 | 0.2047 | 4.00 | 0.70 |
| 23.44 | 218.28 | 0.1023 | 3.79 | 1.87 |
| 23.97 | 234.30 | 0.1535 | 3.71 | 2.01 |
| 24.36 | 1448.66 | 0.1279 | 3.65 | 12.44 |
| 24.92 | 998.60 | 0.1279 | 3.57 | 8.57 |
| 25.44 | 2172.03 | 0.1279 | 3.50 | 18.65 |
| 25.92 | 228.41 | 0.1023 | 3.44 | 1.96 |
| 26.19 | 502.78 | 0.0768 | 3.40 | 4.32 |
| 26.87 | 190.46 | 0.1279 | 3.32 | 1.64 |
| 27.57 | 446.93 | 0.1023 | 3.23 | 3.84 |
| 27.85 | 97.30 | 0.0768 | 3.20 | 0.84 |
| 28.39 | 147.84 | 0.0768 | 3.14 | 1.27 |
| 28.63 | 197.04 | 0.0768 | 3.12 | 1.69 |
| 28.92 | 347.40 | 0.1023 | 3.09 | 2.98 |
| 29.20 | 369.79 | 0.1279 | 3.06 | 3.18 |
| 29.76 | 347.40 | 0.1279 | 3.00 | 2.98 |
| 30.22 | 169.74 | 0.1279 | 2.96 | 1.46 |
| 31.00 | 506.62 | 0.1023 | 2.88 | 4.35 |
| 31.53 | 134.91 | 0.1279 | 2.84 | 1.16 |
| 31.92 | 283.63 | 0.1023 | 2.80 | 2.44 |
| 32.25 | 396.78 | 0.0768 | 2.78 | 3.41 |
| 32.50 | 196.97 | 0.1023 | 2.76 | 1.69 |
| 32.87 | 151.97 | 0.0768 | 2.73 | 1.30 |
| 33.11 | 529.53 | 0.1279 | 2.71 | 4.55 |
| 34.15 | 84.25 | 0.1279 | 2.63 | 0.72 |
| 34.36 | 124.91 | 0.0768 | 2.61 | 1.07 |
| 34.93 | 628.57 | 0.1535 | 2.57 | 5.40 |
| 35.40 | 54.05 | 0.1535 | 2.54 | 0.46 |
| 36.19 | 353.10 | 0.1791 | 2.48 | 3.03 |
| 37.45 | 151.79 | 0.1023 | 2.40 | 1.30 |
| 38.48 | 89.42 | 0.0768 | 2.34 | 0.77 |

Mesylate

In salt screening experiments, BRD4780 mesylate salt Type A was obtained by stirring BRD4780 freebase and equimolar methanesulfonic acid in MTBE at RT for 2 days.

TABLE 36
Mesylate Type A

| | | FWHM | | |
|---|---|---|---|---|
| Pos. [°2θ] | Height [cts] | Left [°2θ] | d-spacing [Å] | Rel. Int. [%] |
| 7.47 | 4167.92 | 0.1023 | 11.84 | 22.66 |
| 9.02 | 4255.07 | 0.1023 | 9.80 | 23.14 |
| 10.32 | 18390.86 | 0.1023 | 8.57 | 100.00 |
| 15.60 | 1170.80 | 0.1023 | 5.68 | 6.37 |
| 16.76 | 132.29 | 0.1535 | 5.29 | 0.72 |
| 17.91 | 2616.76 | 0.1023 | 4.95 | 14.23 |
| 18.06 | 1519.20 | 0.0512 | 4.91 | 8.26 |
| 19.09 | 9819.30 | 0.1023 | 4.65 | 53.39 |
| 19.42 | 1346.84 | 0.1023 | 4.57 | 7.32 |
| 20.67 | 9091.77 | 0.1279 | 4.30 | 49.44 |
| 22.20 | 1202.69 | 0.1023 | 4.00 | 6.54 |
| 22.47 | 329.19 | 0.1279 | 3.96 | 1.79 |
| 23.23 | 178.75 | 0.1279 | 3.83 | 0.97 |
| 23.75 | 506.00 | 0.1023 | 3.75 | 2.75 |
| 24.65 | 402.41 | 0.0768 | 3.61 | 2.19 |
| 25.47 | 468.38 | 0.1023 | 3.50 | 2.55 |
| 26.02 | 200.50 | 0.1023 | 3.42 | 1.09 |
| 26.50 | 522.41 | 0.1023 | 3.36 | 2.84 |
| 27.17 | 1849.27 | 0.1279 | 3.28 | 10.06 |
| 27.61 | 114.08 | 0.1023 | 3.23 | 0.62 |
| 27.94 | 1381.01 | 0.1023 | 3.19 | 7.51 |
| 29.31 | 1054.38 | 0.1279 | 3.05 | 5.73 |
| 29.81 | 792.50 | 0.0768 | 3.00 | 4.31 |
| 30.00 | 915.35 | 0.1279 | 2.98 | 4.98 |
| 30.56 | 116.59 | 0.1023 | 2.93 | 0.63 |
| 31.41 | 1302.21 | 0.1279 | 2.85 | 7.08 |
| 31.95 | 148.08 | 0.1023 | 2.80 | 0.81 |
| 33.05 | 132.67 | 0.0768 | 2.71 | 0.72 |
| 33.52 | 749.41 | 0.1535 | 2.67 | 4.07 |
| 34.00 | 392.39 | 0.1023 | 2.64 | 2.13 |
| 35.53 | 459.23 | 0.1279 | 2.53 | 2.50 |
| 35.87 | 174.53 | 0.1023 | 2.50 | 0.95 |
| 36.19 | 141.05 | 0.1279 | 2.48 | 0.77 |
| 36.47 | 164.65 | 0.0768 | 2.46 | 0.90 |
| 37.07 | 127.20 | 0.1535 | 2.43 | 0.69 |
| 37.73 | 55.64 | 0.1535 | 2.38 | 0.30 |
| 38.05 | 129.02 | 0.1279 | 2.36 | 0.70 |
| 38.42 | 276.75 | 0.1279 | 2.34 | 1.50 |
| 38.81 | 89.12 | 0.1023 | 2.32 | 0.48 |

Besylate

In salt screening experiments, two forms of BRD4780 besylate salt were observed in different solvents, which were named as besylate Type A and besylate Type B. BRD4780 Besylate salt Type A was obtained by stirring BRD4780 freebase and equimolar benzenesulfonic acid in acetone at RT for 2 days. BRD4780 Besylate salt Type B was obtained by stirring BRD4780 freebase and equimolar benzenesulfonic acid in MTBE at RT for 2 days

TABLE 37
Besylate Type A

| | | FWHM | | |
|---|---|---|---|---|
| Pos. [°2θ] | Height [cts] | Left [°2θ] | d-spacing [Å] | Rel. Int. [%] |
| 5.52 | 1090.66 | 0.0768 | 16.00 | 11.12 |
| 7.33 | 3754.98 | 0.0768 | 12.05 | 38.28 |
| 7.60 | 8829.14 | 0.1023 | 11.63 | 90.01 |
| 8.57 | 2767.45 | 0.0768 | 10.32 | 28.21 |
| 11.05 | 1700.46 | 0.1023 | 8.01 | 17.34 |
| 13.89 | 391.46 | 0.1023 | 6.38 | 3.99 |
| 14.45 | 2429.91 | 0.1023 | 6.13 | 24.77 |
| 14.70 | 1074.21 | 0.0768 | 6.02 | 10.95 |
| 15.23 | 109.49 | 0.1023 | 5.82 | 1.12 |
| 16.59 | 713.48 | 0.1279 | 5.34 | 7.27 |
| 17.24 | 91.98 | 0.1023 | 5.14 | 0.94 |
| 18.33 | 198.78 | 0.1023 | 4.84 | 2.03 |
| 19.07 | 9809.23 | 0.1279 | 4.65 | 100.00 |
| 19.47 | 433.03 | 0.1023 | 4.56 | 4.41 |
| 20.79 | 125.31 | 0.0768 | 4.27 | 1.28 |
| 22.16 | 2004.50 | 0.1279 | 4.01 | 20.43 |
| 22.60 | 184.77 | 0.1279 | 3.94 | 1.88 |
| 22.91 | 116.47 | 0.1279 | 3.88 | 1.19 |
| 23.45 | 52.93 | 0.2047 | 3.79 | 0.54 |
| 24.96 | 182.00 | 0.1535 | 3.57 | 1.86 |
| 25.29 | 100.97 | 0.1023 | 3.52 | 1.03 |
| 25.87 | 229.83 | 0.1279 | 3.44 | 2.34 |
| 26.44 | 347.26 | 0.1279 | 3.37 | 3.54 |
| 27.93 | 201.40 | 0.1023 | 3.19 | 2.05 |
| 28.28 | 71.30 | 0.1535 | 3.16 | 0.73 |
| 28.64 | 55.96 | 0.1023 | 3.12 | 0.57 |
| 29.13 | 559.69 | 0.1279 | 3.07 | 5.71 |
| 29.63 | 264.66 | 0.1023 | 3.01 | 2.70 |
| 30.70 | 245.26 | 0.1023 | 2.91 | 2.50 |
| 31.11 | 322.54 | 0.1535 | 2.87 | 3.29 |
| 32.14 | 37.26 | 0.2047 | 2.78 | 0.38 |
| 33.13 | 79.75 | 0.0768 | 2.70 | 0.81 |
| 33.37 | 104.57 | 0.1279 | 2.68 | 1.07 |
| 34.71 | 112.64 | 0.1279 | 2.58 | 1.15 |
| 35.08 | 55.09 | 0.1535 | 2.56 | 0.56 |
| 35.31 | 82.94 | 0.1023 | 2.54 | 0.85 |
| 36.40 | 19.40 | 0.4093 | 2.47 | 0.20 |
| 37.40 | 48.77 | 0.3582 | 2.40 | 0.50 |
| 38.50 | 16.18 | 0.3070 | 2.34 | 0.16 |

TABLE 38
Besylate Type B

| | | FWHM | | |
|---|---|---|---|---|
| Pos. [°2θ] | Height [cts] | Left [°2θ] | d-spacing [Å] | Rel. Int. [%] |
| 5.86 | 98.23 | 0.1535 | 15.08 | 1.64 |
| 7.16 | 2938.63 | 0.1023 | 12.35 | 49.07 |
| 8.10 | 4141.18 | 0.1023 | 10.92 | 69.15 |
| 8.84 | 3280.65 | 0.1023 | 10.01 | 54.78 |
| 12.47 | 454.22 | 0.1023 | 7.10 | 7.58 |
| 14.33 | 1587.83 | 0.1023 | 6.18 | 26.51 |
| 15.37 | 879.46 | 0.1023 | 5.76 | 14.69 |
| 15.88 | 114.19 | 0.0768 | 5.58 | 1.91 |
| 16.20 | 193.41 | 0.1535 | 5.47 | 3.23 |
| 16.85 | 113.81 | 0.1023 | 5.26 | 1.90 |
| 17.26 | 241.38 | 0.1023 | 5.14 | 4.03 |
| 18.00 | 542.86 | 0.1023 | 4.93 | 9.07 |
| 18.34 | 449.82 | 0.1279 | 4.84 | 7.51 |
| 18.69 | 385.96 | 0.1023 | 4.75 | 6.44 |
| 19.28 | 255.32 | 0.1279 | 4.60 | 4.26 |
| 19.82 | 5988.47 | 0.1279 | 4.48 | 100.00 |
| 20.28 | 748.09 | 0.1023 | 4.38 | 12.49 |
| 22.25 | 2164.19 | 0.1023 | 4.00 | 36.14 |
| 22.83 | 392.20 | 0.1023 | 3.90 | 6.55 |
| 22.99 | 315.95 | 0.1023 | 3.87 | 5.28 |
| 23.38 | 71.59 | 0.1535 | 3.81 | 1.20 |
| 23.84 | 59.82 | 0.1535 | 3.73 | 1.00 |
| 24.42 | 619.98 | 0.1279 | 3.65 | 10.35 |
| 25.22 | 279.33 | 0.1023 | 3.53 | 4.66 |
| 25.73 | 135.03 | 0.1023 | 3.46 | 2.25 |
| 26.31 | 160.78 | 0.1279 | 3.39 | 2.68 |
| 26.57 | 186.89 | 0.1023 | 3.36 | 3.12 |
| 27.19 | 311.63 | 0.1535 | 3.28 | 5.20 |
| 27.98 | 191.78 | 0.1279 | 3.19 | 3.20 |
| 28.30 | 144.83 | 0.1279 | 3.15 | 2.42 |
| 28.87 | 405.92 | 0.1279 | 3.09 | 6.78 |
| 29.48 | 101.09 | 0.0768 | 3.03 | 1.69 |
| 31.00 | 264.69 | 0.1023 | 2.88 | 4.42 |
| 31.92 | 136.73 | 0.1023 | 2.80 | 2.28 |
| 32.70 | 394.05 | 0.1535 | 2.74 | 6.58 |
| 33.36 | 92.16 | 0.1535 | 2.69 | 1.54 |
| 34.38 | 518.77 | 0.1535 | 2.61 | 8.66 |
| 36.22 | 39.11 | 0.1535 | 2.48 | 0.65 |
| 38.30 | 64.76 | 0.2558 | 2.35 | 1.08 |

Malonate

In salt screening experiments, BRD4780 malonate salt Type A was obtained by stirring BRD4780 freebase and equimolar malonic acid in MTBE at RT for 2 days.

TABLE 39
Malonate Type A

| | | FWHM | | |
|---|---|---|---|---|
| Pos. [°2θ] | Height [cts] | Left [°2θ] | d-spacing [Å] | Rel. Int. [%] |
| 3.11 | 529.85 | 0.2047 | 28.41 | 2.19 |
| 6.54 | 24146.67 | 0.0768 | 13.51 | 100.00 |
| 12.40 | 136.46 | 0.1023 | 7.14 | 0.57 |
| 13.08 | 221.29 | 0.0768 | 6.77 | 0.92 |
| 13.27 | 486.00 | 0.0768 | 6.67 | 2.01 |
| 14.41 | 95.94 | 0.0768 | 6.15 | 0.40 |
| 15.76 | 302.06 | 0.0768 | 5.62 | 1.25 |
| 15.98 | 920.48 | 0.0768 | 5.55 | 3.81 |
| 16.19 | 690.29 | 0.1023 | 5.47 | 2.86 |
| 18.98 | 93.23 | 0.1535 | 4.68 | 0.39 |
| 19.69 | 3455.88 | 0.1535 | 4.51 | 14.31 |
| 20.00 | 893.35 | 0.0768 | 4.44 | 3.70 |
| 20.62 | 42.84 | 0.1791 | 4.31 | 0.18 |
| 21.04 | 84.42 | 0.2047 | 4.22 | 0.35 |
| 22.04 | 357.65 | 0.1023 | 4.03 | 1.48 |
| 22.37 | 470.09 | 0.1023 | 3.97 | 1.95 |
| 22.65 | 906.59 | 0.1023 | 3.93 | 3.75 |
| 24.13 | 96.06 | 0.1023 | 3.69 | 0.40 |
| 24.56 | 132.41 | 0.0768 | 3.62 | 0.55 |
| 25.77 | 79.33 | 0.1023 | 3.46 | 0.33 |
| 26.34 | 642.80 | 0.1791 | 3.38 | 2.66 |
| 26.86 | 64.28 | 0.1023 | 3.32 | 0.27 |
| 28.00 | 132.75 | 0.1535 | 3.19 | 0.55 |
| 29.07 | 101.78 | 0.1279 | 3.07 | 0.42 |
| 29.48 | 97.08 | 0.1023 | 3.03 | 0.40 |
| 30.07 | 47.03 | 0.1535 | 2.97 | 0.19 |
| 31.61 | 154.99 | 0.1023 | 2.83 | 0.64 |
| 32.22 | 118.87 | 0.1023 | 2.78 | 0.49 |
| 32.49 | 72.58 | 0.4093 | 2.76 | 0.30 |
| 33.21 | 66.77 | 0.2558 | 2.70 | 0.28 |
| 35.04 | 25.38 | 0.2047 | 2.56 | 0.11 |
| 35.85 | 76.37 | 0.1791 | 2.50 | 0.32 |
| 36.74 | 80.13 | 0.1279 | 2.45 | 0.33 |
| 38.63 | 40.46 | 0.2047 | 2.33 | 0.17 |
| 39.10 | 113.52 | 0.0768 | 2.30 | 0.47 |

Benzoate

In salt screening experiments, BRD4780 benzoate salt Type A was obtained by stirring BRD4780 freebase and equimolar benzoic acid in EtOAc at RT for 2 days.

TABLE 40
Benzoate Type A

| | | FWHM | | |
|---|---|---|---|---|
| Pos. [°2θ] | Height [cts] | Left [°2θ] | d-spacing [Å] | Rel. Int. [%] |
| 6.35 | 36623.60 | 0.1023 | 13.91 | 100.00 |
| 6.55 | 5669.84 | 0.0512 | 13.50 | 15.48 |
| 8.12 | 1941.56 | 0.0768 | 10.89 | 5.30 |
| 10.09 | 2206.72 | 0.1023 | 8.76 | 6.03 |
| 10.29 | 730.45 | 0.0768 | 8.59 | 1.99 |
| 12.72 | 15060.61 | 0.1023 | 6.96 | 41.12 |
| 13.39 | 388.17 | 0.1023 | 6.61 | 1.06 |
| 13.81 | 793.57 | 0.1023 | 6.41 | 2.17 |
| 15.31 | 307.13 | 0.0768 | 5.79 | 0.84 |
| 15.47 | 453.33 | 0.0768 | 5.73 | 1.24 |
| 15.98 | 253.18 | 0.0768 | 5.54 | 0.69 |
| 16.29 | 611.28 | 0.1279 | 5.44 | 1.67 |
| 17.22 | 167.35 | 0.1279 | 5.15 | 0.46 |
| 18.41 | 2637.97 | 0.1023 | 4.82 | 7.20 |
| 18.91 | 970.46 | 0.0768 | 4.69 | 2.65 |
| 19.12 | 829.20 | 0.0768 | 4.64 | 2.26 |
| 19.45 | 2672.97 | 0.1279 | 4.56 | 7.30 |
| 19.69 | 912.40 | 0.1279 | 4.51 | 2.49 |
| 19.98 | 225.89 | 0.1023 | 4.44 | 0.62 |
| 20.24 | 186.97 | 0.1279 | 4.39 | 0.51 |
| 20.87 | 247.31 | 0.1535 | 4.26 | 0.68 |
| 21.57 | 311.41 | 0.1023 | 4.12 | 0.85 |
| 22.01 | 133.34 | 0.1279 | 4.04 | 0.36 |
| 22.97 | 5137.89 | 0.1279 | 3.87 | 14.03 |
| 23.63 | 886.98 | 0.1279 | 3.76 | 2.42 |
| 24.13 | 843.05 | 0.1023 | 3.69 | 2.30 |
| 24.69 | 598.92 | 0.1279 | 3.61 | 1.64 |
| 25.59 | 3482.82 | 0.1279 | 3.48 | 9.51 |
| 26.36 | 122.53 | 0.1023 | 3.38 | 0.33 |
| 26.97 | 83.18 | 0.1023 | 3.31 | 0.23 |
| 27.78 | 81.20 | 0.0768 | 3.21 | 0.22 |
| 28.40 | 74.69 | 0.3070 | 3.14 | 0.20 |
| 29.22 | 59.64 | 0.2047 | 3.06 | 0.16 |
| 30.19 | 315.72 | 0.1535 | 2.96 | 0.86 |
| 30.83 | 127.54 | 0.1535 | 2.90 | 0.35 |
| 31.23 | 70.87 | 0.1535 | 2.86 | 0.19 |
| 31.85 | 195.66 | 0.1279 | 2.81 | 0.53 |
| 33.09 | 377.85 | 0.1279 | 2.71 | 1.03 |
| 33.43 | 177.88 | 0.1279 | 2.68 | 0.49 |
| 33.82 | 66.08 | 0.1535 | 2.65 | 0.18 |
| 35.17 | 143.68 | 0.1535 | 2.55 | 0.39 |
| 35.73 | 85.69 | 0.2047 | 2.51 | 0.23 |
| 37.28 | 105.99 | 0.0768 | 2.41 | 0.29 |
| 37.96 | 66.61 | 0.1279 | 2.37 | 0.18 |

Hydrobromide

In salt screening experiments, two forms of BRD4780 HBr salt were observed in different solvents, which were named as HBr salt Type A and HBr salt Type B. BRD4780 HBr salt Type A was obtained by stirring BRD4780 freebase and equimolar HBr in EtOAc at RT for 2 days. BRD4780 HBr salt Type B was obtained by stirring BRD4780 freebase and equimolar HBr in toluene at RT for 2 days, followed by stirring at 5° C. for 6 days.

TABLE 41
HBr Type A

| | | FWHM | | |
|---|---|---|---|---|
| Pos. [°2θ] | Height [cts] | Left [°2θ] | d-spacing [Å] | Rel. Int. [%] |
| 6.80 | 15820.01 | 0.0768 | 12.99 | 100.00 |
| 13.59 | 3641.09 | 0.0768 | 6.52 | 23.02 |
| 14.86 | 2427.99 | 0.1023 | 5.96 | 15.35 |
| 15.12 | 106.44 | 0.0768 | 5.86 | 0.67 |
| 16.97 | 632.47 | 0.0768 | 5.23 | 4.00 |
| 17.42 | 333.21 | 0.1023 | 5.09 | 2.11 |
| 18.51 | 707.48 | 0.1023 | 4.79 | 4.47 |
| 18.90 | 546.65 | 0.1023 | 4.69 | 3.46 |
| 19.29 | 2267.94 | 0.1023 | 4.60 | 14.34 |
| 20.42 | 7565.94 | 0.0768 | 4.35 | 47.83 |
| 21.13 | 205.43 | 0.0768 | 4.20 | 1.30 |
| 21.49 | 211.23 | 0.0768 | 4.13 | 1.34 |
| 21.80 | 1365.55 | 0.1023 | 4.08 | 8.63 |
| 22.12 | 452.15 | 0.1023 | 4.02 | 2.86 |
| 22.49 | 531.29 | 0.1023 | 3.95 | 3.36 |
| 22.83 | 3605.63 | 0.1023 | 3.89 | 22.79 |
| 23.07 | 3238.93 | 0.1023 | 3.85 | 20.47 |
| 24.23 | 728.77 | 0.1023 | 3.67 | 4.61 |
| 24.71 | 391.81 | 0.0768 | 3.60 | 2.48 |
| 25.87 | 309.08 | 0.1023 | 3.44 | 1.95 |
| 26.18 | 624.06 | 0.1279 | 3.40 | 3.94 |
| 27.32 | 10697.98 | 0.1279 | 3.26 | 67.62 |
| 28.18 | 415.30 | 0.1023 | 3.17 | 2.63 |
| 28.44 | 449.46 | 0.1023 | 3.14 | 2.84 |
| 28.81 | 396.07 | 0.1279 | 3.10 | 2.50 |
| 29.21 | 333.92 | 0.1023 | 3.06 | 2.11 |
| 30.26 | 1640.88 | 0.1023 | 2.95 | 10.37 |
| 30.47 | 1169.54 | 0.0768 | 2.93 | 7.39 |
| 30.77 | 305.32 | 0.1279 | 2.91 | 1.93 |
| 31.39 | 133.54 | 0.1279 | 2.85 | 0.84 |
| 32.11 | 93.14 | 0.1023 | 2.79 | 0.59 |
| 32.73 | 68.48 | 0.1535 | 2.74 | 0.43 |
| 33.06 | 224.04 | 0.1023 | 2.71 | 1.42 |
| 33.70 | 373.56 | 0.1535 | 2.66 | 2.36 |
| 34.31 | 10197.51 | 0.1023 | 2.61 | 64.46 |
| 35.19 | 112.17 | 0.1023 | 2.55 | 0.71 |
| 35.64 | 808.56 | 0.1023 | 2.52 | 5.11 |
| 35.87 | 264.79 | 0.0768 | 2.50 | 1.67 |
| 36.23 | 189.46 | 0.0768 | 2.48 | 1.20 |
| 36.50 | 253.42 | 0.0768 | 2.46 | 1.60 |
| 37.07 | 302.37 | 0.1023 | 2.42 | 1.91 |
| 38.24 | 67.25 | 0.2047 | 2.35 | 0.43 |
| 38.99 | 585.46 | 0.1279 | 2.31 | 3.70 |
| 39.61 | 168.53 | 0.0768 | 2.28 | 1.07 |

TABLE 42
HBr Type B

| | | FWHM | | |
|---|---|---|---|---|
| Pos. [°2θ] | Height [cts] | Left [°2θ] | d-spacing [Å] | Rel. Int. [%] |
| 6.80 | 118.76 | 0.0768 | 13.00 | 4.88 |
| 7.35 | 947.24 | 0.0768 | 12.03 | 38.96 |
| 7.82 | 2431.26 | 0.0768 | 11.30 | 100.00 |
| 13.81 | 775.30 | 0.1023 | 6.41 | 31.89 |
| 14.70 | 455.26 | 0.1279 | 6.02 | 18.73 |
| 15.11 | 354.03 | 0.0768 | 5.86 | 14.56 |
| 16.22 | 85.79 | 0.1279 | 5.47 | 3.53 |
| 18.22 | 56.91 | 0.1023 | 4.87 | 2.34 |
| 19.80 | 468.06 | 0.1279 | 4.48 | 19.25 |
| 21.00 | 1667.82 | 0.1023 | 4.23 | 68.60 |
| 22.13 | 587.84 | 0.1535 | 4.02 | 24.18 |
| 22.59 | 666.72 | 0.1023 | 3.94 | 27.42 |
| 23.09 | 140.36 | 0.1535 | 3.85 | 5.77 |
| 23.54 | 149.59 | 0.1023 | 3.78 | 6.15 |
| 23.94 | 318.62 | 0.1023 | 3.72 | 13.11 |
| 24.83 | 121.66 | 0.1535 | 3.59 | 5.00 |
| 25.71 | 214.57 | 0.1535 | 3.46 | 8.83 |
| 26.16 | 75.52 | 0.1535 | 3.41 | 3.11 |
| 26.78 | 336.87 | 0.1279 | 3.33 | 13.86 |
| 27.80 | 437.44 | 0.1535 | 3.21 | 17.99 |
| 28.78 | 201.38 | 0.1023 | 3.10 | 8.28 |
| 29.64 | 210.90 | 0.1535 | 3.01 | 8.67 |
| 30.04 | 113.47 | 0.2047 | 2.97 | 4.67 |
| 31.10 | 120.21 | 0.2047 | 2.88 | 4.94 |
| 31.77 | 118.20 | 0.1023 | 2.82 | 4.86 |
| 33.36 | 151.27 | 0.1023 | 2.69 | 6.22 |
| 35.77 | 156.02 | 0.1535 | 2.51 | 6.42 |
| 36.19 | 64.85 | 0.2047 | 2.48 | 2.67 |
| 38.11 | 55.76 | 0.1535 | 2.36 | 2.29 |

L-Aspartate

BRD4780 L-aspartate salt Type A was re-prepared via stirring BRD4780 freebase and L-aspartic acid (molar ratio of 1:2, acid/freebase) in EtOAc for 4 days on 50 mg scale.

TABLE 43
L-Aspartate Type A

| | | FWHM | | |
|---|---|---|---|---|
| Pos. [°2θ] | Height [cts] | Left [°2θ] | d-spacing [Å] | Rel. Int. [%] |
| 5.59 | 26696.51 | 0.1279 | 15.82 | 100.00 |
| 9.13 | 549.82 | 0.1535 | 9.69 | 2.06 |
| 10.30 | 124.23 | 0.1023 | 8.59 | 0.47 |
| 12.39 | 115.15 | 0.1535 | 7.14 | 0.43 |
| 14.12 | 201.48 | 0.1791 | 6.27 | 0.75 |
| 14.83 | 230.80 | 0.1279 | 5.97 | 0.86 |
| 15.50 | 260.60 | 0.1023 | 5.72 | 0.98 |
| 16.10 | 621.35 | 0.1023 | 5.51 | 2.33 |
| 16.71 | 5491.44 | 0.1279 | 5.31 | 20.57 |
| 17.58 | 194.09 | 0.2047 | 5.04 | 0.73 |
| 18.13 | 421.15 | 0.1535 | 4.89 | 1.58 |
| 19.00 | 211.32 | 0.1279 | 4.67 | 0.79 |
| 19.45 | 338.88 | 0.1535 | 4.56 | 1.27 |
| 20.81 | 298.30 | 0.1023 | 4.27 | 1.12 |
| 21.17 | 721.93 | 0.2047 | 4.20 | 2.70 |
| 21.72 | 795.06 | 0.1279 | 4.09 | 2.98 |
| 22.33 | 2563.59 | 0.1535 | 3.98 | 9.60 |
| 22.85 | 375.29 | 0.1791 | 3.89 | 1.41 |
| 24.91 | 138.24 | 0.2558 | 3.57 | 0.52 |
| 25.28 | 122.39 | 0.4093 | 3.52 | 0.46 |
| 25.56 | 238.76 | 0.1023 | 3.48 | 0.89 |
| 26.20 | 207.92 | 0.1023 | 3.40 | 0.78 |
| 27.99 | 120.96 | 0.1535 | 3.19 | 0.45 |
| 28.24 | 188.34 | 0.1023 | 3.16 | 0.71 |
| 29.67 | 117.19 | 0.2047 | 3.01 | 0.44 |
| 30.11 | 160.98 | 0.2047 | 2.97 | 0.60 |
| 31.15 | 214.02 | 0.1279 | 2.87 | 0.80 |
| 32.51 | 75.27 | 0.6140 | 2.75 | 0.28 |
| 35.52 | 43.93 | 0.2047 | 2.53 | 0.16 |
| 38.02 | 40.85 | 0.6140 | 2.37 | 0.15 |
| 39.52 | 90.73 | 0.2558 | 2.28 | 0.34 |

L-Glutamate

BRD4780 L-glutamate salt Type A was re-prepared via stirring BRD4780 freebase and L-glutamic acid (molar ratio of 1:2, acid/freebase) in IPA/H₂O (19:1, v:v) for 4 days on 50 mg scale. L-glutamate Type B was re-prepared via stirring BRD4780 freebase and L-glutamic acid (molar ratio of 1:2, acid/freebase) in EtOAc for 4 days on 50 mg scale. L-glutamate Type A was re-prepared via stirring 103.3 mg BRD4780 freebase and 95.7 mg L-glutamic acid (molar ratio of 1:1, acid/freebase) in IPA/H₂O (19:1) for 10 days, followed by adding additional 92.6 mg BRD4780 freebase into the suspension and stirred for another 3 days. L-glutamate Type B was re-prepared via stirring 104.0 mg BRD4780 freebase and 95.5 mg L-glutamic acid (molar ratio of 1:1, acid/freebase) in EtOAc for 10 days, followed by adding additional 49.5 mg BRD4780 freebase into the suspension and stirred for another 3 days.

TABLE 44
L-Glutamate Type A

| | | FWHM | | |
|---|---|---|---|---|
| Pos. [°2θ] | Height [cts] | Left [°2θ] | d-spacing [Å] | Rel. Int. [%] |
| 3.11 | 753.37 | 0.3070 | 28.37 | 13.13 |
| 5.00 | 5736.46 | 0.1023 | 17.67 | 100.00 |
| 6.11 | 648.12 | 0.1023 | 14.47 | 11.30 |
| 7.85 | 267.51 | 0.0768 | 11.27 | 4.66 |
| 9.95 | 1465.13 | 0.1791 | 8.89 | 25.54 |
| 12.22 | 542.83 | 0.1791 | 7.24 | 9.46 |
| 15.00 | 286.01 | 0.1023 | 5.91 | 4.99 |
| 15.73 | 250.91 | 0.0768 | 5.64 | 4.37 |
| 18.40 | 358.15 | 0.1535 | 4.82 | 6.24 |
| 20.02 | 843.42 | 0.2047 | 4.43 | 14.70 |
| 21.85 | 716.46 | 0.2047 | 4.07 | 12.49 |
| 23.65 | 115.19 | 0.1535 | 3.76 | 2.01 |
| 24.58 | 246.18 | 0.1791 | 3.62 | 4.29 |
| 25.95 | 76.74 | 0.2558 | 3.43 | 1.34 |
| 27.93 | 96.62 | 0.3070 | 3.19 | 1.68 |
| 29.32 | 120.51 | 0.2047 | 3.05 | 2.10 |
| 30.95 | 65.05 | 0.3070 | 2.89 | 1.13 |
| 31.67 | 49.98 | 0.1535 | 2.83 | 0.87 |
| 34.21 | 78.28 | 0.1535 | 2.62 | 1.36 |
| 35.97 | 47.50 | 0.3070 | 2.50 | 0.83 |
| 37.20 | 25.20 | 0.8187 | 2.42 | 0.44 |

TABLE 45
L-Glutamate Type B

| | | FWHM | | |
|---|---|---|---|---|
| Pos. [°2θ] | Height [cts] | Left [°2θ] | d-spacing [Å] | Rel. Int. [%] |
| 5.54 | 7393.52 | 0.1023 | 15.95 | 100.00 |
| 7.86 | 576.37 | 0.1023 | 11.25 | 7.80 |
| 11.07 | 412.73 | 0.1023 | 7.99 | 5.58 |
| 13.70 | 507.06 | 0.1535 | 6.46 | 6.86 |
| 14.70 | 570.84 | 0.1279 | 6.03 | 7.72 |
| 15.70 | 224.69 | 0.1279 | 5.64 | 3.04 |
| 16.59 | 912.14 | 0.1535 | 5.34 | 12.34 |
| 16.76 | 864.78 | 0.0768 | 5.29 | 11.70 |
| 18.17 | 69.75 | 0.1279 | 4.88 | 0.94 |
| 19.77 | 161.22 | 0.1791 | 4.49 | 2.18 |
| 21.08 | 1425.32 | 0.1279 | 4.21 | 19.28 |
| 22.21 | 1210.24 | 0.2047 | 4.00 | 16.37 |
| 22.65 | 114.39 | 0.1535 | 3.93 | 1.55 |
| 23.17 | 239.95 | 0.1279 | 3.84 | 3.25 |
| 24.50 | 159.60 | 0.1535 | 3.63 | 2.16 |
| 25.62 | 74.52 | 0.2047 | 3.48 | 1.01 |
| 26.74 | 89.53 | 0.2047 | 3.33 | 1.21 |
| 28.27 | 48.42 | 0.4093 | 3.16 | 0.65 |
| 29.65 | 56.05 | 0.1535 | 3.01 | 0.76 |
| 30.30 | 139.31 | 0.1535 | 2.95 | 1.88 |
| 31.32 | 117.63 | 0.1279 | 2.86 | 1.59 |
| 33.47 | 57.11 | 0.3070 | 2.68 | 0.77 |
| 35.69 | 65.80 | 0.1535 | 2.52 | 0.89 |
| 36.32 | 52.74 | 0.3070 | 2.47 | 0.71 |
| 38.19 | 48.94 | 0.4093 | 2.36 | 0.66 |
| 39.08 | 48.77 | 0.2047 | 2.31 | 0.66 |

L-Tartrate

BRD4780 L-tartrate salt Type A was prepared via stirring BRD4780 freebase (50 mg scale) and equimolar L-tartric acid in acetone at RT. L-tartrate Type B was re-prepared via stirring BRD4780 freebase (50 mg scale) and equimolar L-tartric acid in EtOAc at RT.

TABLE 46
L-Tartrate Type A

| | | FWHM | | |
|---|---|---|---|---|
| Pos. [°2θ] | Height [cts] | Left [°2θ] | d-spacing [Å] | Rel. Int. [%] |
| 4.82 | 188.67 | 0.1023 | 18.32 | 5.67 |
| 5.81 | 349.95 | 0.1279 | 15.20 | 10.51 |
| 6.98 | 3328.46 | 0.1023 | 12.67 | 100.00 |
| 7.71 | 789.70 | 0.1023 | 11.47 | 23.73 |
| 8.24 | 900.64 | 0.1023 | 10.73 | 27.06 |
| 9.67 | 1325.98 | 0.1279 | 9.15 | 39.84 |
| 11.65 | 931.91 | 0.1023 | 7.60 | 28.00 |
| 11.93 | 219.29 | 0.1023 | 7.42 | 6.59 |
| 13.00 | 167.02 | 0.0768 | 6.81 | 5.02 |
| 13.48 | 95.01 | 0.1279 | 6.57 | 2.85 |
| 14.00 | 219.30 | 0.1279 | 6.33 | 6.59 |
| 14.64 | 133.92 | 0.1535 | 6.05 | 4.02 |
| 15.05 | 451.38 | 0.1023 | 5.89 | 13.56 |
| 15.72 | 331.81 | 0.1023 | 5.64 | 9.97 |
| 16.49 | 507.97 | 0.1535 | 5.37 | 15.26 |
| 16.78 | 560.28 | 0.1023 | 5.28 | 16.83 |
| 17.91 | 1319.33 | 0.1279 | 4.95 | 39.64 |
| 18.63 | 1272.57 | 0.1279 | 4.76 | 38.23 |
| 19.55 | 104.01 | 0.2047 | 4.54 | 3.12 |
| 20.22 | 341.50 | 0.1023 | 4.39 | 10.26 |
| 20.75 | 230.67 | 0.0768 | 4.28 | 6.93 |
| 21.26 | 943.93 | 0.1279 | 4.18 | 28.36 |
| 23.28 | 187.04 | 0.0768 | 3.82 | 5.62 |
| 24.70 | 67.50 | 0.4093 | 3.60 | 2.03 |
| 25.72 | 249.22 | 0.1535 | 3.46 | 7.49 |
| 26.63 | 274.20 | 0.1023 | 3.35 | 8.24 |
| 28.22 | 61.31 | 0.1535 | 3.16 | 1.84 |
| 29.85 | 25.56 | 0.2558 | 2.99 | 0.77 |
| 32.58 | 82.23 | 0.1791 | 2.75 | 2.47 |
| 34.13 | 16.98 | 0.3070 | 2.63 | 0.51 |
| 35.47 | 56.14 | 0.2558 | 2.53 | 1.69 |
| 38.12 | 44.11 | 0.3070 | 2.36 | 1.33 |

TABLE 47
L-Tartrate Type B

| | | FWHM | | |
|---|---|---|---|---|
| Pos. [°2θ] | Height [cts] | Left [°2θ] | d-spacing [Å] | Rel. Int. [%] |
| 5.57 | 416.73 | 0.1023 | 15.86 | 4.46 |
| 6.24 | 2921.58 | 0.1023 | 14.15 | 31.28 |
| 6.96 | 9340.14 | 0.1279 | 12.69 | 100.00 |
| 9.54 | 413.97 | 0.1279 | 9.27 | 4.43 |
| 11.11 | 632.83 | 0.1023 | 7.97 | 6.78 |
| 11.99 | 101.19 | 0.1279 | 7.38 | 1.08 |
| 12.47 | 253.15 | 0.1279 | 7.10 | 2.71 |
| 13.24 | 172.13 | 0.1279 | 6.69 | 1.84 |
| 13.90 | 131.69 | 0.1023 | 6.37 | 1.41 |
| 14.32 | 85.95 | 0.1023 | 6.18 | 0.92 |
| 14.67 | 76.61 | 0.1535 | 6.04 | 0.82 |
| 15.15 | 251.68 | 0.1535 | 5.85 | 2.69 |
| 15.90 | 1110.28 | 0.1279 | 5.58 | 11.89 |
| 16.25 | 323.39 | 0.1535 | 5.46 | 3.46 |
| 17.47 | 1208.29 | 0.1279 | 5.08 | 12.94 |
| 17.81 | 501.56 | 0.1279 | 4.98 | 5.37 |
| 18.70 | 1563.11 | 0.2558 | 4.74 | 16.74 |
| 19.10 | 674.57 | 0.1279 | 4.65 | 7.22 |
| 19.54 | 579.51 | 0.1279 | 4.54 | 6.20 |
| 21.20 | 262.30 | 0.1279 | 4.19 | 2.81 |
| 22.27 | 356.76 | 0.1279 | 3.99 | 3.82 |
| 23.44 | 56.87 | 0.2558 | 3.80 | 0.61 |
| 24.32 | 304.55 | 0.1279 | 3.66 | 3.26 |
| 24.62 | 202.39 | 0.1279 | 3.62 | 2.17 |
| 25.03 | 126.17 | 0.1023 | 3.56 | 1.35 |
| 25.45 | 832.87 | 0.1535 | 3.50 | 8.92 |
| 27.04 | 123.99 | 0.2047 | 3.30 | 1.33 |
| 27.96 | 118.62 | 0.1279 | 3.19 | 1.27 |
| 28.81 | 143.17 | 0.4093 | 3.10 | 1.53 |
| 29.88 | 190.98 | 0.1279 | 2.99 | 2.04 |
| 30.47 | 168.82 | 0.1535 | 2.93 | 1.81 |
| 31.92 | 61.70 | 0.6140 | 2.80 | 0.66 |
| 32.51 | 60.41 | 0.1535 | 2.75 | 0.65 |
| 33.17 | 74.59 | 0.1791 | 2.70 | 0.80 |
| 34.34 | 52.35 | 0.3070 | 2.61 | 0.56 |
| 35.28 | 44.63 | 0.3070 | 2.54 | 0.48 |
| 36.50 | 46.29 | 0.2047 | 2.46 | 0.50 |
| 37.54 | 31.26 | 0.2558 | 2.40 | 0.33 |

### Example 7: Pharmacological Properties of Exemplary Compounds of the Disclosure

Pharmacokinetic Characterization of Exemplary Salts

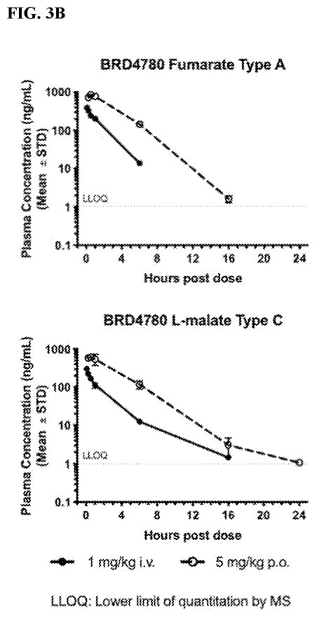

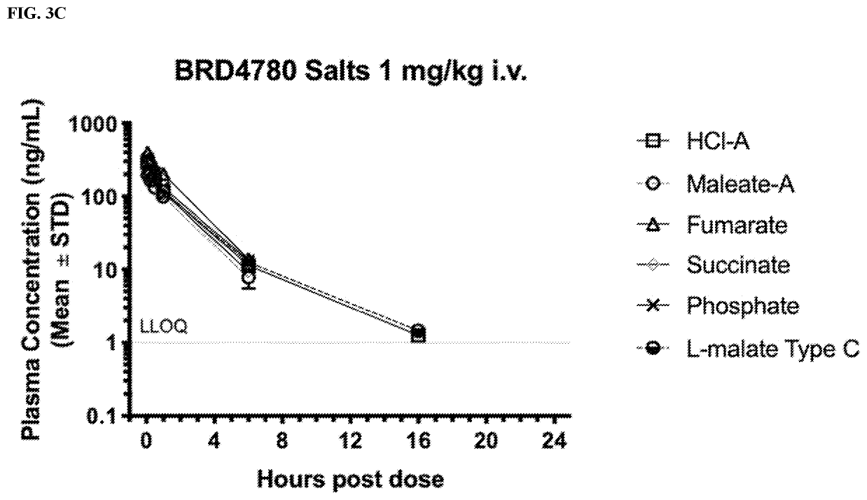

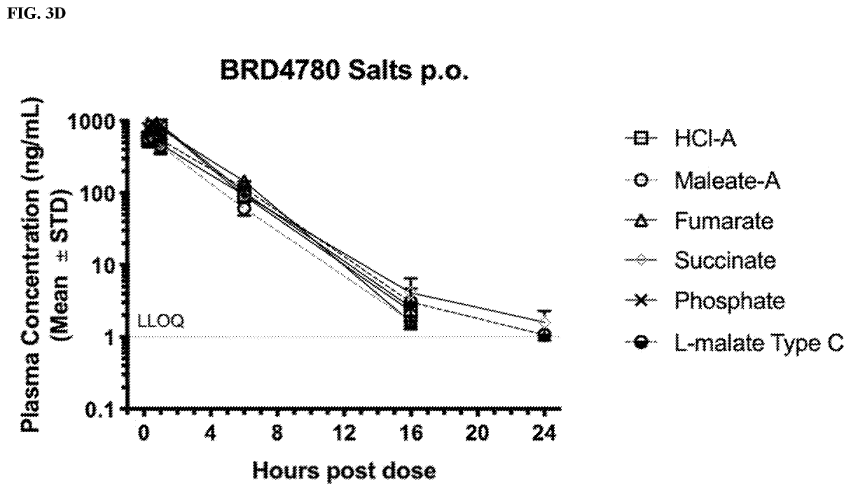

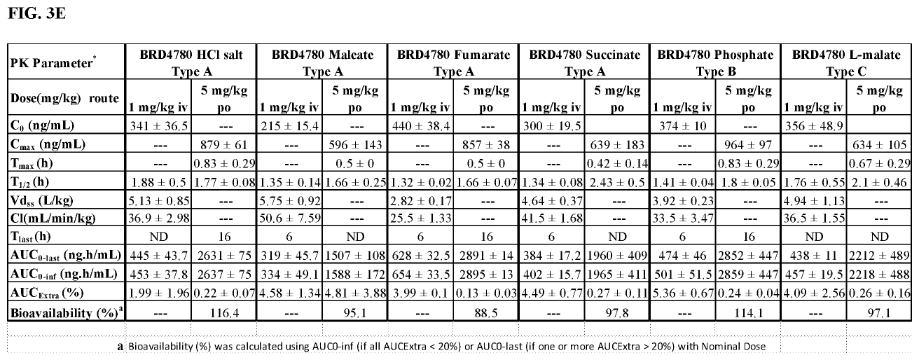

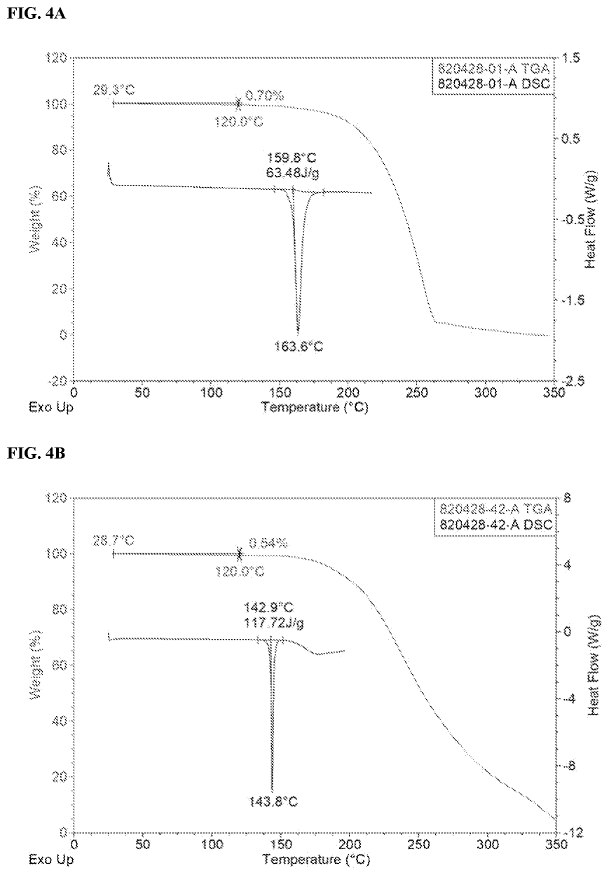

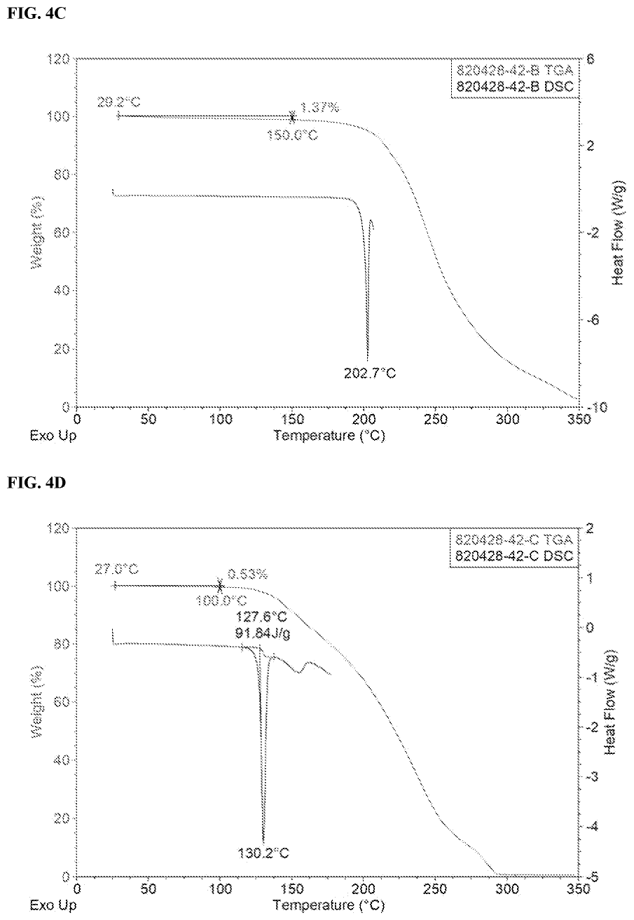

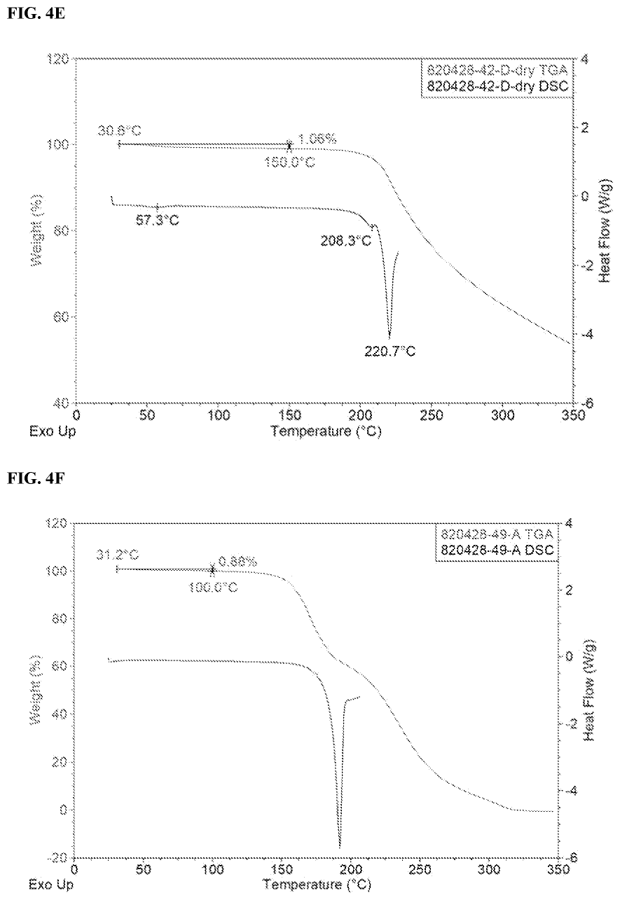

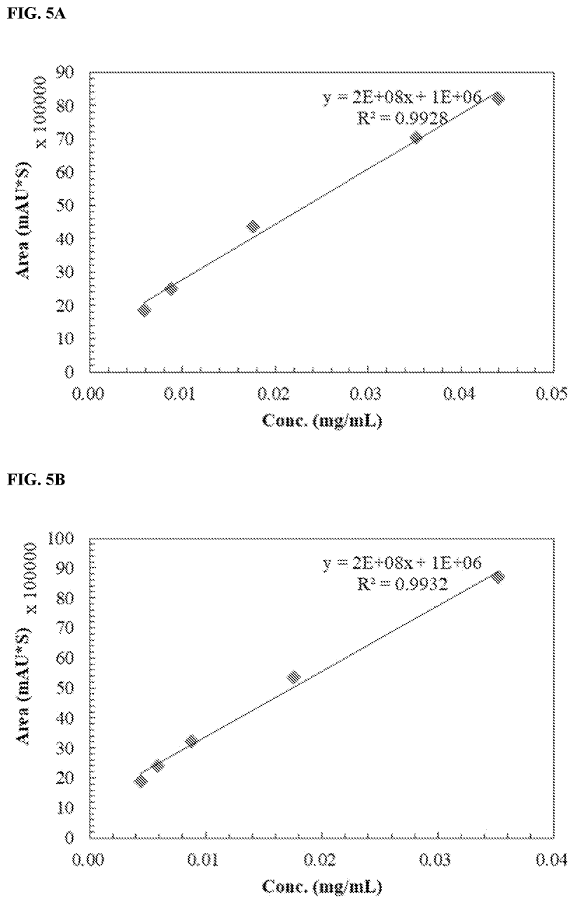

A pharmacokinetic (PK) study was performed by WuXi Apptech (WuXi) (Shanghai, China) in fed male 129S2/SvPasCrl Mice to compare the oral bioavailability of six exemplary salt forms of BRD4780, including HCl, Maleate TypeA, Fumarate Type A, Succinate Type A, Phophate Type B and L-Malate Type C. A single dose of BRD4780 in a salt form was administered at 1.0 mg/kg intravenously (i.v.) or 5.0 mg/kg orally (p.o) in a clear solution of 5% dextrose in water (D5W). Blood was collected serially from n=3 mice per dose group at seven time points post i.v. dose administration (0.083, 0.25, 0.5, 1, 6, 16 and 24 hours) or six time points post p.o. dose administration (0.25, 0.5, 1, 6, 16 and 24 hours) and plasma obtained by centrifugation. Plasma drug concentration was determined by LC-MS/MS and reported as ng/mL in plasma plotted as the mean±standard deviation (FIGS. 3A-3D). Standard pharmacokinetic parameters were calculated (FIG. 3E). All compounds had superior (>50%) oral bioavailability (% F), with reported percent oral bioavailability of 116.4%, 95.1%, 88.5%, 97.8%, 114.1% and 97.1% for the HCl, Maleate Type A, Fumarate Type A, Succinate Type A, Phosphate Type C and L-Malate Type C salt forms of BRD4780, respectively.

### Example 8: Exemplary Preparation of Enantiopure Analogs of Compounds of the Disclosure

BRD4780 was prepared as described in Example 2 as a mixture of enantiomers The absolute stereochemistry of the enantiopure fractions can be assigned by formation of the corresponding Mosher amides (Dale and Mosher. J Am Chem Soc. 95: 512-519) and further confirmed by VCD spectroscopy and single crystal X ray analysis.

Most preparative scale chromatography systems rely on analyte detection by UV-Vis absorbance (PDA detector). On the analytical scale, monitoring of the mobile-phase by mass spectroscopy is available. Potential separation of the (2R, 3R) and (2S, 3S) BRD4780 was profiled across 5 chiral stationary phases (OJ-H, AD-H, AS-H, IC and OD-H) using four different organic solvent systems (MeOH, MeOH+1% TFA, MeOH+0.05% Et₃N and iPrOH) as monitored by MS detection. No separation was observed. Accordingly, a method was pursued in which detection and separation were facilitated by incorporation of an amino protecting group that could be detected by UV absorbance.

During the preparation of BRD4780, the initial Diels-Alder reaction generated an approximately 2:1 mixture of endo and exo isomers of racemic 5-isopropyl-6-nitrobicyclo[2.2.1]hept-2-ene (Munk et al. J Med Chem. 39: 1193-1195). According to the literature preparation of the compound, separation of the endo and exo isomers could not be achieved at this stage and was performed following subsequent reduction to form the racemic mixture of C₂-endo and C₂-exo isopropylbicyclo[2.2.1]heptan-2-amine (Id.). The ability to separate the mixture of 5-isopropyl-6-nitrobicyclo[2.2.1]hept-2-ene stereoisomers was profiled using chiral SFC chromatography as monitored by UV-Vis absorbance (PDA detector). Separation of at least four materials was observed using AD-H stationary phase with iPrOH as a mobile phase (Table 2, entry 10). However, this separation was not useful on the preparative scale due to peak overlap.

To facilitate the detection and separation of BRD4780, a series of nine amino protected compounds were synthesized and profiled for separation across five chiral stationary phases (OJ-H, AD-H, AS-H, IC and OD-H) using four initial solvent systems (MeOH, MeOH+1% TFA, MeOH+0.05% Et₃N and iPrOH). The instant study included four carbamate protecting groups (3: Cbz, 4: FMOC, 5: p-NO₂-Cbz, and 6: p-Br-Cbz), two sulfonamides (7: tosyl and 8: nosyl), the 9: N-dibenzyl, the 10: phthalimido and an acetamide formed from racemic (1S,2R,3R,4R)-3-isopropylbicyclo[2.2.1]hept-5-en-2-amine, 11, (see Table 50) which was prepared by selective reduction of racemic 5-exo-isopropyl-6-endo-nitrobicyclo[2.2.1]hept-2-ene. Where promising separation was observed using one of the systems, in depth methods of development were pursued.

The results of the above separation study are summarized in Table 50. For descriptive purposes, “weak separation” has been defined in Table 50 as two observable peaks with apparent Gaussian peak shape that do not approach baseline separation, “moderate separation” as two observable peaks with apparent Gaussian peak shape with near base-line separation and “well separated” as two observable peaks with apparent Gaussian peak shape that reach full baseline separation. All other cases in which there was no observable separation or in which the peak shape was poorly defined have been described as “no separation.” For the well separated experiments, the Δtᵣ has been reported as a relative indication of separation efficiency.

The tosyl protected amine, 7, and the p-NO₂-Cbz compound 5 showed the best separation on column AD-H, thus they were optimized with other solvents systems. As shown in Table 51, analog 7 showed better separation when using either methanol, basic or acidic solvents (Δtᵣ=0.78 min). Meanwhile, the p-NO₂-Cbz 5 did not show improvement in the enantiomers separation when changing solvents, as iPrOH remained the best mobile phase (Δtᵣ=0.46 min). The p-NO₂-Cbz 5 was used for the gram scale reaction because it was shown that this could be conveniently removed by catalytic hydrogenation.

Next, 5 was synthesized at gram scale in 90% yield using the corresponding chloroformate with sodium bicarbonate in water and dioxane at room temperature, per scheme A below. The enantiomers were cleanly separated during SFC chromatography. Full baseline separation of 25 mg injections was observed on chiralpak AD-H column (250×21 mm, 5 um, 90 mL/min, 9:1 CO₂: IPA) and with high recovery (Fraction, ‘Fr’, 1: 88% of theoretical yield, Fr2: 91% of theoretical yield). Analytical chiral HPLC analysis of the isolated fractions demonstrated a single peak and no absorption at the retention time of the other enantiomer was detected (>99% ee). Deprotection of the p-NO₂-Cbz group proceeded well using PdCl₂ in ethyl acetate under an atmosphere of H₂ giving 51% yield following purification, salt formation and trituration in pentane. Following formation of the HCl salt, the analytical characterization was identical with authentic BRD4780 other than the specific rotation which showed approximately equal and opposite values: Fr1: [a]²⁵_{D}+12.0° (c=0.1, MeOH); Fr2: [a]²⁵_{D}−13.0° (c=0.1, MeOH).

To determine the absolute configuration of BRD4780 enantiomers, Mosher's model was employed. First, the (+/−) BRD4780 was derivatized with optically-pure (S)-Mosher's acid chloride. The resulting diastereomeric mixture of (S)-Mosher amides were analyzed by ¹H NMR spectroscopy and compared to the BRD4780 ¹H NMR spectrum. The BRD4780 C3 proton has a chemical shift of 1.18 ppm while the C3 proton of the mixture of (R)-Mosher amide diastereomers displayed two magnetically inequivalent peaks well resolved and separated with chemical shifts of 0.57 ppm and 0.52 ppm. Then, the (R)- and (S)-Mosher amides were synthesized with both pure BRD4780 enantiomers. For the BRD4780 enantiomer with (2R, 3R) configuration, the C3 proton of the R-Mosher diastereoisomer is more downfield than that of the (S)-Mosher diastereoisomer (Fr2). For the enantiomer with the absolute configuration of (2S, 3S), the C₃ proton of the (R)-Mosher diastereoisomer is more upfield than the amide formed from the (S)-Mosher acid.

The pure enantiomers were profiled in the high-content imaging assay for the ability to reduce the amount of FS MUC1 protein in the cytoplasm (Table 48) following the procedure of Example 4. As was the case for the racemic compound, BRD4780, there was no observed reduction in wild-type MUC1 levels.

TABLE 48
Bioactivity of enantiopure BRD4780 Fr1 and Fr2

| | FS MUC1 | FS MUC1 | FS MUC1 | FS MUC1 |
|---|---|---|---|---|
| Fraction, | cytoplasm IC₅₀ | cytoplasm | cytoplasm IC₅₀ | cytoplasm |
| Optical rotation | (uM) | EMax (%) | (uM) | EMax (%) |
| BRD4780 Fr1 | 0.156 | −42 | 9.2 | 113 |
| BRD4780 Fr2 | 0.175 | −46 | 12.6 | 60 |

In summary, a method for the preparation of (1R,2R,3R,4S)-3-isopropylbicyclo[2.2.1]heptan-2-amine and (1S,2S,3S,4R)-3-isopropylbicyclo[2.2.1]heptan-2-amine, compounds related thereto, and salts thereof has been developed, and the absolute stereo-chemistry of the individual enantiomers has been assigned using the Mosher amide method. The instant procedure relies on derivatization of the BRD4780 amino group, which enabled detection and appeared to be required for efficient separation on the stationary phase identified by methods screening and development. The applicability of this approach has been demonstrated on the hundred milligram scale, and it is projected that the instant process will be applicable to larger scale preparative applications (Caille et al. (2010) Org Process Res Dev. 14: 133-141). The ability of these compounds to remove FS MUC1 protein from cells has been profiled in vitro.

TABLE 49
Summary of Example 14 Compounds

| | Col2: | Col3: | Col4: | Col5: | Col6: |
|---|---|---|---|---|---|
| | OJ-H | AD-H | AS-H | IC | OD-H |
| | 3-50% | 3-50% | 3-50% | 3-50% | 3-50% |
| Compound | iPrOH | iPrOH | iPrOH | iPrOH | iPrOH |
| 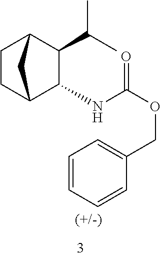 | Moderate separation Δt_{R} = 0.14 min | No separation | No separation | No separation | No separation |
| 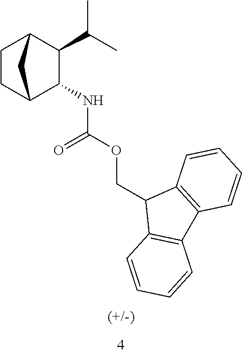 | Weak separation Δt_{R} = 0.06 min | Weak separation Δt_{R} = 0.05 min | No separation | No separation | No separation |
| 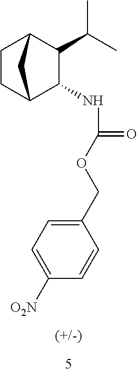 | No separation | Well separated Δt_{R} = 0.46 min | No separation | No separation | Weak separation Δt_{R} = 0.34 min |
| 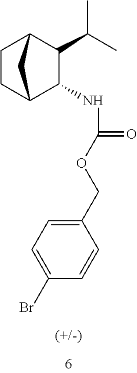 | Moderate separation Δt_{R} = 0.13 min | Moderate separation Δt_{R} = 0.13 min | No separation | No separation | Moderate separation Δt_{R} = 0.21 min |
| 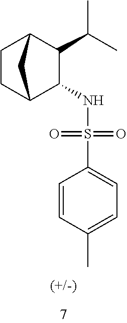 | No separation | Well separated Δt_{R} = 0.27 min | No separation | No separation | No separation |
| 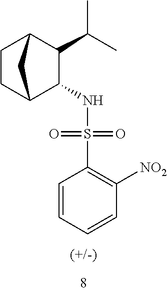 | No separation | No separation | Moderate separation Δt_{R} = 0.15 min | No separation | No separation |
| 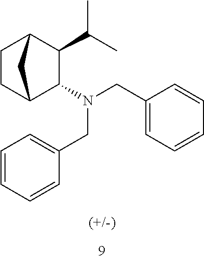 [SMILES: CC(C)[C@@H]1[C@H]2CC[C@H](C2)[C@H]1N(Cc1ccccc1)Cc1ccccc1 ; "(+/-)"] | Weak separation Δt_{R} = 0.08 min | No separation | No separation | No separation | No separation |
| 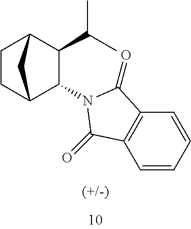 | No separation | No separation | No separation | No separation | No separation |
| 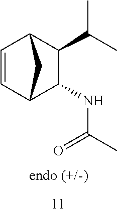 | No separation | Weak separation Δt_{R} = 0.1 min | Weak separation Δt_{R} = 0.15 min | Weak separation Δt_{R} = 0.15 min | No separation |
| 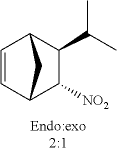 | No separation | Weak separation- multiple peaks from reaction mixture | No separation | No separation | No separation |
| | No | No | No | No | No |
| | separation | separation | separation | separation | separation |

TABLE 50
Optimization of Compounds 5 and 7 with Other Solvents Systems

| | | | | B2: | B3: | |
|---|---|---|---|---|---|---|
| | | Chiral | B1: | MeOH + 0.1% | MeOH + 0.05% | B4: |
| Entry | Compound | Column | MeOH | TFA | Et₃N | iPrOH |
| 1 | 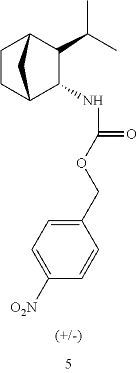 | Col3: AD- H | Moderate separation Δt_{R} = 0.14 min | Moderate separation Δt_{R} = 0.13 min | Moderate separation Δt_{R} = 0.13 min | Well separated Δt_{R} = 0.46 min |
| 2 | 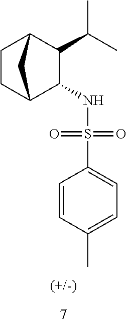 | Col3: AD- H | Well separated Δt_{R} = 0.76 min | Well separated Δt_{R} = 0.78 min | Well separated Δt_{R} = 0.78 min | Moderate separation Δt_{R} = 0.27 min |

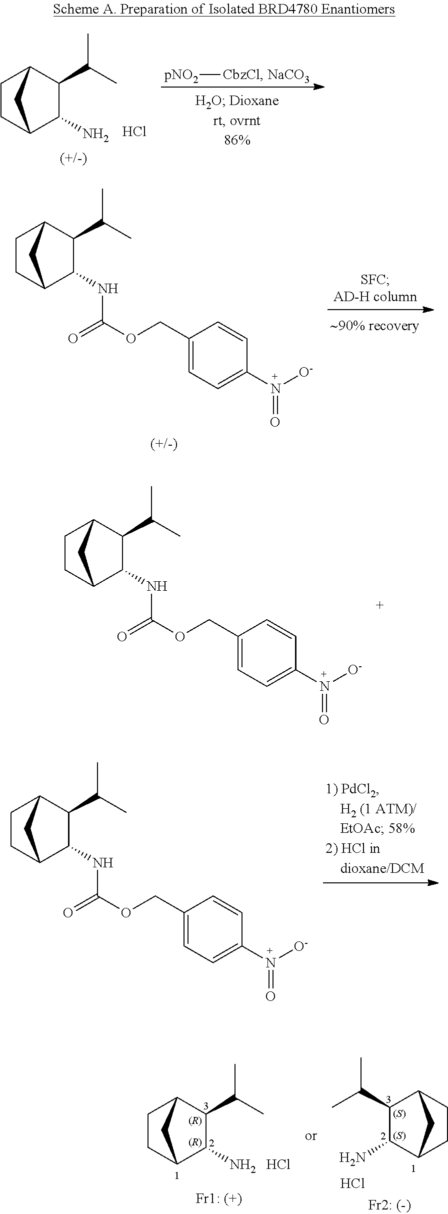

Representative Experimental Processes

Analytical scale SFC conditions: columns: CHIRALCEL OJ-H, AD-H, AS-H, IC and OD-H (250×4.6 mm×5 um); Flow rate: 1.5 mL/min; Mobile phases: MeOH, MeOH+1% TFA, MeOH+0.05% Et₃N and iPrOH; ABPR: 136 Bar; Column oven temp.: 45° C.

Preparative scale SFC conditions for the separation of 3: Column: CHIRALCEL OX-H (250×21 mm×5 um); Flow rate: 85 mL/min; Mobile phase: Line-A: 93% of Liq. CO₂, Line-B: 7% of 0.1% DEA in IPA: Acetonitrile (50:50); Sample injection: 10 mg; ABPR: 100 Bar; Column oven temp.: ambient. Incomplete separation.

Preparative scale SFC conditions for the separation of 5: Column: CHIRALCEL AD-H (250×21 mm×5 um); Flow rate: 90 mL/min; Mobile phase: Line-A: 90% of Liq. C₀₂, Line-B: 10% IPA; Sample injection: 25 mg; ABPR: 100 Bar; Column oven temp.: ambient. Full Baseline separation.

Representative Procedures

rac-benzyl (3-exo-isopropylbicyclo[2.2.1]heptan-2-endo-yl)carbamate, 3

To a stirred solution of rac-3-exo-isopropylnorbornan-2-endo-amine hydrochloride (37 mg, 0.196 mmol, 1.00 eq) and disodium carbonate (22 mg, 0.206 mmol, 1.05 eq) in water (1 mL), at 0° C. was added slowly benzyl carbonochloridate (0.028 mL, 0.196 mmol, 1.00 eq). After 20 min of stirring, additional water (0.5 mL) was added and the reaction mixture was stirred for another hour. After complete addition, diethyl ether was added and the product was extracted 3 times with ether. The combined organic layers were washed with HCl (1 M) and NaOH (1 M), dried with MgSO₄, filtered and concentrated. The crude residue was purified with flash chromatography on silica gel (Hexane/EtOAc) to afford the desired rac-benzyl (3-exo-isopropylbicyclo[2.2.1]heptan-2-endo-yl)carbamate (25 mg, 44% yield) as a solid. ¹H NMR (400 MHz, Chloroform-d) δ 7.36 (m, 5H), 5.11 (m, 2H), 4.79 (d, J=7.9 Hz, 1H), 3.62 (m, 1H), 2.43 (m, 1H), 2.12 (d, J=4.2 Hz, 1H), 1.65-1.57 (m, 2H), 1.51 (m, 4H), 1.22 (dd, J=10.1 Hz, 2.1 Hz, 1H), 1.14 (m, 1H), 0.89 (m, 6H), 0.49 (m, 1H). ¹³C NMR (101 MHz, Chloroform-d) δ 155.90, 136.81, 128.66, 128.21, 66.70, 58.52, 58.42, 41.04, 39.43, 35.61, 32.24, 30.87, 21.86, 21.18, 20.23. MS(ESI): 288.7 [M+H]+

### rac-(9H-fluoren-9-yl)methyl (3-exo-isopropylbicyclo[2.2.1]heptan-2-endo-yl)carbamate, 4

To a stirred solution of rac-(9H-fluoren-9-yl)methyl (3-exo-isopropylbicyclo[2.2.1]heptan-2-endo-yl)carbamate hydrochloride (37 mg, 0.196 mmol, 1.00 eq) in 1,4-dioxane (1 mL), was added sodium carbonate (1.0 M in water, 0.21 mL, 0.206 mmol, 1.05 eq). At 0° C., a solution of 9H-fluoren-9-ylmethyl carbonochloridate (51 mg, 0.196 mmol, 1.00 eq) in dioxane (0.2 ml) was added. The reaction mixture was allowed to warm up to room temperature and was stirred for overnight. Water was poured into the reaction mixture and the product was extracted with EtOAc. The organic layer was dried with MgSO₄, filtered and concentrated. The crude residue was purified with flash chromatography (Hexane/EtOAc 0-30%) to afford the desired rac (9H-fluoren-9-yl)methyl ((1R,2R,3R,4S)-3-isopropylbicyclo[2.2.1]heptan-2-yl)carbamate (30 mg, 41% yield). ¹H NMR (400 MHz, Chloroform-d) δ 7.77 (d, J=7.5 Hz, 2H), 7.64-7.56 (m, 2H), 7.45-7.36 (m, 2H), 7.32 (ddd, J=7.4, 7.4, 1.2 Hz, 2H), 4.79 (d, J=7.5 Hz, 1H), 4.44 (m, 2H), 4.23 (m, 1H), 3.59 (m, 1H), 2.42 (s, 1H), 2.13 (m, 1H), 1.68-1.50 (m, 1H), 1.48-1.30 (m, 3H), 1.22 (s, 2H), 0.95-0.83 (m, 6H), 0.50 (ddd, J=9.8, 5.4, 2.0 Hz, 1H). 13C NMR (101 MHz, DMSO-d6) δ 155.61, 143.93, 143.88, 140.72, 127.60, 127.00, 125.25, 125.21, 120.10, 65.03, 58.12, 54.29, 46.84, 40.60, 39.52, 38.78, 36.22, 35.03, 31.74, 29.94, 21.75, 20.79, 19.73. MS(ESI): 376.5 [M+H]

### rac-4-nitrobenzyl (3-exo-isopropylbicyclo[2.2.1]heptan-2-endo-yl)carbamate, 5

To a vial containing rac-3-exo-isopropylnorbornan-2-endo-amine hydrochloride (1.00 g, 5.27 mmol, 1.00 eq) and (4-nitrophenyl)methyl carbonochloridate (1.19 g, 5.53 mmol, 1.05 eq) were added dioxane (25 mL) and sodium carbonate (1.0 M in water, 5.53 mL, 5.53 mmol, 1.05 eq). The reaction mixture was stirred at room temperature for 18 hours. Water was poured into the reaction mixture and the product was extracted with EtOAc. The organic layer was dried with MgSO₄, filtered and concentrated. The crude residue was purified with flash chromatography (silica gel, Hexane/EtOAc 0-40%) to afford the desired product rac-4-nitrobenzyl (3-exo-isopropylbicyclo[2.2.1]heptan-2-endo-yl)carbamate (1.57 g, 90% yield). ¹H NMR (400 MHz, Chloroform-d) δ 8.22 (d, J=8.5 Hz, 2H), 7.50 (d, J=8.4 Hz, 2H), 5.18 (m, 2H), 4.83 (d, J=7.9 Hz, 1H), 3.59 (m, 1H), 2.43 (m, 1H), 2.15 (d, J=3.6 Hz, 1H), 1.60-1.52 (m, 2H), 1.49-1.33 (m, 4H), 1.23-1.10 (m, 2H), 0.89 (m, 6H), 0.50 (m, 1H). MS (ESI): 333.3 [M+H]+

### rac-4-bromobenzyl (3-exo-isopropylbicyclo[2.2.1]heptan-2-endo-yl)carbamate, 6

To a stirred solution of rac-3-exo-isopropylnorbornan-2-endo-amine hydrochloride (30 mg, 0.158 mmol, 1.00 eq) and disodium carbonate (34 mg, 0.316 mmol, 2.00 eq) in 1,4-dioxane (1 mL) was added (4-bromophenyl)methyl carbonochloridate (24 uL, 0.158 mmol, 1.00 eq).

The reaction mixture was stirred at room temperature for 24 hours. The reaction mixture was filtered and the filtrate was concentrated. The crude residue was purified with flash chromatography (Hexane/EtOAc 0-25%) to afford the desired rac-4-bromobenzyl (3-exo-isopropylbicyclo[2.2.1]heptan-2-endo-yl)carbamate (9 mg, 16%) as a white solid. ¹H NMR (400 MHz, Chloroform-d) δ 7.51 (d, J=8.0 Hz, 2H), 7.24 (d, J=8.0 Hz, 2H), 5.03 (m, 2H), 4.76 (d, J=7.9 Hz, 1H), 3.58 (m, 1H), 2.42 (s, 1H), 2.13 (d, J=3.6 Hz, 1H), 1.64-1.58 (m, 3H), 1.49-1.32 (m, 4H), 1.19 (dd, J=10.3, 2.0 Hz, 1H), 1.16-1.07 (m, 1H), 0.88 (m, 6H), 0.51-0.43 (m, 1H).

¹³C NMR (101 MHz, Chloroform-d) δ 155.69, 145.43, 131.78, 129.84, 65.84, 58.48, 58.45, 41.02, 39.41, 35.60, 35.41, 32.23, 30.82, 21.86, 21.17, 20.21. MS (ESI): 336.2/368.3 [M+H]+

### rac-N-(3-exo-isopropylbicyclo[2.2.1]heptan-2-endo-yl)-4-methylbenzenesulfonamide, 7

To a stirred solution of rac-3-exo-isopropylnorbornan-2-endo-amine hydrochloride (39 mg, 0.254 mmol, 1.00 eq) in dry dichloromethane (1 mL), at 0° C. and under N₂ was added triethylamine (0.071 mL, 0.509 mmol, 2.00 eq) and a solution of 4-methylbenzenesulfonyl chloride (53 mg, 0.280 mmol, 1.10 eq) in DCM (0.2 ml). The reaction mixture was allowed to warm up to room temperature and was stirred for 2.5 days. Water was poured into the reaction mixture and the product was extracted with DCM. The organic layer was dried with MgSO₄, filtered and concentrated. The crude residue was purified with flash chromatography (silica gel, Hexane/EtOAc 0-15%) to afford the desired rac-N-(3-exo-isopropylbicyclo[2.2.1]heptan-2-endo-yl)-4-methylbenzenesulfonamide (36 mg, 57% yield). ¹H NMR (400 MHz, Chloroform-d) δ 7.77 (d, J=8.2 Hz, 2H), 7.28 (d, J=8.2 Hz, 2H), 4.74 (d, J=6.7 Hz, 1H), 3.13 (ddd, J=6.8, 5.3, 3.9 Hz, 1H), 2.42 (s, 3H), 2.11-2.01 (m, 2H), 1.56-1.42 (m, 2H), 1.34-1.17 (m, 3H), 1.10 (m, 2H), 0.82 (d, J=6.5 Hz, 3H), 0.69 (d, J=6.6 Hz, 3H), 0.51 (ddd, J=9.5, 5.1, 2.1 Hz, 1H). ¹³C NMR (101 MHz, Chloroform-d) δ 143.39, 137.88, 129.68, 127.42, 60.42, 58.72, 40.78, 38.88, 35.45, 32.18, 30.59, 21.80, 21.69, 20.90, 20.00. MS (ESI): 306.1 [M−H]−

### rac-N-(3-exo-isopropylbicyclo[2.2.1]heptan-2-endo-yl)-2-nitrobenzenesulfonamide, 8

To a stirred solution of rac-3-exo-isopropylnorbornan-2-endo-amine hydrochloride (30 mg, 0.196 mmol, 1.00 eq) in dry dichloromethane (1 mL), at 0° C. and under N₂ was added triethylamine (0.055 mL, 0.391 mmol, 2.00 eq) and a solution of 2-nitrobenzenesulfonyl chloride (48 mg, 0.215 mmol, 1.10 eq) in DCM (0.2 ml). The reaction mixture was allowed to warm up to room temperature and was stirred for 1 hour. Water was poured into the reaction mixture and the product was extracted with DCM. The organic layer was dried with MgSO₄, filtered and concentrated to afford the desired rac-N-(3-exo-isopropylbicyclo[2.2.1]heptan-2-endo-yl)-2-nitrobenzenesulfonamide (37 mg, 56% yield). ¹H NMR (400 MHz, Chloroform-d) δ 8.19-8.12 (m, 1H), 7.88-7.81 (m, 1H), 7.79-7.68 (m, 2H), 5.41 (d, J=7.0 Hz, 1H), 3.37 (ddd, J=7.0, 5.4, 4.0 Hz, 1H), 2.25-2.04 (m, 2H), 1.63-1.42 (m, 2H), 1.42-1.07 (m, 6H), 0.85 (d, J=6.6 Hz, 3H), 0.70 (d, J=6.6 Hz, 3H), 0.63 (ddd, J=9.7, 5.2, 2.1 Hz, 1H). MS (ESI): 337.2 [M−H]−

### rac-(N,N-dibenzyl-3-exo-isopropylbicyclo[2.2.1]heptan-2-endo-amine, 9

To stirred a solution of rac-3-exo-isopropylnorbornan-2-endo-amine hydrochloride (0.3 g, 1.960 mmol, 1.0 eq) in N,N-dimethylformamide (3 mL) and potassium carbonate (0.541 g, 3.92 mmol, 2 eq) was added and reaction mass was stirred for 15 min at room temperature. To this, benzyl bromide (0.268 g, 1.568 mmol, 0.8 eq) was added drop wise and reaction mass was stirred at room temperature for 1 h. After completion of reaction, reaction mixture was diluted with water (10 mL) and extracted with dichloromethane (2×10 mL). The combined organic was dried over anhydrous sodium sulfate, filtered and concentrated to get crude material, which was purified using silica gel column chromatography (10% ethyl acetate in hexanes) to get (0.1 g, 0.299 mmol, 15%) of the product. MS (ESI): 334.2 [M+H]+.

### rac-2-(3-exo-isopropylbicyclo[2.2.1]heptan-2-endo-yl)isoindoline-1,3-dione, 10

To a pressure vessel rac-3-exo-isopropylnorbornan-2-endo-amine hydrochloride (0.150 g, 0.980 mmol, 1 eq.) in N,N-dimethylacetamide (2 mL) was added isobenzofuran-1,3-dione (0.156 g, 1.043 mmol, 1.6 eq) and the reaction mixture was stirred at 180° C. for 3 h. After completion of reaction, reaction mixture was diluted with water and extracted with ethyl acetate (3×20 mL) and the combined organic layer was dried over anhydrous sodium sulphate, filtered and concentrated to get crude, which was purified by silica gel column chromatography (3% ethyl acetate in hexanes) the desired product (160 mg, 0.564 mmol, 58%). ¹H NMR (400 MHz, Chloroform-d) δ 7.86 (m, 2H), 7.74 (m, 2H), 4.13 (m, 1H), 2.53-2.48 (m, 2H), 2.41 (m, 1H), 1.78-1.68 (m, 1H), 1.68-1.61 (m, 2H), 1.46-1.40 (m, 2H), 1.30-1.24 (m, 2H), 0.98 (d, J=6.5 Hz, 3H), 0.76 (d, J=6.6 Hz, 3H). MS (ESI): 284.1 [M−H]⁺.

### Rac-N-(3-exo-isopropylbicyclo[2.2.1]hept-5-en-2-endo-yl)acetamide, 11

To a stirred solution of crude ac-5-exo-isopropyl-6-endo-nitro-bicyclo[2.2.1]hept-2-ene (endo:exo NO₂ is ˜2:1) (5.39 g, 27.6 mmol, 1.00 eq) in (1:1) mixture of methanol (50 mL) and aqueous solution of saturated ammonium formate (50 mL) was added lot wise zinc dust (9.02 g, 138 mmol, 5.00 eq) over a period of 10 minutes at room temperature. The resulting reaction mixture was stirred at room temperature for 12 h. After completion of the reaction, reaction mass was filtered through celite pad and washed with methanol (2×30 mL). The organic layer was basified with saturated ammonium bicarbonate (150-160 mL) till pH=9-10. The resultant aqueous layer was extracted with dichloromethane (2×90 mL), and the combined organic layers were dried over sodium sulphate, filtered through celite, and evaporated in under vacuum at low temperature to get impure product crude rac-3-isopropylbicyclo[2.2.1]hept-5-en-2-amine (4 g, 26.45 mmol, 96%) and used in next step with our further purification. MS (ESI): 152.2 [M+H]⁺

To a stirred solution of the crude rac-3-isopropylbicyclo[2.2.1]hept-5-en-2-amine (2.00 g, 13.2 mmol, 1.00 eq) and triethyl amine (4.6 mL, 33.1 mmol, 2.50 eq) in toluene (30 mL) was added acetyl chloride (1.4 mL, 19.8 mmol, 1.50 eq) at 0° C. The reaction mixture was stirred at room temperature for 12 h. After completion of reaction, the reaction was diluted by addition of water (30 mL), and the layers were separated. The aqueous layer was extracted with dichloromethane (2×50 mL) and the combined organics were dried over anhydrous sodium sulphate, filtered through celite and evaporated to provide crude material, which was purified by silica gel chromatography (25% ethyl acetate in hexanes) to provide N-[rac-(2R,3R)-3-isopropyl-2-bicyclo [2.2.1]hept-5-enyl]acetamide, 11 (endo isomer, 0.65 g, 3.36 mmol, 25%). The rac-N-(3-exo-isopropylbicyclo[2.2.1]hept-5-en-2-endo-yl)acetamide eluted after the endo isomer was also isolated (exo isomer, 0.55 g, 2.85 mmol, 22%). ¹H NMR (400 MHz, Chloroform-d) δ 6.42 (dd, J=5.8, 3.2 Hz, 1H), 6.06 (dd, J=5.7, 2.8 Hz, 1H), 5.15 (bs, 1H), 4.16 (m, 1H), 3.00 (m, 1H), 2.73 (m, 1H), 1.93 (s, 3H), 1.68 (s, 2H), 1.59-1.41 (m, 1H), 1.33-1.25 (m, 1H), 0.98 (m, 6H). MS (ESI): 194.2 [M+H]⁺

Representative Deprotection of Enantiomerically Pure 5. (+) and (−) BRD4780

To a stirred solution of PdCl₂ (250 mg, w/w) in ethyl acetate (2.5 mL) was added a solution of enantiopure 4-nitrobenzyl ((1R,2R,3R,4S)-3-isopropylbicyclo[2.2.1]heptan-2-yl)carbamate (250 mg, 0.752 mmol) in ethyl acetate (2.5 mL)) under nitrogen atmosphere. The reaction mass was stirred at room temperature for 2 h under hydrogen purging. The reaction mixture was filtered through celite bed and washed with ethyl acetate (2×15 mL). The filtrate was concentrated, dissolved in 2 mL of dichloromethane and cooled to 0° C. 4M hydrochloric acid in 1,4-dioxane (0.2 mL) was added drop wise and reaction mixture was stirred at room temperature for 15 min, concentrated and triturated with n-pentane to get (1R,2R,3R,4S)-3-isopropylbicyclo[2.2.1]heptan-2-amine hydrochloride (73 mg, 0.385 mmol, 51%). NMR and MS characterization were identical to authentic racemic BRD4780. Fraction 1: [a]²⁵_{D}+12.0° (c=0.1, MeOH); Fraction 2: [a]²⁵_{D}−13.0° (c=0.1, MeOH).

### BRD4780 (R) Mosher amide, Fr1. (R)-3,3,3-trifluoro-N-((1S,2S,3S,4R)-3-isopropylbicyclo[2.2.1]heptan-2-yl)-2-methoxy-2-phenylpropanamide

To a stirred solution of (1S,2S,3S,4R)-3-isopropylbicyclo[2.2.1]heptan-2-amine hydrochloride (10 mg, 0.053 mmol, 1.00 eq) and N,N-diethylethanamine (22 uL, 0.158 mmol, 3.00 eq) in dichloromethane (0.50 mL) was added (2S)-3,3,3-trifluoro-2-methoxy-2-phenyl-propanoyl chloride (15 mg, 0.0580 mmol, 1.10 eq). The reaction mixture was stirred at room temperature for 1 hour. Then, the reaction mixture was concentrated and purified with flash chromatography (silica gel, Hexane/EtOAC 0-30%) to afford the desired (R)-3,3,3-trifluoro-N-((1S,2S,3S,4R)-3-isopropylbicyclo[2.2.1]heptan-2-yl)-2-methoxy-2-phenylpropanamide (10 mg, 51% yield). ¹H NMR (400 MHz, Chloroform-d) δ 7.55-7.49 (m, 2H), 7.40-7.35 (m, 3H), 6.64 (d, J=8.0 Hz, 1H), 3.83 (m, 1H), 3.45 (q, J=1.6 Hz, 3H), 2.50 (m, 1H), 2.16 (d, 3.6 Hz, 1H), 1.61 (m, 1H), 1.46 (m, 3H), 1.37 (m, 1H), 1.24-1.19 (m, 2H), 0.87 (d, J=6.6 Hz, 3H), 0.75 (d, J=6.6 Hz, 3H), 0.52 (ddd, J=9.9, 5.2, 2.0 Hz, 1H). 13C NMR (101 MHz, Chloroform-d) δ 165.68, 132.90, 129.53, 128.53, 127.75, 125.35, 122.47 58.24, 56.79, 55.26, 41.10, 39.54, 35.81, 32.09, 30.74, 21.90, 21.31, 20.06.

### BRD4780-(S) Mosher amide, Fr1. (S)-3,3,3-trifluoro-N—((S,2S,3S,4R)-3-isopropylbicyclo[2.2.1]heptan-2-yl)-2-methoxy-2-phenylpropanamide

To a stirred solution of (1S,2S,3S,4R)-3-isopropylbicyclo[2.2.1]heptan-2-amine hydrochloride (10 mg, 0.0527 mmol, 1.00 eq) and N,N-diethylethanamine (22 uL, 0.158 mmol, 3.00 eq) in dichloromethane (0.50 mL) was added (2R)-3,3,3-trifluoro-2-methoxy-2-phenyl-propanoyl chloride (15 mg, 0.0580 mmol, 1.10 eq). The reaction mixture was stirred at room temperature for 1 hour. Then, the reaction mixture was concentrated and purified with flash chromatography (silica gel, Hexane/EtOAC 0-30%) to afford the desired (R)-3,3,3-trifluoro-N-((1S,2S,3S,4R)-3-isopropylbicyclo[2.2.1]heptan-2-yl)-2-methoxy-2-phenylpropanamide (8 mg, 31% yield). ¹H NMR (400 MHz, Chloroform-d) δ 7.55-7.49 (m, 2H), 7.43-7.35 (m, 3H), 6.63 (d, J=7.7 Hz, 1H), 3.83 (m, 1H), 3.44 (q, J=1.6 Hz, 3H), 2.50 (m, 1H), 2.16 (d, 3.6 Hz, 1H), 1.59 (m, 2H), 1.51-1.45 (m, 1H), 1.45-1.36 (m, 1H), 1.24-1.11 (m, 3H), 0.92 (d, J=6.5 Hz, 3H), 0.87 (d, J=6.6 Hz, 3H), 0.58 (ddd, J=10.0, 5.3, 2.0 Hz, 1H). ¹³C NMR (101 MHz, Chloroform-d) δ 165.90, 133.10, 129.55, 128.60, 127.81, 125.35, 122.47, 58.22, 56.93, 55.21, 40.71, 39.52, 35.75, 32.24, 30.78, 21.94, 21.23, 20.08.

### BRD4780-(R) Mosher amide, Fr2. (R)-3,3,3-trifluoro-N-((1R,2R,3R,4S)-3-isopropylbicyclo[2.2.1]heptan-2-yl)-2-methoxy-2-phenylpropanamide

To a stirred solution of (1R,2R,3R,4S)-3-isopropylbicyclo[2.2.1]heptan-2-amine hydrochloride (12 mg, 0.0527 mmol, 1.00 eq) and N,N-diethylethanamine (22 uL, 0.158 mmol, 3.00 eq) in dichloromethane (0.50 mL) was added (2S)-3,3,3-trifluoro-2-methoxy-2-phenyl-propanoyl chloride (15 mg, 0.0580 mmol, 1.10 eq). The reaction mixture was stirred at room temperature for 2 hours. Then, the reaction mixture was concentrated and purified with flash chromatography (silica gel, Hexane/EtOAC 0-30%) to afford the desired (R)-3,3,3-trifluoro-N-((1S,2S,3S,4R)-3-isopropylbicyclo[2.2.1]heptan-2-yl)-2-methoxy-2-phenylpropanamide (14 mg, 60% yield). ¹H NMR (400 MHz, Chloroform-d) δ 7.56-7.47 (m, 2H), 7.42-7.33 (m, 3H), 6.63 (d, J=7.9 Hz, 1H), 3.87-3.80 (m, 1H), 3.44 (d, J=1.6 Hz, 3H), 2.49 (m, 1H), 2.16 (d, J=4.2 Hz, 1H), 1.65-1.52 (m, 2H), 1.49 (m, 1H), 1.44-1.29 (m, 1H), 1.23-1.12 (m, 3H), 0.92 (d, J=6.5 Hz, 3H), 0.87 (d, J=6.6 Hz, 3H), 0.58 (ddd, J=10.0, 5.3, 1.9 Hz, 1H). ¹³C NMR (101 MHz, Chloroform-d) δ 165.89, 133.11, 129.53, 128.58, 127.82, 125.36, 122.48, 58.23, 56.93, 55.20, 40.72, 39.53, 35.75, 32.24, 30.78, 21.92, 21.23, 20.07.

### BRD4780-(S) Mosher amide, Fr2. (S)-3,3,3-trifluoro-N-((1R,2R,3R,4S)-3-isopropylbicyclo[2.2.1]heptan-2-yl)-2-methoxy-2-phenylpropanamide

To a stirred solution of (1R,2R,3R,4S)-3-isopropylbicyclo[2.2.1]heptan-2-amine hydrochloride (12 mg, 0.0527 mmol, 1.00 eq) and N,N-diethylethanamine (22 uL, 0.158 mmol, 3.00 eq) in dichloromethane (0.50 mL) was added (2R)-3,3,3-trifluoro-2-methoxy-2-phenyl-propanoyl chloride (15 mg, 0.0580 mmol, 1.10 eq). The reaction mixture was stirred at room temperature for 2 hours. Then, the reaction mixture was concentrated and purified with flash chromatography (silica gel, Hexane/EtOAC 0-30%) to afford the desired (R)-3,3,3-trifluoro-N-((1S,2S,3S,4R)-3-isopropylbicyclo[2.2.1]heptan-2-yl)-2-methoxy-2-phenylpropanamide (14 mg, 60% yield). ¹H NMR (400 MHz, Chloroform-d) δ 7.55-7.49 (m, 2H), 7.41-7.35 (m, 3H), 6.64 (d, J=8.2 Hz, 1H), 3.83 (m, 1H), 3.45 (q, J=1.6 Hz, 3H), 2.50 (m, 1H), 2.16 (d, J=3.8 Hz, 1H), 1.61 (m, 2H), 1.52-1.41 (m, 2H), 1.36 (m, 1H), 1.22 (m, 2H), 0.87 (d, J=6.5 Hz, 3H), 0.75 (d, J=6.6 Hz, 3H), 0.52 (ddd, J=9.9, 5.2, 2.0 Hz, 1H). ¹³C NMR (101 MHz, Chloroform-d) δ 165.68, 132.91, 129.52, 128.53, 127.75, 125.36, 122.48, 58.25, 56.80, 55.24, 41.10, 39.55, 35.81, 32.09, 30.74, 21.89, 21.31, 20.06.

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

We claim:

1. A compound having a structure represented by formula I: 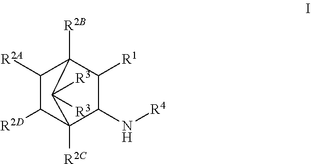
or a pharmaceutically acceptable salt thereof, wherein
R¹ is alkyl, cycloalkyl, aryl, or heteroaryl;
each of R^{2A}, R^{2B}, R^{2C}, and R^{2D} is independently selected from H or alkyl;
R³ are both H or both alkyl;
R⁴ is H or C(O)OR⁵ (i.e., to form a carbamate);
R⁵ is aralkyl or heteroaralkyl;
wherein
the compound is not selected from 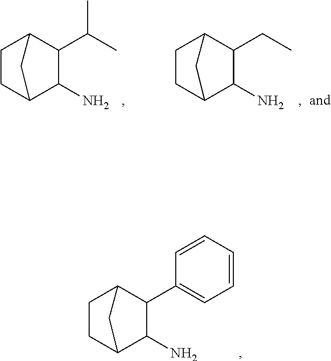
or a hydrochloride salt thereof.

2. The compound of claim 1, wherein the compound is not a salt of

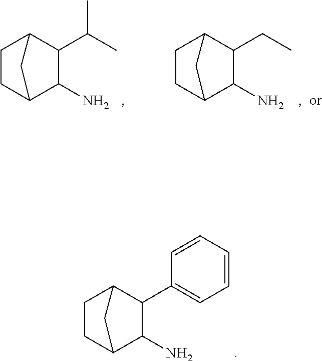

3. The compound of claim 1 or 2, wherein the compound has a structure represented by formula Ia, Ib, Ic, or Id: 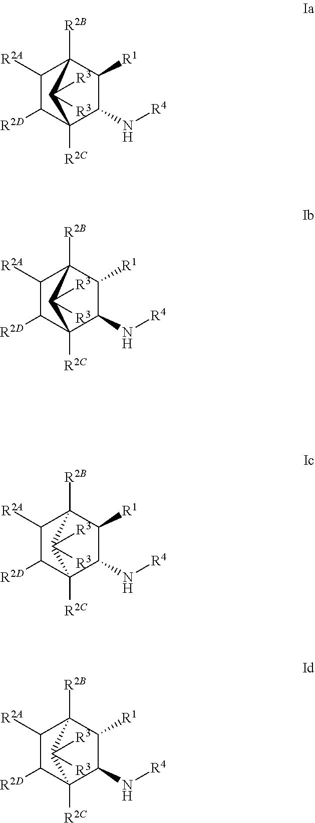
or a pharmaceutically acceptable salt thereof.

4. The compound of any one of claims 1-3, wherein the compound has an enantiomeric excess (ee) or diastereomeric excess (de) greater than 95%, 96%, 97%, 98%, or 99%.

5. The compound of any one of claims 1-4, wherein the compound is substantially free of one enantiomer and/or of one or more (preferably all) other diastereomers.

6. The compound of any one of claims 1-5, wherein the compound is a single enantiomer or a single diastereomer.

7. The compound of any one of claims 1-6, wherein R³ are both H.

8. The compound of any one of claims 1-6, wherein R⁴ is H.

9. The compound of any one of claims 1-6, wherein R¹ is alkyl.

10. The compound of claim 9, wherein R¹ is methyl, trifluoromethyl, ethyl, propyl, isopropyl, isobutyl, or tertiary-butyl.

11. The compound of any one of claims 1-8, wherein R¹ is cycloalkyl.

12. The compound of claim 11, wherein R¹ is cyclopropyl, cyclobutyl, cyclopentyl, or cyclohexyl.

13. The compound of any one of claims 1-8, wherein R¹ is aryl.

14. The compound of claim 13, wherein R¹ is phenyl.

15. The compound of any one of claims 1-8, wherein R¹ is heteroaryl.

16. The compound of claim 15, wherein R¹ is thiophenyl.

17. The compound of any one of claims 1-16, wherein R¹ is substituted with alkyl, alkenyl, alkynyl, halo, hydroxyl, carboxyl, acyl, acetyl, ester, thioester, alkoxy, phosphoryl, amino, amide, cyano, nitro, azido, alkylthio, alkenyl, alkynyl, cycloalkyl, alkylsulfonyl, or sulfonamide.

18. The compound of any one of claims 1-17, wherein R^{2A} is H.

19. The compound of any one of claims 1-18, wherein R^{2B} is H.

20. The compound of any one of claims 1-19, wherein R^{2C} is H.

21. The compound of any one of claims 1-20, wherein R^{2D} is H.

22. A compound selected from: 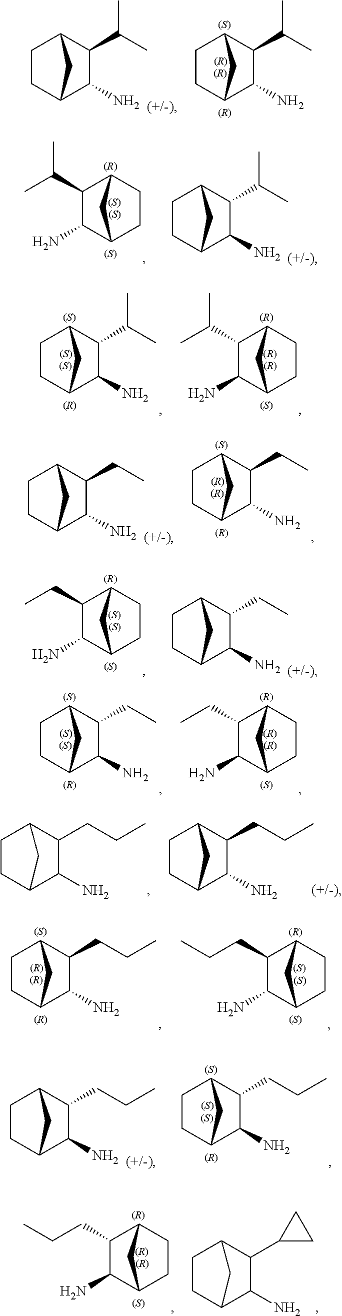 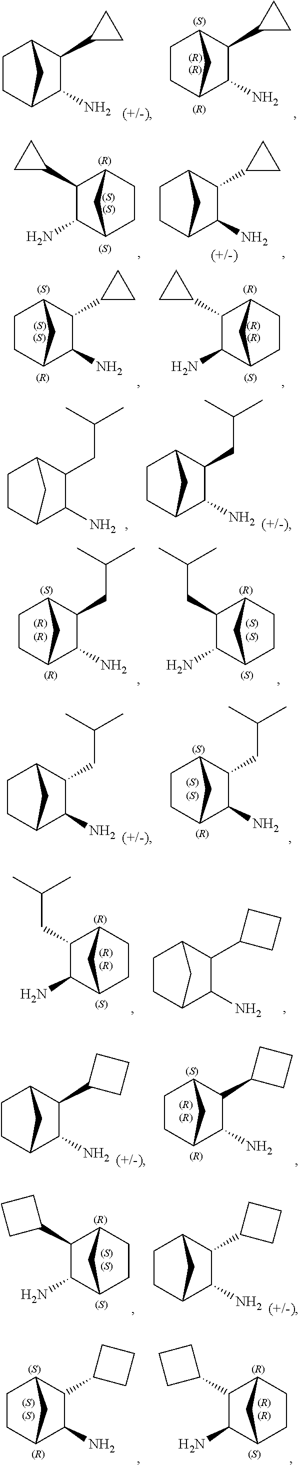 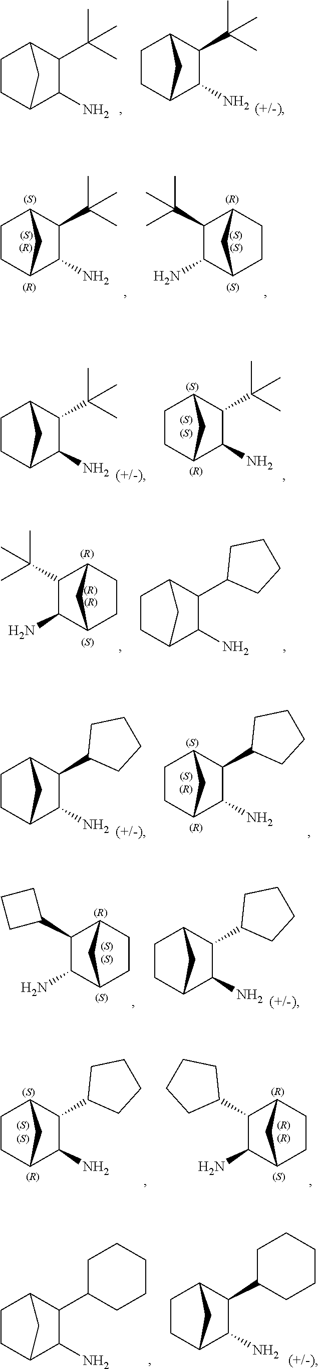 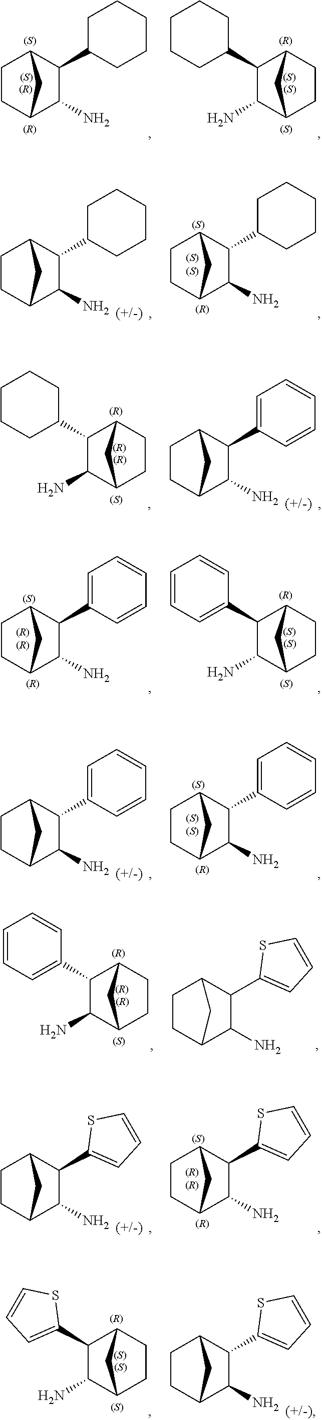 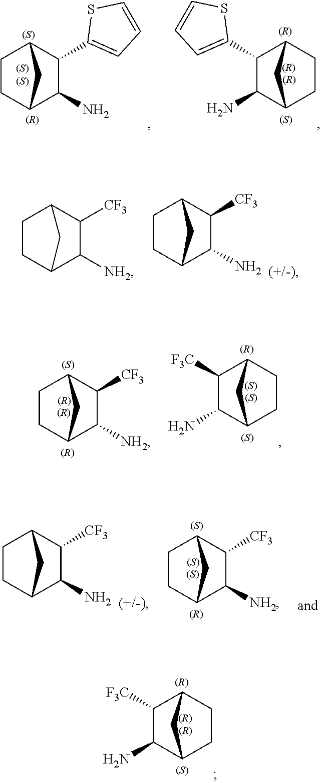
or a pharmaceutically acceptable salt thereof.

23. The compound any one of claims 1-22, wherein the salt is a hydrochloride, maleate, phosphate, fumarate, citrate, malate (e.g., L-malate), lactate, succinate, adipate, acetate, tosylate, mesylate, besylate, benzoate, hydrobromide, aspartate (e.g., L-aspartate), glutamate (e.g., L-glutamate), or tartrate (e.g., L-tartrate).

24. The compound of any one of claims 1-23, wherein the compound is a maleate, phosphate, fumarate, citrate, malate (e.g., L-malate), lactate, succinate, adipate, acetate, tosylate, mesylate, besylate, benzoate, hydrobromide, aspartate (e.g., L-aspartate), glutamate (e.g., L-glutamate), or tartrate (e.g., L-tartrate) salt.

25. The compound of any one of claims 1-24, wherein the compound is a maleate, fumarate, succinate, L-malate, or phosphate salt.

26. The compound of any one of claims 1-25, wherein the compound is a maleate salt.

27. A crystalline form of a compound having a structure represented by formula IIa:

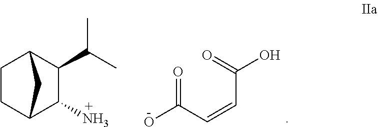

28. The crystalline form of claim 27, wherein the crystalline form has 20 values of about 23.2, about 18.3, about 18.6, about 14.0, about 13.7, about 24.4, about 20.7, or about 18.9.

29. The crystalline form of claim 27, wherein the crystalline form has 20 values of about 23.2, about 18.3, about 18.6, about 14.0, and about 13.7.

30. The crystalline form of claim 27, wherein the crystalline form has 20 values of about 23.2, about 18.3, about 18.6, about 14.0, about 13.7, about 24.4, and about 20.7.

31. The crystalline form of claim 27, wherein the crystalline form has 20 values of about 23.2, about 18.3, and about 18.6, about 14.0, about 13.7, about 24.4, about 20.7, and about 18.9.

32. The crystalline form of claim 27, wherein the crystalline form has 20 values substantially similar to those recited in Table 22.

33. The crystalline form of claim 27, wherein the crystalline form has an XRD pattern substantially similar to one of those depicted in FIG. 2A.

34. A crystalline form of a compound having a structure represented by formula IIb:

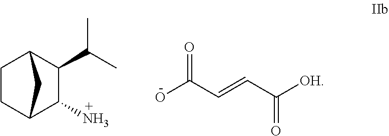

35. The crystalline form of claim 34, wherein the crystalline form has 20 values of about 22.1, about 19.5, about 23.5, about 27.4, about 33.4, about 11.0, about 21.6, or about 18.5.

36. The crystalline form of claim 34, wherein the crystalline form has 20 values of about 22.1, about 19.5, about 23.5, about 27.4 and about 33.4.

37. The crystalline form of claim 34, wherein the crystalline form has 20 values of about 22.1, about 19.5, about 23.5, about 27.4 about 33.4, about 11.0, and about 21.6.

38. The crystalline form of claim 34, wherein the crystalline form has 20 values of about 22.1, about 19.5, about 23.5, about 27.4, about 33.4, about 11.0, about 21.6, and about 18.5.

39. The crystalline form of claim 34, wherein the crystalline form has 20 values substantially similar to those recited in Table 26.

40. The crystalline form of claim 34, wherein the crystalline form has an XRD pattern substantially similar to that depicted in FIG. 2C.

41. A crystalline form of a compound having a structure represented by formula IIc:

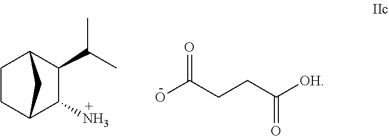

42. The crystalline form of claim 41, wherein the crystalline form has 20 values of about 19.5, about 21.8, about 14.4, about 10.9, about 19.2, about 14.8, about 27.3, or about 22.1.

43. The crystalline form of claim 41, wherein the crystalline form has 20 values of about 19.5, about 21.8, about 14.4, about 10.9, and about 19.2.

44. The crystalline form of claim 41, wherein the crystalline form has 20 values of about 19.5, about 21.8, about 14.4, about 10.9, about 19.2, about 14.8, or about 27.3.

45. The crystalline form of claim 41, wherein the crystalline form has 20 values of about 19.5, about 21.8, about 14.4, about 10.9, about 19.2, about 14.8, about 27.3, and about 22.1.

46. The crystalline form of claim 41, wherein the crystalline form has 20 values substantially similar to those recited in Table 31.

47. The crystalline form of claim 41, wherein the crystalline form has an XRD pattern substantially similar to that depicted in FIG. 2G.

48. A crystalline form of a compound having a structure represented by formula IId:

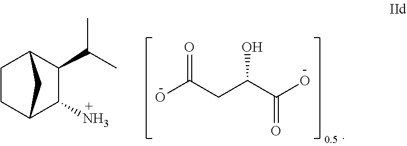

49. The crystalline form of claim 48, wherein the crystalline form has 20 values of about 7.7, about 23.1, about 16.1, about 14.4, about 21.8, about 24.7, about 17.5, or about 12.4.

50. The crystalline form of claim 48, wherein the crystalline form has 20 values of about 7.7, about 23.1, about 16.1, about 14.4, and about 21.8.

51. The crystalline form of claim 48, wherein the crystalline form has 20 values of about 7.7, about 23.1, about 16.1, about 14.4, about 21.8, about 24.7, and about 17.5.

52. The crystalline form of claim 48, wherein the crystalline form has 20 values of about 7.7, about 23.1, about 16.1, about 14.4, about 21.8, about 24.7, about 17.5, and about 12.4.

53. The crystalline form of claim 48, wherein the crystalline form has 20 values substantially similar to those recited in Table 29.

54. The crystalline form of claim 48, wherein the crystalline form has an XRD pattern substantially similar to that depicted in FIG. 2E.

55. A crystalline form of a compound having a structure represented by formula IIe:

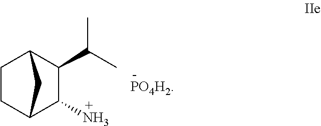

56. The crystalline form of claim 55, wherein the crystalline form has 20 values of about 6.9, about 20.8, about 17.1, about 17.4, about 23.9, about 19.6, about 13.6, or about 19.9.

57. The crystalline form of claim 55, wherein the crystalline form has 20 values of about 6.9, about 20.8, about 17.1, about 17.4, and about 23.9.

58. The crystalline form of claim 55, wherein the crystalline form has 20 values of about 6.9, about 20.8, about 17.1, about 17.4, and about 23.9, about 19.6, and about 13.6.

59. The crystalline form of claim 55, wherein the crystalline form has 20 values of about 6.9, about 20.8, about 17.1, about 17.4, about 23.9, about 19.6, about 13.6, and about 19.9.

60. The crystalline form of claim 55, wherein the crystalline form has 20 values substantially similar to those recited in Table 25.

61. The crystalline form of claim 55, wherein the crystalline form has an XRD pattern substantially similar to that depicted in FIG. 2B.

62. A pharmaceutical composition comprising a compound or crystalline form of any one of claims 1-61 wherein R⁴ is H and a pharmaceutically acceptable excipient.

63. A method of making a compound represented by formula V according to Scheme I: 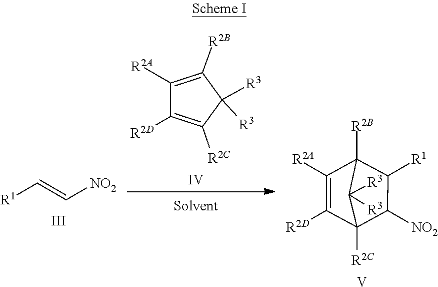
wherein
R¹ is alkyl, cycloalkyl, aryl, or heteroaryl;
each of R^{2A}, R^{2B}, R^{2C}, and R^{2D} is independently selected from H or alkyl;
R³ are both H or both alkyl; and
the solvent is an aromatic solvent, a heteroaromatic solvent, an organic acid, an alcohol, halogenated alcohol, an ether, a nitrile, a formamide, alkylamine, or a protic polar solvent.

64. The method of claim 63, wherein the method is represented by Scheme Ia:

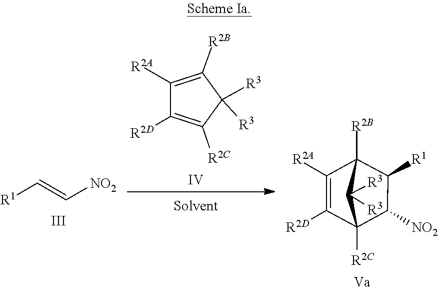

65. The method of claim 63, wherein the method is represented by Scheme Ib:

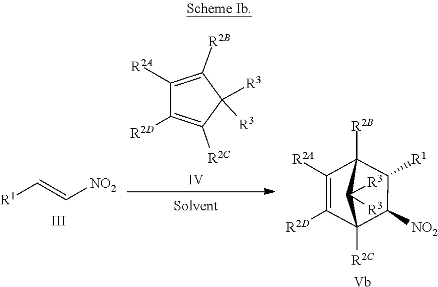

66. The method of claim 63, wherein the method is represented by Scheme Ic:

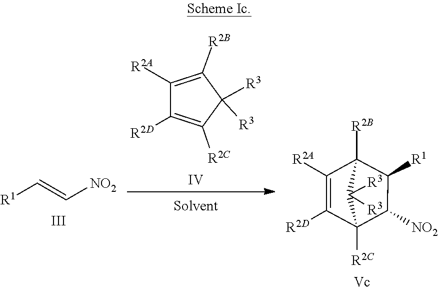

67. The method of claim 63, wherein the method is represented by Scheme Id:

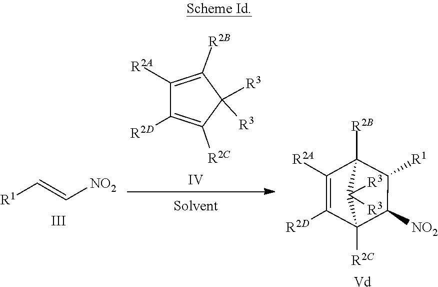

68. The method of any one of claims 63-67, wherein the solvent is an aromatic solvent.

69. The method of claim 68, wherein the aromatic solvent is toluene.

70. The method of any one of claims 63-67, wherein the solvent is a heteroaromatic solvent.

71. The method of claim 70, wherein the heteroaromatic solvent is pyridine.

72. The method of any one of claims 63-67, wherein the solvent is an organic acid.

73. The method of claim 72, wherein the organic acid is acetic acid, trifluoroacetic acid, butyric acid, tertiary butyric acid, toluenesulfonic acid, or benzoic acid.

74. The method of any one of claims 63-67, wherein the solvent is an alcohol.

75. The method of claim 74, wherein the alcohol is methanol, ethanol, or isopropanol.

76. The method of any one of claims 63-67, wherein the solvent is a halogenated alcohol.

77. The method of claim 76, wherein the halogenated alcohol is a fluorinated alcohol.

78. The method of claim 76 or 77, wherein the halogenated alcohol is difluoroethanol or hexafluoroisopropanol.

79. The method of any one of claims 63-67, wherein the solvent is an ether.

80. The method of claim 79, wherein the ether is tetrahydrofuran.

81. The method of any one of claims 63-67, wherein the solvent is a nitrile.

82. The method of claim 81, wherein the nitrile is acetonitrile.

83. The method of any one of claims 63-67, wherein the solvent is a formamide.

84. The method of claim 83, wherein the formamide is dimethyl formamide.

85. The method of any one of claims 63-67, wherein the solvent is an alkylamine.

86. The method of claim 85, wherein the alkylamine is diethylamine, trimethylamine, or 1,8-diazabicyclo(5.4.0)undec-7-ene (DBU).

87. The method of any one of claims 63-67, wherein the solvent is a protic polar solvent.

88. The method of claim 87, wherein the protic polar solvent is water.

89. The method of any one of claims 63-88, wherein the solvent further comprises an additive.

90. The method of claim 89, wherein the additive is an organic acid.

91. The method of claim 90, wherein the organic acid is acetic acid.

92. The method of any one of claims 63-91, wherein the method is performed in a temperature range from 20° C. to 100° C.

93. The method of any one of claims 63-92, wherein the method is performed at about 30° C., about 35° C., about 40° C., about 45° C., or about 50° C.

94. The method of any one of claims 63-93, wherein the method is performed at about 40° C.

95. The method of any one of claims 63-94, wherein the method is performed for 24 to 144 hours.

96. The method of any one of claims 63-95, wherein the method is performed for between 48 to 96 hours.

97. The method of any one of claims 63-96, wherein the method is performed for about 72 hours.

98. The method of any one of claims 63-97, wherein the method further comprises a step represented by Scheme II: 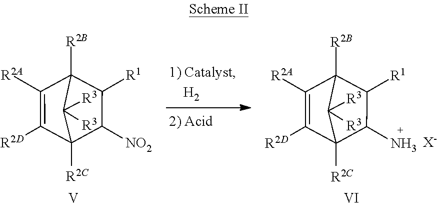
wherein
X⁻ is an organic or inorganic anion;
the catalyst is a noble metal oxide (e.g., platinum oxide), a nickel-aluminum alloy (e.g., Raney nickel), or a noble metal on carbon (e.g., palladium on carbon); and
the acid is a Brønsted acid.

99. The method of any one of claims 63-97, wherein the method further comprises a step represented by Scheme IIa: 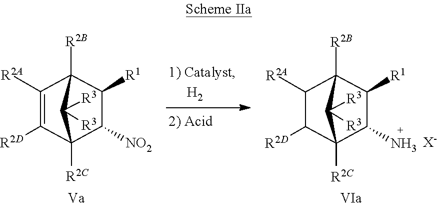
wherein
X⁻ is an organic or inorganic anion;
the catalyst is a noble metal oxide (e.g., platinum oxide), a nickel-aluminum alloy (e.g., Raney nickel), or a noble metal on carbon (e.g., palladium on carbon); and
the acid is a Brønsted acid.

100. The method of any one of claims 63-97, wherein the method further comprises a step represented by Scheme IIb: 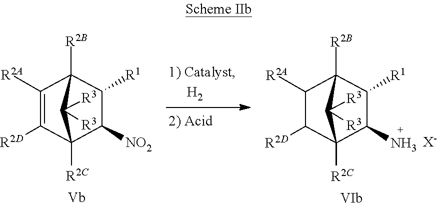
wherein
X⁻ is an organic or inorganic anion;
the catalyst is a noble metal oxide (e.g., platinum oxide), a nickel-aluminum alloy (e.g., Raney nickel), or a noble metal on carbon (e.g., palladium on carbon); and
the acid is a Brønsted acid.

101. The method of any one of claims 63-97, wherein the method further comprises a step represented by Scheme IIc: 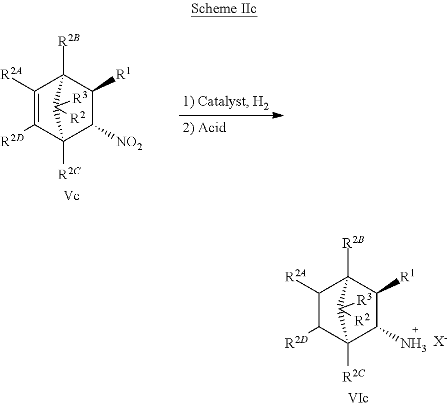
wherein
X⁻ is an organic or inorganic anion;
the catalyst is a noble metal oxide (e.g., platinum oxide), a nickel-aluminum alloy (e.g., Raney nickel), or a noble metal on carbon (e.g., palladium on carbon); and
the acid is a Brønsted acid.

102. The method of any one of claims 63-97, wherein the method further comprises a step represented by Scheme IId: 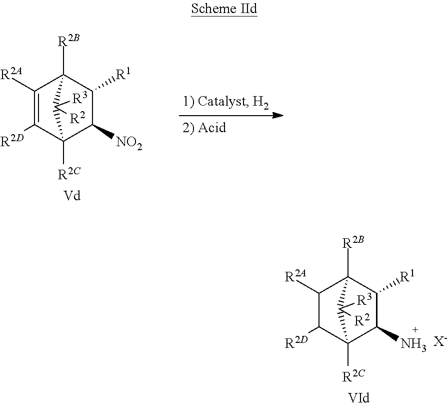
wherein
X⁻ is an organic or inorganic anion;
the catalyst is a noble metal oxide (e.g., platinum oxide), a nickel-aluminum alloy (e.g., Raney nickel), or a noble metal on carbon (e.g., palladium on carbon); and
the acid is a Brønsted acid.

103. The method of any one of claims 98-102, wherein the catalyst is palladium on carbon.

104. The method of any one of claims 98-102, wherein the acid is a hydrogen halide.

105. The method of any one of claims 98-104, wherein the acid is HCl (e.g., HCl in ethyl acetate or diethylether).

106. The method of any one of claims 100-102, wherein the acid is maleic acid.

107. A method of making a compound represented by formula VV according to Scheme III: 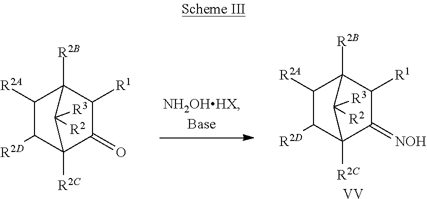
wherein
R¹ is alkyl, cycloalkyl, aryl, or heteroaryl;
each of R^{2A}, R^{2B}, R^{2C}, and R^{2D} is independently selected from H or alkyl;
R³ are both H or both alkyl; and
X is an inorganic anion (e.g., chloride); and
the base is an inorganic base (e.g., sodium acetate).

108. The method of claim 107, wherein the method is represented by Scheme IIIa:

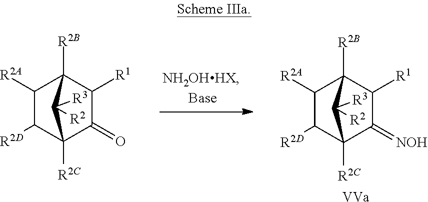

109. The method of claim 107, wherein the method is represented by Scheme IIIb:

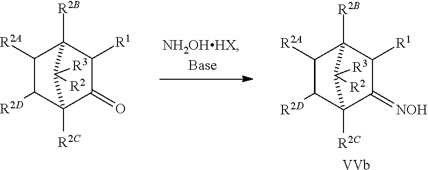

110. A method of making a compound represented by formula VI according to Scheme IV: 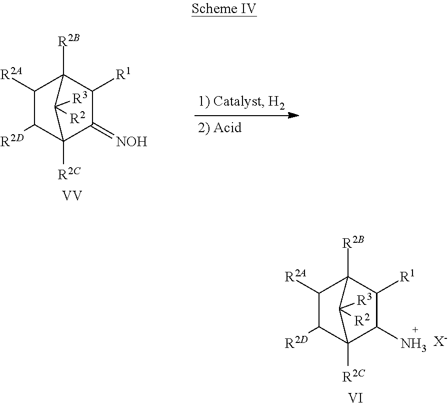
wherein
R¹ is alkyl, cycloalkyl, aryl, or heteroaryl;
each of R^{2A}, R^{2B}, R^{2C}, and R^{2D} is independently selected from H or alkyl;
R³ are both H or both alkyl; and
X⁻ is an organic or inorganic anion;
the catalyst is a noble metal oxide (e.g., platinum oxide); and
the acid is a Brønsted acid (e.g., acetic acid).

111. The method of claim 110, wherein the method is represented by Scheme IVa:

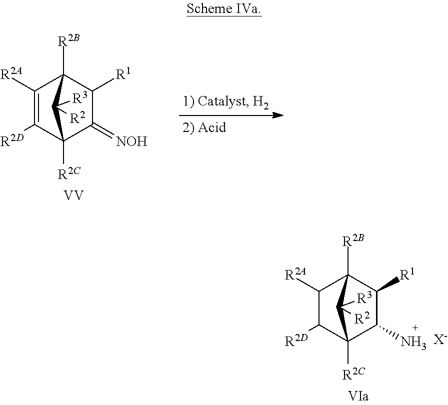

112. The method of claim 110, wherein the method is represented by Scheme IVb:

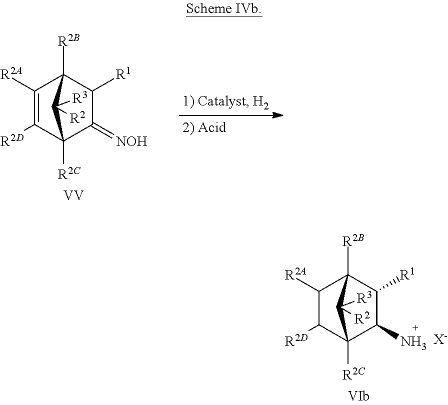

113. The method of claim 110, wherein the method is represented by Scheme IVc:

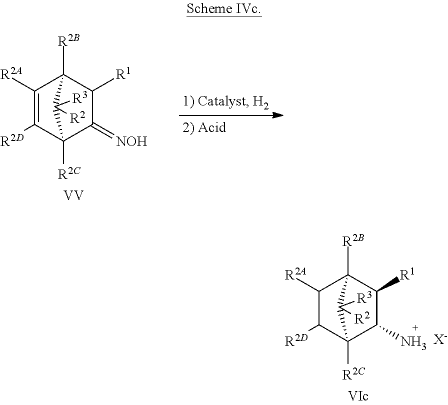

114. The method of claim 110, wherein the method is represented by Scheme IVd:

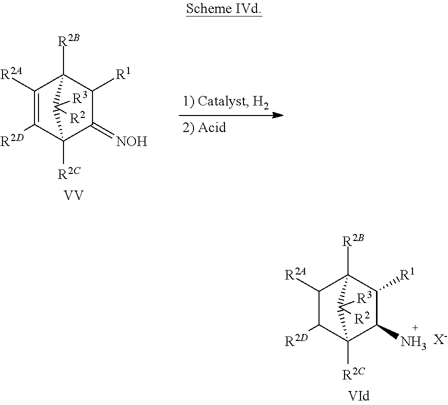

115. The method of any one of claims 110-114, wherein the catalyst is platinum oxide.

116. The method of any one of claims 63-115, wherein the compound has an enantiomeric excess (ee) greater than 95%, 96%, 97%, 98%, or 99%.

117. The method of any one of claims 63-116, wherein the compound is substantially free of one enantiomer and/or of one or more (preferably all) other diastereomers.

118. The method of any one of claims 63-117, wherein the compound is a single enantiomer or a single diastereomer.

119. The method of any one of claims 63-118, wherein R¹ is alkyl.

120. The method of claim 119, wherein R¹ is methyl, trifluoromethyl, ethyl, propyl, isopropyl, isobutyl, or tertiary-butyl.

121. The method of any one of claims 63-118, wherein R¹ is cycloalkyl.

122. The method of claim 121, wherein R¹ is cyclopropyl, cyclobutyl, cyclopentyl, or cyclohexyl.

123. The method of any one of claims 63-118, wherein R¹ is aryl.

124. The method of claim 123, wherein R¹ is phenyl.

125. The method of any one of claims 63-118, wherein R¹ is heteroaryl.

126. The method of claim 125, wherein R¹ is thiophenyl.

127. The method of any one of claims 63-126, wherein R¹ is substituted with alkyl, alkenyl, alkynyl, halo, hydroxyl, carboxyl, acyl, acetyl, ester, thioester, alkoxy, phosphoryl, amino, amide, cyano, nitro, azido, alkylthio, alkenyl, alkynyl, cycloalkyl, alkylsulfonyl, or sulfonamide.

128. The method of any one of claims 63-127, wherein R^{2A} is H.

129. The method of any one of claims 63-128, wherein R^{2B} is H.

130. The method of any one of claims 63-129, wherein R^{2C} is H.

131. The method of any one of claims 63-130, wherein R^{2D} is H.

132. The method of any one of claims 98-131, wherein the compound is represented by formula VI is a salt of a compound selected from 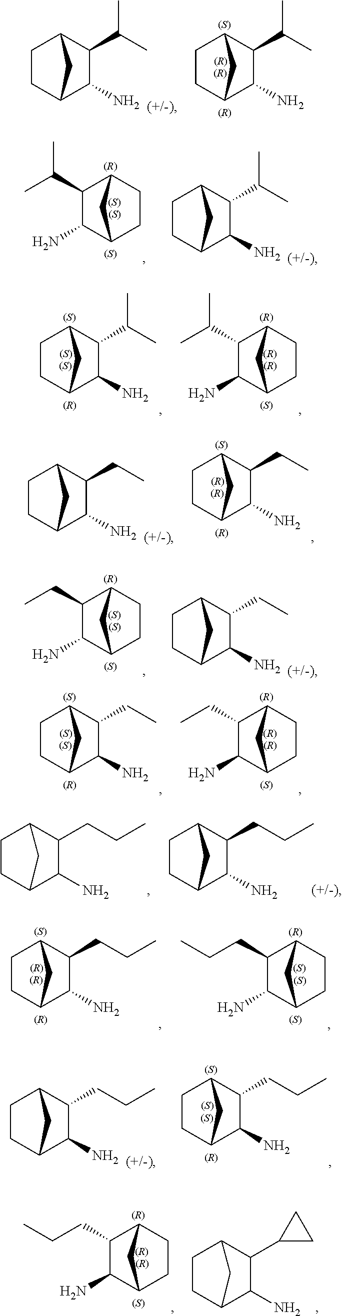 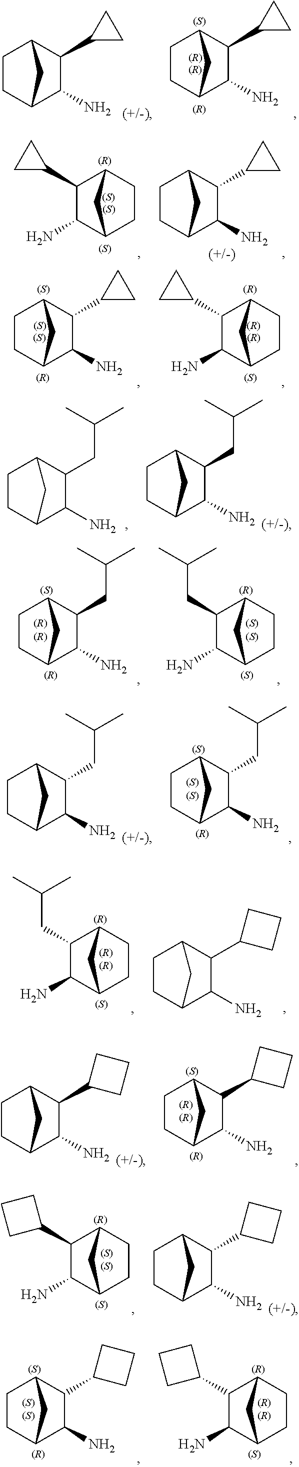 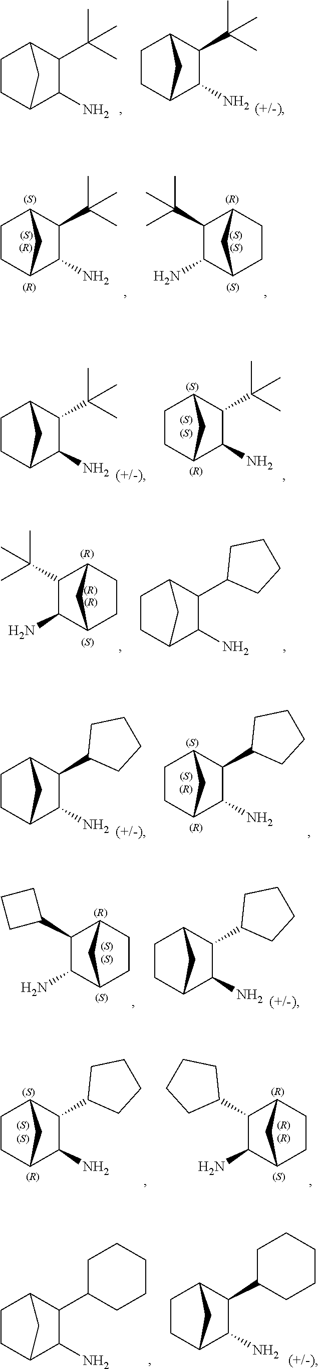 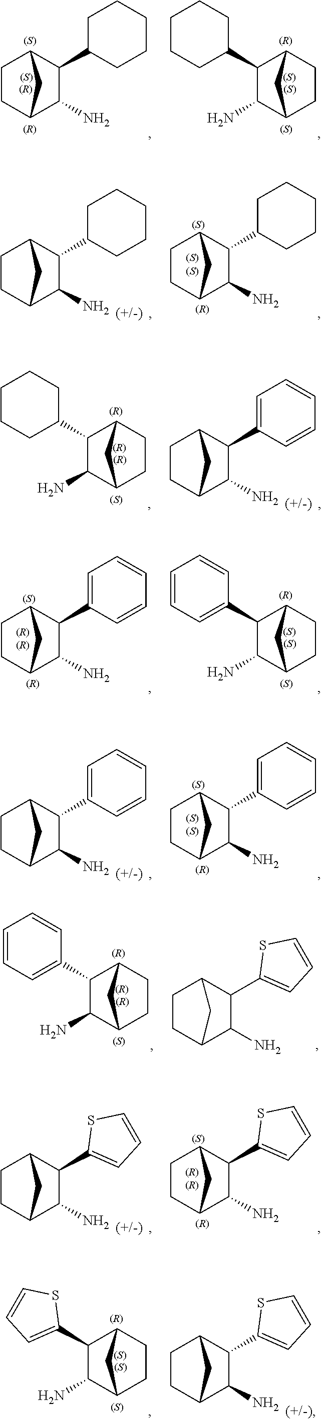 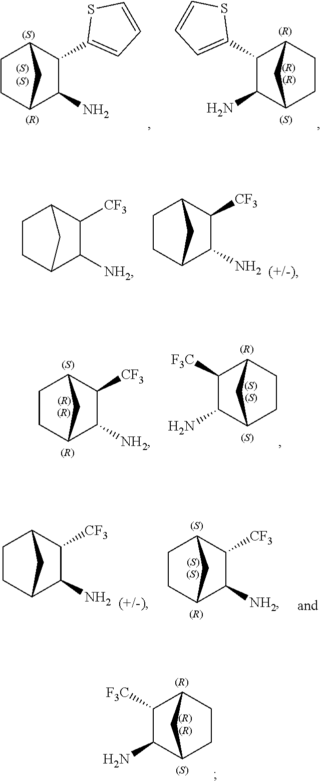
or a pharmaceutically acceptable salt thereof.

133. The method of any one of claims 98-132, wherein X⁻ is chloride, acetate, maleate, phosphate, fumarate, citrate, malate (e.g., L-malate), lactate, succinate, adipate, acetate, tosylate, mesylate, besylate, benzoate, hydrobromide, aspartate (e.g., L-aspartate), glutamate (e.g., L-glutamate), or tartrate (e.g., L-tartrate).

134. The method of any one of claims 63-133, wherein the method further comprises a step represented by Scheme V: 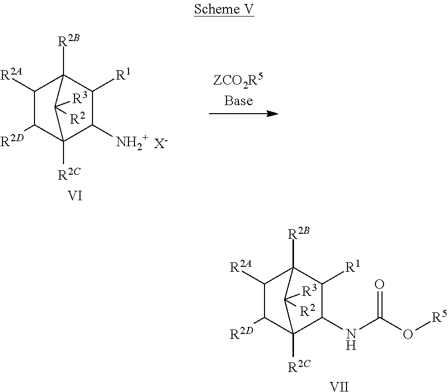
wherein
Z is halo;
R⁵ is aralkyl or heteroaralkyl;
Base is a carbonate base or a nitrogenous base.

135. The method of any one of claims 63-133, wherein the method further comprises a step represented by Scheme Va: 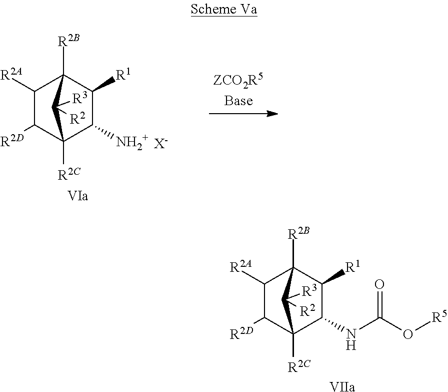
wherein
Z is halo;
R⁵ is aralkyl or heteroaralkyl;
Base is a carbonate base or a nitrogenous base.

136. The method of any one of claims 63-133, wherein the method further comprises a step represented by Scheme Vb: 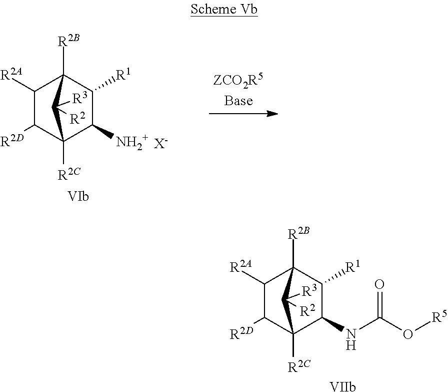
wherein
Z is halo;
R⁵ is aralkyl or heteroaralkyl;
Base is a carbonate base or a nitrogenous base.

137. The method of any one of claims 63-133, wherein the method further comprises a step represented by Scheme Vc: 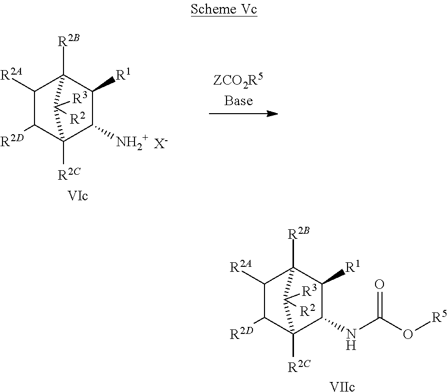
wherein
Z is halo;
R⁵ is aralkyl or heteroaralkyl;
Base is a carbonate base or a nitrogenous base.

138. The method of any one of claims 63-133, wherein the method further comprises a step represented by Scheme Vd: 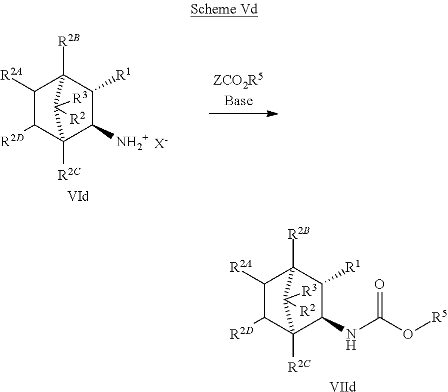
wherein
Z is halo;
R⁵ is aralkyl or heteroaralkyl;
Base is a carbonate base or a nitrogenous base.

139. The method of any one of claims 134-138, wherein Z is chloro.

140. The method of any one of claims 134-139, wherein R³ is aralkyl.

141. The method of any one of claims 134-140, wherein R³ is benzyl.

142. The method of any one of claims 134-141, wherein R³ is substituted with alkyl, alkenyl, alkynyl, halo, hydroxyl, carboxyl, acyl, acetyl, ester, thioester, alkoxy, phosphoryl, amino, amide, cyano, nitro, azido, alkylthio, alkenyl, alkynyl, cycloalkyl, alkylsulfonyl, or sulfonamide.

143. The method of any one of claims 134-142, wherein R³ is substituted with nitro.

144. The method of any one of claims 134-143, wherein R³ is substituted with nitro at the para-position.

145. The method of any one of claims 134-144, wherein the nitrogenous base is a secondary or tertiary alkylamine (e.g., trimethylamine or diisopropylamine).

146. The method of any one of claims 134-144, wherein the carbonate base is sodium carbonate, potassium carbonate, calcium carbonate, or cesium carbonate.

147. The method of any one of claims 134-146, wherein the carbonate base is sodium carbonate.

148. The method of any one of claims 134-146, wherein the method further comprises a solvent.

149. The method of claim 148, wherein the solvent is a mixture of water and a halogenated solvent.

150. The method of claim 149, wherein the halogenated solvent is dichloromethane.

151. A method of treating or preventing a toxic proteinopathy with a compound, crystalline form or pharmaceutical composition of any one of claims 1-62; wherein R⁴ is H.

152. A method for selecting a treatment for a toxic proteinopathy resulting from mutant protein accumulation in the early secretory pathway in a subject in need thereof, the method comprising:
(a) identifying a subject as having or being at risk of developing a toxic proteinopathy resulting from mutant protein accumulation in the early secretory pathway; and
(b) selecting a compound, crystalline form or pharmaceutical composition of any one of claims 1-62 wherein R⁴ is H as a treatment for the subject identified as having or being at risk of developing a toxic proteinopathy resulting from mutant protein accumulation in the early secretory pathway.

153. The method of claim 151 or 152, wherein the toxic proteinopathy is selected from a neurodegenerative disease, MUC1-associated kidney disease, autosomal dominant kidney disease caused by uromodulin mutations, and a form of retinitis pigmentosa (RP) caused by a rhodopsin mutation.

154. The method of claim 153, wherein the toxic proteinopathy is MUC1-associated kidney disease.

155. The method of any one of claims 152-154, wherein the subject has one or more of the following: end-stage renal disease, urinalysis revealing minimal protein and no blood, slowly progressive kidney failure, hyperglycemia and/or gout.

156. The method of any one of claims 152-155, wherein the subject has been identified to be in need of dialysis or kidney transplantation.

157. The method of claim 152 or 153, wherein the subject has one or more of the following symptoms of RP: night blindness; tunnel vision (due to loss of peripheral vision); latticework vision; photopsia (blinking/shimmering lights); photophobia (aversion to bright lights); development of bone spicules in the fundus; slow adjustment from dark to light environments and vice versa; blurring of vision; poor color separation; loss of central vision; and/or blindness.

158. The method of any one of claims 152, 155 or 156, wherein step (b) comprises identifying the presence in the subject of a mutation in MUC1, UMOD and/or rhodopsin, optionally wherein the MUC1 mutation is a MUC1 frameshift mutation, the UMOD mutation is a C126R UMOD mutation and/or the rhodopsin mutation is a P23H rhodopsin mutation.

159. The method of any one of claims 152-158, further comprising: (c) administering the selected compound, crystalline form or pharmaceutical composition of any one of claims 1-62 wherein R⁴ is H to the subject.

160. The method of any one of claims 152-159, wherein the subject is human.

161. A method for treating or preventing a proteinopathy resulting from mutant protein accumulation in the early secretory pathway in a subject, the method comprising:
identifying a subject as having or at risk of developing a proteinopathy resulting from mutant protein accumulation in the early secretory pathway in a subject; and
administering a compound, crystalline form or pharmaceutical composition of any one of claims 1-62 wherein R⁴ is H to the subject in an amount sufficient to cause reduction or improvement of a symptom of the proteinopathy resulting from mutant protein accumulation in the early secretory pathway in the subject,
thereby treating or preventing the proteinopathy resulting from mutant protein accumulation in the early secretory pathway in the subject.

162. The method of claim 161, wherein said compound causes release of MUC1, UMOD and/or rhodopsin from the early secretory compartment, optionally wherein said compound causes release of MUC1, UMOD and/or rhodopsin from the endoplasmic reticulum (ER), from COPI-coated vesicles, from COPII-coated vesicles and/or from the Golgi apparatus.

163. The method of claim 161 or 162, wherein the proteinopathy is selected from a neurodegenerative disease, MUC1-associated kidney disease, autosomal dominant kidney disease caused by uromodulin mutations, a form of retinitis pigmentosa (RP) caused by a rhodopsin mutation, pulmonary alveolar proteinosis or ApoL1-positive kidney disease, and type II diabetes, optionally wherein the neurodegenerative disease is selected from Alzheimer's disease (AD) and other dementias; Parkinson's disease (PD) and PD-related disorders; lysozyme amyloidosis; dialysis amyloidosis; cystic fibrosis; cataracts; odontogenic tumor amyloid; familial British dementia; hereditary cerebral hemorrhage with amyloidosis (Icelandic); familial amyloidotic neuropathy or senile systemic/cardiomyopathy; ApoAII amyloidosis; familial amyloidosis of the Finnish type (FAF); fibrinogen amyloidosis; inclusion body myositis/myopathy; hereditary lattice corneal dystrophy; prion disease (including, e.g., Creutzfeldt-Jakob Disease, variant Creutzfeldt-Jakob Disease, Bovine Spongiform Encephalopathy (BSE), Kuru, Gerstmann-Straussler-Scheinker disease, fatal familial insomnia (FFI), scrapie, and other animal TSEs); motor neuron diseases (MND; including, e.g., Amyotrophic Lateral Sclerosis (ALS), Primary Lateral Sclerosis (PLS), Progressive Bulbar Palsy (PBP), Pseudobulbar Palsy, Progressive Muscular Δtrophy, Spinal Muscular Δtrophy (Type 1, Type 2, Type 3, Type 4), and Kennedy's Disease); and spinocerebellar ataxia (SCA).

164. The method of claim 163, wherein the proteinopathy is MUC1-associated kidney disease.

165. The method of any one of claims 161-164, wherein the symptom of the proteinopathy is selected from end-stage renal disease, urinalysis revealing minimal protein and no blood, slowly progressive kidney failure, hyperglycemia, gout, a need for dialysis or kidney transplantation, night blindness; tunnel vision (optionally due to loss of peripheral vision); latticework vision; photopsia (blinking/shimmering lights); photophobia (aversion to bright lights); development of bone spicules in the fundus; slow adjustment from dark to light environments and vice versa; blurring of vision; poor color separation; loss of central vision; and/or blindness.

166. The method of any one of claims 161-165, wherein the subject has a mutation in MUC1, UMOD and/or rhodopsin, optionally wherein the MUC1 mutation is a MUC1 frameshift mutation, the UMOD mutation is a C₁₂₆R UMOD mutation and/or the rhodopsin mutation is a P23H rhodopsin mutation.

167. The method of any one of claims 161-166, wherein the pharmaceutical composition comprising a compound of claim 66 wherein R⁴ is H is administered to the subject via the oral route (P.O.).

168. The method of any one of claims 161-166, wherein the compound or crystalline form of any one of claims 1-65 wherein R⁴ is H comprises a pharmaceutically-acceptable carrier/excipient.

169. A method for reducing or eliminating accumulation of a mutant protein in the ER lumen of a cell, in COPI and/or COPII vesicles of a cell, in the cis-Golgi lumen of a cell, in the medial cisternae of the Golgi of a cell, and/or in the trans-Golgi network (TGN) of a cell, the method comprising administering a compound, crystalline form or pharmaceutical composition of any one of claims 1-66 wherein R⁴ is H to the environment of a cell in an amount sufficient to reduce or eliminate accumulation of the mutant protein in the ER lumen of the cell, in COPI and/or COPII vesicles of the cell, in the cis-Golgi lumen of the cell, in the medial cisternae of the Golgi of the cell, and/or in the trans-Golgi network (TGN) of the cell, thereby reducing or eliminating accumulation of the mutant protein in the ER lumen of the cell, in COPI and/or COPII vesicles of the cell, in the cis-Golgi lumen of the cell, in the medial cisternae of the Golgi of the cell, and/or in the trans-Golgi network (TGN) of the cell.

170. The method of claim 169, wherein the mutant protein is selected from a MUC1 frameshift mutant protein, a UMOD pathogenic variant and a rhodopsin mutant, optionally wherein the MUC1 mutation is a MUC1 frameshift mutation, the UMOD mutation is a C126R UMOD mutation and/or the rhodopsin mutation is a P23H rhodopsin mutation.

171. A pharmaceutical composition for treating a subject having or at risk of developing a proteinopathy comprising a therapeutically effective amount of a compound or crystalline form of any one of claims 1-61 wherein R⁴ is H and a pharmaceutically acceptable carrier.

172. The pharmaceutical composition of claim 171, wherein the proteinopathy is selected from a neurodegenerative disease, MUC1-associated kidney disease, autosomal dominant kidney disease caused by uromodulin mutations and a form of retinitis pigmentosa (RP) caused by a rhodopsin mutation.

173. The pharmaceutical composition of claim 171 or claim 172, wherein the subject is human.

174. A method of treating or preventing MUC1-associated kidney disease (MKD) in a subject in need thereof, the method comprising administering to the subject a compound, crystalline form or pharmaceutical composition of any one of claims 1-62 wherein R⁴ is H as a first agent and a second agent selected from vitamin D, a phosphate binder, a blood pressure medication and a diuretic, thereby treating or preventing MKD in the subject.
